(19) United States
(12) Patent Application Publication
(10) Pub. No.: US 2011/0263526 A1
(43) Pub. Date: Oct. 27, 2011

(54) Nitric Oxide Releasing Prodrugs of Therapeutic Agents

(75) Inventor: Apparao SATYAM, Mumbai (IN)

(73) Assignee: PIRAMAL LIFE SCIENCES LIMITED, Mumbai (IN)

(21) Appl. No.: 13/092,245

(22) Filed: Apr. 22, 2011

Related U.S. Application Data
(60) Provisional application No. 61/327,175, filed on Apr. 23, 2010.

(51) Int. Cl. A61K 31/7072; A61K 31/58; A61K 31/55; A61K 31/495; A61K 31/4439; A61K 31/455; A61K 31/403; A61K 31/404; A61K 31/40; A61K 31/265; C07H 19/06; C07J 71/00; C07D 223/26; C07D 295/14; C07D 401/12; C07D 213/80; C07D 209/52; C07D 209/26; C07D 207/34; C07D 493/04; C07C 323/56; A61P 25/18; C07C 69/96; C07C 319/22; C07C 68/02; A61P 29/00; A61P 9/00; A61P 37/08; A61P 35/00; A61P 25/24; A61P 25/08; A61P 31/04; A61P 31/12; A61P 31/10; A61P 33/06; A61P 3/10; A61P 1/04; A61P 39/06; A61P 3/02; A61P 9/06; A61P 9/02; A61P 9/08; A61P 11/08; A61K 31/621

(52) U.S. Cl. 514/50; 514/166; 514/172; 514/217; 514/255.04; 514/338; 514/356; 514/412; 514/420; 514/423; 514/510; 536/28.53; 540/67; 540/591; 544/396; 546/273.7; 546/318; 548/452; 548/500; 548/537; 549/464; 558/275

(57) ABSTRACT

The present invention relates to nitric oxide releasing prodrugs of known drugs or therapeutic agents which are represented herein as compounds of formula (I) wherein the drugs or therapeutic agents contain one or more functional groups independently selected from a carboxylic acid, an amino, a hydroxyl and a sulfhydryl group. The invention also relates to processes for the preparation of the nitric oxide releasing prodrugs (the compounds of formula (I)), to pharmaceutical compositions containing them and to methods of using the prodrugs.

### INCORPORATION BY REFERENCE

This application is a non-provisional application and claims benefit under 35 U.S.C. 119(e) of Ser. No. 61/327,175, filed on 23 Apr. 2010.

Any foregoing applications and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Citation or identification of any document in this application is not an admission that such document is available as prior art to the present invention.

### FIELD OF THE INVENTION

The present invention relates to nitric oxide releasing prodrugs of known drugs or therapeutic agents which are represented herein as compounds of formula (I) wherein the drugs or therapeutic agents contain one or more functional groups independently selected from the group consisting of a carboxylic acid, an amino, a hydroxyl or a sulfhydryl group. The invention also relates to processes for the preparation of the nitric oxide releasing prodrugs (the compounds of formula (I)), to pharmaceutical compositions containing them and to methods of using the prodrugs. The present invention also relates to a bio-cleavable linker of formula (IA) capable of forming a covalent linkage with a drug or a therapeutic agent (designated herein as D) containing one or more functional groups independently selected from a carboxylic acid, an amino, a hydroxyl or a sulfhydryl group and also processes for their synthesis.

### BACKGROUND OF THE INVENTION

Many drugs (therapeutic agents) have undesirable properties, for instance, low oral drug absorption, toxicity, poor patient compliance etc., that may become pharmacological, pharmaceutical, or pharmacokinetic barriers in clinical drug application. Among the various approaches to minimize the undesirable drug properties, while retaining the desirable therapeutic activity, the chemical approach using drug derivatisation offers perhaps the highest flexibility and has been demonstrated as an important means of improving drug efficacy (Hyo-Kyung Han and Gordon L. Amidon AAPS PharmSci. 2000; 2 (1), 48-58.).

The conventional approach that is adapted to minimize the toxic side effects associated with the therapeutic agents has been to derivatise one or more functional groups present in the therapeutic agent or the drug molecule. The derivatives are then assessed for their therapeutic efficacy as well as toxicity. The carboxylic acid group is often present as an active functional group for derivatisation in several therapeutic agents. Non-steroidal anti-inflammatory drugs (NSAIDs) represent the best characterized class of drugs for therapeutic agents containing a carboxylic acid group as an active functional group. NSAIDs are also the most commonly used drugs to relieve pain, symptoms of arthritis and soft tissue inflammation. Most patients with rheumatoid arthritis receive NSAIDs as first-line treatment which is continued for prolonged periods. Although, NSAIDs provide anti-inflammatory and analgesic effects, they also have adverse effects on the upper gastrointestinal (GI) tract. The occurrence of GI toxicity appears to be strictly correlated to the mechanism of action of these drugs, namely the inhibition of the enzyme cyclooxygenase. In fact, inhibition of platelet cyclooxygenase, which causes prolonged bleeding time, and inhibition of cyclooxygenase in gastrointestinal mucosa, which results in a decreased synthesis of cytoprotective gastric prostaglandins, represent the major cause of serious gastrointestinal toxicity (Symposium on “New Anti-inflammatory agents: NO-NSAIDs and COX-2 inhibitors” part of the 11^{th} international conference on “Advances in prostaglandin and leukotrine research: Basic science and new clinical applications” held in Florence (Italy), Jun. 4-8, 2000). This problem has been solved by derivatisation of carboxylic acid group of NSAIDs into its ester and amide derivatives.

Another common approach to minimize adverse effects of the known drugs or therapeutic agents consists of attaching a carrier group to the therapeutic agents to alter their physicochemical properties and then subsequent enzymatic or non-enzymatic mechanism to release the active drug molecule (therapeutic agent). The therapeutic agent is linked through a covalent linkage with specialized non-toxic protective groups or carriers or promoieties in a transient manner to alter or eliminate undesirable properties associated with the parent drug to produce a carrier-linked prodrug.

Indeed, a more recent strategy for devising a gastric-sparing NSAID involves chemically coupling a nitric oxide (NO) releasing moiety to the parent NSAID. Nitric oxide is one of the most important mediators of mucosal defense, influencing such factors as mucus secretion, mucosal blood flow, ulcer repair and the activity of a variety of mucosal immunocytes (Med Inflammation, 1995; 4: 397-405). Compounds that release nitric oxide in small amounts over a prolonged period of time may also be very useful for the prevention of gastrointestinal injury associated with shock and with the use of drugs that have ulcerogenic effects (Muscara M. N.; Wallace J. L. American Journal of Physiology, Gastrointestinal and liver physiology, 1999; 39:G1313-1316). Nitric oxide has been reported to play a critical role in maintaining the integrity of the gastroduodenal mucosa and exerts many of the same effects as endogenous prostaglandins (Drugs Fut 2001; 26(5): 485).

Several mechanisms are considered to understand the protective effect of nitric oxide in the stomach including vasodilation of local mucosal blood vessels, inhibition of leukocyte adhesion and inhibition of caspase enzyme activity. The inactivation of caspase(s) appears to be an important factor in the GI tolerance of nitric oxide releasing NSAIDs (NO-NSAIDs). Caspases are a family of cysteine proteases that resemble interleukin-113 (IL-113) converting enzyme (ICE). These enzymes fall into two broad groups, i.e. caspase-1-like (including caspase-1, -4 and -5) and caspase-3-like enzymes. Caspase-1 is primarily involved in cytokine release, cleaving pro-IL-1β to produce IL-1β. The ability of a range of NO-NSAIDs to inhibit cytokine formation and caspase-1 (ICE) activity, thereby reducing the formation of pro-inflammatory IL-1β provides a possible explanation for the reduced gastric damaging effect of these compounds (J. E. Keeble and P. K. Moore, British Journal of Pharmacology, 2002; 137: 295-310).

In recent years, several NO-releasing non-steroidal anti-inflammatory drugs (NO-NSAIDs) have been synthesized by an ester linkage formed through coupling of a NO-releasing chemical spacer group to the carboxylic acid moiety of a conventional NSAID. The use of various aliphatic, aromatic or heterocyclic chemical spacers makes it possible to alter various physicochemical properties and kinetics of nitric oxide release [Berguad et al., Ann., N.Y. Acad. Sci. 1962: 360-371 (2002)]. The first NO-aspirin drug NCX 4016, which was synthesized relatively recently, consists of an aspirin molecule linked by an ester bond to a molecular spacer, which in turn, is linked to a nitro-oxy ester group (Dig Liver Dis 2003; 35 (suppl 2): 9-19). A number of NO-NSAID hybrid compounds, namely NO-naproxen (HCT 3012), NO-flurbiprofen (HCT 1026), NO-ibuprofen, NO-diclofenac and NO-indomethacin have been disclosed in the patent numbers EP 722434B1, U.S. Pat. No. 6,613,784B1 and U.S. Pat. No. 7,220,749B2 respectively. European Patent EP 722434B1 discloses nitric esters of the derivatives of propionic acid, 1-(p-chlorobenzoyl)-5-methoxy-2-methyl-3-indolylacetic acid and S-benzoyl-1,2-dihydro-3H-pyrrolo[1,2-a]pyrrole-1-carboxylic acid having anti-inflammatory and/or analgesic activity. U.S. Pat. No. 6,613,784B1 discloses nitro derivatives of NSAIDs, for instance, flurbiprofen, indomethacin, aspirin, naproxen and diclofenac. U.S. Pat. No. 7,220,749B2 discloses novel nitrosated and/or nitrosylated derivatives of COX-2 selective inhibitors. U.S. Patent Application Publication no. 20080293781A1 describes O-acyl salicylic acid derivatives bearing a NO donor moiety.

Further, NO releasing COX-2 (cyclooxygenase-2) inhibitors comprising NO-releasing moieties attached through a chemical linker to the COX-2 inhibitor compounds have been reported in the art. U.S. Pat. No. 7,199,154 B2 discloses nitrosated or nitrosylated prodrugs for COX-2 selective inhibitors that are useful for treating COX-2 mediated diseases or conditions and which can be administered alone or in combination with low-dose aspirin. The compounds are effective in treating chronic COX-2 mediated diseases or conditions, reducing the risk of thrombotic cardiovascular events and possibly renal side effects and at the same time reduce the risk of GI ulceration and bleeding. US Patent Application Publication no. 20060058363 A1 discloses nitric-oxide releasing prodrugs of celebrex and valdecoxib which are useful in the treatment of COX-2 mediated diseases. The compounds may be used as a combination therapy with low-dose aspirin to treat COX-2 mediated diseases or conditions while simultaneously reducing the risk of thrombotic cardiovascular events.

Nitric oxide (NO) also plays an important role in numerous physiological and patho-physiological conditions, e.g. blood pressure regulation, inflammation, infection, and the onset and progression of malignant diseases (Lirk, P., Hoffmann, G., and Rieder, J. Curr. Drug Targets Inflamm. Allergy 2002; 1: 89-108). NO deficiency is recognized to be a crucial factor in the initiation and progression of many cardiovascular diseases and delivery of supplementary NO in the form of NO-donor drugs has long been an attractive therapeutic strategy (Ian L Megson, David J Webb, Expert Opin. Investig. Drugs, 2002; 11(5): 587-601). In recent years, with the advent of NO-NSAID approach and because of the beneficial biochemical and pharmacological properties of nitric oxide, the strategy of linking NO-releasing moieties has been extended to a wide array of therapeutic agents selected from cardiovascular drugs, for instance, Angiotensin converting enzyme (ACE) inhibitors, calcium antagonists and beta-blockers, antitumor agents, antihistamines, glucocorticoids, etc. The aim of this strategy is to synthesize prodrugs that retain the pharmacological activity of the parent drug molecule coupled with the benefits of the biological actions of NO in reducing the adverse effects of the parent drug molecule.

Another class of therapeutic agents which are well-known for their anti-inflammatory and immunosuppressive effects are glucocorticoids. Due to their beneficial therapeutic effects, glucocorticoids are useful for the treatment of a variety of inflammation related disorders and immune system disorders, especially autoimmune diseases such as rheumatoid arthritis. However, their therapeutic application is limited due to adverse effects and toxicity associated with their use. The adverse effects caused by glucocorticoids include hypertension, peptic ulcers, gastrointestinal bleeding, increased risk for infections, osteoporosis and hyperglycemia (Schacke H et al., Pharmacol Ther 2002; 96:23-43).

U.S. Pat. Nos. 6,610,676 and 7,524,836B2 disclose nitrate esters and nitroxy derivatives of steroidal compounds having anti-inflammatory, immunodepressive and angiostatic activity or gastrointestinal activity. The compounds are useful in the treatment of morbid conditions wherein the steroids are generally used and confer greater benefit in terms of better tolerability and efficacy. PCT Application Publication WO2007099548A1 discloses 11β-hydroxyandrosta-4-3-one compounds which possess useful anti-inflammatory activity whilst having insignificant or no noteworthy side-effects at efficacious doses. PCT Application Publication WO2008095809A1 discloses derivatives of known corticosteroids, containing a NO-releasing moiety which are useful in the treatment of illnesses wherein the known corticosteroid, parent or precursor steroid, is generally applied, with increased benefit in terms of pharmacological profile and fewer or milder side effects than those of the parent corticosteroids. The compounds are useful in the treatment of inflammatory diseases, respiratory diseases, and autoimmune disorders among other disorders.

The approach and possibility of combining a few classes of drugs bearing different functional groups susceptible to derivatisation with NO-donating moieties has been described by Manlio Bolla et al., in Curr. Topic. Med. Chem. 2005; 5: 707-720. The review paper discloses four chemically different NO-donating linkers hybridized with different drugs possessing a derivatisable function. Free carboxylic acids, alcohols (including phenols), thiols, and amines have been demonstrated to be exploitable for such an approach.

The NO-releasing derivatives and prodrugs of various therapeutic agents known in the art are in different phases of clinical development and there are reports suggesting that a few of them have been suspended because of toxicity problems. Therefore, there is a clear need for new, alternative and better NO-releasing nitrate ester prodrug compounds which can exhibit improved therapeutic properties. A thorough investigation by the present inventor led to the discovery of nitric oxide releasing prodrugs or prodrug compounds which can be obtained through derivatisation of a known drug or a therapeutic agent containing one or more functional groups independently selected from carboxylic acid, hydroxyl, amino or sulfhydryl functional groups. The nitric oxide releasing prodrugs of the present invention are useful in the treatment of diseases or disorders that is characteristic of the parent drug molecule from which the prodrug is derived. The nitric oxide releasing prodrugs of the invention exhibit comparable or superior therapeutic effects compared to the parent drug molecule. The nitric oxide releasing prodrugs of known drugs or therapeutic agents as described in the present invention are expected to be safe to administer and have comparable or superior oral bioavailability compared to the parent drug molecules from which the prodrugs are derived. Further, owing to the strategy that is used to devise the nitric oxide releasing prodrugs of the present invention, the prodrugs or at least certain prodrugs encompassed in the present invention are expected to be devoid of genotoxicity at a concentration at which the compounds are expected to be used for the treatment of the medical conditions or diseases for the treatment of which the parent drug molecule is used.

Moreover, the nitric oxide releasing prodrugs of the invention are expected to overcome adverse effects, for instance, gastrointestinal (GI) toxicity and cardiovascular risks associated with the parent drug molecule.

### SUMMARY OF THE INVENTION

In one aspect, the present invention provides compounds of the following formula (I), which are prodrugs of known drugs or therapeutic agents;

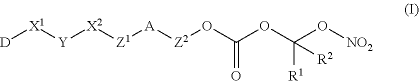

wherein:
D is a drug containing one or more functional groups independently selected from a carboxylic acid, an amino, a hydroxyl or a sulfhydryl group capable of forming a covalent bio-cleavable linkage with a biocleavable linker;
X¹ is a bond, oxygen, sulphur or NR³;
X² is a bond, oxygen or NR³;
R³ is a bond or hydrogen;
Y is C═O or a spacer group selected from:

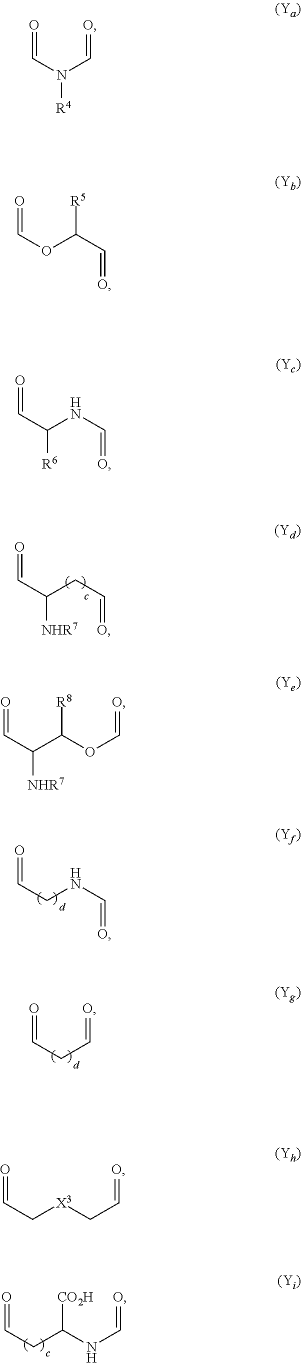

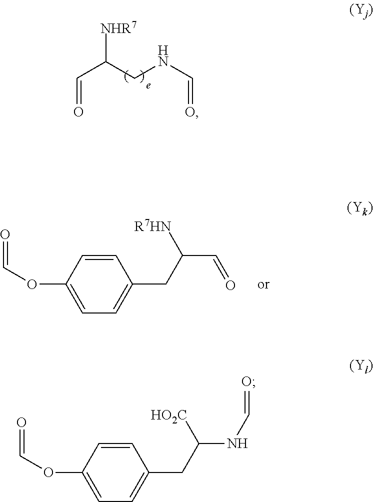

wherein in the spacer groups of formulae (Yₐ) to (Yₗ):
R⁴ is a bond, hydrogen, alkyl or a metal ion selected from sodium, potassium or calcium;
R⁵ is hydrogen, C₁₋₆ alkyl or phenyl;
R⁶ is hydrogen or a group (which is a side-chain group of naturally occurring amino acids) selected from:
—CH₃, —CH(CH₃)₂, —CH₂CH(CH₃)₂, —CH(CH₃)CH₂CH₃, —CH₂CO₂H, —CH₂CH₂CO₂H, —CH₂OH, —CH(CH₃)OH, —CH₂SH, —CH₂CH₂SCH3, —CH₂CH₂CH₂CH₂NH₂, —C₆H₅, —CH₂C₆H₅, —CH₂C₆H₄-p-OH, —CH₂CH₂CH₂NHC(═NH)NH₂, —CH₂C(═O)NH₂, —CH₂CH₂C(═O)NH₂, —CH₂-indol-3-yl or —CH₂-imidazole;
X³ is oxygen, sulphur, SO, SO₂ or NR³;
R⁷ is hydrogen or a group selected from acetyl, benzoyl, alkyloxycarbonyl, benzyloxycarbonyl, 9-fluorenylmethyloxy carbonyl or its pharmaceutically acceptable ammonium salts;
R⁸ is hydrogen or C₁₋₆ alkyl;
c is an integer from 0 to 2;
d is an integer from 1 to 5;
e is an integer from 1 to 4.
   Z¹ is (CH₂)ₐ; where a is an integer from 0 to 3;
   Z² is (CH₂)_{b}; where b is an integer from 0 to 3;
   A is a bond, S, SO, SO₂, S—S, CH═CH, D-isosorbide skeleton, 1,4-anhydroerythritol skeleton, cycloalkylene, CR⁹R¹⁰, C₆-C₁₀-arylene, a 5- or 6-membered heteroarylene or a 5- or 6-membered heterocyclylene wherein said arylene, heteroarylene and heterocyclylene may be unsubstituted or substituted by one or more substituents independently selected from the group consisting of C₁₋₆ alkyl, C₁₋₆ alkoxy, hydroxy, trifluoromethyl, cyano, amino and halogen;
   R⁹ and R¹⁰ are independently hydrogen or alkyl; or R⁹ and R¹⁰ taken together with the carbon atom to which they are attached form a cycloalkyl or a heterocyclic ring;
   R¹ is hydrogen and R² is alkyl, cycloalkyl, aryl or aralkyl; or R² is hydrogen and R¹ is alkyl, cycloalkyl, aryl or aralkyl;
   with the proviso that:
a) when A is S, then a and b is 3; or
b) when A is D-isosorbide skeleton or 1,4-anhydroerythritol skeleton, then a and b is 0;
   in all its stereoisomeric forms and mixtures thereof in all ratios or pharmaceutically acceptable salts thereof.

In another aspect, the present invention provides a bio-cleavable linker of formula (IA) capable of forming a covalent linkage with a drug (designated herein as D) containing one or more functional groups independently selected from a carboxylic acid, an amino, a hydroxyl or a sulfhydryl group:

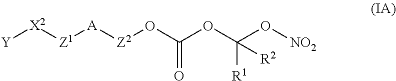

wherein the variables Y, X², Z¹, A, Z², R¹ and R² are as defined in respect of the compounds of formula (I). The linker of formula (IA) is non-toxic and facilitates release of nitric oxide and serves as an important intermediate in the processes for the synthesis of nitric oxide releasing prodrugs of formula (I) which are the prodrugs of known drugs or therapeutic agents.

In yet another further aspect, the present invention provides processes for the preparation of the compounds of formula (I).

In yet another further aspect, the present invention provides processes for the preparation of the bio-cleavable linker of formula (IA).

In yet another aspect, the present invention provides a pharmaceutical composition comprising the compound of formula (I) as an active ingredient and at least one pharmaceutically acceptable excipient.

In yet another further aspect, the present invention provides a method for the treatment of diseases or disorders in a subject by administering a therapeutically effective amount of the compound of the formula (I) to the subject.

In yet another further aspect, the present invention provides the compounds of formula (I), which are the prodrugs of known drugs or therapeutic agents, for use in the treatment of diseases or disorders capable of being treated by the parent drugs or therapeutic agents from which the prodrugs are derived.

### BRIEF DESCRIPTION OF THE DRAWINGS

FIG. 1 depicts the different pathways for oxidation and reduction of nitrates, nitrite and NO in the human body.

### DETAILED DESCRIPTION OF THE INVENTION

The present invention encompasses compounds of formula (I), as described herein, which are nitric oxide releasing prodrugs of known drugs or therapeutic agents useful in the treatment of diseases or disorders that are characteristic of the drugs from which the prodrugs of the present invention are derived.

In general, the present invention provides prodrugs of known drugs or therapeutic agents represented herein by the compounds of formula (I) which primarily constitutes the following elements:
(a) a drug containing one or more functional groups independently selected from a carboxylic acid, an amino, a hydroxyl or a sulfhydryl group capable of forming a covalent bio-cleavable linkage with a linker;
(b) a linker;
(c) optionally a spacer; and
(d) a nitrooxy (ONO₂) group.

The strategy for providing the prodrugs represented herein by the compounds of formula (I) is applicable to any drug or therapeutic agent which possesses a functional group such as a carboxylic acid, an amino, a hydroxy or a sulfhydryl group capable of covalently binding to a linker. The linker is a bi- or multi-functional moiety having the desired covalent binding properties.

The prodrugs [the compounds of formula (I)] of the present invention would undergo enzymatic cleavage in a manner such that the parent drugs and effective amounts of nitric oxide are released in vivo and that the oral bioavailability of the parent drugs is nearly maintained. The prodrugs [the compounds of formula (I)] of the present invention are expected to be safe to administer and may have oral bioavailability comparable or superior to that of the parent drug molecule.

Unless otherwise indicated, the following definitions are set forth to illustrate and define the meaning and scope of the various terms used to describe the invention herein and the appended claims. These definitions should not be interpreted in the literal sense as they are not general definitions and are relevant only for this application.

As used herein, the term “prodrug or prodrugs” refers/refer to a compound/compounds which upon administration to a subject in need thereof undergoes chemical conversion by metabolic or chemical processes to release the parent drug in vivo from which the prodrug is derived.

As used herein, the term “drug” or “drugs” or “therapeutic agents” or “drug molecules” or “parent drug” or “parent drug molecules” are used interchangeably. The term “drug” or “drugs” as used herein refers to any compound, substance, medicament or active ingredient having a therapeutic or pharmacological effect, and which is suitable for administration to a mammal, e.g., a human. More particularly, in the context of the present invention all the known drugs or therapeutic agents containing one or more functional groups independently selected from a carboxylic acid, an amino, a hydroxyl, or a sulfhydryl group that are capable of forming a covalent bio-cleavable linkage with a linker. The term “drug” or “drugs” as used herein also encompasses within its scope the “investigational drug(s)” or “investigational agent(s)” which refer to any new drug or agent currently under clinical investigation, particularly those investigational drugs or agents that contain one or more functional groups independently selected from a carboxylic acid, an amino, a hydroxyl or a sulfhydryl group capable of forming a covalent bio-cleavable linkage with a linker, which may later be established as therapeutically active agents by the regulatory bodies of different countries, are also encompassed within the scope of the term “drugs” or “therapeutic agents” as used herein. For example, when the drug or the therapeutic agent or the parent drug molecule contained in the compounds of formula (I) can be selected from anti-inflammatory and analgesic agents, cardiovascular agents, anti-allergic agents, anti-cancer agents, anti-depressants, anti-convulsant agents, anti-bacterial agents, anti-fungal agents, anti-viral agents, anti-malarial agents, anti-diabetic agents, anti-ulcer agents, anti-oxidants or vitamins.

As used herein, the term “linker” or “linkers” or “bio-cleavable linkers” refers/refer to a chemical moiety or moieties which forms/form a covalent linkage with the reactive carboxylic acid, amino, hydroxyl or sulfhydryl group of the drug or therapeutic agent to obtain a prodrug of the drug. This linker may be cleaved from the prodrug by chemical means, by enzymatic means, or by both the means. The linker may be pharmacologically inert or may itself provide added beneficial pharmacological activity.

As used herein, the term “alkyl”, alone or as part of a substituent of other groups, means a branched or straight-chain monovalent alkyl radical, preferably having one to six carbon atoms such that the alkyl group is designated as C₁₋₆-alkyl. This term is further exemplified by such radicals as methyl, ethyl, n-propyl, isopropyl, n-butyl, s-butyl, t-butyl. Unless stated otherwise, the “term” alkyl includes unsubstituted alkyl groups as well as alkyl groups substituted by one or more substituents. A substituted alkyl refers to an alkyl residue in which one or more hydrogen atoms are optionally replaced with substituents, for example, halogen, hydroxyl, alkoxyl, carbonyl, amino, nitro, nitrooxy, alkylthio, sulfhydryl, carbamate, sulphamate, sulphonate or an aryl group.

As used herein, the term “amino” functional group of drug or therapeutic agent refer to derivatisable primary and secondary amines (both acyclic and cyclic) which also include drugs containing derivatisable NH-containing functional groups such as amide-NH, sulfonamide-NH, carbamate-NH, sulfamate-NH, hydrazide-NH, hydrazone-NH, semicarbazone-NH, thiosemicarbazone-NH, urea-NH, and also encompass drug molecules with derivatisable NH-containing heterocyclic sub-structures such as aziridine, azitidine, dihydropyridine, indole, imidazole, benzimidazole, thiozole, benzothiozole, oxazole, benzoxazole, pyrrole, pyrrazol, benzopyrrozole, pyrrolidine, piperidine, triazole, benzotriazoles, tetrazole, and benzotetrazole.

As used herein, the term “hydroxyl” or “hydroxy” functional group of drugs or therapeutic agents refer to drugs containing derivatisable hydroxyl groups [i.e., these hydroxyl (OH) groups can be primary, secondary, tertiary or phenolic in nature] including hydroxyl groups of hydroxamic acids and ketoximes of keto-containing drug molecules.

As used herein, the term “sulfhydryl” groups of drugs or therapeutic agents refer to drugs containing derivatisable free sulfhydryl (SH) groups and these can be primary, secondary, tertiary and thiophenolic in nature.

As used herein, the term “halogen” refers to fluorine, bromine, chlorine or iodine.

As used herein, the term “halide” refers to fluoride, chloride, bromide, and iodide.

As used herein, the term “aryl” refers to a monocyclic or polycyclic aromatic hydrocarbon system having 6 to 14 carbon atoms, preferably 6 to 10 ring carbon atoms, in which at least one carbocyclic ring is present that has a conjugated pi-electron system. Examples of (C₆-C₁₄) aryl ring system include phenyl, naphthyl, biphenyl or anthracenyl, particularly preferred aryl ring system include phenyl and naphthyl. Unless stated otherwise, the aryl ring system, for example, phenyl, naphthyl or anthracenyl, can be optionally substituted with one or more identical or different substituents selected from the groups consisting of alkyl, halogen, hydroxyl, alkoxy, nitro, amino, trihaloalkyl, carbonyl (such as carboxyl, formate, carbamide, ester, ketone, aldehyde), carbamate, sulphamate, sulphonate, sulphate or a sulfhydryl group. The aryl residue can be bonded via any desired position and in substituted aryl, the substituents can be located in any desired position. For instance, in mono-substituted phenyl residue, the substituent can be present in 2-, 3-, 4- or 5-position. If the phenyl group carries two substituents, they can be located in 2,3-position, 2,4-position, 2,5-position, 2,6-position, 3,4-position or 3,5-position.

As used herein, the term “arylene”, by itself or as part of another substituent means, unless otherwise stated, a divalent aryl radical having 6 to 14 ring carbon atoms, preferably 6 to 10 ring carbon atoms. The arylene group can have a single ring (e.g., phenyl), multiple rings (e.g., biphenyl), or multiple condensed rings in which at least one is aromatic, (e.g., 1, 2, 3, 4-tetrahydronaphthyl, naphthyl), which is optionally substituted with one or more groups selected from, e.g., halogen, alkyl, alkoxy, trifluoromethyl. Representative arylene groups include, by way of example, 1,2-phenylene, 1,3-phenylene, 1,4-phenylene, naphthalene-1,5-diyl, naphthalene-2,7-diyl, and the like.

As used herein, the term “cycloalkyl” refers to a saturated mono-, bi- or polycyclic ring system containing a specified number of carbon atoms. Unless otherwise stated, cycloalkyl rings containing 3 to 7 carbon atoms are preferred. Representative cycloalkyl groups include cyclopropyl, cyclobutyl, cyclopentyl, cyclohexyl, cycloheptyl and the like. Further, unless otherwise stated, the term cycloalkyl includes unsubstituted cycloalkyl or cycloalkyl which is optionally substituted with any one of the substitutents mentioned above for aryl and the substitution can be in any desired position. Cycloalkyl group comprises a saturated cycloalkyl ring system which does not contain any double bond within the rings and partially unsaturated cycloalkyl ring systems which may contain one or more double bonds within the ring system that is stable and provided that the double bonds are not located in a manner that an aromatic system results.

As used herein, the term “cycloalkylene” refers to a divalent saturated carbocyclic hydrocarbon group. Unless otherwise defined, such cycloalkylene groups typically contain from 3 to 10 carbon atoms. Representative cycloalkylene groups include, by way of example, cyclopropane-1,2-diyl, cyclobutyl-1,2-diyl, cyclobutyl-1,3-diyl, cyclopentyl-1,2-diyl, cyclopentyl-1,3-diyl, cyclohexyl-1,2-diyl, cyclohexyl-1,3-diyl, cyclohexyl-1,4-diyl, and the like.

As used herein, the term “aralkyl” refers to an alkyl group substituted with an aryl group, wherein the term alkyl group is as defined above. Representative aralkyl groups include —(CH₂)_{g}-phenyl (wherein g is an integer from 1 to 2) such as benzyl, phenethyl and the like.

As used herein, the terms “heterocyclyl” or “heterocyclic ring” refer to a saturated, partially unsaturated or aromatic monocyclic or polycyclic heterocyclic ring system containing 3 to 14 ring atoms of which 1, 2, 3 or 4 are identical or different heteroatoms selected from the group consisting of nitrogen, oxygen and sulphur. The heterocyclyl ring, for example, has 1 or 2 oxygen atoms and/or 1 or 2 sulphur atoms and/or 1 or 2 nitrogen atoms. In monocyclic groups, heterocyclic ring preferably is a 3-membered, 4-membered, S-membered, 6-membered or 7-membered ring, more preferably a S- or 6-membered ring comprising one to three hetero atoms selected from the group consisting of nitrogen, oxygen and sulphur. Representative examples of saturated heterocyclic rings include pyrrolidinyl, piperidinyl, piperazinyl, tetrahydrofuryl, oxazolidinyl, dioxanyl and pyranyl. Representative examples of unsaturated heterocyclic rings are furyl, thienyl, pyridinyl, pyrrolyl, N-methylpyrrolyl, oxazolyl, isoxazolyl, pyrazolyl, imidazolyl, tetrazolyl, triazolyl, oxadiazolyl, thiadiazolyl, thiazolyl, pyrimidinyl, pyrazinyl and pyridazinyl.

In polycyclic groups, the term “heterocycle” or “heterocyclic ring” preferably comprises two fused rings (bicyclic), one of which is a S- or a 6-membered heterocyclic ring and the other is a 5- or 6-membered heterocyclic ring. Representative examples of polycyclic saturated heterocycle are indolinyl, 1,2,3,4-tetrahydroquinolinyl and 1,2,3,4-tetrahydroisoquinolinyl. Representative examples of polycyclic unsaturated heterocycle are quinolinyl, isoquinolinyl, benzoxazolyl, benzthiazolyl, benzofuranyl, thionaphthyl and indolyl. Unless stated otherwise, the heterocycle or heterocyclic group can be unsubstituted or substituted on the ring carbon atoms with one or more substituents. Each suitable ring nitrogen atom in the heterocycle or heterocyclic ring can independently of the other be unsubstituted i.e., carry a hydrogen atom or can be substituted. Suitable examples of substituents for the heterocyclic ring carbon and/or the nitrogen atoms are: amino, halo, hydroxyl, alkyl, haloalkyl, cyano, nitro, sulfhydryl and carboxyl.

As used herein, the term “heteroarylene” refers to a divalent aromatic group having a single ring or two fused rings containing at least one heteroatom, typically 1 to 3 heteroatoms, selected from the group consisting of nitrogen, oxygen or sulfur in the ring. Unless otherwise defined, such heteroarylene groups typically contain from 5 to 10 total ring atoms. Representative examples of heteroarylene groups include, divalent species of pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like, where the point of attachment is at any available carbon or nitrogen ring atom.

As used herein, the term “side chain group of naturally occurring amino acids” is intended to refer to the side chains of α-amino acids selected from the group consisting of alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. The side-chain group of naturally occurring amino acids being the group represented as R⁶ in the spacer group of formula Y_{c}, the sub-group that is defined in the variable Y in respect of the compounds of formula (I).

As used herein, the term “amino protecting group” is intended to refer to a group that can be selectively attached to the nitrogen atom by chemical modification of an amino group so as to selectively inhibit participation of the amino group in chemical reactions. After the completion of said chemical reactions the amino protecting group may be selectively removed. Exemplary amino-protecting groups include, carbamates (urethanes) such as methyl, ethyl, 9-fluorenylmethyl (i.e., Fmoc or 9-fluorenylmethoxycarbonyl), 2,2,2-trichloroethyl (i.e., Troc or trichloroethoxycarbonyl, 2-trimethylsilylethyl (i.e., Teoc or trimethylsilylethoxycarbonyl), 2-phenylethyl, 2-chloroethyl, 1,1-dimethyl-2,2,2-trichloroethyl, t-butyl (i.e., BOC or tert-butoxycarbonyl), benzyl (i.e., Cbz or Z or benzyloxycarbonyl), 1-adamantyl, 2-adamantyl, p-methoxybenzyl, p-nitrobenzyl, p-chlorobenzyl, 2,4-dichlorobenzyl, 4-methylsulfinylbenzyl, 9-anthrylmethyl, diphenylmethyl, 2-methylthioethyl, 2-methylsulfonylethyl, 4-methylthiophenyl, 4-azidobenzyl, 3,5-dimethoxybenzyl, o-nitrobenzyl, 2-iodoethyl, phenyl, etc., and amides such as formyl, acetyl, chloroacetyl, trichloroacetyl, trifluoroacetyl, phenylacetyl, benzoyl, o-nitrophenylacetyl, o-nitrobenzoyl, bromoacetyl, iodoacetyl, methoxyacetyl, etc., and cyclic imides such as phthalimide, etc., and N-alkyl and N-aryl amines such as N-methyl, N-t-butyl, N-allyl, N-cyanomethyl, N-benzyl, N-4-methoxybenzyl, N-2.4-dimethoxybenzyl, N-diphenylmethyl, N-bis(4-methoxyphenyl)methyl, N-triphenylmethyl (Tr), N-[(methoxyphenyl)diphenylmethyl] (MMTr), etc., and imine derivatives such as N-1,1-dimethylthiomethyleneamine, N-benzylideneamine, N-p-methoxybenzylideneamine, N-diphenylmethyleneamine, etc. Additional examples of amino protecting groups listed in T. W. Greene, “Protective Groups in Organic Synthesis”, John Wiley and Sons, New York, N.Y., 1991 are incorporated herein as a reference. Also, the procedures for the formation and cleavage of the above mentioned amino protecting groups are based on the known methods and their relevant references are cited in T. W. Greene, “Protective Groups in Organic Synthesis”, John Wiley and Sons, New York, N.Y., 1991 and incorporated herein as a reference.

As used herein, the term “hydroxyl protecting group” or “hydroxy protecting group”, is intended to refer to a group that can be selectively attached to the oxygen atom by chemical modification of the hydroxyl group so as to selectively inhibit the participation of the hydroxyl group in chemical reactions. After said chemical reactions the hydroxy protecting group may be selectively removed. Examples of hydroxyl and phenolic-protecting groups include, ether groups such as the alkyl ether group selected from methyl ether, methoxymethyl ether, methylthiomethyl ether, tert-buylthiomethyl ether, triphenylmethyl, tetrahydropyranyl (THP), (phenyldimethylsilyl)methoxy methyl ether, benzyloxymethyl ether, p-methoxybenzyloxy-methyl ether, o-nitrobenzyloxymethyl, p-nitrobenzyloxymethyl, t-butoxymethyl ether, menthoxymethyl ether, 2-methoxyethoxymethyl ether, siloxymethyl ether, ethoxyethyl ether, 1-(2-chloroethoxy)-ethyl ether, 2,2,2-trichloroethoxymethyl ether, 2-(trimethylsilyl)ethoxymethyl ether; and isopropyl ether, the aryl ether group is selected from phenyl ether, p-chlorophenyl ether, p-methoxyphenyl ether, 2,4-dinitrophenyl ether, benzyl ether, p-methoxybenzyl ether, o-nitrobenzyl ether, and 2,6-dichlorobenzyl ether, the alkylsilyl ether groups selected from trimethyl-, triethyl- and triisopropyl-silyl ethers, mixed alkylsilyl ether groups selected from dimethylisopropylsilyl ether, tert-butyldimethylsilyl ether and diethylisopropylsilyl ether; and the ester groups selected from acetate ester, formate ester, benzylformate ester, mono-, di-, and trichloroacetate ester, trifluoroacetate ester, methoxyacetate ester, triphenylmetoxyacetate ester, benzoate ester, phenylacetate ester, pivalate ester, phenoxyacetate ester, p-chlorophenoxyacetate, 2-iodobenzoate, 4-azidobutyrate, 4-nitro-4-methylpentanoate, o-(dibromomethyl)benzoate, 2-formylbenzenesulfonate, 4-(methylth iomethoxy)butyrate, 2-(methylthiomethoxymethyl)benzoate, 2-[(chloroacetoxymethyl)ethyl]benzoate, 2-L2-(benzyloxy)ethyl]benzoate, 2-L2-(4-methoxybenzyloxy)ethyl]benzoate, monosuccinate, o-(methoxycarbonyl)benzoate, nitrate, benzyloxycarbonate, benzyl, ethyl or methyl carbonate, methoxymethyl carbonate, 9-fluorenylmethyl carbonate, 2,2,2-trichloroethyl carbonate, 2-(trimethylsilyl)ethyl carbonate, 2-(phenylsulfonyl)ethyl carbonate, 2-(methylthiomethoxy)ethyl carbonate, 2-(4-nitrophenyl)ethyl carbonate, methyl dithiocarbonate, 9-fluorenyl methoxycarbonate, t-butoxycarbonate, trichloroethylcarbonate, 2-danysylethyl carbonate, 2-(4-nitrophenyl)ethyl carbonate, 2-(2,4-dinitrophenyl)ethyl carbonate, 2-cyano-1-phenylethyl carbonate, S-benzyl thiocarbonate, 4-ethoxy-1-naphthyl carbonate, borates carbamates, sulfonates and sulphamate. Examples of protecting groups for 1,2-diols, 1,3-diols, 2-hydroxybenzenethiols and catechols include, cyclic acetals and ketals such as methylene acetal, ethylidene acetal, t-butylmethylidene ketal, 1-t-butylethylidene ketal, 1-phenylethylidene ketal, 1-(4-methoxyphenyl)ethylidene acetal, trichloroethylidene acetal, acrolein acetal, isopropylidene ketal (acetonide), cyclopentylidene ketal, cyclohexylidene ketal, cycloheptylidene ketal, benzylidene acetal, p-methoxybenzylidene acetal, 2,4-dimethoxybenzylidene acetal, 3,4-dimethoxybenzylidene acetal, 2-nitrobenzylidene acetal, 4-nitrobenzylidene acetal, mesitylene acetal, 1-naphthaldehyde acetal, benzophenone ketal, o-xylyl ether, camphor ketal, cyclic ortho esters such as methoxymethylene acetal, ethoxymethylene acetal, dimethoxymethylene ortho ester, 1-methoxyethylene ortho ester, 1-ethoxyethylene ortho ester, methylidne ortho ester, phthalido ortho ester, 2-oxacyclopentylidene ortho ester, butane-2,3-bisacetal, cyclohexane-1,2-diacetal, dispiroketal, silyl derivatives such as di-t-butylsilylene group, dialkylsilylene groups, 1,3-(1,1,3,3-tetraisopropyldisiloxanylidene group, 1,1,3,3-tetra-t-butoxydisiloxaneylidene group, cyclic carbonates, cyclic boronates, phenyl boronate and o-acetamidophenyl boronate. Additional examples of hydroxyl protecting groups are described in T. W. Greene, “Protective Groups in Organic Synthesis”, John Wiley and Sons, New York, N.Y., 1991. Also, the procedures for the formation and cleavage of the above mentioned hydroxyl protecting groups are based on the known methods and their relevant references are cited in T. W. Greene, “Protective Groups in Organic Synthesis”, John Wiley and Sons, New York, N.Y., 1991 and incorporated herein as a reference.

As used herein, the term “carboxyl protecting group” or “carboxylic acid protecting group” is intended to refer to a group that selectively blocks the oxygen functionality within a carboxylic acid group so as to inhibit participation of the carboxylic acid group in chemical reactions. Examples of such carboxylic acid protecting groups include, for example unsubstituted and substituted alkyl esters such as methyl, ethyl, t-butyl, benzyl, 9-fluorenylmethyl, methoxymethyl, methylthiomethyl, methoxyethoxymethyl, 2-(trimethylsilyl)ethoxymethyl, benzyloxymethyl, pivaloyloxymethyl, phenylacetoxymethyl, triisopropylsiliylmethyl, cyanomethyl, acetol (hydroxy acetone), phenacyl, p-bromophenacyl, p-chlorophenacyl, p-methoxyphenacyl, carboxamidomethyl (Cam), etc., and 2-subtituted ethyl esters such as 2,2,2-trichloroethyl, 2-haloethyl, 2-(trimethylsilyl)ethyl, 2-methylthioethyl, 2-cyanoethyl, cyclopentyl, cyclohexyl, allyl, phenyl, etc., and substituted benzyl esters such as triphenylmethyl (trityl), diphenylmethyl (Dpm), 9-anthrylmethyl, p-methoxybenzyl, etc., and silyl esters such as trimethylsilyl (TMS), triethylsilyl (TES), t-butyldimethylsilyl (TBDMS), i-propyldimethylsilyl, phenyldimethylsilyl, di-t-butylmethylsilyl (DTBMS), and triisopropylsilyl (TIPS). Additional examples of carboxylic acid protecting groups are described in T. W. Greene, “Protective Groups in Organic Synthesis”, John Wiley and Sons, New York, N.Y., 1991. Also, the procedures for the formation and cleavage of the above mentioned carboxyl protecting groups are based on the known methods and their relevant references are cited in T. W. Greene, “Protective Groups in Organic Synthesis”, John Wiley and Sons, New York, N.Y., 1991 and incorporated herein as a reference.

As used herein, the term “sulfhydryl protecting group” or “thiol protecting group” is intended to refer to a group that selectively blocks the thiol (SH) functionality so as to inhibit participation of the thiol group in chemical reactions. Examples of such thiol protecting groups include, thioethers such as S-alkyl, S-benzyl, S-p-methoxybenzyl, S-o- or p-hydroxy- or acetoxybenzyl, S-p-nitrobenzyl, S-2,4,6-trimethyl/trimethoxybenzyl, S-4-picolyl, S-2-quinolinomethyl, S-9-Anthrylmethyl, S-9-Fluorenylmethyl, S-xanthenyl, S-diphenylmethyl, S-substituted diphenylmethyl, S-triphenylmethyl, S-bis(4-methoxyphenyl)methyl, S-bis(4-methoxyphenyl)phenylmethyl (DMTr), S-t-butyl, S-1-Adamantyl, S-2-(4′-pyridyl)ethyl, S-2-cyanoethyl, S-2-(trimethylsilyl)ethyl, S-2,2-bis(carboethoxy)ethyl, etc., and monothio acetals such as S-acetamidomethyl, S-trimethylacetamidomethyl, S-benzamidomethyl, S-allyloxycarbonylaminomethyl, S-phenylacetamidomethyl, S-phthalimidomethyl, S-methoxymethyl, S-isobutoxymethyl, S-benzyloxymethyl, S-2-tetrahydropyranyl, etc., and dithioacetals such as S-benzylthiomethyl, S-phenylthiomethyl, etc., and silyl thioethers such as triisopropylsilyl, etc., and thioesters such S-acetyl, S-benzoyl, S-trifluoroacetyl, etc., and thiocarbonates such as S-2,2,2-trichloroethoxycarbonyl, S-t-butoxycarbonyl, S-benzyloxycarbonyl, etc., and thiocarbamates such as S-(N-ethylcarbamate), S-(N-methoxymethylcarbamate), etc., and unsymmetrical disulfides such as S-ethyl disulfide, S-t-butyl disulfide, substituted S-phenyl disulfide, etc., and sulfenyl derivatives such as S-sulfonate, S-sulfenylthiocarbonate, S-3-nitro-2-pyridinesulfenyl sulfide, etc., and protection of dithiols as dithio acetals and ketals such as S,S′-methylene, S,S-isopropylidene and S,S′ benzylidene derivatives. Also, protection of 1,2-aminothiols as thiozolidine derivatives. The procedures for the formation and cleavage of the above mentioned sulfhydryl protecting groups are based on the known methods and their relevant references are cited in T. W. Greene, “Protective Groups in Organic Synthesis”, John Wiley and Sons, New York, N.Y., 1991 and incorporated herein as a reference.

The term “leaving groups” or “LGs” include, but are not limited to, (substituted) alkoxy, aryloxy, nitrogen containing unsaturated heterocycles such as N-oxybenzotriazole, imidazolyl, o- or p-nitrophenoxy, pentachloro-phenoxy, N-oxysuccinimide, N,N′-dicyclohexylisoure-O-yl, N-hydroxy-N-methoxyamino, and the like; acetates, formates, sulfonates such as methanesulfonate, ethanesulfonate, benzenesulfonate, or p-toluenesulfonate, and the like; and halides such as fluoride, chloride, bromide, or iodide.

The term “coupling agent” or “carbonyl activating agent” refers to a reagent that converts the carbonyl of a carboxylic acid group into one that is more susceptible to nucleophilic attack and includes, but is not limited to, such reagents as those found in “The Peptides”, Gross and Meienhofer, Eds., Academic Press (1979), Ch. 2, and M. Bodanszky, “Principles of Peptide Synthesis”, 2.sup.nd Ed., Springer-Verlag Berlin Heidelberg, 1993, hereafter referred to as “The Peptides” and “Peptide Synthesis” respectively. Carbonyl group (i.e., aldehyde or keto group) of the drugs or drug molecules may be converted first to aldoxime, ketoxime, hydrazone, semicarbazone and the like, before coupling to the linker. Specifically, carbonyl activating agents include thionyl bromide, thionyl chloride, oxalyl chloride, and the like; esters of alcohols such as nitrophenol, pentachlorophenol, and the like; and compounds such as 1,1′-carbonyldiimidazole (CDI), benzotriazole, imidazole, N-hydroxysuccinimide, dicyclohexylcarbodiimide (DCC), 1-Ethyl-(3-dimethylaminopropyl)carbodiimide (EDAC), phosgene or its equivalents, N,N-dimethylaminopyridine (DMAP) and the like.

The terms “phosgene or its equivalents” refer to phosgene or it equivalents such as diphosgene, triphosgene, N,N′-Carbonyldiimidazole (CDI), N,N′-Dicyclohexylcarbodiimide (DSC), 1,1-Bis[6-(trifluoromethyl)benzotrazolyl]-carbonate (BTBC), alkoxycarbonyl chlorides, o/p-nitrosubstituted phenoxycarbonyl chlorides, and the like.

The term “suitable solvent” refers to a solvent that is inert to the ongoing reaction and sufficiently solubilizes the reactants to effect the desired reaction. Examples of suitable solvents include but are not limited to, dichloromethane, chloroform, 1,2-dichloroethane, diethyl ether, tert-butylmethyl ether, acetonitrile, ethyl acetate, 1,3-dimethyl-2-imidazolidinone, tetrahydrofuran, dimethylformamide, benzene, toluene, xylene, N,N-dimethylacetamide, N-methylpyrrolidine, chlorobenzene, dimethylsulfoxide, dimethoxyethane, water, methanol, ethanol, isopropanol, pyridine, nitromethane, and the like or mixtures thereof.

The term “suitable base” refers to a base, which acts as a proton trap for any protons, which may be produced as a byproduct of the desired reaction, or to a base, which provides a reversible deprotonation of an acidic proton from the substrate and is reactive enough to effect the desired reaction without significantly effecting any undesired reactions. Examples of such bases include, but are not limited to, suitable metal carbonates, bicarbonates, and hydroxides (e.g., lithium, sodium, potassium, magnesium, calcium and the like), sodium/potassium/calcium hydride, sodium/potassium alkoxide (i.e., methoxide, ethoxide, tert-butoxide and the like), triethylamine, diisopropylethylamine, N-methylpyrrolidine, N-methylmorpholine, tetramethylguinidine, or aromatic nitrogen containing heterocycles such as pyridine, 4-(dimethylamino)pyridine (DMAP), and the like.

The term “suitable oxidizing agent” refers to a suitable agent that causes oxidation of a molecule. The term “oxidation” in chemistry refers to either elimination of hydrogen or replacement of hydrogen atom bonded to carbon with another more electronegative element such as oxygen. A more general definition of oxidation involves an increase in oxidation state and loss of one or more electrons from an atom or group. Examples of oxidation include transformations such as conversion of: an alcohol to a carbonyl compound (i.e., to aldehydes or ketones), aldehydes or ketones to carboxylic acid, aromatics to phenols, phenols to quinones, alkenes to diols, epoxides or ketones, sulfides to sulfoxides and sulfones, metals to metal cations and so on. Examples of “suitable oxidizing agents” include, but not limited to, chromium reagents such as chromium trioxide, chromium trioxide-pyridine, pyridinium chlorochromate (PCC), pyridinium dichromate (PDC), oxidations involving dimethyl sulfoxide and an activating agent such as oxalyl chloride or trifluoroacetic anhydride (Swern oxidation), DCC and an acid catalyst (Moffat oxidation), acetic anhydride or pyridine-sulfur trioxide, Dess-Martin Periodinane, Oxone, Oxammonium salts, metal derivatives such as aluminum triisopropoxide, cyclopentadienyl zirconium reagent (Cp₂ZrH₂), manganese dioxide, silver carbonate, silver (I) oxide, silver (II) oxide, permanganate reagents such as potassium permanganate, trimethylcetylammonium permanganate and n-butyl permanganate, molybdenum reagents such as ammonium molybdate [(NH₄)₆Mo₇O₂₄.2H₂O], cerium (IV) reagents such as ceric ammonium sulfate and ceric ammonium nitrate, peroxides such as hydrogen peroxide and t-butyl hydroperoxide (TBHP), per acids such as peracetic acid, trifluoroperacetic acid, perbenzoic acid and m-chloroperbenzoic acid, potassium persulfate, N-bromosuccinimide, osmium tetroxide, ozone, sodium periodate, ruthinium tetroxide, lead tetraacetate, selenium dioxide, and so on.

The term “suitable reducing agent” refers to a suitable agent that causes reduction to a molecule. The term “reduction” in chemistry is generally defined as a decrease in oxidation state and a gain of one or more electrons. Examples of reduction include transformations such as conversion of: aldehydes or ketones or acids or esters or epoxides to alcohols, amides or azides or imides or imines or nitriles or nitro groups or oximes to amines, alkenes or alkynes to alkanes, sulfonate esters or halocarbons to alkanes, cations to corresponding metal atoms, disulfide to sulfhydryl and sulfone or sulfoxide to sulfide. Examples of “suitable reducing agents” include, but not limited to lithium aluminum hydride, sodium borohydride, potassium borohydride, sodium hydride, metal trialkoxyaluminum hydrides [LiAlH(OR)₃] such as [LiAlH(OMe)₃], [LiAlH(OEt)₃] and [LiAlH(O^{t}Bu)₃], Red-Al (sodium bis(2-methoxyethoxy)aluminum hydride, diisobutylaluminum hydride (Dibal or DIBAL-H)lithium triethylborohydride (Super-Hydride™), zinc borohydride, metal/ammonium acyloxyborohydrdes [M BH₄₋ₙ(O₂R)ₙ] such as potassium triacetoxyborohydride, sodium triacetoxyborohydride, tetramethylammonium triacetoxyborohydride, potassium tri-sec-butylborohydride (K-Selectride™), lithium tri-sec-butylborohydride (L-Selectride™), sodium cyanoborohydride, boranes such diborane (B₂H₆), borane complex of dimethylsulfide (H₃B.SMe₂), bis(1,2-dimethylpropyl)borane (disiamylborane), 9-borabicyclo[3.3.1]nonane (9-BBN), and catalytic reductions/hydrogenations using metal catalysts such as platinum oxide, Pt/C. Pd oxide, Pd hydroxide/C, Ni-borides, NiC, Raney Ni, copper chromite, platinum black, Pt/Rh oxide, Pd/BaCO₃, Pd/C, Rh/C, Ni—Cu, Raney Ni W1, Raney Ni W2, Raney Ni W3, Raney Ni W4, Raney Ni W5, Raney Ni W6, Raney Ni W7, Raney Ni W8 and Raney cobalt, Li-Liq. NH₃, Na-Liq. NH₃, Zn dust, ZnCl₂, Zinc amalgam [Zn(Hg)], Tin compounds such as tributyltin hydride (Bu₃SnH), SnCl₂, Aluminum isopropoxide [Al(O—ⁱPr)₃], aluminum amalgam (Al/Hg), silanes such as Et₃SiH, PhMe₂SiH, Ph₂SiH₂ and so on.

The term “pharmaceutically acceptable salts” refers to the salts of the compound of formula (I) of the invention which are toxicologically acceptable and pharmaceutically utilisable salts. The compound of formula (I), which contains a basic functionality, can be used according to the invention in the form of their addition salts of organic or inorganic acids. The pharmaceutically acceptable acid addition salts of the prodrugs i.e. the compounds of formula (I) include salts which retain the biological effectiveness and properties of the free bases and which are not biologically or otherwise undesirable. Examples of suitable inorganic acids include hydrochloric acid, hydrobromic acid, sulphuric acid, nitric acid, phosphoric acid, perchloric acid, boric acid, and other inorganic acids known in the art. Examples of organic acids include: acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfanilic acid, 2-acetoxy benzoic acid, toluenesulphonic acid, methane sulphonic acid, ethane disulphonic acid, isethionic acid, ketoglutaric acid, benzenesulphonic acid and other organic acids known in the art.

The compound of formula (I), which contains acidic group, can be used according to the invention as base addition salts. Examples of pharmaceutically acceptable base addition salts include those salts derived from inorganic bases such as alkali earth metal salts like sodium, potassium, lithium, alkaline earth metal salts like calcium, magnesium, aluminium salts or salts of organic bases such as lysine, arginine, triethylamine, dibenzylamine, piperidine or as salts with ammonia. Particularly preferred are the ammonium salts of the prodrugs of the present invention i.e. the compounds of formula (I). The pharmaceutically acceptable salts of the present invention can be synthesized from the subject compound which contains a basic or acidic moiety by conventional chemical methods. Generally the salts are prepared by contacting the free base or acid with stoichiometric amounts or with an excess of the desired salt-forming inorganic or organic acid or base in a suitable solvent or dispersant or by anion exchange or cation exchange with other salts. Suitable solvents are, for example, ethyl acetate, ether, alcohols, acetone, tetrahydrofuran (THF), dioxane or mixtures of these solvents.

In a first embodiment, the invention relates to compounds of the formula (I), which are prodrugs of known drugs or therapeutic agents;

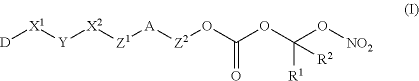

wherein
D is a drug containing one or more functional groups independently selected from a carboxylic acid, an amino, a hydroxyl or a sulfhydryl group capable of forming a covalent bio-cleavable linkage with a linker of formula IA (as described herein);
X¹ is a bond, oxygen, sulphur, or NR³;
X² is a bond, oxygen or NR³;
R³ is a bond or hydrogen;
Y is C═O or a spacer group selected from:

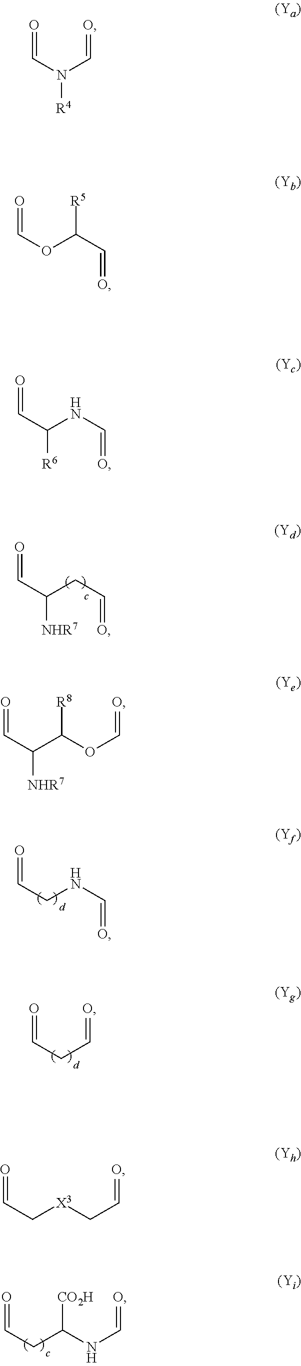

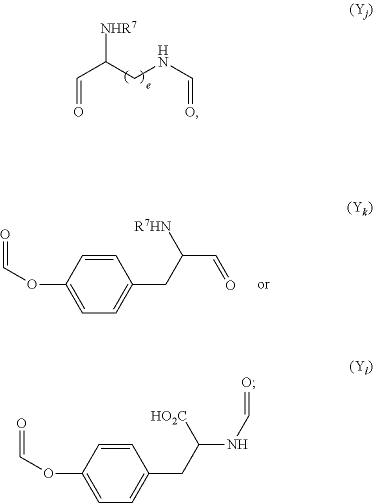

where in the spacer groups of formulae (Yₐ) to (Yₗ):
R⁴ is a bond, hydrogen, alkyl or a metal ion;
R⁵ is hydrogen, C₁₋₆ alkyl or phenyl;
R⁶ is hydrogen or a side-chain group of naturally occurring amino acids selected from:
—CH₃, —CH(CH₃)₂, —CH₂CH(CH₃)₂, —CH(CH₃)CH₂CH₃, —CH₂CO₂H, —CH₂CH₂CO₂H, —CH₂OH, —CH(CH₃)OH, —CH₂SH, —CH₂CH₂SCH3, —CH₂CH₂CH₂CH₂NH₂, —C₆H₅, —CH₂C₆H₅, —CH₂C₆H₄-p-OH, —CH₂CH₂CH₂NHC(═NH)NH₂, —CH₂C(═O)NH₂, —CH₂CH₂C(═O)NH₂, —CH₂-indol-3-yl or —CH₂-imidazole;
X³ is oxygen, sulphur, SO, SO₂ or NR³;
R⁷ is hydrogen or an amino protecting group selected from: acetyl, benzoyl, alkyloxycarbonyl, benzyloxycarbonyl, 9-fluorenylmethyloxy carbonyl or its pharmaceutically acceptable ammonium salts;
R⁸ is hydrogen or C₁₋₆ alkyl;
c is an integer from 0 to 2;
d is an integer from 1 to 5;
e is an integer from 1 to 4;
   Z¹ is (CH₂)ₐ; where a is an integer from 0 to 3;
   Z² is (CH₂)_{b}; where b is an integer from 0 to 3;
   A is selected from: a bond, S, SO, SO₂, S—S, CH═CH, D-isosorbide skeleton, 1,4-anhydroerythritol skeleton, cycloalkylene, CR⁹R¹⁰, C₆-C₁₀-arylene, a 5- or 6-membered heteroaylene or a 5- or 6-membered heterocyclylene wherein said arylene, heteroarylene and heterocyclylene may be unsubstituted or substituted by one or more substituents independently selected from the group consisting of C₁₋₆ alkyl, C₁₋₆ alkoxy, hydroxy, trifluoromethyl, cyano, amino and halogen;
   R⁹ and R¹⁰ are independently selected from: hydrogen or C₁₋₆ alkyl; or R⁹ and R¹⁰ taken together with the carbon atom to which they are attached form a cycloalkyl or a heterocyclic ring;
   R¹ is hydrogen; and R² is alkyl, cycloalkyl, aryl or aralkyl; or R² is hydrogen; and R¹ is alkyl, cycloalkyl, aryl or aralkyl;
   with the provisos that:
a) when A is S, then a and b is 3; or
b) when A is D-isosorbide skeleton or 1,4-anhydroerythritol skeleton, then a and b is 0; and
   in all its stereoisomeric forms and pharmaceutically acceptable salts thereof.

It would be understood by a person having skill in the art to which this invention relates that the functional groups namely the carboxylic acid, amino, hydroxyl and sulfhydryl groups contained in the drug “D” in the compounds of formula (I) participate in the formation of a linkage with the linker represented herein by the compound of formula IA through the variable “X¹” or ‘Y’ which constitute part of the formula (I) represented herein. In other words, the variable X¹ and Y (in part) are derived from the carboxylic acid or amino or hydroxyl or sulfhydryl functional groups of the drug “D” from which the nitric oxide releasing prodrugs of the present invention i.e. the compounds of formula (I), are derived. For instance, the variables X¹ and Y in the compound of formula (I) represents the chemical functionality on the drug “D” represented by carboxylic acid (X¹=bond and Y═C(O)), amino (X¹═NR³ and Y═C(O)), hydroxyl (X¹=oxygen) and sulfhydryl (X¹=sulphur) functional groups which are involved in the formation of covalent linkage with the cleavable linker of formula (IA).

In a second embodiment, the invention encompasses a compound of formula (I), wherein:

each of D, X¹, Z¹ and Z² are as defined in the first embodiment herein above;

### Y is C═O;

X² is oxygen;
A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH, D-isosorbide skeleton, 1,4-anhydroerythritol skeleton, cycloalkyl or CR⁹R¹⁰;
where R⁹ and R¹⁰ are independently selected from hydrogen or C₁₋₆ alkyl; or R⁹ and R¹⁰ taken together with the carbon atom to which they are attached constitute a cycloalkyl group or a 5- or 6-membered heterocyclic ring containing one to two hetero atoms selected from oxygen, sulfur or nitrogen;
R¹ is hydrogen and R² is alkyl, cycloalkyl, aryl or aralkyl; or R² is hydrogen and R¹ is alkyl, cycloalkyl, aryl or aralkyl;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a third embodiment, the invention encompasses a compound of formula (I), wherein: each of D, X¹, Z¹ and Z² is as defined in the first embodiment herein above;

each of Y and X² is as defined in the second embodiment herein above;
A is selected from a bond, CH═CH or CR⁹R¹⁰; where R⁹ and R¹⁰ are independently selected from: hydrogen or C₁₋₆ alkyl; or R⁹ and R¹⁰ taken together with the carbon atom to which they are attached form a cycloalkyl or a 5- or 6-membered heterocyclic ring;
R¹ and R² are as defined in the second embodiment hereinabove;
in all its stereoisomeric forms and pharmaceutically acceptable salts thereof.

In a fourth embodiment, the invention encompasses a compound of formula (I), wherein:

each of D, X¹, Z¹ and Z² is as defined in the first embodiment herein above;
each of Y and X² is as defined in the second embodiment herein above;
A is selected from S, SO, SO₂ or S—S; provided that when A is S, then a and b is 3;
R¹ and R² are as defined in the second embodiment hereinabove;
in all its stereoisomeric forms and pharmaceutically acceptable salts thereof.

In a fifth embodiment, the invention encompasses a compound of formula (I), wherein:

each of D, X¹, Z¹ and Z² is as defined in the first embodiment herein above;
each of Y and X² is as defined in the second embodiment herein above;
A is selected from 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton or cycloalkyl; provided that when A is D-isosorbide skeleton or 1,4-anhydroerythritol skeleton, then a and b is 0;
R¹ and R² are as defined in the second embodiment hereinabove;
in all its stereoisomeric forms and pharmaceutically acceptable salts thereof.

In a sixth embodiment, the invention encompasses a compound of formula (I), wherein:

each of D, X¹, Z¹ and Z² is as defined in the first embodiment hereinabove;
each of X² and Y is as defined in the second embodiment hereinabove;
A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH or CR⁹R¹⁰; where R⁹ and R¹⁰ are independently selected from hydrogen or C₁₋₆ alkyl;
provided that when A is S, then a and b is 3;
R¹ is hydrogen and R² is alkyl; or R² is hydrogen and R¹ is alkyl;
in all its stereoisomeric forms and pharmaceutically acceptable salts thereof.

In a seventh embodiment, the invention encompasses a compound of formula (I), wherein:

D is a drug containing a carboxylic acid group capable of forming a covalent bio-cleavable linkage with a linker of formula (IA) (as described herein);
X¹ is a bond;
X², Y, Z¹, Z², A, R¹ and R² are as defined in the first embodiment hereinabove;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In an eighth embodiment, the invention encompasses a compound of formula (I), wherein:

each of D and X¹ is as defined in the seventh embodiment hereinabove;
each of X², Y, Z¹, Z², A, R¹ and R² is as defined in the second embodiment hereinabove;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a ninth embodiment, the invention encompasses a compound of formula (I), wherein: D, the drug containing a carboxylic acid group capable of forming a covalent bio-cleavable linkage with a linker, referred to in the first, second, third, fourth, fifth, sixth, seventh and eighth embodiments, is selected from an anti-inflammatory and analgesic agent, a cardiovascular agent, an antiallergic agent, an anticancer agent, an antidepressant, an anticonvulsant agent, an antibacterial agent, an antifungal agent, an antiviral agent, an antimalarial agent, an antidiabetic agent, an antiulcer agent, a vitamin or an antioxidant.

In this embodiment, other variables X¹, X², Y, Z¹, Z²; A, R¹ and R² in the compounds of formula (I) are as defined hereinabove; provided that
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a tenth embodiment, the invention encompasses a compound of formula (I), wherein: D, the drug containing a carboxylic acid group capable of forming a covalent bio-cleavable linkage with a linker, is selected from an anti-inflammatory and analgesic agent, a cardiovascular agent, an antiallergic agent, an anticancer agent, an antidepressant, an anticonvulsant agent, an antibacterial agent, an antifungal agent, an antiviral agent, an antimalarial agent, an antidiabetic agent, an antiulcer agent, a vitamin or an antioxidant;

X¹ is a bond;

### Y is C═O;

### X² is O;

Z¹ and Z² are as defined in the first embodiment hereinabove;
A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH, D-isosorbide skeleton, 1,4-anhydroerythritol skeleton, cycloalkyl or CR⁹R¹⁰;
where R⁹ and R¹⁰ are independently selected from hydrogen or C₁₋₆ alkyl; or R⁹ and R¹⁰ taken together with the carbon atom to which they are attached constitute a cycloalkyl group or a 5- or 6-membered heterocyclic ring containing one to two hetero atoms selected from oxygen, sulfur or nitrogen;
R¹ is hydrogen and R² is alkyl, cycloalkyl, aryl or aralkyl; or R² is hydrogen; and R¹ is alkyl, cycloalkyl, aryl or aralkyl;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In an eleventh embodiment, the invention encompasses a compound of formula (I), wherein: D, the drug containing a carboxylic acid group capable of forming a covalent bio-cleavable linkage with a linker, is selected from an anti-inflammatory and analgesic agent, a cardiovascular agent, an antiallergic agent, an anticancer agent, an antidepressant, an anticonvulsant agent, an antibacterial agent, an antifungal agent, an antiviral agent, an antimalarial agent, an antidiabetic agent, an antiulcer agent, a vitamin or an antioxidant;

each of X¹, Y, X², Z¹, Z², R¹ and R² are as defined in the tenth embodiment hereinabove;
A is selected from a bond, CH═CH or CR⁹R¹⁰;
wherein, R⁹ and R¹⁰ are independently selected from hydrogen or C₁₋₆ alkyl; or R⁹ and R¹⁰ taken together with the carbon atom to which they are attached constitute a cycloalkyl group;
in all its stereoisomeric forms and pharmaceutically acceptable salts thereof.

In a twelfth embodiment, the invention encompasses a compound of formula (I), wherein:

D, the drug containing a carboxylic acid group capable of forming a covalent bio-cleavable linkage with a linker, is selected from an anti-inflammatory and analgesic agent, a cardiovascular agent, an antiallergic agent, an anticancer agent, an antidepressant, an anticonvulsant agent, an antibacterial agent, an antifungal agent, an antiviral agent, an antimalarial agent, an antidiabetic agent, an antiulcer agent, a vitamin or an antioxidant;
each of X¹, Y, X², Z¹, Z², R¹ and R² is as defined in the tenth embodiment hereinabove;
A is selected from S, SO, SO₂ or S—S; provided that when A is S, then a and b is 3;
and in all its stereoisomeric forms and pharmaceutically acceptable salts thereof.

In a thirteenth embodiment, the invention encompasses a compound of formula (I), wherein:

D, the drug containing a carboxylic acid group capable of forming a covalent bio-cleavable linkage with a linker, is selected from an anti-inflammatory and analgesic agent, a cardiovascular agent, an antiallergic agent, an anticancer agent, an antidepressant, an anticonvulsant agent, an antibacterial agent, an antifungal agent, an antiviral agent, an antimalarial agent, an antidiabetic agent, an antiulcer agent, a vitamin or an antioxidant;
each of X¹, Y, X², Z¹, Z², R¹ and R² is as defined in the tenth embodiment hereinabove;
A is selected from 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton or cycloalkyl; provided that when A is D-isosorbide skeleton or 1,4-anhydroerythritol skeleton, then a and b is 0;
in all its stereoisomeric forms and pharmaceutically acceptable salts thereof.

In a fourteenth embodiment, the invention encompasses a compound of formula (I), wherein:

D, the drug containing a carboxylic acid group capable of forming a covalent bio-cleavable linkage with a linker, is selected from an anti-inflammatory and analgesic agent, a cardiovascular agent, an antiallergic agent, an anticancer agent, an antidepressant, an anticonvulsant agent, an antibacterial agent, an antifungal agent, an antiviral agent, an antimalarial agent, an antidiabetic agent, an antiulcer agent, a vitamin or an antioxidant;
X¹ is a bond;
Y is a spacer group as defined in the first embodiment hereinabove;

### X² is O;

Z¹ and Z² are as defined in the first embodiment hereinabove;
A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH, D-isosorbide skeleton, 1,4-anhydroerythritol skeleton, cycloalkyl or CR⁹R¹⁰; where R⁹ and R¹⁰ are independently selected from hydrogen or C₁₋₆ alkyl; or R⁹ and R¹⁰ taken together with the carbon atom to which they are attached constitute a cycloalkyl group or a 5- or 6-membered heterocyclic ring containing one to two hetero atoms selected from oxygen, sulfur or nitrogen;
R¹ is hydrogen and R² is alkyl, cycloalkyl, aryl or aralkyl; or R² is hydrogen; and R¹ is alkyl, cycloalkyl, aryl or aralkyl;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a fifteenth embodiment, in the compound of formula (I) the anti-inflammatory and analgesic agent referred to in the ninth, tenth, eleventh, twelfth, thirteenth and fourteenth embodiments hereinabove is generically selected from an opioid, steroids (glucocorticoids) or a non-steroidal anti-inflammatory drug (NSAID(s)) and is specifically selected from aceclofenac, acemetacin, acetamidocaproic acid, acetylsalicylsalicylic acid, actarit, alclofenac, 3-alminoprofen, amfenac, 3-amino-4-hydroxybutyric acid, aspirin (acetylsalycilic acid), balsalazide, bendazac, benoxaprofen, bromprofen, bromfenac, 5-bromosalicylic acid acetate, bucloxic acid, bumadizone, butibufen, carprofen, cinchophen, cinmetacin, clidanac, clometacin, clonixin, clopirac, diacerein, diclofenac, diflunisal, dipyrocetyl, enfenamic acid, enoxolone, etodolac, felbinac, fenbufen, fenclozic acid, fendosal, fenoprofen, fentiazac, flufenamic acid, flunoxaprofen, fluocortolone-21-acid, flurbiprofen, fosfosal, gentisic acid, ibufenac, ibuprofen, indomethacin, indoprofen, isofezolac, isoxepac, ketoprofen, ketorolac, lonazolac, loxoprofen, meclofenamic acid, mefenamic acid, mesalamine, metiazinic acid, mofezolac, naproxen, niflumic acid, olsalazine, oxaceprol, oxaprozin, pirazolac, pirprofen, pranoprofen, protizinic acid, salicysulfuric acid, salicylamide o-acetic acid, salsalate, sulfasalazine, sulindac, suprofen, suxibuzone, tiaprofenic acid, tolfenamic acid, tolmetin, tropesin, ximoprofen, zaltoprofen or zomepirac.

The representative example of an anti-inflammatory and analgesic agent is a NSAID that is selected from aspirin, diclofenac, diflunisal, etodolac, fenoprofen, flurbiprofen, ibuprofen, indomethacin, ketoprofen, ketorolac, naproxen, sulindac or tolmetin.

Further in the fifteenth embodiment, the invention encompasses a compound of formula (I); wherein the cardiovascular agent referred to in the ninth, tenth, eleventh, twelfth, thirteenth and fourteenth embodiments hereinabove is generically selected from an antihypertensive agent such as an angiotensin converting enzyme (ACE) inhibitor, a beta-blocker, sartan (angiotensin II blockers), an antithrombotic and vasoactive agent, an anti-hyperlipidemic drug (including HMG-CoA-reductase inhibitor (statins), fibrate, an antianginal agent, an antiarrhythmic agent, an antihypotensive agent, a diuretic, a vasodilator or vasoprotectant and is specifically selected from acifran, acipimox, acetylsalicylic acid, alacepril, gama-aminobutyric acid, angiotensin, argatroban, atorvastatin, benazepril, benfurodil hemisuccinate, beraprost, bezafibrate, bumetanide, candesartan, capobenic acid, captopril, carmoxirole, caronapril, chromocarb, cilazapril, ciprofibrate, clinofibrate, clofibric acid, dalteparin, daltroban, delapril, dextrothyroxine, eicosapentaenoic acid, eledoisin, enalapril, enalaprilat, enoxaparin, eprosartan, ethacrynic acid, fluvastatin, fosinopril, furosemide, gemfibrozil, iloprost, imidapril, indobufen, isbogrel, heparin, lamifiban, limaprost, lisinopril, lotrafiban, meglutol, melagatran, mercamphamide, mercaptomerin sodium, mercumallylic acid, mersalyl, methyldopa, moexipril, moveltipril, nadroparin, omapatrilat, ozagrel, oxiniacic acid, perindopril, piretanide, privastatin sodium, prostaglandin E₁ quinapril, ramipril, reviparin sodium salt, ridogrel, sampatrilat, saralasin, satigrel, spirapril, taprostene, telmisartan, temocapril, thyropropic acid, ticrynafen, tinzaparin, tirofiban, trandolapril, triflusal, valsartan, xanthinol niacinate or xenbucin.

A representative example of the cardiovascular agent is an ACE-inhibitor that is selected from benazepril, enalapril, enalaprilat, lisinopril, perindopril, quinapril, ramipril, ramiprilate, trandolapril, alacepril, captopril, ceronapril, cilazapril, delapril, fosinopril, imidapril, moexipril, moveltipril, omapatrilat, sampatrilat, spirapril or temocapril.

Another representative example of the cardiovascular agent is a sartan that is selected from candesartan, olmesartan, telmisartan or valsartan.

Yet another representative example of the cardiovascular agent is an antithrombotic and vasoactive agent that is selected from acetylsalicylic acid, argatroban, beraprost, dalteparin, daltroban, enoxaparin, iloprost, indobufen, isbogrel, heparin, lamifiban, lotrafiban, melagatran, nadroparin, ozagrel, reviparin sodium salt, ridogrel, satigrel, taprostene, tinzaparin, tirofiban or triflusal.

Yet another representative example of the cardiovascular agent is an anti-hyperlipidemic agent (statin and fibrate) that is selected from atorvastatin, bezafibrate, cerivastatin, ciprofibrate, clinofibrate, clofibric acid, fluvastatin, gemfibrozil, pitavastatin, or pravastatin.

Yet another representative example of the cardiovascular agent is an antianginal agent such as limaprost.

Yet another representative example of the cardiovascular agent is an antiarrhythmic agent such as capobenic acid.

Yet another representative example of the cardiovascular agent is an antihypotensive agent such as angiotensin.

Yet another representative example of the cardiovascular agent is a diuretic that is selected from bumetanide, ethacrynic acid, furosemide, mercamphamide, mercaptomerin sodium, mercumallylic acid, mersalyl, piretanide or ticrynafen.

Yet another representative example of the cardiovascular agent is a vasodilator that is selected from benfurodil hemisuccinate, beraprost, eledoisin, iloprost, prostaglandin E₁ or xanthinol niacinate.

Yet another representative example of the cardiovascular agent is a vasoprotectant such as chromocarb.

Still further, in the fifteenth embodiment, the invention encompasses a compound of formula (I); wherein the antiallergic agent referred to in the ninth, tenth, eleventh, twelfth, thirteenth and fourteenth embodiments hereinabove is generically selected from a steroidal bronchodilator, a mast cell stabilizer or an antihistamine and is specifically selected from acrivastine, amlexanox, bepotastine, cetirizine, fexofenadine, levocetirizine, lodoxamide, montelukast sodium, nedocromil, olopatadine, pentigetide or tranilast.

A representative example of the antiallergic agent is an antihistamine that is selected from acrivastine, bepotastine, cetirizine, fexofenadine, levocabastine, levocetirizine or montelukast sodium.

Still further, in the fifteenth embodiment, the invention encompasses a compound of formula (I); wherein the anticancer agent referred to in the ninth, tenth, eleventh, twelfth, thirteenth and fourteenth embodiments hereinabove is selected from acitretin (etretin), aminolevulinic acid, amsilarotene, butyric acid, eflornithine hydrochloride, melphalan, methotrexate, minodronate (minodronic acid), retinoic acids (including 13-cis retinoic and all trans-retinoic acids), sulindac, tamibarotene or valproic acid.

Still further, in the fifteenth embodiment, the invention encompasses a compound of formula (I); wherein the antidepressant (including antimaniacs and antipsychotics) referred to in the ninth, tenth, eleventh, twelfth, thirteenth and fourteenth embodiments hereinabove is generically selected from an antimaniac or an antipsychotic agent and is specifically selected from amineptine, gabapentin, 5-hydroxytryptophan (oxitriptan), pregabalin, tianeptine, valproic acid or vigabatrin.

Still further, in the fifteenth embodiment, the invention encompasses a compound of formula (I); wherein the anticonvulsant referred to in the ninth, tenth, eleventh, twelfth, thirteenth and fourteenth embodiments hereinabove is selected from gabapentin, pregabalin, tiagabine, valproic acid or vigabatrin.

Still further, in the fifteenth embodiment, the invention encompasses a compound of formula (I); wherein the antibacterial referred to in the ninth, tenth, eleventh, twelfth, thirteenth and fourteenth embodiments hereinabove is selected from acediasulfone, amdinocillin, p-aminosalicylic acid, amoxicillin, amphomycin, ampicillin, apalcillin, apicycline, aspoxicillin, azidocillin, azlocillin, aztreonam, bacitracin, balofloxacin, benzoylpas, benzylpenicillin, betamipron, biapenem, carbenicillin, carindacillin, carumonam, cefaclor, cefadroxil, cefalexin, cefamandole, cefatiam, cefatrizine, cefazedone, cefazolin, cefbuperazone, cefclidin, cefdinir, cefditoren, cefepime, cefetamet, cefixime, cefmenoxime, cefmetazole, cefminox, cefodizime, cefonicid, cefoperazone, ceforanide, cefoselis, cefotaxime, cefotetan, cefotiam, cefoxitin, cefozopran, cefpimizole, cefpiramide, cefpirome, cefroxadine, cefsulodin, ceftazidime, cefteram, ceftezole, ceftibuten, ceftizoxime, ceftriaxone, cefprozil, cefuroxime, cefuzonam, cephacetrile sodium, cephalexin, cephaloglycin, cephaloridine, cephalosporin C, cephalothin, cephapirin sodium, cephradine, cilastatin, cinoxacin, ciproflaxacin, clavulinic acid, clavulanate, clinafloxacin, clometocillin, cyclacillin, dicloxacillin, difloxacin, enoxacin, epicillin, ertapenem, fenbenicillin, fleroxacin, flomoxef, floxacillin, flumequine, fosfomycin, fropenem, fusidic acid, garenoxacin, gatifloxacin, gemifloxacin, grepafloxacin, hetacillin, hydnocarpic acid, imipenem, lomefloxacin, loracarbef, lymecycline, merbromin, meropenem, metampicillin, methicillin, mezlocillin, miloxacin, moxalactam, moxifloxacin, nadifloxacin, nafcillin, nalidixic acid, negamycin, noprysulfamide, norfloxacin, ofloxacin, opiniazide, oxacillin, oxolinic acid, panipenem, pazufloxacin, pefloxacin, penicillin(s), penimepicycline, phenethicillin, phthalylsulfacetamide, phthalylsulfathiazole, pipemidic acid, piperacillin, piromidic acid, propicillin, prulifloxacin, quinacillin, ritipenem, rosoxacin, rufloxacin, salazosulfadimidine, salbactam, sitafloxacin, sparfloxacin, succinylsulfathiazole, succisulfone, sulbenicillin, sulfachrysoidine, sulfaloxic acid, 4-sulfanilamidosalicylic acid, sulfanilic acid, tazobactam, teicoplanin, temocillin, ticarcillin, tigemonam, tosufloxacin, trovafloxacin, tyrocidine or vancomycin.

A representative example of the antibacterial agent is selected from amoxicillin, ampicillin, cefadroxil, cefalexin, cefixime, cefotaxime, cefuroxime, cephalexin, ciproflaxacin, gatifloxacin, nadifloxacin, nalidixic acid, norfloxacin, ofloxacin, oxacillin, panipenem, salbactam or vancomycin.

Still further, in the fifteenth embodiment, the invention encompasses a compound of formula (I); wherein the antifungal agent referred to in the ninth, tenth, eleventh, twelfth, thirteenth and fourteenth embodiments hereinabove is selected from amphotericin B, azaserine, benzoic acid, candicidin, lucensomycin, natamycin, nystatin, propionic acid, salicylic acid or undecylenic acid (10-undecenoic acid).

Still further, in the fifteenth embodiment, the invention encompasses a compound of formula (I); wherein the antiviral agent referred to in the ninth, tenth, eleventh, twelfth, thirteenth and fourteenth embodiments hereinabove is selected from foscarnet sodium or zanamivir.

Still further, in the fifteenth embodiment, the invention encompasses a compound of formula (I); wherein the antimalarial agent referred to in the ninth, tenth, eleventh, twelfth, thirteenth and fourteenth embodiments hereinabove is artesumate.

Still further, in the fifteenth embodiment, the invention encompasses a compound of formula (I); wherein the antidiabetic agent referred to in the ninth, tenth, eleventh, twelfth, thirteenth and fourteenth embodiments hereinabove is selected from mitiglinide, nateglinide or repaglinide.

Still further, in the fifteenth embodiment, the invention encompasses a compound of formula (I); wherein the antiulcer agent (including proton pump inhibitor) referred to in the ninth, tenth, eleventh, twelfth, thirteenth and fourteenth embodiments hereinabove is selected from acetoxolone, arbaprostil, carbenoxolone, cetraxate, ecabet, S-methylmethionine, proglumide, rebamipide, rosaprostol, rotraxate, sofalcone or trimoprostil.

Still further, in the fifteenth embodiment, the invention encompasses a compound of formula (I); wherein the vitamin referred to in the ninth, tenth, eleventh, twelfth, thirteenth and fourteenth embodiments hereinabove is selected from biotin (vitamin H or coenzyme R), folic acid (vitamin M), menadoxime, nicotinic acid (niacin), pantothenic acid or vitamin B₅ (a member of the B complex vitamins).

Still further, in the fifteenth embodiment, the invention encompasses a compound of formula (I); wherein the antioxidant (including free radical scavengers) referred to in ninth, tenth, eleventh, twelfth, thirteenth and fourteenth embodiments hereinabove is selected from α-lipoic acid, L-Carnitine, N-acetyl L-cysteine, N-acetyl carnosine, raxofelast, tetomilast or SCMC-Lys (S-carboxymethyl-L-cysteine Lysine salt. H₂O).

For the purpose of this invention, the fifteenth embodiment also encompasses a compound of formula (I); wherein the drug containing carboxylic acid group is generically selected from the drugs that fall under several other therapeutic areas (including those drugs that are classified on the basis of their mechanism of action) and is specifically selected from an abortifacient/interceptive such as prostaglandin E₂; an anesthetic selected from ecgonidine, ecgonine, hydroxydione sodium or gama-hydroxybutyrate (gama-hydroxybutyric acid); an anthelmintic selected from antimony sodium thioglycollate, kainic acid or stibocaptate; an antiacne agent selected from adapalene, isotretinoin or all-trans retinoic acid, an antiamebic agent selected from thiocarbamizine or thiocarbarsone; an antiarthritic or antirheumatic agent selected from actarit, bucillamine, diacerein, gold sodium thiomalate, lobenzarit, allocupreide sodium, clobuzarit or penicillamine; an antiasthmatic agent selected from amlexanox, cilomilast (ariflo), cromolyn, domitroban, montelukast, nedocromil, ramatroban or seratrodast; an antigout/ucosuric agent selected from carprofen, probenecid, orotic acid, oxycinchophen or ticrynafen; an antidiuretic agent such as oxycinchophen; an antiglaucoma agent such as unoprostone; an antihypothyroid agent selected from tiratricol or thyroxine; an antiprostatic hypertrophy agent such as epristeride; an antiprotozoal agent selected from eflornithine or fumagillin; an antipsoriatic agent such acitretin; an antiseptic agent such as mandelic acid; an anxiolytic agent selected from calcium n-carbamoylaspartate or clorazepic acid; an astringent such as bismuth subgallate; a cathartic/laxative such as sennosides; choleretic agents selected from cholic acid, cicrotoic acid, clanobutin, cyclobutyrol, cynarin(e), dehydrocholic acid, deoxycholic acid, dimecrotic acid, exiproben, fencibutirol, florantyrone, menbutone, 3-(o-methoxyphenyl)-2-phenylacrylic acid, sincalide, tocamphyl or trepibutone; an enzyme cofactor such as pantothenic acid; an estrogen such as methallenestril; a gastroprokinetic agent selected from alvimopan or loxiglumide; a hemostatic agent selected from ε-aminocaproic acid or tranexamic acid; a hepatoprotectant selected from S-adenosylmethionine, betaine, orazamide, timonacic (thioproline), methionine, protoporphyrin IX, thioctic acid or tiopronin; an immunomodulator selected from bucillamine, ubenimex, pidotimod, procodazole, romurtide or thymopentin; immunosuppressant selected from brequinar or mycophenolic acid; a mucolytic selected from acetylcysteine, carbocysteine, erdosteine, letosteine or stepronin; a muscle relaxant selected from baclofen or carisoprodol; a nootropics/Cognitive enhancer selected from cetylcarnitine, hexacyclonate sodium or leteprinim; a prostaglandin analog selected from beraprost, carboprost, limaprost, prostacyclin, prostaglandin E₁, prostaglandin E₂, prostaglandin F_{2α}, rosaprostol, sulprostone, trimoprostil or unoprostone; a sedative/hypnotic chloral selected from betainem or calcium 2-ethylbutanoate; a dopamine receptor agonist such as carmoxirole; a 5α-Reductase inhibitor such as epristeride; a reverse transcriptase inhibitor such as foscarnet sodium; thromboxane A₂-receptor antagonist selected from altroban, domitroban, ramatroban, ridogrel or seratrodast and a thromboxane A₂-synthase inhibitor selected from isbogrel, ozagrel or ridogrel.

In a sixteenth embodiment, the invention encompasses a compound of formula (I), wherein D, the drug containing a carboxylic acid group capable of forming a covalent bio-cleavable linkage with a linker, is a non-steroidal anti-inflammatory drug (NSAID);

X¹ is a bond;
Y is C═O or a spacer group as defined in the first embodiment hereinabove;
X² is oxygen;
each of Z¹, Z², A, R¹ and R² is as defined in the second embodiment hereinabove; and
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a seventeenth embodiment, the invention encompasses a compound of formula (I), wherein D, the drug or a therapeutic agent containing a carboxylic acid group capable of forming a covalent bio-cleavable linkage with a linker, is a non-steroidal anti-inflammatory drug (NSAID);

X¹ is a bond;

### Y is C═O;

X² is oxygen;
each of Z¹, Z², R¹ and R² is as defined in the first embodiment hereinabove; and
A is selected from a bond, CH═CH or CR⁹R¹⁰; where R⁹ and R¹⁰ are independently selected from hydrogen or C₁₋₆ alkyl; or R⁹ and R¹⁰ taken together with the carbon atom to which they are attached constitute a cycloalkyl group;
in all its stereoisomeric forms and pharmaceutically acceptable salts thereof.

In an eighteenth embodiment, the invention encompasses a compound of formula (I), wherein:

wherein D, the drug containing a carboxylic acid group capable of forming a covalent bio-cleavable linkage with a linker, is a non-steroidal anti-inflammatory drug (NSAID);
each of X¹, Y, X², Z¹, Z², R¹ and R² is as defined in the seventeenth embodiment hereinabove;
A is selected from S, SO, SO₂ or S—S; provided that when A is S, then a and b is 3;
in all its stereoisomeric forms and pharmaceutically acceptable salts thereof.

In a nineteenth embodiment, the invention encompasses a compound of formula (I), wherein:

D, the drug containing a carboxylic acid group capable of forming a covalent bio-cleavable linkage with a linker, is a non-steroidal anti-inflammatory drug (NSAID);
each of X¹, Y, X², Z¹, Z², R¹ and R² is as defined in the seventeenth embodiment hereinabove;
A is selected from 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton or cycloalkyl; provided that when A is D-isosorbide skeleton or 1,4-anhydroerythritol skeleton, then a and b is 0; and
in all its stereoisomeric forms and pharmaceutically acceptable salts thereof.

In twentieth embodiment, the invention encompasses a compound of formula (I), wherein the non-steroidal anti-inflammatory drug (NSAID) referred to in the sixteenth, seventeenth, eighteenth and nineteenth embodiments is as defined in the fifteenth embodiment hereinabove. A representative example of the non-steroidal anti-inflammatory drug (NSAID) is selected from aspirin, diclofenac, naproxen, indomethacin, sulindac, flurbiprofen, ketoprofen, ibuprofen or mesalamine.

In a twenty-first embodiment, the invention encompasses a compound of formula (I), wherein: D is a drug containing an amino group capable of forming a covalent bio-cleavable linkage with a linker;

X¹ is NR³; wherein R³ is a bond or hydrogen;

### Y is C═O;

X²; Y, Z¹; Z²; A, R¹ and R² are as defined in the second embodiment hereinabove; and
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a twenty-second embodiment, the invention encompasses a compound of formula (I), wherein: each of D and X¹ is as defined in the twenty-first embodiment hereinabove;

each of X², Y, Z¹ and Z² is as defined in the second embodiment hereinabove;
A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH or CR⁹R¹⁰; where R⁹ and R¹⁰ are independently selected from hydrogen, C₁₋₆ alkyl;
provided that when A is S, then a and b is 3;
R¹ is hydrogen and R² is alkyl; or R² is hydrogen and R¹ is alkyl;
in all its stereoisomeric forms and pharmaceutically acceptable salts thereof.

In a twenty-third embodiment, the invention encompasses a compound of formula (I), wherein: each of D, X¹, X², Y, Z¹ and Z² is as defined in the twenty-second embodiment hereinabove,

A is selected from a bond, CH═CH or CR⁹R¹⁰; where R⁹ and R¹⁰ are independently selected from hydrogen or C₁₋₆ alkyl;

R¹ is hydrogen and R² is alkyl, cycloalkyl, aryl or aralkyl; or R² is hydrogen and R¹ is alkyl, cycloalkyl, aryl or aralkyl;

in all its stereoisomeric forms and pharmaceutically acceptable salts thereof.

In a twenty-fourth embodiment, the invention encompasses a compound of formula (I), wherein: each of D, X¹, X², Y, Z¹ and Z² is as defined in the twenty-second embodiment hereinabove,

A is selected from S, SO, SO₂ or S—S; provided that when A is S, then a and b is 3;
R¹ is hydrogen and R² is alkyl, cycloalkyl, aryl or aralkyl; or R² is hydrogen and R¹ is alkyl, cycloalkyl, aryl or aralkyl;
in all its stereoisomeric forms and pharmaceutically acceptable salts thereof.

In a twenty-fifth embodiment, the invention encompasses a compound of formula (I), wherein: D, the drug containing an amino group capable of forming a covalent bio-cleavable linkage with a linker, referred to in the first, second, third, fourth, fifth, sixth, twenty-first, twenty-second, twenty-third, and twenty-fourth embodiments herein above, is selected from an antiinflammatory and analgesic drug, a cardiovascular drug, an antiallergic agent, an anticancer agent, an antidepressant, an anticonvulsant agent, an antibacterial agent, an antiviral agent, an antifungal agent, an antimalarial agent, an antidiabetic agent an antiulcer agent, an antioxidant or a vitamin. The twenty-fifth embodiment also encompasses within its scope a drug containing an amino group wherein the drug is selected from several other therapeutic areas (including those drugs that are classified on the basis of their mechanism of action).

In this embodiment, other variables X¹; X², Y, Z¹, Z²; A, R¹ and R² in the compounds of formula (I) are as defined above; provided that
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In twenty-sixth embodiment, the invention encompasses a compound of formula (I), wherein: the antiinflammatory and analgesic drug referred to in the twenty-fifth embodiment hereinabove is generically selected from an opioid, a steroid (glucocorticoid) or a non-steroidal anti-inflammatory drug (NSAID(s)) and is specifically selected from aceclofenac, acetaminophen, acetaminosalol, actarit, alminoprofen, amfenac, aminochlorthenoxazin, 3-amino-4-hydroxybutyric acid, ampiroxicam, aminopropylon, anileridine, antrafenine, benorylate, benzpiperylon, p-bromoacetanilide, bromfenac, bucetin, bucolome, bufexamac, bumadizone, butacetin, capsaicine, carprofen, carsalam, celecoxib, clonixin, dezocine, diclofenac, difenamizole, difenpiramide, enfenamic acid, etersalate, ethenzamide, ethoxazene, etodolac, etofenamate, fepradinol, flipirtine, floctafenine, flufenamic acid, glafenine, ibuproxam, isoladol, isonixin, isoxicam, p-lactophenetide, lornoxicam, meclofenamic acid, mefenamic acid, meloxicam, mesalamine, mofebutazone, nifenazone, niflumic acid, nimesulide, norlevorphanol, normorphine, oxametacine, paranyline, parecoxib, parsalmide, phenacetin, phenazopyridine, phenocoll, phenopyrazone, phenylramidol, piketoprofen, piminodine, piperylone, piroxicam, piritramide, propacetamol, ramifenazone, salverine, salacetamide, salicylamide, salicylamide o-acetic acid, sulfasalazine, talniflumate, tenidap, terofenamate, tinoridine, tenoxicam, tolfenamic acid and valdecoxib. Preferred examples of antiinflammatory drugs include acetaminophen, bromfenac, celecoxib, diclofenac, etodolac, meloxicam, mesalamine, nimesulide, paracoxib, phenacetin or valdecoxib.

A representative example of the antiinflammatory and analgesic drug is selected from acetaminophen, bromfenac, celecoxib, diclofenac, etodolac, meloxicam, mesalamine, nimesulide, paracoxib, phenacetin or valdecoxib.

Further in the twenty-sixth embodiment, the cardiovascular agent referred to in the twenty-fifth embodiment hereinabove is generically selected from an antihypertensive agent such as an angiotensin converting enzyme (ACE) inhibitor, a beta-blocker, a sartan (angiotensin II blockers), an antithrombotic and vasoactive agent, an anti-hyperlipidemic drug (including HMG-CoA-reductase inhibitor (statins), fibrate, an antianginal agent, an antiarrhythmic agent, an antihypotensive agent, a calcium channel blocker, a cardiotonic agent, a cardioprotective agent, a diuretic or a vasodilator and is specifically selected from acadesine, acebutolol, acecamide, adenosine, alacepril, alfuzosin, alprenolol, althiazide, amanozine, ambuside, amezinium methyl sulfate, amiloride, gama-aminobutyric acid, aminometradine, 2-amino-4-picoline, amisometradine, amlodipine, amosulalol, aminone, angiotensin, aranidipine, argatroban, arotinolol, atenolol, azosemide, bamethan, barnidipine, benazepril, bendazol, bendroflumethiazide, benfluorex, benidipine, benzalbutyramide, benzylhydrochlorothiazide, benzthiazide, betahistine, bethanidine, betaxolol, bevantolol, bidisomide, bisoprolol, bopindolol, bosentan, bradykinin, bucindolol, bucladesine, bucumolol, budralazine, bufeniode, bufetolol, bufuralol, bumetanide, bunazosin, bunitrolol, bupranolol, butalamine, butazolamide, buthiazide, butidrine, butofilolol, cadralazine, candesartan, capobenic acid, carazolol, cariporide, carmoxirole, caronapril, carteolol, carvedilol, celiprolol, cetamolol, chloraminophenamide, chlorazanil, chlormerodrin, chlorothiazide, chlorthalidone, ciclosidomine, cifenline, cilazapril, cilnidipine, cilostazol, clofenamide, clonidine, clopamide, cloranolol, clorexolone, cyclopenthiazide, cyclothiazide, debrisoquin, delapril, denopamine, diazoxide, dihydralazine, dilevalol, dimetofrine, disopyramide, disulfamide, dobutamine, docarpamine, dofetilide, dopamine, dopexamine, doxazosin, droprenilamine, edeserpidine, efonidipine, eledoisin, elgodipine, enalapril, enalaprilat, encamide, endralazine, enoxaparin, enoximone, epanolol, erythrophleine, esmolol, ethiazide, ethoxzolamide, etifelmin, etilefrin, etiroxate, fasudil, felodipine, fendiline, fenoldopam, fenquizone, flecamide, furosemide, gepefrine, guanabenz, guanacline, guanazodine, guanethidine, guanochlor, guanadrel, guanfacine, guanoxabenz, guanoxan, heptaminol, hydracarbazine, hydralazine, hydrochlorothiazide, hydroflumethiazide, ibopamine, imidapril, imolamine, indapamide, indecamide, indenolol, indoramin, irbesartan, isoxsuprine, isradipine, itramin tosylate, kallidin, ketanserin, labetalol, lacidipine, lamifiban, landiolol, lercanidipine, levosimendan, lidoflazine, lisinopril, lofexidine, loprinone, losartan, lotrafiban, manidipine, mebutamate, mecamylamine, mefruside, melagatran, meobentine, mephentermine, mepindolol, metaraminol, methazolamide, methoxamine, methyclothiazide, methyldopa, methyl 4-pridyl ketone thiosemicarbazone, meticrane, metipranolol, metolazone, metoprolol, mexiletine, mibefradil, midodrine, milrinone, minoxidil, moexipril, molsidomine, monatepil, moprolol, moricizine, moveltipril, moxonidine, muzolimine, nadolol, nadoxolol, nebivolol, nicardipine, nicorandil, nifedipine, nifenalol, nilvadipine, nimodipine, nipradilol, nisoldipine, nitrendipine, norepinephrine, nylidrin, olmesartan, oxprenolol, oxyfedrine, pamabrom, paraflutizide, penbutolol, pentisomide, perhexyline, perindopril, pheniprazine, phentolamine, pholedrine, picotamide, pildralazine, pilsicamide, pimethylline, pimobendan, pinacidil, pindolol, piretanide, plafibride, polythiazide, practolol, prazosin, prenalterol, prenylamine, procainamide, pronethalol, propafenone, propranolol, quinapril, quinethazone, ramipril, ranolazine, raubasine, rescimetol, rescinnamine, reserpiline, reserpine, rilmenidine, roxifiban, sampatrilat, saralasin, sematilide, sotalol, spirapril, sulfinalol, sulmazole, suloctidil, synephrine, syrosingopine, talinolol, tasosartan, teclothiazide, temocapril, terazosin, terodiline, tertatolol, theobromine, tiamenidine, tilisolol, timolol, tinofedrine, tirofiban, tocamide, todralazine, tolazoline, toliprolol, tolonidine, torsemide, trandolapril, triamterene, trichlormethiazide, trimazosin, trimetazidine, tripamide, urapidil, valsartan, vesnarinone, viquidil, xamoterol, xemilofiban, xibenolol, ximelagatran or xipamide.

A representative example of the cardiovascular agent is an ACE inhibitor that is selected from alacepril, benazepril, ceronapril, cilazapril, delapril, enalapril, enalaprilat, imidapril, lisinopril, moexipril, moveltipril, omapatrilat, perindopril, quinapril, ramipril, spirapril, temocapril or trandolapril.

Another representative example of the cardiovascular agent is a beta-blocker that is selected from atenolol, bupranolol, carvedilol, labetalol, metipranolol, metoprolol, nadolol, pindolol, propranolol or timolol.

Another representative example of the cardiovascular agent is a sartan (angiotensin II blocker) that is selected from Irbesartan, losartan, olmesartan or valsartan;

Yet another representative example of the cardiovascular agent is an antithrombotic and vasoactive agent that is selected from argatroban, cilostazol, droprenilamine, enoxaparin, lamifiban, lotrafiban, melagatran, perhexyline, picotamide, plafibride, roxifiban, suloctidil, tirofiban, xemilofiban or ximelagatran.

Yet another representative example of the cardiovascular agent is an antianginal agent that is selected from amlodipine, bevantolol, bucumolol, bufuralol, elgodipine, imolamine, molsidomine, nicardipine, nicorandil, nifedipine, nifenalol, nipradilol, oxyfedrine, pronethalol, ranolazine, sotalol, terodiline, toliprolol or trimetazidine.

Yet another representative example of the cardiovascular agent is an antiarrhythmic agent that is selected from acecamide, adenosine, bidisomide, bufetolol, butidrine, capobenic acid, cifenline, cloranolol, disopyramide, dofetilide, encamide, esmolol, flecamide, indecamide, landiolol, meobentine, mexiletine, moricizine, nadoxolol, pentisomide, pilsicamide, practolol, procainamide, propafenone, sematilide, tocamide, tilisolol or xibenolol.

Yet another representative example of the cardiovascular agent is an antihypotensive agent that is selected from amezinium methyl sulfate, angiotensin, dimetofrine, dopamine, etifelmin, etilefrin, gepefrine, heptaminol, mephentermine, metaraminol, methoxamine, midodrine, norepinephrine, pholedrine or synephrine.

Yet another representative example of the cardiovascular agent is a calcium channel blocker that is selected from amlodipine, aranidipine, barnidipine, benidipine, cilnidipine, efonidipine, elgodipine, felodipine, fendiline, isradipine, lacidipine, lercanidipine, lidoflazine, manidipine, mibefradil, monatepil, nicardipine, nifedipine, nilvadipine, nimodipine, nisoldipine, nitrendipine, perhexyline, prenylamine or terodiline.

Yet another representative example of the cardiovascular agent is a cardiotonic agent that is selected from 2-amino-4-picoline, aminone, bucladesine, denopamine, dobutamine, docarpamine, dopamine, dopexamine, enoximone, erythrophleine, ibopamine, levosimendan, loprinone, milrinone, pimobendan, prenalterol, sulmazole, vesnarinone or xamoterol.

Yet another representative example of the cardiovascular agent is a cardioprotective agent that is selected from acadesine or cariporide.

Yet another representative example of the cardiovascular agent is a diuretic agent that is selected from althiazide, amanozine, ambuside, amiloride, aminometradine, amisometradine, azosemide, bendroflumethiazide, benzthiazide, bumetanide, butazolamide, buthiazide, chloraminophenamide, chlorazanil, chlormerodrin, chlorothiazide, chlorthalidone, clofenamide, clorexolone, cyclothiazide, disulfamide, ethiazide, ethoxzolamide, fenquizone, furosemide, hydrochlorothiazide, mefruside, methazolamide, methyclothiazide, meticrane, metolazone, muzolimine, pamabrom, paraflutizide, piretanide, polythiazide, quinethazone, teclothiazide, theobromine, torsemide, triamterene, trichlormethiazide or xipamide.

Yet another representative example of the cardiovascular agent is a vasodilator that is selected from bamethan, bendazol, betahistine, bradykinin, butalamine, droprenilamine, eledoisin, fasudil, fendiline, isoxsuprine, itramin tosylate, kallidin, lidoflazine, nimodipine, nylidrin, pimethylline, prenylamine, suloctidil, tinofedrine, tolazoline, trimetazidine or viquidil.

Still further in the twenty-sixth embodiment, the antiallergic agent referred to in the twenty-fifth embodiment hereinabove is generically selected from a steroidal bronchodilator, mast cell stabilizer or an antihistamine; and is specifically selected from amlexanox, antazoline, astemizole, bambuterol, cetoxime, clobenzepam, desloratadine, epinastine, mizolastine, oxatomide, pemirolast, pentigetide, pifatidine (roxatidine acetate hydrochloride), repirinast, salbutamol, salmeterol, suplatast, tazanolast, tranilast, tritoqualine or traxanox.

A representative example of the antiallergic agent is an antihistamine that is selected from antazoline, astemizole, cetoxime, clobenzepam, desloratadine, epinastine, mizolastine, pifatidine (roxatidine acetate hydrochloride) or tritoqualine.

Still further in the twenty-sixth embodiment, the anticancer agent referred to in the twenty-fifth embodiment hereinabove is selected from 9-aminocamptothecin, aminolevulinic acid, 3-aminopyridine-2-carboxaldehyde thiosemicarbazone (3-ap), 3-aminopyridine-4-methyl-2-carboxaldehyde thiosemicarbazone (3-amp/triapine/ocx-191/ocx-0191), amsacrine, ancitabine, anthramycin, azacitidine, bicalutamide, bisantrene, bleomycins, bropirimine, buserelin, carboplatin, carboquone, carmofur, carmustine, carubicin, chlorozotocin, cisplatin, cladribine, cyclophosphamide, cytarabine, dacarbazine, dactinomycin, daunorubicin, decitabine, defosfamide, demecolcine, diaziquone, 6-diazo-5-oxo-l-norleucine (don), docetaxel, doxorubicin, ecteinascidins, edatrexate, efaproxiral, eflornithine, eniluracil, epirubicin, erlotinib, fluorouracil, gefitinib, gemcitabine, goserelin, histamine, hydroxyurea, idarubicin, ifosfamide, imatinib, improsulfan, lanreotide, leuprolide, liarozole, lobaplatin, lomustine, lonafarnib, mannomustine, marimastat, melphalan, 6-mercaptopurine, methotrexate, methyl aminolevulinate, miboplatin, mitoguazone, mitoxantrone, nilutamide, nimustine, nolatrexed, oxaliplatin, pemetrexed, pentostatin, peplomycin, perfosfamide, phenamet, pirarubicin, piritrexim, prinomastat, procarbazine, puromycin, raltitrexed, tariquidar, temozolomide, thiamiprine, thioguanine, tiazofurin, tipifarnib, tirapazamine, troxacitabine, trimetrexate, uracil mustard (uramustine), vindesine or zorubicin.

A representative example of the anticancer agent is selected from 9-aminocamptothecin, bicalutamide, carboplatin, cyclophosphamide, cytarabine, daunorubicin, docetaxel, doxorubicin, fluorouracil, gemcitabine, idarubicin, leuprolide, melphalan, methotrexate, tirapazamine, troxacitabine, vindesine or zorubicin.

Still further in the twenty-sixth embodiment, the antidepressant referred to in the twenty-fifth embodiment hereinabove also includes an antimaniac and antipsychotic agent and is specifically selected from S-adenosylmethionine, amineptine, amisulpride, amoxapine, aripiprazole, benperidol, caroxazone, carpipramine, clocapramine, clomacran, clospirazine, clozapine, demexiptiline, desipramine, droperidol, duloxetine, fencamine, fluoxetine, fluspirilene, fluvoxamine, 5-hydroxytryptophan (oxitriptan), indalpine, indeloxazine hydrochloride, iproclozide, iproniazid, isocarboxazid, levophacetoperane, maprotiline, metapramine, milnacipran, minaprine, moclobemide, molindone, mosapramine, nemonapride, nialamide, nomifensine, nortriptyline, octamoxin, olanzapine, oxypertine, paroxetine, pimozide, pipamperone, protriptyline, reboxetine, remoxipride, rolipram, roxindole, sertindole, sertraline, spiperone, sulpiride, sultopride, tianeptine, timiperone, tofenacin, tranylcypromine, viloxazine, benmoxine, rolicyprine or ziprasidone.

A representative example of the antidepressant is selected from desipramine, duloxetine, fluoxetine, fluvoxamine, moclobemide, nortriptyline, paroxetine, reboxetine or sertraline. A representative example of the antidepressant includes an antimanic and antipsychotic agent that is selected from aripiprazole, clozapine, olanzapine or ziprasidone.

Still further in the twenty-sixth embodiment, the anticonvulsant agent referred to in the twenty-fifth embodiment hereinabove is selected from acetylpheneturide, albutoin, 4-amino-3-hydroxybutyric acid, atrolactamide, n-benzyl-3-chloropropionamide, buramate, carbamazepine, cinromide, clonazepam, decimemide, dimethadione, doxenitoin, ethosuximide, ethotoin, felbamate, fosphenyloin, gabapentin, lamotrigine, levetiracetam, licarbazepine, mephenyloin, mephobarbital, metharbital, methetoin, 5-methyl-5-(3-phenanthryl)hydantoin, 3-methyl-5-phenylhydantoin, nitrazepam, oxcarbazepine, oxicarbamazepine, phenacemide, phenetharbital, pheneturide, phenobarbital, phenylmethylbarbituric acid, phenyloin, phethenylate sodium, pregabalin, primidone, progabide, remacemide, rufinamide, suclofenide, sulthiame, talampanel, tetrantoin, topiramate, valpromide, vigabatrin or zonisamide.

A representative example of the anticonvulsant agent is selected from carbamazepine, felbamate, gabapentin, lamotrigine, levetiracetam, licarbazepine, oxcarbazepine, pregabalin, topiramate, valpromide, vigabatrin or zonisamide.

Still further in the twenty-sixth embodiment, the antibacterial agent referred to in the twenty-fifth embodiment hereinabove is selected from acedapsone, acediasulfone, acetosulfone sodium, ambazone, amikacin, p-aminosalicylic acid, p-aminosalicylic acid hydrazide, amoxicillin, amphomycin, ampicillin, apalcillin, apicycline, arbekacin, aspoxicillin, azidamfenicol, azidocillin, azlocillin, aztreonam, bacampicillin, bacitracin, balofloxacin, bambermycins, benzoylpas, benzylsulfamide, betamipron, brodimoprim, 5-bromosalicylhydroxamic acid, butirosin, capreomycin, carbenicillin, carindacillin, carumonam, cefaclor, cefadroxil, cefamandole, cefatiam, cefatrizine, cefazedone, cefazolin, cefbuperazone, cefdinir, cefcapene pivoxil, cefclidin, cefditoren, cefepime, cefetamet, cefixime, cefmenoxime, cefmetazole, cefminox, cefodizime, cefonicid, cefoperazone, ceforanide, cefoselis, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin c, cephalothin, cephapirin sodium, cephradine, chloramine-B, chloramine-T, chloramphenicol, chlortetracycline, cilastatin, ciproflaxacin, clinafloxacin, clindamycin, clometocillin, clomocycline, cloxacillin, colistin, cyacetacide, cyclacillin, cycloserine, dalfopristin, dapsone, demeclocycline, deoxydihydrostreptomycin, dibekacin, dicloxacillin, dihydrostreptomycin, dirithromycin, doxycycline, enoxacin, enviomycin, epicillin, ertapenem, ethambutol, ethionamide, fenbenicillin, flomoxef, floxacillin, N2-forimicins, formylsulfisomidine, furazolium chloride, furonazide, garenoxacin, gatifloxacin, gemifloxacin, gentamycin, glyconiazide, n4-beta-d-glucosylsulfanilamide, gramicidin(s), grepafloxacin, guamecycline, hetacillin, imipenem, isepamicin, isoniazid, kanamycin(s), lenampicillin, lincomycin, linezolide, lomefloxacin, loracarbef, lymecycline, mafenide, meclocycline, meropenem, metampicillin, methacycline, methicillin, 4′-(methylsulfamoyl)sulfanilanilide, mezlocillin, micronomicin, mikamycin, minocycline, morphazinamide, moxalactam, moxifloxacin, nafcillin, negamycin, neomycin, netilmicin, nifuradene, nitrofurantoin, noprysulfamide, norfloxacin, novobiocin, opiniazide, oxacillin, oxytetracycline, panipenem, paromomycin, pazufloxacin, penamecillin, penethamate hydriodide, penicillin(s), penimepicycline, pexiganan, phenethicillin, phenyl aminosalicylate, phthalylsulfacetamide, phthalylsulfathiazole, picloxydine, pipacycline, pipemidic acid, piperacillin, pivampicillin, pivcefalexin, polymyxin, porfiromycin, primycin, pristinamycin, protionamide, pyrazinamide, quinacillin, quinupristin, ramoplanin, ribostamycin, rifabutin, rifalazil, rifamide, rifamycin sv, rifampin, rifapentine, rifaximin, ristocetin, ritipenem, rolitetracycline, salazosulfadimidine, salinazid, sancycline, sisomicin, sitafloxacin, solasulfone, sparfloxacin, spectinomycin, streptolydigin, streptomycin, streptonicozid, subathizone, 4,4′-succinylsulfathiazole, succisulfone, sulbenicillin, sulfachrysoidine, sulfanilic acid, 2-p-sulfanilylanilinoethanol, sulfinyldianiline, sulfoxone sodium, 4′-sulfanilylsulfanilamide, sulfoniazide, sulfabenzamide, sulfacetamide, sulfachlorpyridazine, sulfacytine, sulfadiazine, sulfadicramide, sulfadimethoxine, sulfadoxine, sulfaethidole, sulfaguanidine, sulfaguanole, sulfalene, sulfaloxic acid, sulfamerazine, sulfameter, sulfamethazine, sulfamethizole, sulfamethomidine, sulfamethoxazole, sulfamethoxypyridazine, sulfamethylthiazole, sulfametrole, sulfamidochrysoidine, sulfamoxole, sulfanilamide, 4-sulfanilamidosalicylic acid, p-sulfanilylbenzylamine, sulfanilylurea, n-sulfanilyl-3,4-xylamide, sulfaperine, sulfaphenazole, sulfaproxyline, sulfapyrazine, sulfasomizole, sulfasymazine, sulfathiazole, sulfathiourea, sulfisomidine, sulfisoxazole, sultamicillin, sulfatolamide, talampicillin, taurolidine, teicoplanin, temocillin, tetroxoprim, thiamphenicol, thiazosulfone, thiacetazone, thiostrepton, ticarcillin, tigemonam, tiocarlide, tobramycin, tosufloxacin, trimethoprim, trospectomycin, trovafloxacin, tuberactinomycin, tyrocidine, vancomycin, viomycin or virginiamycin.

A representative example of the anti-bacterial agent is selected from amoxicillin, ampicillin, cefadroxil, cefalexin, cefixime, cefotaxime, cefuroxime, cephalexin, chloramphenicol, chlortetracycline, ciproflaxacin, clavulanate, clinafloxacin, clindamycin, dapsone, doxycycline, ethambutol, gatifloxacin, gentamycin, nadifloxacin, nalidixic acid, norfloxacin, oflaxacin, oxacillin, panipenem, penicillins, salbactam, streptomycin, sultamicillin or vancomycin.

Still further in the twenty-sixth embodiment, the antifungal agent referred to in the twenty-fifth embodiment hereinabove is selected from acrisorcin (9-aminoacrindine compound with 4-hexylresorcinol (1:1)), amphotericin B, anidulafungin, azaserine, bromosalicylchloranilide, buclosamide, candicidin, caspofungin, chlordantoin, exalamide, flucytosine, loflucarban, lucensomycin, magenta I, mepartricin, micafungin, natamycin, nystatin, perimycin, pyrroInitrin, salicylanilide or tubercidin.

Still further in the twenty-sixth embodiment, the antiviral agent referred to in the twenty-fifth embodiment hereinabove is selected from abacavir, acyclovir, adefovir, amantadine, amidinomycin, amprenavir, atazanavir, atevirdine, capravirine, cidofovir, delavirdine, didanosine, dideoxyadenosine, efavirenz, emtricitabine, entecavir, famciclovir, ganciclovir, imiquimod, indinavir, lamivudine, lopinavir, mantadine, methisazone, 5-(methylamino)-2-deoxyuridine (madu), moroxydine, nelfinavir, nevirapine, oseltamivir, penciclovir, resiquimod, ribavirin, rimantadine, ritonavir, saquinavir, stallimycin, tenofovir, tipranavir, trimetazidine, tromantadine, valacyclovir, valganciclovir, vidarabine, zalcitabine or zanamivir.

A representative example of the antiviral agent is selected from abacavir, acyclovir, adefovir, amprenavir, cidofovir, didanosine, efavirenz, emtricitabine, famciclovir, ganciclovir, indinavir, lamivudine, lopinavir, nelfinavir, nevirapine, oseltamivir, penciclovir, ritonavir, saquinavir, trimetazidine, valacyclovir, valganciclovir, vidarabine, zalcitabine or zanamivir.

Still further in the twenty-sixth embodiment, the antimalarial agent referred to in the twenty-sixth embodiment hereinabove is selected from amodiaquine, chlorguanide, chloroquine, chlorproguanil, cycloguanil, hydroxychloroquine, mefloquine, 3-methylarsacetin, pamaquine, plasmocid, primaquine, pyronaridine, quinocide or tafenoquine

Still further in the twenty-sixth embodiment, the antidiabetic agent referred to in the twenty-fifth embodiment hereinabove is selected from acetohexamide, buformin, carbutamide, chlorpropamide, fidarestat, glibornuride, gliclazide, glimepiride, glipizide, gliquidone, glisoxepid, glyburide, glybuthiazol(e), glybuzole, glyhexamide, glymidine, glypinamide, metformin, phenformin, pioglitazone, repaglinide, rosiglitazone, tolazamide, tolbutamide, tolcyclamide, troglitazone or voglibose.

Still further in the twenty-sixth embodiment, the antiulcer agent referred to in the twenty-fifth embodiment hereinabove includes a proton pump inhibitor and said antiulcer agent is selected from aldioxa, benexate HCl, carbenoxolone, cetraxate, cimetidine, ebrotidine, ecabapide, esaprazole, esomeprazole, famotidine, irsogladine, lafutidine, lansoprazole, leminoprazole, 5-methylmethionine, nizatidine, omeprazole, pantoprazole, pirenzepine, polaprezinc, rabeprazole, ranitidine, rebamipide, rotraxate, roxatidine, telenzepine or troxipide.

Still further in the twenty-sixth embodiment, the antioxidant referred to in the twenty-fifth embodiment hereinabove includes a free radical scavenger and the antioxidant is selected from BTX-51072 (4,4-dimethyl-3,4-dihydro-2H-1,2-benzoselenazine), carnosine, melatonin, (+)-R-pramipexole, SCMC-Lys (S-carboxymethyl-L-cysteine Lysine salt H₂O), stobadine or zeatin.

Still further in the twenty-sixth embodiment, the vitamin referred to in the twenty-fifth embodiment hereinabove is selected from acetiamine (diacethiamine or D.A.T.), benfotiamine (s-benzoylthiamine monophosphate or BTMP), biotin (vitamin H or coenzyme R), bisbentiamine (O-benzoylthiamine disulfide), cetotiamine (O,S-dicarbethoxythiamine or DCET), cobamamide (vitamin B₂ coenzyme), cyanocobalamin (vitamin B₁₂), folic acid (vitamin M), fursultiamine (thiamine tetrahydrofurfuryl disulfide), hydroxocobalamin (vitamin B₁₂ₐ), nicotinamide, octotiamine, prosultiamine, thiamine (vitamin B₁) or vitamin K₅.

As has been indicated hereinabove that the twenty-fifth embodiment also encompasses within its scope a compound of formula (I) wherein the drug or therapeutic agent containing an amino group is selected from the drugs belonging to several other therapeutic areas (including those drugs that are classified on the basis of their mechanism of action). Thus, for the purpose of this invention, the twenty-sixth embodiment also encompasses a compound of formula (I); wherein the drug containing amino group is generically selected from the class of drugs falling under several other therapeutic areas (including those drugs that are classified on the basis of their mechanism of action) and is specifically selected from: an abortifacient/interceptive such as sulprostone; an anesthetic selected from ambucaine, benoxinate, benzocaine, betoxycaine, bupivacaine, butacaine, butamben, butanilicaine, butethamine, carticaine, chloroprocaine hydrochloride, dibucaine hydrochloride, dimethocaine, diperodon hydrochloride, etidocaine, etoxadrol, β-eucaine, euprocin, hexylcaine hydrochloride, hydroxytetracaine, isobutyl p-aminobenzoate, ketamine, lidocaine, leucinocaine mesylate, mepivacaine, meprylcaine, metabutoxycaine, octacaine, orthocaine, pentobarbital, piridocaine, prilocalne, procaine, proparacaine, propoxycaine hydrochloride, pyrrocaine, ropivacaine, tetracaine hydrochloride, thialbarbital, thiamylal, tolycaine, tricaine, trimecaine or urethan; an anorexic agent selected from a minorex, chlorphentermine, clobenzorex, cloforex, clortermine, n-ethylamphetamine, fenfluramine, fenproporex, mefenorex, norpseudoephedrine, pentorex, phenmetrazine, phentermine, picilorex or methamphetamine; an anthelmintic agent selected from albendazole, amocarzine, amphotalide, becanthone, cyclobendazole, diphenane, hycanthone, kainic acid, lucanthone, mebendazole, niridazole, nitazoxanide, oxamniquine, pelletierine, piperazine, quinacrine, thiabendazole or thymyl N-isoamylcarbamate; an agent for treating alopecia such as finasteride; an antiamebic agent selected from carbarsone, dehydroemetine, diphetarsone, emetine, thiocarbarsone, glycobiarsol or tetracycline; an antiandrogen agent such as flutamide or nilutamide; an antiarthritic/antirheumatic agent selected from glucosamine, leflunomide or penicillamine; an antiasthmatic agent selected from domitroban, formoterol, pranlukast, ramatroban, suplatast tosylate, traxanox, zafirlukast or zileuton; an antidiarrheal agent selected from alkofanone, racecadotril or zaldaride; an antidiuretic selected from desmopressin, felypressin, lypressin, ornipressin, terlipressin or vasopressin; an antiemetic agent selected from alizapride, aprepitant, azasetron, bromopride, clebopride, dolasetron, domperidone, granisetron, itasetron, methallatal, metoclopramide, metopimazine, pipamazine, ramosetron, trimethobenzamide or tropisetron; an antiglaucoma agent selected from acetazolamide, brinzolamide, dorzolamide, befunolol, bimatoprost, brimonidine or levobunolol; an antigout agent selected from allopurinol, carprofen, colchicine or orotic acid; an antihyperthyroid agent selected from propylthiouracil or thiobarbital; an antihypothyroid agent such as thyroxine; an antimigraine agent selected from almotriptan, alpiropride, eletriptan, ergotamine, frovatriptan, lisuride, methysergide, naratriptan, rizatriptan, sumatriptan or zolmitriptan; an antimuscarinic/mydriatic agent selected from ambutonium bromide, aminopentamide, benzetimide, buzepide, camylofine, darifenacin, fenpiverinium bromide or isopropamide iodide; an antiosteoporotic agent selected from alendronic acid, incadronic acid or pamidronic acid; an antiprostatic agent used for treating hypertrophy selected from doxazosin, epristeride, mepartricin, tamsulosin or terazosin; an antiprotozoal agent selected from acetarsone, Acranil®, aminitrozole, anisomycin, azanidazole, benznidazole, eflornithine, hydroxystilbamidine, lauroguadine, melarsoprol, mepartricin, n-methylglucamine, nitazoxanide, oxophenarsine hydrochloride, pentamidine, propamidine, puromycin, pyrimethamine, quinapyramine, stilbamidine, suramin sodium, tenonitrozole, trypan red or tryparsamide; an antipsoriatic agent such as 6-azauridine; an antiseptic agent selected from aminacrine, aminoquinuride, bisdequalinium chloride, chlorhexidine, chloroazodin, dequalinium chloride, dibromopropamidine, dodecarbonium chloride, ethacridine, hexamidine, hexetidine, iodopyrrole, laurolinium acetate, nitroakridin 3582, noxythiolin, oxymethurea or triclocarban; an antispasmodic agent selected from ambutonium bromide, aminopentamide, buzepide, camylofine, darifenacin, drotaverine, etomidoline, fenalamide, fenpiverinium bromide, hydramitrazine, isopropamide iodide, nicofetamide, octamylamine, phenamacide hydrochloride, pramiverin, proglumide, racefemine or tiropramide; an antitussive agent selected from alloclamide, benzonatate or fominoben; an anxiolytic agent selected from abecarnil, azacyclonol, benzoctamine, bromazepam, calcium N-carbamoylaspartate, chlordiazepoxide, clorazepic acid, cloxazolam, cyclarbamate, emylcamate, ethyl etifoxine, flesinoxan, hydroxyphenamate, loflazepate, lorazepam, mecloralurea, meprobamate, mexazolam, nordazepam, oxazepam, oxazolam, tybamate or valnoctamide; a cathartic agent/laxative selected from bisoxatin acetate or oxyphenisatin acetate; a choleretic agent selected from osalmid or sincalide; a cholinergic agent selected from bethanechol chloride, eptastigmine, eseridine, guanidine, dexpanthenol, carbachol or physostigmine; a decongestant selected from amidephrine, cyclopentamine, ephedrine, epinephrine, fenoxazoline, indanazoline, naphazoline, nordefrin, octodrine, oxymetazoline, phenylephrine, phenylpropanolamine, phenylpropylmethylamine, propylhexedrine, pseudoephedrine, tetrahydrozoline, tramazoline, tuaminoheptane, tymazoline or xylometazoline; an emetic such as cephaeline; an enzyme cofactor selected from acetiamine, benfotiamine, bisbentiamine, cetotiamine, dexpanthenol, fursultiamine, octotiamine, pantothenic acid, prosultiamine, sapropterin, thiamine, thiamine diphosphate or thiamine disulfide; an agent that acts as an expectorant selected from ambroxol or bromhexine; a gastroprokinetic agent selected from piboserod, alvimopan, cinitapride, cisapride, loxiglumide, mosapride, prucalopride, renzapride or tegaserod; a hemostatic agent selected from adrenalone, cephalins, aminocaproic acid, carbazochrome sodium sulfonate, ethamsylate, tranexamic acid, tolonium chloride or vapreotide; a hepatoprotectant selected from s-adenosylmethionine, citiolone, orazamide, timonacic (thioproline), methionine, protoporphyrin ix or tiopronin; an immunomodulator selected from bropirimine, thalidomide, ubenimex, bucillamine, imiquimod, leflunomide, mitoxantrone, pidotimod, procodazole, romurtide or thymopentin; an immunosuppressant selected from azathioprine, gusperimus or mizoribine; a mucolytic agent selected from carbocysteine, erdosteine, letosteine, mecysteine or stepronin; a muscle relaxant selected from afloqualone, baclofen, carisoprodol, chlorphenesin carbamate, chlorzoxazone, mephenoxalone, methocarbamol, phenprobamate, tizanidine, hexacarbacholine bromide, metaxalone or dantrolene; a mydriatic selected from phenylephrine hydrochloride or yohimbine; a narcotic antagonist such as amiphenazole; a neuroprotective agent selected from aptiganel, licostinel, repinotan, riluzole, citicoline or memantine; a drug used as a nootropic/cognitive enhancer selected from amphetamine, atomoxetine, bemegride, bifemelane, dextroamphetamine, etifelmin, etryptamine, fencamfamine, fenethylline, fenozolone, ipidacrine, leteprinim, mefexamide, methylphenidate, modafinil, nebracetam, nefiracetam, oxiracetam, pemoline, pipradrol, piracetam, posatirelin, pramiracetam, sulbutiamine, tacrine or velnacrine; a drug which acts as a respiratory stimulant such as almitrine; a drug which is used as a sedative/hypnotic selected from acecarbromal, allobarbital, amobarbital, amphenidone, aprobarbital, apronalide, barbital, brallobarbital, bromisovalum, butalbital, butallylonal, butethal, butoctamide, carbromal, carbubarb, carfimate, cyclobarbital, cyclopentobarbital, dexmedetomidine, diethylbromoacetamide, ectylurea, enallylpropymal, ethinamate, febarbamate, 5-furfuryl-5-isopropylbarbituric acid, glutethimide, haloxazolam, heptabarbital, hexethal sodium, hexobarbital, methitural, methyprylon, narcobarbital, nealbarbital, niaprazine, pentobarbital, phenallymal, piperidione, propallylonal, proxibarbal, reposal, rilmazafone, secobarbital sodium, talbutal, tetrabarbital, valdetamide, vinbarbital sodium or vinylbital; a vulnerary such as allantoin; a drug that acts as an α-adrenergic agonist selected from adrafinil, adrenalone, amidephrine, apraclonidine, budralazine, clonidine, cyclopentamine, dexmedetomidine, dimetofrine, dipivefrin, ecabapide, ephedrine, epinephrine, fenoxazoline, guanabenz, guanfacine, hydroxyamphetamine, ibopamine, indanazoline, isometheptene, mephentermine, metaraminol, methoxamine, methylhexaneamine, midodrine, mivazerol, modafinil, moxonidine, naphazoline, norepinephrine, norfenefrine, octodrine, octopamine, oxymetazoline, phenylephrine hydrochloride, phenylpropanolamine, phenylpropylmethylamine, pholedrine, propylhexedrine, pseudoephedrine, rilmenidine, synephrine, talipexole, tetrahydrozoline, tiamenidine, tramazoline, tuaminoheptane, tymazoline, tyramine or xylometazoline; a drug that acts as a β-adrenergic agonist selected from albuterol (salbutamol), bambuterol, bitolterol, carbuterol, clenbuterol, clorprenaline, denopamine, dioxethedrine, dopexamine, ephedrine, epinephrine, ethylnorepinephrine, fenoterol, formoterol, hexoprenaline, ibopamine, isoetharine, isoproterenol, mabuterol, metaproterenol, methoxyphenamine, oxyfedrine, pirbuterol, prenalterol, procaterol, protokylol, reproterol, rimiterol, ritodrine, salmeterol, soterenol, terbutaline, tretoquinol, tulobuterol or xamoterol; a drug that acts as an α-adrenergic blocker selected from amosulalol, arotinolol, doxazosin, ergoloid mesylates, fenspiride, idazoxan, indoramin, labetalol, monatepil, prazosin, tamsulosin, terazosin, tolazoline, trimazosin or yohimbine; a drug that acts as a β-adrenergic blocker selected from acebutolol, amosulalol, alprenolol, arotinolol, atenolol, befunolol, betaxolol, bevantolol, bisoprolol, bopindolol, bucindolol, bucumolol, bufetolol, bufuralol, bunitrolol, bupranolol, butidrine, butofilolol, carazolol, carteolol, carvedilol, celiprolol, cetamolol, cloranolol, dilevalol, esmolol, indenolol, labetalol, landiolol, levobunolol, mepindolol, metipranolol, metoprolol, moprolol, nadolol, nadoxolol, nebivolol, nifenalol, nipradilol, oxprenolol, penbutolol, pindolol, practolol, pronethalol, propranolol, sotalol, sulfinalol, talinolol, tertatolol, tilisolol, timolol, toliprolol or xibenolol; a dopamine receptor agonist selected from bromocriptine, cabergoline, carmoxirole, dopexamine, fenoldopam, ibopamine, pergolide, pramipexole, quinagolide, ropinirole, roxindole or talipexole; a dopamine receptor antagonist selected from amisulpride, amisulpride, clebopride, domperidone, metoclopramide or mosapramine; an α-glucosidase inhibitor selected from acarbose or voglibose; a matrix metalloproteinase inhibitor such as batimastat; a monoamine oxidase inhibitor selected from iproclozide, iproniazid, isocarboxazid, lazabemide, moclobemide, mofegiline, octamoxin, phenelzine, phenoxypropazine, pivalylbenzhydrazine or tranylcypromine; a neutral endopeptidase inhibitor such as ecadotril; a potassium channel blocker such as fampridine; a prolactin inhibitor selected from metergoline or terguride; a protease inhibitor selected from camostat, gabexate, nafamostat or sepimostat; 5α-Reductase inhibitor such as dutasteride; a reverse transcriptase inhibitor such as stavudine; a serotonin receptor agonist such as eltoprazine; a serotonin receptor antagonist such as alosetron; and a thromboxane A₂-receptor antagonist such as daltroban.

In a twenty-seventh embodiment, the invention encompasses a compound of formula (I), wherein: D is a drug containing a hydroxyl group capable of forming a bio-cleavable covalent linkage with a linker;

X¹ is oxygen;
each of X², Y, Z¹; Z², A, R¹ and R² is as defined in the first embodiment hereinabove;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a twenty eighth embodiment, the invention encompasses a compound of formula (I), wherein: D and X¹ are as defined in the twenty seventh embodiment hereinabove; each X², Y, Z¹, Z², A, R¹ and R² is as defined in the second embodiment hereinabove;

with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a twenty ninth embodiment, the invention encompasses a compound of formula (I), wherein: D and X¹ are as defined in the twenty seventh embodiment hereinabove;

each of X², Y, Z¹ and Z² is as defined in the second embodiment hereinabove;
A is selected from 1,2-phenylene, 1,3-phenylene, 1,4-phenylene, 2,3-pyridine, 3,4-pyridine, 2,4-pyridine, 2,5-pyridine or 2,6-pyridine;
R¹ is hydrogen and R² is alkyl, cycloalkyl, aryl or aralkyl; or R² is hydrogen and R¹ is alkyl, cycloalkyl, aryl or aralkyl;
in all its stereoisomeric forms and pharmaceutically acceptable salts thereof.

In a thirtieth embodiment, the invention encompasses a compound of formula (I), wherein: each of D, X¹, X², Y, Z¹ and Z² is as defined in the twenty eighth embodiment hereinabove,

A is selected from a bond, CH═CH or CR⁹R¹⁰; where R⁹ and R¹⁰ are independently selected from hydrogen or C₁₋₆ alkyl;

R¹ is hydrogen and R² is alkyl, cycloalkyl, aryl or aralkyl; or R² is hydrogen and R¹ is alkyl, cycloalkyl, aryl or aralkyl;

in all its stereoisomeric forms and pharmaceutically acceptable salts thereof.

In a thirty-first embodiment, the invention encompasses a compound of formula (I), wherein: each of D, X¹, X², Y, Z¹ and Z² is as defined in the twenty eighth embodiment hereinabove,

A is selected from S, SO, SO₂ or S—S; provided that when A is S, then a and b is 3;

R¹ is hydrogen and R² is alkyl, cycloalkyl, aryl or aralkyl; or R² is hydrogen and R¹ is alkyl, cycloalkyl, aryl or aralkyl;

in all its stereoisomeric forms and pharmaceutically acceptable salts thereof.

In a thirty-second embodiment, the invention encompasses a compound of formula (I), wherein: D, the drug containing a hydroxyl group capable of forming a covalent bio-cleavable linkage with a linker, referred to in the twenty-seventh, twenty-eighth, twenty-ninth, thirtieth and thirty-first embodiments, is selected from an antiinflammatory and analgesic drug, a cardiovascular drug, a glucocorticoid, an antiallergic agent, anticancer agent, an antidepressant, an anticonvulsant agent, an antibacterial agent, an antifungal agent, an antiviral agent, an antimalarial agent, an antidiabetic agent, an antiulcer agent, an antioxidant or a vitamin. The thirty-second embodiment also encompasses within its scope a drug containing a hydroxyl group is selected from the drugs that belong to several other therapeutic areas (including those drugs that are classified on the basis of their mechanism of action). In this embodiment, other variables X¹, X², Y, Z¹ and Z²; A, R¹ and R² in the compounds of formula (I) are as defined above; with the provisos that:
a) when A is S, then a and b is 3; or
b) when A is D-isosorbide skeleton or 1,4-anhydroerythritol skeleton, then a and b is 0; and
   in all its stereoisomeric forms and pharmaceutically acceptable salts thereof.

In thirty-third embodiment, the invention encompasses a compound of formula (I), wherein D, the drug containing a hydroxyl group capable of forming a covalent bio-cleavable linkage with a linker, is a glucocorticoid;

X¹ is a bond;
X² oxygen;
Y is spacer group as defined in the first embodiment hereinabove,
Z¹, Z², A, R¹ and R² are as defined in the second embodiment hereinabove; and
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a thirty-fourth embodiment, the invention encompasses a compound of formula (I), wherein each of D, X¹ and X² is as defined in the thirty-third embodiment hereinabove;

Y is a spacer group selected from:

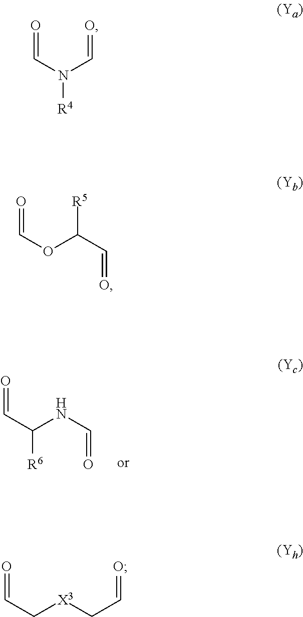

Z¹, Z², A, R¹ and R² are as defined in the second embodiment hereinabove; and
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a thirty-fifth embodiment, the invention encompasses a compound of formula (I), wherein: the glucocorticoid referred to in the thirty-third and thirty-fourth embodiments hereinabove is selected from 21-acetoxypregnenolone, alclometasone, algestone, amcinonide, beclomethasone, betamethasone, budesonide, chloroprednisone, ciclesonide, clobetasol, clobetasone, clocortolone, cloprednol, corticosterone, cortisone, deflazacort, desonide, desoximetasone, dexamethasone, diflorasone, diflucortolone, difluprednate, enoxolone, fluazacort, flucloronide, fludrocortisone, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 21-diethylaminoacetate, prednisone, prednival, prednylidene, rimexolone, triamcinolone or triamcinolone acetonide.

A representative example of the glucocorticoid is selected from betamethasone, budesonide, dexamethasone, hydrocortisone, fludrocortisone, fluticasone, prednisolone or triamcinolone.

In a thirty-sixth embodiment, the antiinflammatory and analgesic drug referred to in the thirty-second embodiment is generically selected from an opioid, a steroid (i.e., glucocorticoids) or a non-steroidal anti-inflammatory drug (NSAIDs) and is specifically selected from acetaminophen, acetaminosalol, 21-acetoxypregnenolone, alclometasone, alfa-aluminum bis(acetylsalicylate), 3-amino-4-hydroxybutyric acid, balsalazide, benzylmorphine, bisabolol, bucetin, budesonide, bufexamac, buprenorphine, butorphanol, capsaicine, chlorobutanol, ciramadol, codeine, deflazacort, diflorasone, desomorphine, desonide, desoximetasone, dezocine, diflorasone, diflucortolone, diflunisal, difluprednate, dihydrocodeine, dihydromorphine, dihydroxyaluminum acetylsalicylate, dimepheptanol, ditazol, enoxolone, eptazocine, ethylmorphine, etofenamate, eugenol, fendosal, fepradinol, floctafenine, fluazacort, fluocinonide, fluocortin butyl, fluprednidene acetate, gentisic acid, glafenine, glucametacin, halcinonide, halobetasol propionate, halometasone, halopredone acetate, hydrocortisone, hydromorphone, hydroxypethidine, ibuproxam, isoladol, isoxicam, ketobemidone, p-lactophenetide, levorphanol, lornoxicam, loteprednol etabonate, mazipredone, meloxicam, meptazinol, mesalamine, metazocine, metopon, mometasone furoate, morphine, nalbuphine, norlevorphanol, normorphine, olsalazine, oxaceprol, oxametacine, oxycodone, oxymorphone, oxyphenbutazone, pentazocine, perisoxal, piroxicam, phenazocine, phenoperidine, phenylramidol, phenylsalicylate, rimexolone, salacetamide, salicin, salicylamide, salsalate, sulfasalazine, tenoxicam, tixocortol, tramadol, viminol or ximoprofen,

A representative example of the antiinflammatory and analgesic drug (consisting of glucocorticoids, NSAIDs and opioids) is selected from acetaminophen, balasalazide, budesonide, codeine, deflazacort, desomorphine, diflunisal, dihydrocodeine, dihydromorphine, eugenol, glucametacin, halobetasol propionate, halometasone, hydrocortisone, hydromorphone, levorphanol, meloxicam, mesalamine, mometasone furoate, morphine, norlevorphanol, normorphine, olsalzine, oxycodone, oxymorphone, piroxicam, sulfasalazine or tramadol.

Still further in the thirty-sixth embodiment, the cardiovascular agent referred to in the thirty-second embodiment is generically selected from an antihypertensive agent such as an angiotesnsin converting enzyme (ACE) inhibitor, a beta-blocker, a sartan (i.e., angiotensin II blockers), an antithrombotic and vasoactive agent, an anti-hyperlipidemic agent (including HMG-CoA-reductase inhibitors (i.e., statins)), a fibrate, an antianginal agent, an antiarrhythmic agent, an antihypotensive agent, a calcium channel blocker, a calcium regulator, a cardiotonic agent, a cardioprotective agent, a diuretic, a vasodilator or a vasoprotectant; and is specifically selected from acadesine, acebutolol, ajmaline, alprenolol, ambuside, amosulalol, angiotensin, arotinolol, atenolol, atorvastatin, bamethan, benzarone, benziodarone, beraprost, betaxolol, bevantolol, bisoprolol, bosentan, bradykinin, brovincamine, bucindolol, bucumolol, bufeniode, buflomedil, bufuralol, bunitrolol, bupranolol, butofilolol, cadralazine, calcifediol, calcitriol, canrenone (hydroxyl of its ketoxime), carazolol, l-carnitine (levocarnitine), carteolol, carvedilol, celiprolol, cerivastatin, cetamolol, chlorthalidone, chromocarb, cicletanine, clobenfurol, clobenoside, convallatoxin, cyclandelate, denopamine, deslanoside, digitalin, dihydrotachysterol, dilevalol, dimetofrine, diosmin, dobesilate calcium, dobutamine, dopamine, dopexamine, efloxate, eledoisin, enoximone, epanolol, erythrophleine, escin, etafenone, ethacrynic acid, etilefrin, ezetimibe, fenofibrate, fenoldopam, fluvastatin, furazabol, gepefrine, gitoxin, guanoxabenz, heptaminol, ibudilast, ifenprodil, iloprost, indenolol, ipriflavone, isosorbide, isoxsuprine, kallidin, khellin, labetalol, lanatosides, leucocyanidin, levcromakalim, limaprost, losartan, lovastatin, meglutol, mannitol, mepindolol, metaraminol, methoxamine, methyldopa, metipranolol, metoprolol, mevastatin, midodrine, moprolol, nadolol, naftopidil, nebivolol, neriifolin, nicomol, nicotinyl alcohol, nifenalol, nipradilol, norepinephrine, nylidrin, oleandrin, olmesartan, oxprenolol, oxyfedrine, penbutolol, pentrinitrol, perhexyline, phenactropinium chloride, phentolamine, pholedrine, pildralazine, pindolol, pirifibrate, pitavastatin, pravastatin sodium, prenalterol, probucol, pronethalol, propranolol, proscillaridin, prostaglandin e₁, protheobromine, protoveratrines, ouabain, quercetin, ranolazine, rescimetol, resibufogenin, rutin sampatrilat, scillaren, scillarenin, simvastatin, sotalol, spironolactone, sulfinalol, suloctidil, synephrine, talinolol, tertatolol, thyropropic acid, ticrynafen, timolol, tinofedrine, toliprolol, tricromyl, trimazosin, troxerutin, ubiquinones, vincamine, viquidil, xamoterol, xanthinol niacinate or xipamide.

A representative example of the cardiovascular agent is a beta-blocker that is selected from atenolol, bupranolol, carvedilol, labetalol, metipranolol, metoprolol, nadolol, pindolol, propranolol or timolol.

Another representative example of the cardiovascular agent is a sartan selected from losartan or olmesartan.

Another representative example of the cardiovascular agent is an antithrombotic and vasoactive agent that is selected from beraprost, clinprost, dalteparin, dipyridamole, enoxaparin, ifenprodil, iloprost, heparin, lamifiban, nadroparin, reviparin sodium salt, suloctidil, taprostene, tinzaparin, xanthinol niacinate or ximelagatran.

Yet another representative example of the cardiovascular agent is an anticoagulant that is selected from acenocoumarol, anisindione, bromindione, clorindione, coumetarol, dicumarol, diphenadione, ethyl biscoumacetate, ethylidene dicoumarol, fluindione, heparin, phenindione, phenprocoumon, tioclomarol or warfarin.

Yet another representative example of the cardiovascular agent is an anti-hyperlipidemic agent (i.e., statins, fibrates, etc.) that is selected from atorvastatin, cerivastatin, ezetimibe, fenofibrate, fluvastatin, lovastatin, mevastatin, pirifibrate, pitavastatin, pravastatin sodium or simvastatin;

Yet another representative example of the cardiovascular agent is an antianginal agent that is selected from bevantolol, bucumolol, bufuralol, limaprost, nifenalol, nipradilol, oxyfedrine, pronethalol, ranolazine, sotalol or toliprolol.

Yet another representative example of the cardiovascular agent is an antiarrhythmic agent that is selected from adenosine, amiodarone, bufetolol, butidrine, cloranolol, dofetilide, esmolol, hydroquinidine, landiolol, lorajmine, nadoxolol, pirmenol, practolol, prajmaline, propafenone, pyrinoline, quinidine, tilisolol or xibenolol.

Yet another representative example of the cardiovascular agent is an antihypertensive agent that is selected from angiotensin, dimetofrine, dopamine, etilefrin, gepefrine, heptaminol, metaraminol, methoxamine, midodrine, norepinephrine, pholedrine or synephrine.

Yet another representative example of the cardiovascular agent is a calcium channel blocker such as etafenone.

Yet another representative example of the cardiovascular agent is a calcium regulator that is selected from calcifediol, calcitriol, dihydrotachysterol or ipriflavone.

Yet another representative example of the cardiovascular agent is a cardiotonic agent that is selected from convallatoxin, denopamine, deslanoside, digitalin, dobutamine, dopamine, dopexamine, enoximone, erythrophleine, gitoxin, lanatosides, neriifolin, oleandrin, ouabain, prenalterol, proscillaridin, resibufogenin, scillaren, scillarenin, ubiquinones or xamoterol.

Yet another representative example of the cardiovascular agent is a cardioprotective agent is acadesine.

Yet another representative example of the cardiovascular agent a diuretic that is selected from ambuside, canrenone, chlorthalidone, ethacrynic acid, isosorbide, mannitol, protheobromine, spironolactone, ticrynafen or xipamide.

Yet another representative example of the cardiovascular agent is a vasodilator that is selected from bamethan, benziodarone, beraprost, bosentan, bradykinin, brovincamine, bufeniode, buflomedil, clobenfurol, cyclandelate, efloxate, eledoisin, etafenone, ibudilast, ifenprodil, iloprost, isoxsuprine, kallidin, khellin, nicotinyl alcohol, nylidrin, pentrinitrol, perhexyline, prostaglandin E₁, suloctidil, tinofedrine, tricromyl, vincamine, viquidil or xanthinol niacinate.

Yet another representative example of the cardiovascular agent is a vasoprotectant that is selected from benzarone, chromocarb, clobenoside, diosmin, dobesilate calcium, escin, leucocyanidin, quercetin, rutin or troxerutin.

Still further in the thirty-sixth embodiment, the antiallergic agent referred to in the thirty-second embodiment is generically selected from a steroidal bronchodilator, a mast cell stabilizer or an antihistamine and is specifically selected from amlexanox, bambuterol, beclomethasone, cetoxime, ciclesonide, ebastine, fexofenadine, flunisolide, fluticasone and its approved esters, n-hydroxyethylpromethazine chloride, hydroxyzine, ibudilast, methyl prednisolone, montelukast sodium, pentigetide, repirinast, roxatidine, salbutamol, salmeterol, suplatast, terfenadine or tranilast.

A representative example of the antiallergic agent is an antihistamine that is selected from cetoxime, ciclesonide, ebastine, n-hydroxyethylpromethazine chloride, hydroxyzine, fexofenadine, roxatidine or terfenadine.

Still further in the thirty-sixth embodiment, the anticancer agent referred to in the thirty-second embodiment is selected from aclacinomycins, ancitabine, anthramycin, arzoxifene, azacitidine, bicalutamide, bleomycins, bropirimine, broxuridine, buserelin, calusterone, capecitabine, carubicin, CC-1065 (NSC 298223), chlorozotocin, chromomycins, cladribine, cytarabine, daunorubicin, decitabine, defosfamide, diethylstilbestrol, docetaxel, doxifluridine, doxorubicin, droloxifene, dromostanolone, ecteinascidins, enocitabine, epirubicin, epitiostanol, estramustine, etanidazole, etoposide, fenretinide, flavopiridol, formestane, fosfestrol, fulvestrant, gemcitabine, hydroxyurea, idarubicin, irinotecan, leuprolide, marimastat, melengestrol, menogaril, 6-mercaptopurine, miltefosine, minodronate (minodronic acid), mitobronitol, mitolactol, mopidamol, nitracrine, nogalamycin, nordihydroguaiaretic acid (masoprocol), olivomycins, paclitaxel and other known paclitaxel analogs, pentostatin, peplomycin, perfosfamide, pirarubicin, podophyllotoxin, prinomastat, puromycin, ranimustine, resveratrol, roquinimex, rubitecan, seocalcitol, streptonigrin, streptozocin, temoporfin, teniposide, tenuazonic acid, tiazofurin, topotecan, troxacitabine, valrubicin, vinblastine, vincristine, vindesine, vinorelbine, zorubicin or zosuquidar.

A representative example of the anticancer agent is selected from bicalutamide, capecitabine, CC-1065 (NSC 298223), cytarabine, daunorubicin, docetaxel, doxorubicin, estramustine, etoposide, flavopiridol, gemcitabine, idarubicin, irinotecan, leuprolide, paclitaxel and other active paclitaxel analogs such as docetaxel, podophyllotoxin, resveratrol, topotecan, vinblastine or vincristine.

Still further in the thirty-sixth embodiment, the antidepressant referred to in the thirty-second embodiment is generically selected from an antimanic and antipsychotic agent and is specifically selected from acetophenazine, S-adenosylmethionine, befloxatone, bromperidol, bupropion, butaperazine, carphenazine, clopenthixol (cis-isomer), clospirazine, dixyrazine, fenpentadiol, fluanisone, flupentixol (cis-form), fluphenazine, fluspirilene, haloperidol, 5-hydroxytryptophan (oxitriptan), hypericin, melperone, moperone, mosapramine, opipramol, penfluridol, pericyazine, perimethazine, perphenazine, pipamperone, piperacetazine, pipotiazine, pyrisuccideanol, quetiapine, roxindole, spiperone, sultopride, timiperone, toloxatone, tramadol, trifluperidol or venlafaxine.

A representative example of the antidepressant is selected from bupropion, tramadol or venlafaxine.

A representative example of the antidepressant is an antimaniac and antipsychotic agent that is selected from haloperidol, quetiapine or trifluperidol.

Still further in the thirty-sixth embodiment, the anticonvulsant referred to in the thirty-second embodiment is selected from 4-amino-3-hydroxybutyric acid, atrolactamide, buramate or ganaxolone.

Still further in the thirty-sixth embodiment, the antibacterial agent referred to in the thirty-second embodiment is selected from amikacin, p-aminosalicylic acid, p-aminosalicylic acid hydrazide, amoxicillin, apalcillin, apicycline, arbekacin, aspoxicillin, azidamfenicol, azithromycin, bambermycins, benzoylpas, biapenem, 5-bromosalicylhydroxamic acid, butirosin, cefadroxil, cefamandole, cefatrizine, cefbuperazone, cefdinir, cefminox, cefonicid, cefoperazone, cefoselis, cefpiramide, cefprozil, chloramphenicol, chloroxylenol, chlorquinadol, chlortetracycline, clofoctol, clomocycline, cloxacillin, cloxyquin, clarithromycin, clindamycin, colistin, dalfopristin, demeclocycline, deoxydihydrostreptomycin, diathymosulfone, dibekacin, dihydrostreptomycin, dirithromycin, doxycycline, enviomycin, ertapenem, erythromycin and its ester derivatives, ethambutol, flomoxef, forimicins, fropenem, fusidic acid, gentamycin, glyconiazide, glucosulfone sodium, n4-beta-d-glucosylsulfanilamide, gramicidin(s), guamecycline, imipenem, isepamicin, josamycin, kanamycin(s), leucomycins, lincomycin, lymecycline, meclocycline, merbromin, meropenem, methacycline, micronomicin, midecamycins, mikamycin, minocycline, miokamycin, moxalactam, nadifloxacin, neomycin, netilmicin, nifurpirinol, nifurtoinol, nitroxoline, novobiocin, oleandomycin, oxytetracycline, panipenem, paromomycin, phenyl aminosalicylate, pipacycline, polymyxin, primycin, pristinamycin, quinupristin, ramoplanin, ribostamycin, rifabutin, rifalazil, rifamide, refampicin, rifamycin sv, rifampin, rifapentine, rifaximin, ristocetin, ritipenem, rokitamycin, rolitetracycline, rosaramicin, roxarsone, roxithromycin, salazosulfadimidine, salinazid, sancycline, sisomicin, spectinomycin, spiramycin, streptolydigin, streptomycin, streptonicozid, sulfaloxic acid, 4-sulfanilamidosalicylic acid, 2-p-sulfanilylanilinoethanol, teicoplanin, telithromycin, thiamphenicol, thiostrepton, tobramycin, trospectomycin, tuberactinomycin, tyrocidine, vancomycin, viomycin, virginiamycin, xanthocillin or xibornol.

A representative example of the anti-bacterial agent is selected from amoxicillin, azithromycin, cefadroxil, cefpiramide, chloramphenicol, clarithromycin, clindamycin, cloxacillin, doxycycline, ethambutol, nadifloxacin, neomycin, oxytetracycline, panipenem, refampicin, rifaximin, spiramycin, streptomycin or vancomycin.

Still further in the thirty-sixth embodiment, the antifungal agent referred to in the thirty-second embodiment is selected from acrisorcin (9-aminoacrindine compound with 4-hexylresorcinol (1:1)), amphotericin B, anidulafungin, bromosalicylchloranilide, buclosamide, candicidin, caspofungin, chlorphenesin, ciclopirox, dermostatin, griseofulvin, filipin, fluconazole, fungichromin, mepartricin, micafungin, natamycin, nystatin, lucensomycin, pecilocin, perimycin, posaconazole, ravuconazole, rubijervine, salicylanilide, siccanin, 2,4,6-tribromo-m-cresol, tubercidin, viridian or voriconazole.

Still further in the thirty-sixth embodiment, the antiviral agent referred to in the thirty-second embodiment is selected from abacavir, acyclovir, adefovir, amprenavir, atazanavir, cidofovir, didanosine, dideoxyadenosine, edoxudine, emtricitabine, entecavir, floxuridine, ganciclovir, idoxuridine, indinavir, kethoxal, lamivudine, lopinavir, 5-(methylamino)-2-deoxyuridine (madu), nelfinavir, nevirapine, penciclovir, podophyllotoxin, resiquimod, ribavirin, ritonavir, saquinavir, sorivudine, stavudine, tenofovir, tipranavir, trifluridine, tromantadine, valganciclovir, vidarabine, zalcitabine, zanamivir or zidovudine.

A representative example of the antiviral agent is selected from abacavir, acyclovir, adefovir, amprenavir, cidofovir, didanosine, emtricitabine, ganciclovir, indinavir, lamivudine, lopinavir, nelfinavir, nevirapine, penciclovir, ritonavir, saquinavir, stavudine, tenofovir, valganciclovir, vidarabine, zalcitabine, zanamivir or zidovudine.

Still further in the thirty-sixth embodiment, the antimalarial agent referred to in the thirty-second embodiment is selected from amodiaquine, arteflene, artemisinin alcohol, bebeerines, cinchonidine, cinchonine, dihydroartemisinin, fosmidomycin, gentiopicrin, halofantrine, hydroxychloroquine, lumefantrine, mefloquine, pyronaridine, quinine or yingzhaosu A.

Still further in the thirty-sixth embodiment, the antidiabetic agent referred to in the thirty-second embodiment is selected from acarbose, acetohexamide, miglitol, troglitazone or voglibose.

Still further in the thirty-sixth embodiment, the antiulcer agent (including proton pump inhibitors) referred to in the thirty-second embodiment is selected from arbaprostil, enprostil, misoprostol, ornoprostil, gama-oryzanol A, plaunotol, rebamipide, rioprostil, rosaprostol, spizofurone (i.e., hydroxyl of its oxime derivative), telenzepine, teprenone (i.e., hydroxyl of its oxime derivative) or trimoprostil.

Still further in the thirty-sixth embodiment, the antioxidant (including free radical scavengers) referred to in the thirty-second embodiment is selected from N-acetyl carnosine, ascorbic acid, BN-82451, L-carnitine (levocarnitine), curcumin, dexanabinol, edaravone, (−) epigallocatechin gallate, emoxipin, hydroxytyrosol, idebenone, luteolin, nicanartine, NZ-419, oxyresveratrol, probucol (including probucol prodrugs such as AGI-1067 and AGI-1096), quercetin, reductic acid, silybin, SCMC-Lys, tempol (4-hydroxy-tempo), alfa-tocopherol (vitamin E) or zeatin.

Still further in the thirty-sixth embodiment, the vitamin referred to in the thirty-second embodiment is selected from ascorbic acid, cobamamide (vitamin B₂ coenzyme), cyanocobalamin (vitamin B₁₂), ergosterol (provitamine D), fursultiamine (thiamine tetrahydrofurfuryl disulfide), hydroxocobalamin (vitamin B₁₂ₐ)_{,} 1α-hydroxycholecalciferol, (1α-hydroxyvitamin D₃), inositol (vitamin B complex), menadiol (dihydrovitamin K₃), menaquinones or vitamin K₂ (hydroxyl of its ketoxime), methylcobalamin, octotiamine, pantothenic acid (vitamin B₅), phylloquinone (hydroxyl of its ketoxime), prosultiamine (dithiopropylthiamine or DTPT or TPD), pyridoxine hydrochloride (vitamine B₆ hydrochloride), pyridoxal 5-phosphate, riboflavin (vitamin B₂ or vitamin G or lactoflavin), riboflavin monophosphate (vitamin B₂ phosphate), vitamin A, vitamin D₂, vitamin D₃, vitamin K₅, thiamine (vitamin B₁), thiamine disulfide (vitamin B₁ disulfide) or α-tocopherol (vitamin E supplement).

As has been indicated hereinabove that the twenty-second embodiment also encompasses within its scope a compound of formula (I) wherein the drug containing a hydroxyl group is selected from the group of drugs belonging to several other therapeutic areas (including those drugs that are classified on the basis of their mechanism of action). Thus, for the purpose of this invention, the twenty-sixth embodiment also encompasses a compound of formula (I); wherein the drug containing hydroxyl group is generically selected from drugs falling under several other therapeutic areas (including those drugs that are classified on the basis of their mechanism of action) and is specifically selected from: an abortifacient/interceptive selected from epostane, gemeprost, mifepristone, prostaglandin E₂ or sulprostone; an anabolic agent selected from androisoxazole, androstenediol, bolandiol, bolasterone, clostebol, ethylestrenol, formebolone, mestanolone, methandriol, methenolone, methyltrienolone, nandrolone, norbolethone, oxabolone, quinbolone or trenbolone; an androgen selected from boldenone, cloxotestosterone, fluoxymesterone, mesterolone, methandrostenolone, 17-methyltestosterone, 17α-methyltestosterone 3-cyclopentyl enol ether, norethandrolone, normethandrone, oxandrolone, oxymesterone, oxymetholone, stanolone, stanozolol, testosterone or tiomesterone; an anesthetic selected from biphenamine, chloral hydrate, ecgonine, γ-hydroxybutyrate (γ-hydroxybutyric acid), hydroxytetracaine, ketamine, lidocaine, methohexital sodium, orthocaine, oxethazaine, pentobarbital, polidocanol, pregnan-3-ol-20-one, propofol, propipocaine, salicyl alcohol, thialbarbital, thiamylal or thiobutabarbital; an anorexic agent selected from diethylpropion, norpseudoephedrine, diphemethoxidine, metamfepramone or mazindol; an anthelmintic agent selected from aspidin, aspidinol, becanthone, dichlorophen, 4-hexylresorcinol, ivermectin, niclosamide, oxantel, triclofenol piperazine, hycanthone, lucanthone, oxamniquine or trichlorfon; an anti-acne agent selected from algestone acetophenide or cioteronel; an anti-alopecia agent selected from cioteronel, cioteronel or finasteride; an antiamebic agent selected from arsthinol, bialamicol, chlorbetamide, chlorphenoxamide, diloxanide, 8-hydroxy-7-iodo-5-quinolinesulfonic acid, iodoquinol, thiocarbamizine, glycobiarsol, secnidazole or tetracycline; an antiandrogen agent selected from bicalutamide, bifluranol, cioteronel, cyproterone, delmadinone acetate, nilutamide, osaterone or oxendolone; an antiarthritic/antirheumatic agent selected from aurothioglucose, glucosamine, bucillamine or kebuzone; an antiasthmatic agent selected from beclomethasone, budesonide, cromolyn, dexamethasone, formoterol, flunisolide, ibudilast, ketotifen, montelukast, nedocromil, oxatomide, pranlukast, seratrodast, suplatast tosylate, tiaramide, traxanox, triamcinolone acetonide, zafirlukast or zileuton; an antidiarrheal agent selected from catechin, loperamide or mebiquine; an antidiuretic drug selected from desmopressin, lypressin, ornipressin, oxycinchophen, terlipressin or vasopressin; an antiemetic agent selected from diphenidol, nabilone, ondansetron, oxypendyl or tetrahydrocannabinols; an antiglaucoma agent selected from bimatoprost, latanoprost, levobunolol, travoprost or unoprostone; an antigout/uricosuric agent selected from allopurinol, benzbromarone, colchicine, sulfinpyrazone or oxycinchophen; an antihyperparathyroid drug selected from doxercalciferol, maxacalcitol or paricalcitol; an antihyperthyroid drug such as thibenzazoline; an antihypothyroid drug selected from tiratricol or thyroxine; an antimigraine agent selected from methysergide or flumedroxone acetate; an antimuscarinic/mydriatic agent selected from atropine, benactyzine, benzilonium bromide, bevonium methyl sulfate, biperiden, butropium bromide, n-butylscopolammonium bromide, cimetropium bromide, cinnamedrine, clidinium bromide, cyclodrine, cyclopentolate, dexetimide, difemerine, eucatropine, fentonium bromide, flavoxate, flutropium bromide, glycopyrrolate, hexocyclium methyl sulfate, homatropine, hyoscyamine, ipratropium bromide, mepenzolate bromide, methscopolamine bromide, oxybutynin, oxyphencyclimine, oxyphenonium bromide, oxitropium bromide, penthienate bromide, phenglutarimide, pipenzolate bromide, piperilate, poldine methylsulfate, procyclidine, scopolamine, scopolamine n-oxide, telenzepine, tiemonium iodide, tiotropium bromide, tolterodine, tridihexethyl iodide, trihexyphenidyl hydrochloride, tropicamide or trospium chloride; an antiosteoporotic agent selected from alendronic acid, etidronic acid, ibandronic acid, pamidronic acid, raloxifene, risedronic acid or zoledronic acid; an antiprostatic hypertrophy agent selected from gestonorone caproate, mepartricin, osaterone or oxendolone; an antiprotozoal agent selected from acetarsone, Acranil®, anisomycin, hydroxystilbamidine, melarsoprol, mepartricin, N-methylglucamine, metronidazole, nifuroxime, oxophenarsine hydrochloride, puromycin or secnidazole; an antipruritic agent selected from camphor, dichlorisone, halometasone, 3-hydroxycamphor, menthol, phenol or polidocanol; an antipsoriatic agent selected from anthralin, 6-azauridine, calcipotriene, chrysarobin, maxacalcitol, pyrogallol or tacalcitol; an antiseborrheic agent selected from chloroxine, piroctone, resorcinol or tioxolone; an antiseptic agent selected from acetomeroctol, benzoxonium chloride, bibrocathol, broxyquinoline, cethexonium bromide, 4-chloro-m-cresol, dichlorobenzyl alcohol, ethylhydrocupreine, hexachlorophene, 8-hydroxyquinoline, isopropyl alcohol, mandelic acid, meralein sodium, mercurophen, 2-naphthyl salicylate, nitroakridin 3582, noxythiolin, oxymethurea, phenoxyethanol, polynoxylin, pyrocatechol, α-terpineol, thymol or triclosan; an antispasmodic agent selected from amprotropine phosphate, benactyzine, benzilonium bromide, bevonium methyl sulfate, butropium bromide, n-butylscopolammonium bromide, cimetropium bromide, cinnamedrine, clidinium bromide, difemerine, fentonium bromide, flopropione, glycopyrrolate, hexocyclium methyl sulfate, hyoscyamine, levomepate, mepenzolate bromide, methscopolamine bromide, oxyphencyclimine, oxyphenonium bromide, penthienate bromide, phloroglucinol, pipenzolate bromide, piperilate, poldine methylsulfate, propenzolate, rociverine, sultroponium, tiemonium iodide, tridihexethyl iodide, tropenzile, flavoxate, tricromyl or trospium chloride; an antitussive agent selected from chlophedianol, clobutinol, cyclexanone, dropropizine, drotebanol, eprazinone, pholcodine, zipeprol, amicibone, morclofone or normethadone; an antiulcerative agent selected from acetoxolone, aldioxa, carbenoxolone, enprostil, misoprostol, ornoprostil, plaunotol, rioprostil, rosaprostol, rotraxate, teprenone, trimoprostil, spizofurone or γ-oryzanol; an anxiolytic agent selected from azacyclonol, clorazepic acid (enol-form), enciprazine, ethyl loflazepate (enol-form), flesinoxan, flutazolam, hydroxyphenamate, hydroxyzine, lorazepam, mecloralurea or oxazepam; an astringent selected from alkannin, baicalein, bismuth subgallate or tannic acid; a cathartic drug/laxative selected from aloe-emodin, aloin, bisoxatin acetate, cellulose ethyl hydroxyethyl ether, colocynthin, danthron, emodin, frangulin, glucofrangulin, oxyphenisatin acetate, phenolphthalein, phenolphthalol, sennosides or phenoltetrachlorophthalein; a choleretic agent selected from alibendol, cholic acid, cyclobutyrol, cyclovalone, cynarin(e), dehydrocholic acid, deoxycholic acid, α-ethylbenzyl alcohol, exiproben, febuprol, fencibutirol, fenipentol, hymecromone, menbutone, osalmid, 4,4′-oxydi-2-butanol, 4-salicyloylmorpholine, taurocholic acid, vanitiolide, trepibutone or metochalcone; a cholinergic agent selected from muscarine, edrophonium chloride or dexpantheno; a contraceptive or progestogen drug selected from allylestrenol, anagestone, chlormadinone acetate, delmadinone acetate, demegestone desogestrel, dienogest, dimethisterone, drospirenone, dydrogesterone, elcometrine, ethinyl estradiol, ethisterone, ethynodiol, etonogestrel, fluorogestone acetate, gestodene, gestonorone caproate, 17-hydroxy-16-methylene-Δ⁶-progesterone, 17α-hydroxyprogesterone, lynestrenol, medrogestone, medroxyprogesterone, megestrol acetate, mestranol, norethindrone, norethynodrel, norgesterone, norgestimate, norgestrel, norgestrienone, norvinisterone, pentagestrone, progesterone, promegestone or trengestone; a decongestant drug selected from amidephrine, cafaminol, ephedrine, epinephrine, nordefrin, oxymetazoline, phenylephrine, phenylpropanolamine or pseudoephedrine; an emetic agent selected from apocodeine or cephaeline; an enzyme cofactor selected from dexpanthenol, fursultiamine, octotiamine, pantothenic acid, prosultiamine, pyridoxal 5-phosphate, pyridoxine hydrochloride, riboflavin, riboflavin monophosphate, sapropterin, thiamine or thiamine disulfide; an estrogen drug selected from benzestrol, colpormon, dienestrol (trans-trans-form), equilenin, equilin, estradiol, estriol, estrone, ethinyl estradiol, hexestrol, mestranol, methestrol, moxestrol, mytatrienediol, quinestradiol or quinestrol; an expectorant drug selected from ambroxol, guaiacol, iodinated glycerol or guaifenesin; a gastroprokinetic drug such as alvimopan; a hemostatic agent selected from adrenalone, algin, aminochromes, carbazochrome salicylate, carbazochrome sodium sulfonate, cephalins, cotamine, ellagic acid, ethamsylate, oxidized cellulose or vapreotide; a hepatoprotective drug selected from S-adenosylmethionine, catechin or silymarin; an immunomodulator selected from amiprilose, lisofylline, ubenimex, inosine pranobex, bropirimine, lentinan, mitoxantrone, romurtide or thymopentin; an immunosuppressant selected from everolimus, gusperimus, mizoribine, mycophenolic acid, rapamycin or tacrolimus; a mucolytic selected from domiodol or sobrerol; a muscle relaxant drug selected from chlorzoxazone, eperisone, idrocilamide, inaperisone, mephenesin, methocarbamol, tolperisone or dantrolene; a mydriatic drug such as yohimbine; a narcotic antagonist agent selected from cyclazocine, levallorphan, nalmefene, nalorphine, naloxone or naltrexone; a neuroprotective agent selected from lubeluzole or citicoline; a nootropics/cognition enhancer drug selected from bemegride, choline alfoscerate, curcumin, donepezil, ethamivan, exifone, hexacyclonate sodium, homocamfin, idebenone, nizofenone, oxiracetam, pipradrol, propentofylline pyritinol, pyrovalerone, sabeluzole, sulbutiamine or velnacrine; a prostaglandin analog selected from beraprost, carboprost, clinprost, enprostil, gemeprost, latanoprost, limaprost, misoprostol, ornoprostil, prostacyclin, prostaglandin E₁, prostaglandin E₂, prostaglandin F_{2α}, rioprostil, rosaprostol, trimoprostil or unoprostone; a respiratory stimulating agent selected from dimefline, lobeline, mepixanox or pimeclone; a sedative/hypnotic drug selected from aldol, allobarbital, amobarbital, aprobarbital, apronalide, barbital, brallobarbital, butabarbital sodium, butalbital, butallylonal, butethal, butoctamide, carbubarb, chloral formamide, α-chloralose, cinolazepam, cyclobarbital, cyclopentobarbital, doxefazepam, ectylurea, enallylpropymal, ethchlorvynol, febarbamate, 5-furfuryl-5-isopropylbarbituric acid, glutethimide, haloxazolam, heptabarbital, hexethal sodium, hexobarbital, hexapropymate, homofenazine, lormetazepam, methyprylon, narcobarbital, nealbarbital, pentaerythritol chloral, pentobarbital, phenallymal, piperidione, propallylonal, propiomazine proxibarbal, pyrithyldione reposal, secobarbital sodium, talbutal, temazepam, tetrabarbital, 2,2,2-trichloroethanol, vinbarbital sodium or vinylbital; a vulnerary drug selected from allantoin, chitin, dextranome or thioglycerol; an α-adrenergic agonist agent selected from adrafinil, dipivefrin, hydroxyamphetamine, mivazerol, norfenefrine, octopamine, pseudoephedrine, pholedrine, synephrine or tyramine; a β-adrenergic agonist agent selected from albuterol (salbutamol), bitolterol, carbuterol, clenbuterol, clorprenaline, dioxethedrine, etafedrine, ethylnorepinephrine, fenoterol, hexoprenaline, isoetharine, isoproterenol, mabuterol, metaproterenol, pirbuterol, procaterol, protokylol, reproterol, rimiterol, ritodrine, soterenol, terbutaline, tretoquinol, tulobuterol or xamoterol; an α-adrenergic blocker drug selected from labetalol, naftopidil or trimazosin; a dopamine receptor agonist drug selected from apomorphine, quinagolide or ropinirole; a dopamine receptor antagonist drug such as iloperidone; a gonad-stimulating agent selected from epimestrol or LH-RH; a 5-Lipoxygenase inhibiting agent such as tenidap; a matrix metalloproteinase inhibiting agent selected from batimastat or prinomastat; a monoamine oxidase inhibiting agent such as toloxatone; a NMDA receptor antagonist such as licostinel; a prolactin inhibiting agent such as bromocriptine; a reverse transcriptase inhibiting agent such as zalcitabine; a serotonin receptor agonist such as ergotamine; a serotonin receptor antagonist selected from dolasetron or ketanserin and a topoisomerase I inhibitor such as 9-aminocamptothecin.

In a thirty-seventh embodiment, the invention encompasses a compound of formula (I), wherein: D is a drug containing a sulfhydryl group capable of forming a bio-cleavable covalent linkage with a linker;

X¹ is sulphur;

### Y is C═O;

each X²; Z¹; Z²; A, R¹ and R² is as defined in the first embodiment hereinabove;
with the provisos that:
a) when A is S, then a and b is 3; or
b) when A is D-isosorbide skeleton or 1,4-anhydroerythritol skeleton, then a and b is 0; and
   in all its stereoisomeric forms and pharmaceutically acceptable salts thereof.

In a thirty-eighth embodiment, the invention encompasses a compound of formula (I), wherein: each of D and X¹ is as defined in the thirty-seventh embodiment hereinabove;

Each of X²; Y, Z¹; Z²; A, R¹ and R² is as defined in the second embodiment hereinabove;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a thirty-ninth embodiment, the invention encompasses a compound of formula (I), wherein: each of D and X¹ is as defined in the thirty-seventh embodiment hereinabove;

each of X²; Y, Z¹; Z² is as defined in the second embodiment hereinabove;
A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH or CR⁹R¹⁰; where R⁹ and R¹⁰ are independently selected from hydrogen or C₁₋₆ alkyl;
provided that when A is S, then a and b is 3;
R¹ is hydrogen and R² is alkyl; or R² is hydrogen and R¹ is alkyl;
in all its stereoisomeric forms and pharmaceutically acceptable salts thereof.

In a fortieth embodiment, the invention encompasses a compound of formula (I), wherein: each of D, X¹, X², Y, Z¹ and Z² is as defined in the thirty-eighth embodiment hereinabove,

A is selected from a bond, CH═CH or CR⁹R¹⁰; where R⁹ and R¹⁰ are independently selected from hydrogen or C₁₋₆ alkyl;
R¹ is hydrogen and R² is alkyl, cycloalkyl, aryl or aralkyl; or R² is hydrogen and R¹ is alkyl, cycloalkyl, aryl or aralkyl;
in all its stereoisomeric forms and pharmaceutically acceptable salts thereof.

In a forty-first embodiment, the invention encompasses a compound of formula (I), wherein: each of D, X¹, X², Y, Z¹ and Z² is as defined in the thirty-eighth embodiment hereinabove,

A is selected from S, SO, SO₂ or S—S; provided that when A is S, then a and b is 3
R¹ is hydrogen and R² is alkyl, cycloalkyl, aryl or aralkyl; or R² is hydrogen and R¹ is alkyl, cycloalkyl, aryl or aralkyl;
in all its stereoisomeric forms and pharmaceutically acceptable salts thereof.

In forty-second embodiment, the invention encompasses a compound of formula (I), wherein D, the drug containing a sulfhydryl group referred to in the thirty-seventh, thirty-eighth, thirty-ninth, fourtieth and forty-first embodiments, is selected from cardiovascular agents or glucocorticoids. The forty-second embodiment also encompasses within its scope a drug containing a sulfhydryl selected from the drugs that belong to several other therapeutic areas (including those drugs that are classified on the basis of their mechanism of action). In this embodiment, other variables X¹, X², Y, Z¹, Z², A, R¹ and R² in the compounds of formula (I) are as defined above; with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In forty-third embodiment, the cardiovascular agent referred to in the forty-second embodiment is selected from captopril or omapatrilat. Further, in this embodiment the glucocorticoid referred to in the forty-second embodiment is selected from tixocortol.

For the purpose of this invention, the forty-second embodiment also encompasses a compound of formula (I); wherein the drug containing sulfhydryl group is generically selected from the group of drugs falling under several other therapeutic areas (including those drugs that are classified on the basis of their mechanism of action) and is specifically selected from an anesthetic selected from buthalital sodium hydroxydione sodium, thialbarbital (Intranarcon), thiamylal, thiobutabarbital or thiopental sodium; an antiarthritic/antirheumatic agent selected from bucillamine or penicillamine; an antihyperthyroid drug selected from methimazole, propylthiouracil or thiobarbital; an antiseborrheic agent such as pyrithione; an antiseptic drug selected from noxythiolin or thiocresol; a hepatoprotective agent such as tiopronin; an immunomodulator such as bucillamine or a vulnerary drug such as thioglycerol.

In a specific embodiment, the invention encompasses a bio-cleavable linker represented herein by the compounds of formula (IA) which is capable of forming bio-cleavable covalent linkage with a drug having a carboxylic acid, hydroxyl, amino or sulfhydryl group:

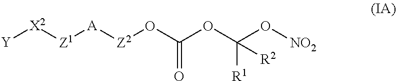

X² is a bond, oxygen or NR³;
R³ is a bond or hydrogen;
Y is C═O or a spacer group selected from:

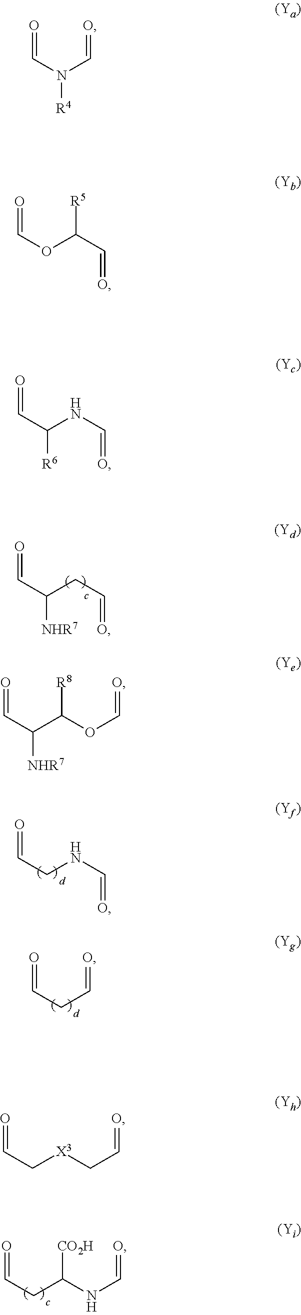

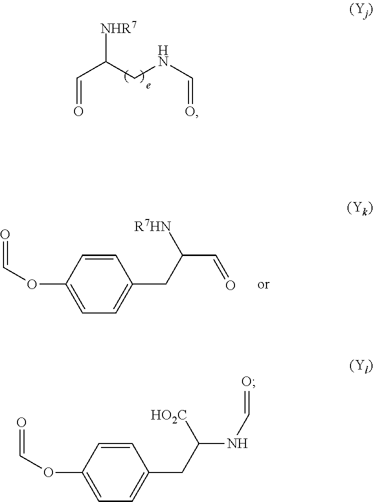

where in the spacer groups of formulae (Yₐ) to (Yₗ):
R⁴ is a bond, hydrogen, alkyl or a metal ion;
R⁵ is hydrogen, methyl or phenyl;
R⁶ is hydrogen or a side-chain group of naturally occurring amino acids selected from:
—CH₃, —CH(CH₃)₂, —CH₂CH(CH₃)₂, —CH(CH₃)CH₂CH₃, —CH₂CO₂H, —CH₂CH₂CO₂H, —CH₂OH, —CH(CH₃)OH, —CH₂SH, —CH₂CH₂SCH3, —CH₂CH₂CH₂CH₂NH₂, —C₆H₅, —CH₂C₆H₅, —CH₂C₆H₄-p-OH, —CH₂CH₂CH₂NHC(═NH)NH₂, —CH₂C(═O)NH₂, —CH₂CH₂C(═O)NH₂, —CH₂-indol-3-yl or —CH₂-imidazole;
X³ is oxygen, sulphur, SO, SO₂ or NR³;
R⁷ is hydrogen or an amino protecting group selected from: acetyl, benzoyl, alkyloxycarbonyl, benzyloxycarbonyl, 9-fluorenylmethyloxycarbonyl or its pharmaceutically acceptable ammonium salts;
R⁸ is hydrogen or methyl;
c is an integer from 0 to 2;
d is an integer from 1 to 5;
e is an integer from 1 to 4;
   Z¹ is (CH₂)ₐ; where a is an integer from 0 to 3;
   Z² is (CH₂)_{b}; where_b is an integer from 0 to 3;
   A is selected from: bond, S, SO, SO₂, S—S, CH═CH, D-isosorbide skeleton, 1,4-anhydroerythritol skeleton, cycloalkylene, CR⁹R¹⁰, C₆-C₁₀-arylene, a 5- or 6-membered heteroarylene or a 5- or 6-membered heterocyclylene wherein said arylene, heteroarylene and heterocyclylene may be unsubstituted or substituted by one or more substituents independently selected from the group consisting of C₁₋₆ alkyl, C₁₋₆ alkoxy, hydroxy, trifluoromethyl, cyano, amino and halogen;
   R⁹ and R¹⁰ are independently selected from: hydrogen or C₁₋₆ alkyl; or R⁹ and R¹⁰ taken together with the carbon atom to which they are attached form a cycloalkyl or a heterocyclic ring;

R¹ is hydrogen and R² is alkyl, cycloalkyl, aryl or aralkyl; or R² is hydrogen and R¹ is alkyl, cycloalkyl, aryl or aralkyl;

with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In an embodiment of the specific embodiment, the invention encompasses a compound of formula (IA), wherein:

X² is oxygen;

### Y is C═O;

A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH, D-isosorbide skeleton, 1,4-anhydroerythritol skeleton, cycloalkyl or CR⁹R¹⁰;
where R⁹ and R¹⁰ are independently selected from hydrogen or C₁₋₆ alkyl; or R⁹ and R¹⁰ taken together with the carbon atom to which they are attached constitute a cycloalkyl group or a 5- or 6-membered heterocyclic ring containing one to two hetero atoms selected from oxygen, sulfur or nitrogen;
R¹ is hydrogen and R² is alkyl, cycloalkyl, aryl or aralkyl; or R² is hydrogen and R¹ is alkyl, cycloalkyl, aryl or aralkyl;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In a further embodiment of the specific embodiment, the invention encompasses a compound of formula (IA), wherein:

X² is oxygen;

### Y is C═O;

A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH, D-isosorbide skeleton, 1,4-anhydroerythritol skeleton, cycloalkyl or CR⁹R¹⁰;
where R⁹ and R¹⁰ are independently selected from hydrogen or C₁₋₆ alkyl; or R⁹ and R¹⁰ taken together with the carbon atom to which they are attached constitute a cycloalkyl group or a 5- or 6-membered heterocyclic ring containing one to two hetero atoms selected from oxygen, sulfur or nitrogen;
R¹ is hydrogen and R² is alkyl; or R² is hydrogen and R¹ is alkyl;
with the provisos that:
a) when A is S, then a and b is 3; or
b) when A is D-isosorbide skeleton or 1,4-anhydroerythritol skeleton, then a and b is 0;
   in all its stereoisomeric forms and pharmaceutically acceptable salts thereof.

In yet another embodiment of the specific embodiment, the invention encompasses a compound of formula (IA), wherein

X² is oxygen;

### Y is C═O;

A is selected from a bond, 1,2-phenylene, 1,3-phenylene, 1,4-phenylene, 2,3-pyridine, 3,4-pyridine, 2,4-pyridine, 2,5-pyridine, 2,6-pyridine, S, SO, SO₂, S—S, CH═CH or CR⁹R¹⁰; where R⁹ and R¹⁰ are independently selected from hydrogen or C₁₋₆ alkyl; provided that when A is S, then a and b is 3.
R¹ is hydrogen and R² is alkyl; or R² is hydrogen and R¹ is alkyl;
in all its stereoisomeric forms and pharmaceutically acceptable salts thereof.

In yet another further embodiment of the specific embodiment, the invention encompasses a compound of formula (IA), wherein

X² is oxygen;

### Y is C═O;

A is selected from a bond, CH═CH or CR⁹R¹⁰; where R⁹ and R¹⁰ are independently selected from hydrogen or C₁₋₆ alkyl;
R¹ is hydrogen and R² is alkyl; or R² is hydrogen and R¹ is alkyl;
in all its stereoisomeric forms and pharmaceutically acceptable salts thereof.

It would be understood by a person of skill in the art that in the compounds of formula (IA) when Y is “CO” or designate any other group that contain a “CO”, then the “CO” must have been derived from a carboxyl-containing drug D.

In specific embodiments, the invention encompasses a compound of formula (I) from the following compounds:

(a) Compounds of formula (I) wherein D is a drug containing a carboxylic acid group:

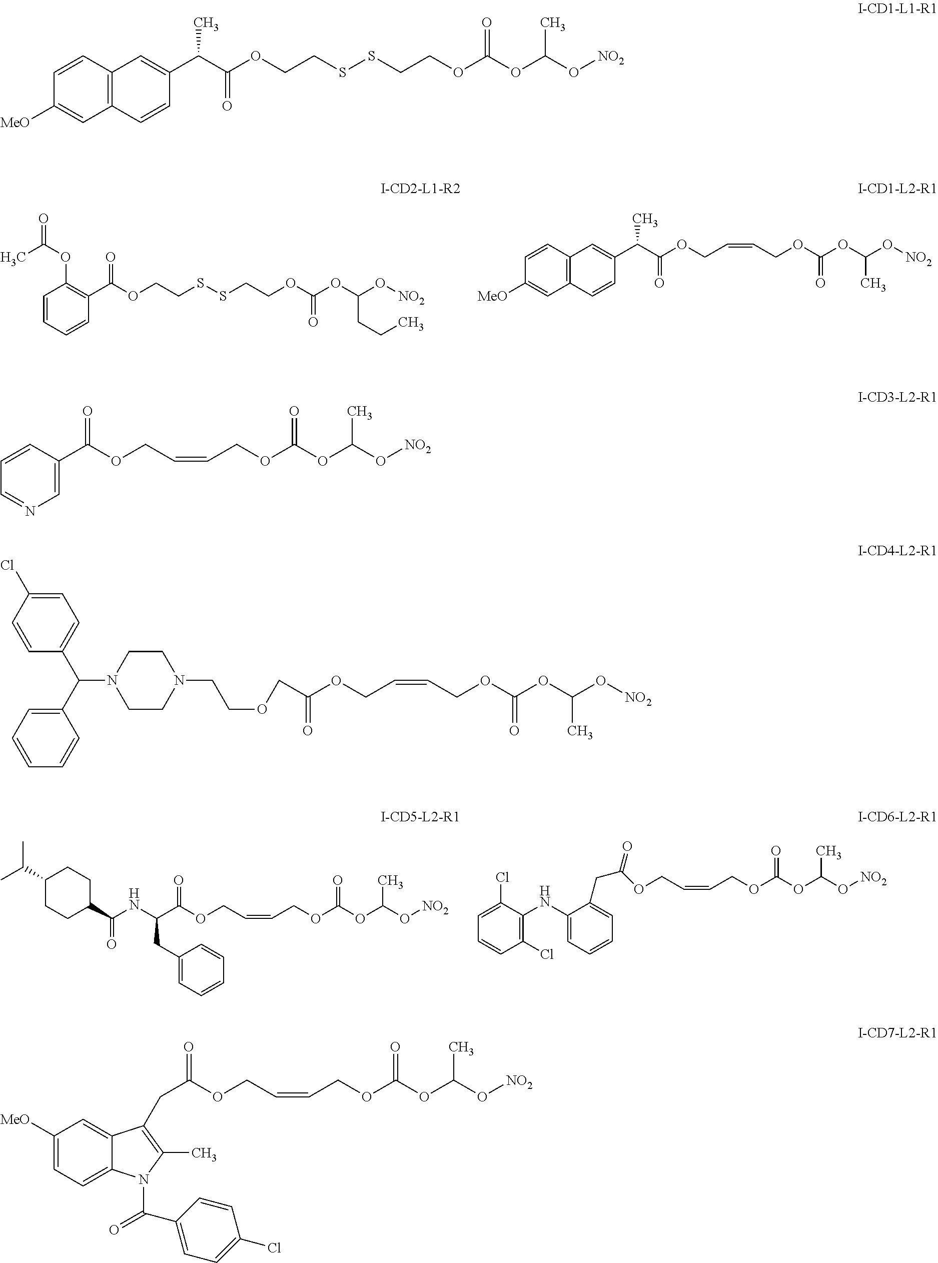

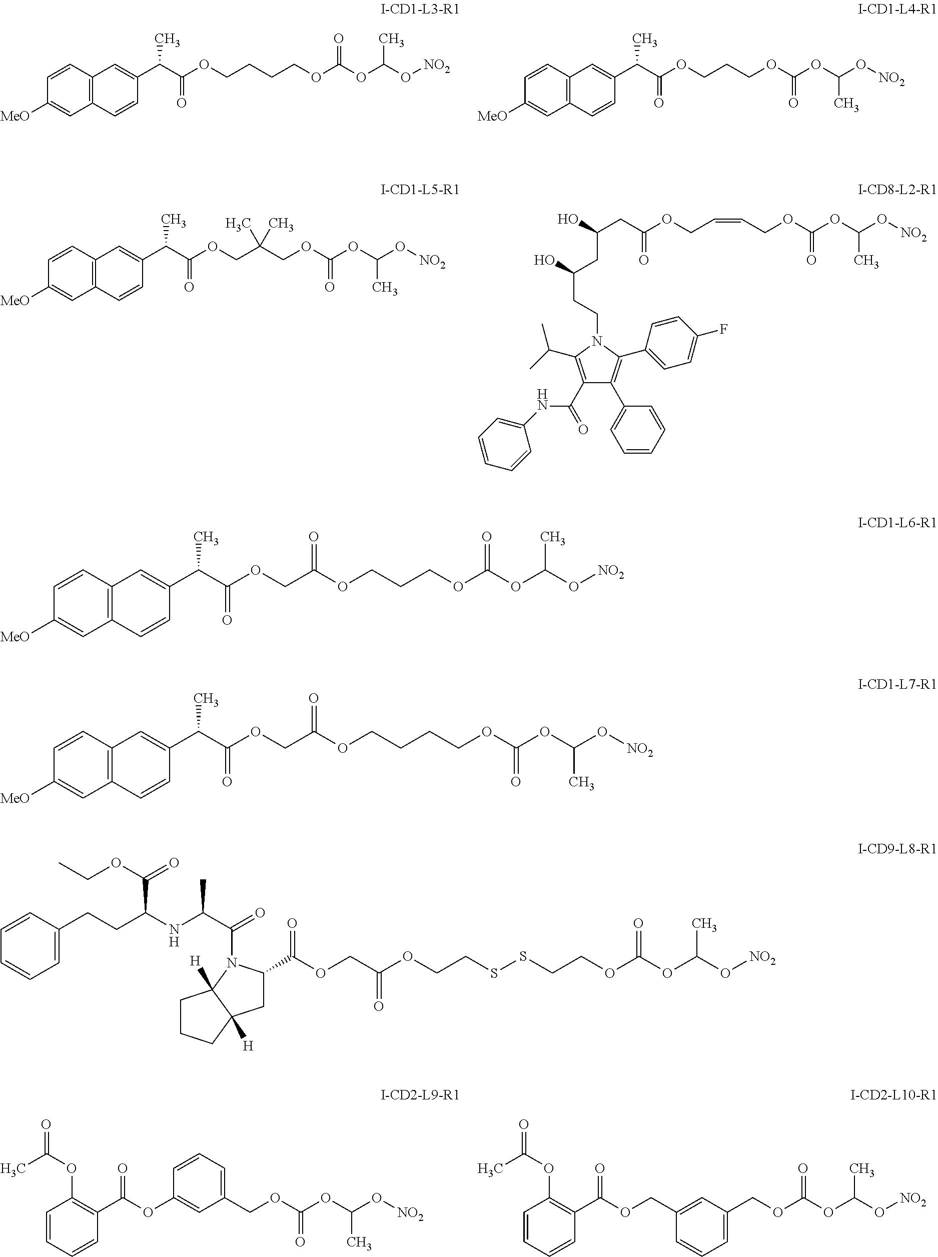

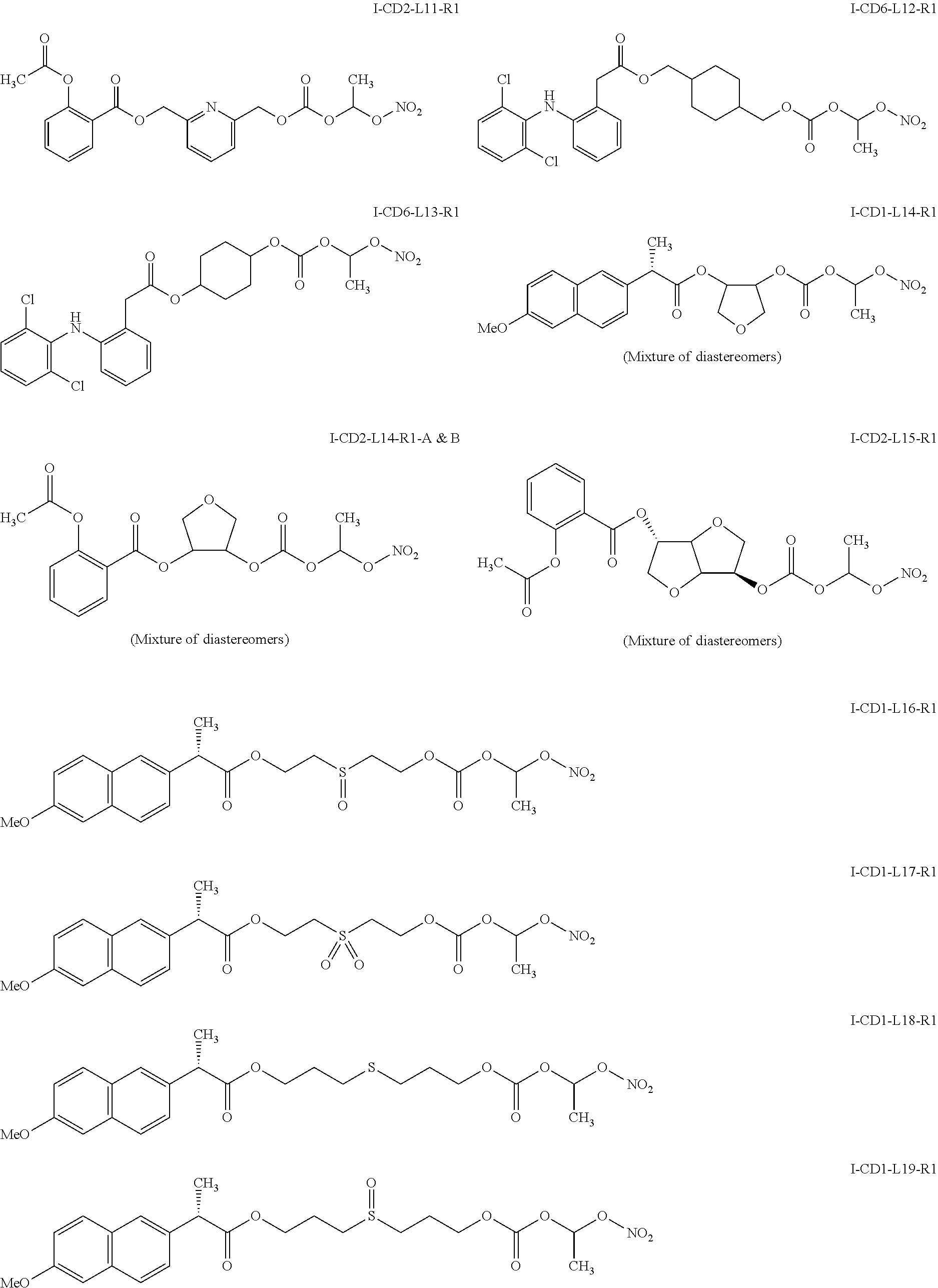

(b) Compounds of formula (I) wherein D is a drug containing an amino group:

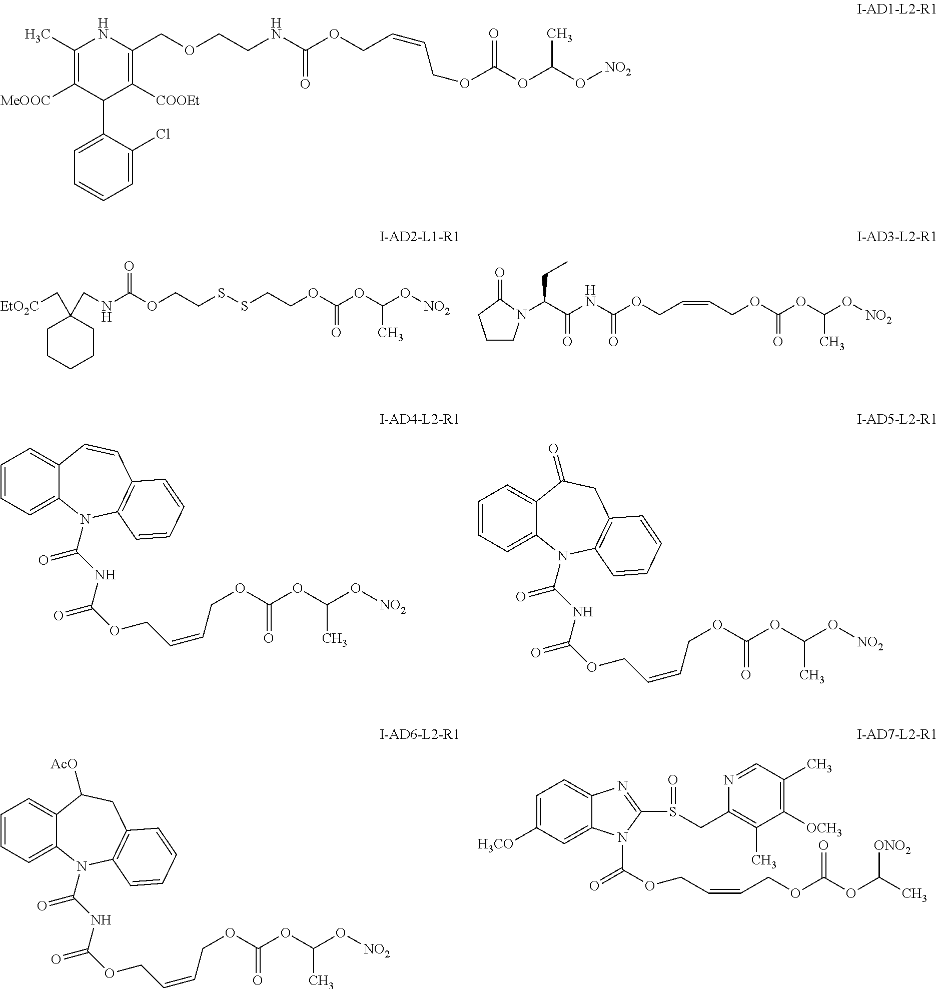

(c) Compounds of formula (I) wherein D is a drug containing a hydroxy group:

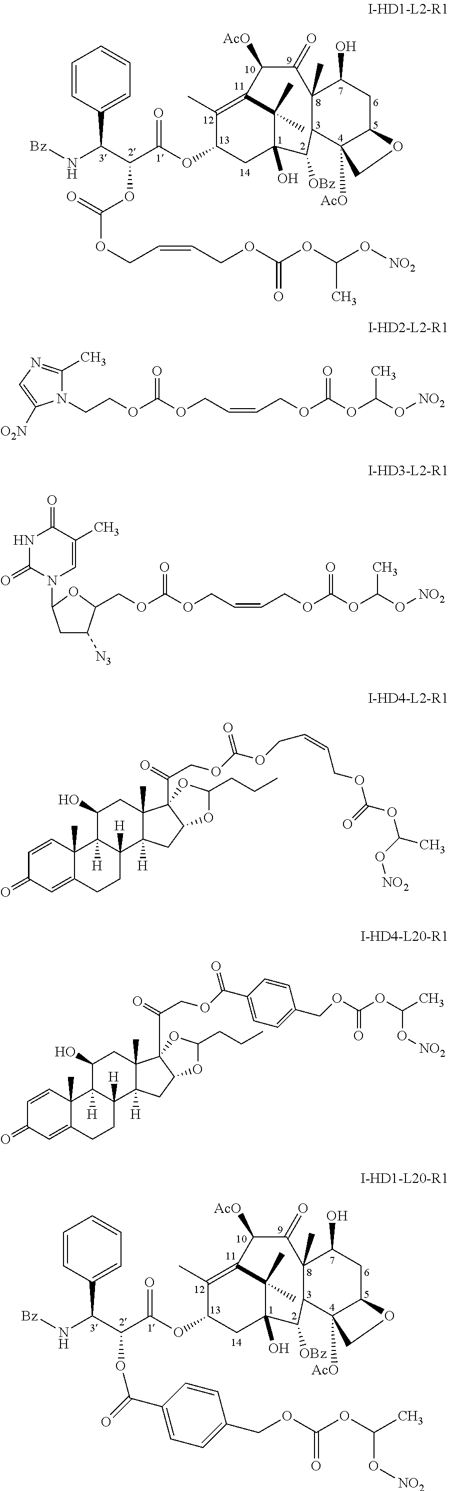

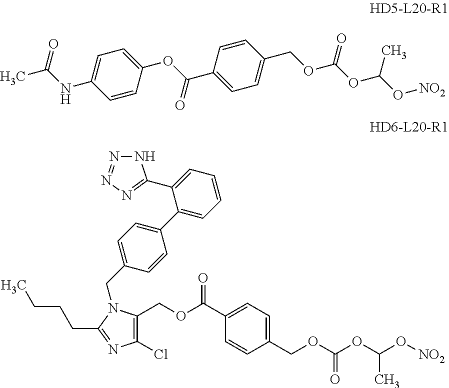

In a specific embodiment, the invention encompasses linker compounds of formula (IA) from the following group of representative linkers:

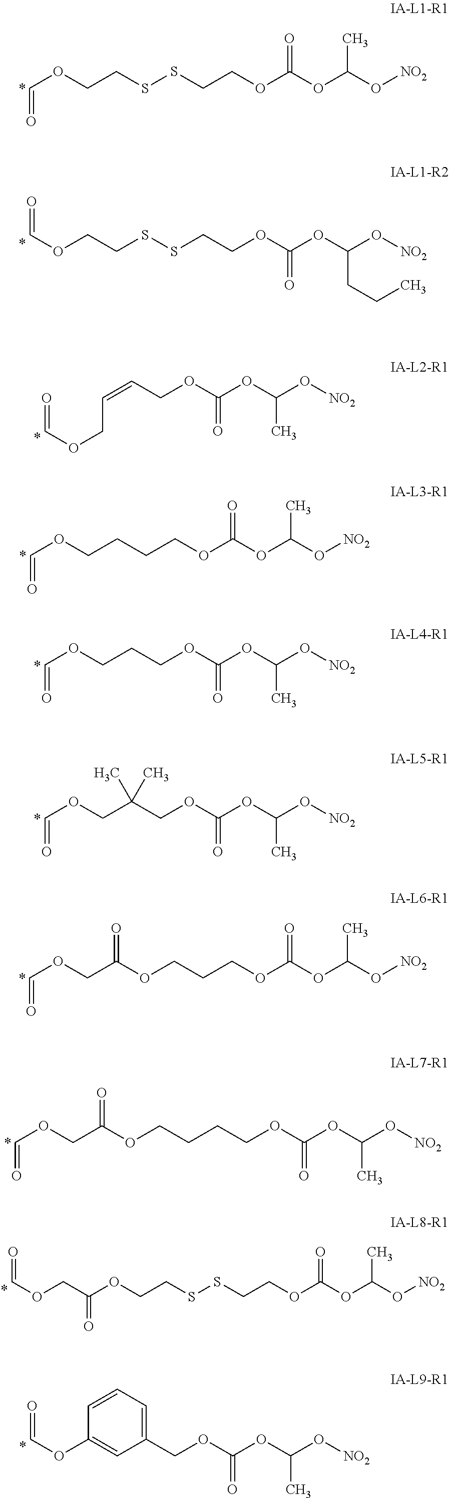

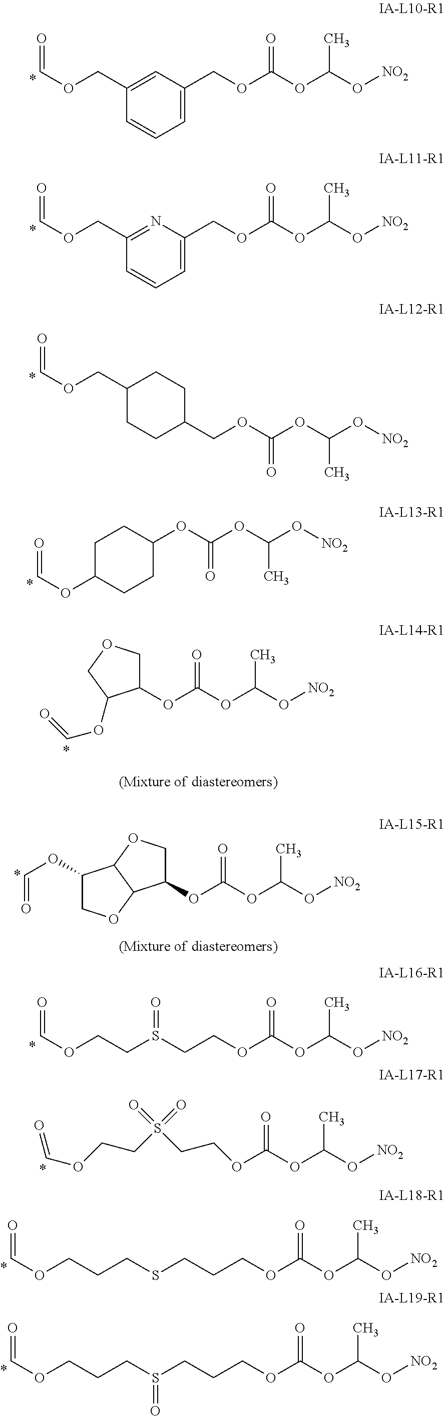

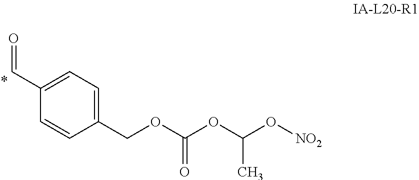

* Point of attachment to a suitable drug residue.

The compound of formula (I) and the bio-cleavable linker of formula (IA) contain asymmetric or chiral centers, and therefore exist in different stereoisomeric forms. In the structures shown herein, where the stereochemistry of any particular chiral atom is not specified, then all stereoisomers are contemplated and included as the compounds of the invention. The term “chiral” refers to molecules which have the property of non-superimposability of the mirror image cohort, while the term “achiral” refers to molecules which are superimposable on their mirror image partner. It is intended that all stereoisomeric forms of the compounds of the invention, including but not limited to, diastereomers and enantiomers, as well as mixtures thereof such as racemic mixtures, form part of the present invention. Thus, compound of formula (I) and the linker of formula (IA) according to the present invention which can exist as enantiomers can be present in enantiomerically pure form, both as levorotatory and as dextrorotatory antipodes, in the form of racemates and in the form of mixtures of the two enantiomers in all ratios. In the case of cis/trans isomerism the compound of formula (I) and the bio-cleavable linker of formula (IA) includes both cis and trans form as well as mixtures of these forms in all ratios, preferably exists in cis form. The preparation of individual stereoisomers of the compounds of the present invention i.e. the compound of formula (I) and the bio-cleavable linker of formula (IA), can be carried out, if desired, by separation of a mixture by methods known in the art. For instance, the racemic forms can be resolved by physical methods, such as fractional crystallisation or separation by chiral column chromatography. The individual optical isomers can be synthesised in the optically pure form by the use of enzymes or through asymmetric synthesis. If, for instance, a particular enantiomer of the compound of formula (I) of the present invention is desired, it may be prepared by derivatisation with a chiral auxiliary whereby the resulting diastereomeric mixture is separated and the auxiliary group cleaved to provide the pure desired enantiomer. In case, the compound of formula (I) contains a basic functional group such as amino or an acidic functional group such as carboxyl, diastereomeric salts are formed with an appropriate optically active acid or base, respectively. Consequently, compounds of formula I can exist in enantiomeric or diastereomeric forms or in mixtures thereof. The processes for preparation can utilize racemates, enantiomers or diastereomers as starting materials. When diastereomeric or enantiomeric products are prepared, they can be separated by conventional methods for example, chromatographic techniques or fractional crystallization.

The present invention also relates to processes for the preparation of the compounds of formula (I) or pharmaceutically acceptable salts thereof. The compound of formula (I) may be prepared by any of the general schemes 1-21 as outlined herein below. Unless otherwise specified, the groups A, Z¹, Z², R¹, R², R³, R⁴, R⁵, R⁶, R⁷, R⁸, R⁹, R¹⁰ X¹, X², X³, a, b, c, d, e are as defined in respect of formula (I) and/or formula (IA) above. The starting materials and reagents employed in the processes for preparation of compounds of formula (I) may be commercially available or may be prepared by processes known in the art.

The symbols as used herein with particular reference to the processes for the preparation of the compounds of formula (I) as illustrated in the following schemes 1-21, are as described herein below:
The drug containing carboxylic acid group is designated as Dₐ (D-COOH) and its derivatives are designated as Dₐ₁ and Dₐ₂ respectively.
The drug containing an amino group (D-Y—X¹H, wherein Y is a bond, C═O, SO₂ or O(CO); X¹═NR³ wherein R³ is a bond or H) is designated in general as D_{b}. Further, the drug containing a hydroxyl or sulfhydryl group (D-X¹H, wherein X¹═O or S) is designated herein as D_{c}. The derivatives of the drug D_{b} are designated herein as D_{b1} and D_{b2} respectively. The derivatives of the drug D_{c} are designated herein as D_{c1}, D_{c2}, D_{c3}, D_{c4}, D_{c5}, D_{c6}, D_{c7}, D_{c8}, D_{c9}, D_{c10}, D_{c11}, D_{c12}, D_{c13}, D_{c14}, D_{c15}, D_{c16}, D_{c17}, D_{c18}, D_{c19}, D_{c20}, D_{c21}, D_{c22}, D_{c23}, D_{c24}, D_{c25} and D_{c26} respectively.
The starting material or the precursors to the linker are denoted herein by the symbols Lₐ, L_{b}, Lₐ₁, Lₐ₂, Lₐ₃, L_{b1}, Lₐ′, L_{c}, L_{d}, L_{f} and L_{g}, respectively.
The linker is denoted herein by the symbol L₁ and its derivative is denoted herein by the symbol Lₑ.
The aldehyde, R¹C(═O)R² (wherein, R¹ and R² are as defined above), the starting material for the preparation of the α-chloroformate of formula (X) is denoted herein by the symbol Sₐ.
The precursor for the spacer groups are denoted herein by the symbols S_{b}, S_{c}, S_{d}, Sₑ, S_{f}, S_{g}, Sₕ, Sᵢ, Sⱼ, Sₖ and Sₗ, respectively.
The derivative of the spacer group precursor S_{b} is denoted herein by the symbol S_{b1}.
The linker group obtained by coupling the linker L₁ with the spacer group precursor or its derivatives (S_{b1}, S_{c}, S_{d}, Sₕ, Sᵢ, Sⱼ and Sₖ) are denoted herein by the symbols L_{g1}, Lₕ, Lᵢ, Lₖ, Lᵢ, Lₘ and Lₙ, respectively.
The linker group obtained by coupling the spacer group precursor Sₗ and the linker derivative Lₑ is denoted herein by the symbol Lₙ.
The intermediates obtained by coupling the drug, D (as defined herein) with the a) linker precursors (as defined above), b) linker (as defined above), c) linker derivatives d) spacer precursors (as defined above); and e) linker groups obtained by coupling spacer precursors or its derivatives and the linker L1 (as defined above) are denoted by the symbols Iₐ, I_{b}, I_{c}, I_{d}, Iₑ, I_{f}, I_{g}, Iₕ, Iᵢ, Iᵢ₁, Iⱼ, Iⱼ₁, Iₖ, Iₗ, Iₘ and Iₙ, respectively.

In one embodiment, the processes for the preparation of the compounds of formula (I), wherein D is a drug containing a carboxylic acid functional group is provided herein below. One such process for the preparation of the compound of formula (I), wherein D is a drug containing a carboxylic acid group, consists of the following reaction steps as outlined in the following Scheme 1:

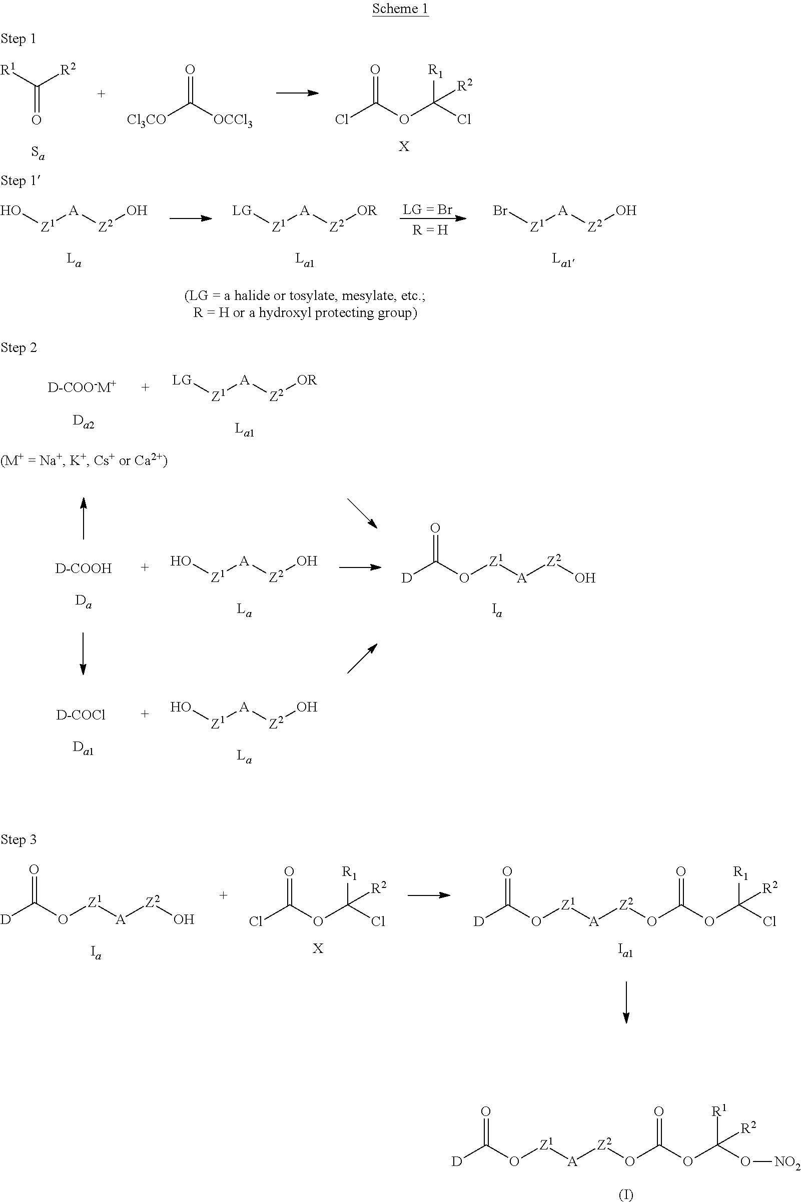

### Step 1

This process step involves reacting an aldehyde represented by formula (Sₐ) (wherein, R¹ and R² are as defined in any of the embodiments of the present invention), with triphosgene (or phosgene or diphosgene or any other phosgene substitutes known to those skilled in the art) in the presence of a suitable organic base for example, pyridine at −10° to 40° C. according to the method described in M. J. Coghlam and B. A. Caley, Tetrahedron Letters, 1989, 2033-2036, to obtain the chloroformate of formula (X).

### Step 1′

In this step, the linker Lₐ is converted to Lₐ₁ wherein one of the hydroxyl group is converted to a leaving group (LG) such as a halide or tosylate or mesylate and the other hydroxyl group is either left unprotected or is protected by a suitable hydroxyl protecting group and the processes used for the said conversions are generally known to those skilled in the art of organic synthesis.

### Step 2

In this step, the drug containing carboxylic acid group Dₐ (D-COOH) is treated with carbonyl chloride, for example oxalyl chloride, in the presence of an organic solvent, for example, dichloromethane and dimethylformamide in catalytic amount to form a reactive carbonyl derivative such as the acid chloride of formula Dₐ₁. Also, the carboxylic acid group of the drug Dₐ is converted to its carboxylate metal salt (Dₐ₂), for example, to a cesium salt. The drug containing carboxylic acid group (Dₐ) or its reactive acid chloride (Dₐ₁) is then directly coupled with the compound of formula (Lₐ) in the presence of a coupling agent, for example, N,N-dicyclohexylcarbodiimide (DCC) and an organic base, for example, triethylamine to form a compound intermediate (Iₐ). The reaction of the drug containing carboxylate metal salt (Dₐ₂) with linker intermediate Lₐ₁ (as obtained in step 1′ above) in the presence of an organic solvent, for example dimethylformamide (DMF) to obtain a compound of formula (Iₐ).

### Step 3

The compound intermediate (Iₐ) as obtained in step 2 above is further reacted with the chloroformate (X) obtained in step 1 above in the presence of an organic base, for example, pyridine and an organic solvent, for example, dichloromethane (DCM) to obtain the intermediate compound (Iₐ₁). The resulting compound (Iₐ₁) is subjected to nitration using silver nitrate in the presence of an organic solvent, for example, acetonitrile to form the compound of formula (I), and if desired, the compound of formula (I) is converted to its pharmaceutically acceptable salt.

In scheme 1, the variables D, R¹, R², Z¹, Z² and A are as defined in any of the embodiments of the present invention with reference to the compounds of formula (I) wherein D constitutes a drug containing carboxylic acid group.

Alternatively, the compounds of formula (I), wherein D is a drug containing a carboxylic acid group, can be prepared in accordance with a process involving the reaction steps depicted in the following Scheme 2.

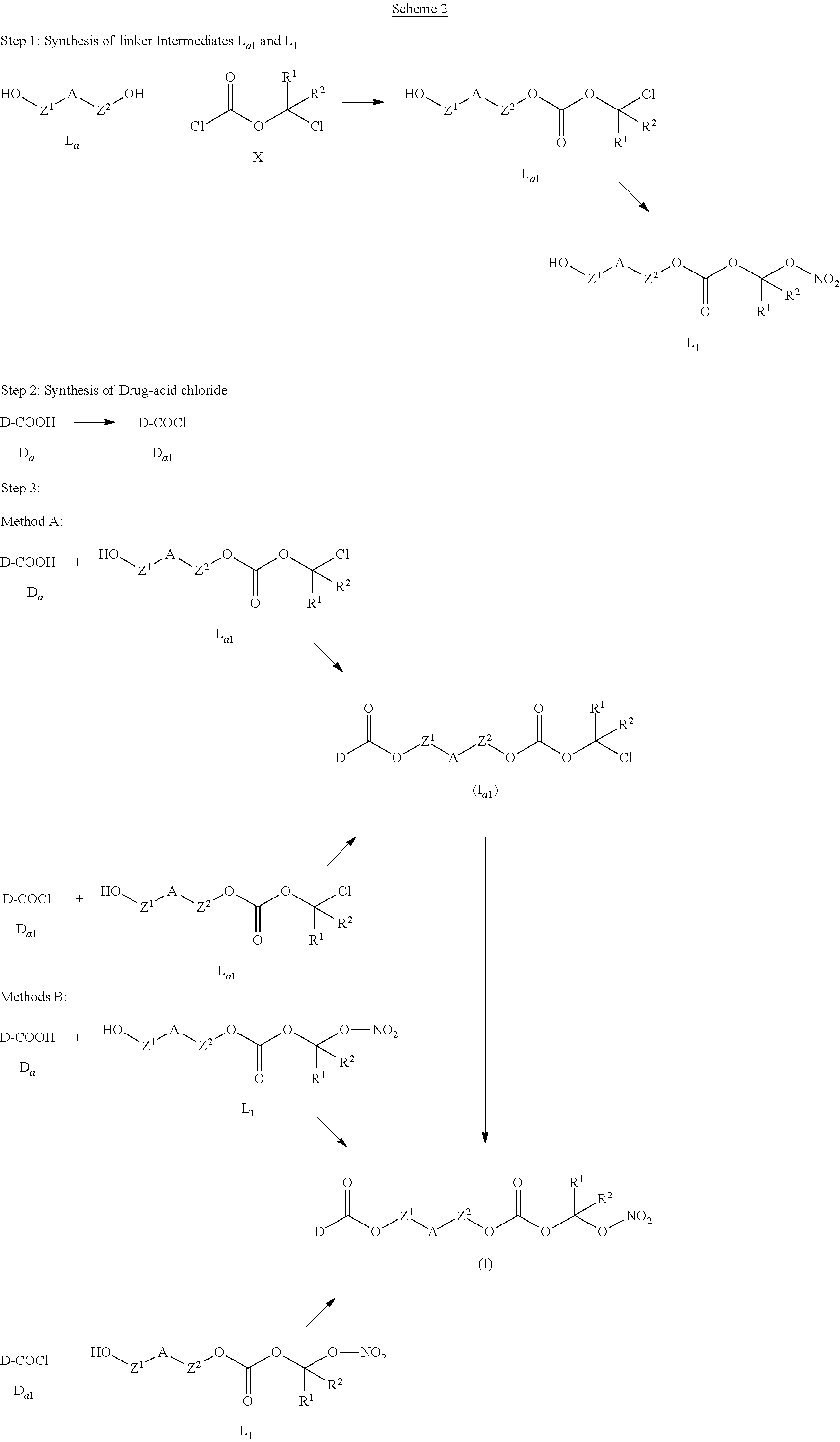

### Step 1

In this step, the linker (L₁) containing ONO₂ group is produced by: (i) reacting α-chloroformate (X) (as obtained in step 1 of Scheme 1) with a compound of formula (Lₐ) in the presence of a base, for example, pyridine and a solvent, for example, dichloromethane (DCM) to obtain the compound of formula (Lₐ₁); and (ii) subjecting the resultant compound of formula (Lₐ₁) to nitration using silver nitrate in the presence of an organic solvent, for example, acetonitrile.

### Step 2

In this step, the drug containing carboxylic acid group Dₐ is converted to its reactive carbonyl derivative such as an acid chloride of formula (Dₐ₁) as depicted in Step 2, Scheme 1.

### Step 3

### Method A:

The drug Dₐ is directly coupled with the linker of formula (Lₐ₁), as obtained in step 1 above, in the presence of a coupling agent, for example, N,N-dicyclohexylcarbodiimide (DCC) and an organic base, for example, 4-dimethylaminopyridine (DMAP) to form the compound of formula (Iₐ₁). Alternatively, treatment of the acid chloride (Dₐ₁) with the linker of formula (Lₐ₁) in the presence of a base, for example triethylamine also gives the compound of formula (Iₐ₁). Finally, the resulting compound (Iₐ₁) is subjected to nitration using silver nitrate in the presence of an organic solvent, for example acetonitrile to form the compound of formula (I), and if desired, the compound of formula (I) is converted to its pharmaceutically acceptable salt.

### Method B:

In this method, the drug (Dₐ) is directly coupled with the linker of formula (L₁), as obtained in step 1 above, in the presence of a coupling agent, for example, N,N-dicyclohexylcarbodiimide (DCC) and an organic base, for example, 4-dimethylaminopyridine (DMAP) to form the compound of formula (I). Alternatively, treatment of acid chloride (Dₐ₁) with the linker of formula (L₁) in the presence of a base, for example triethylamine also gives the compound of formula (I), and if desired, the compound of formula (I) is converted to its pharmaceutically acceptable salt.

In scheme 2, the variables D, R¹, R², Z¹, Z² and A are as defined in any of the embodiments of the present invention with reference to the compounds of formula (I) wherein D constitutes a drug containing carboxylic acid group.

Another process for the preparation of compound of formula (I), wherein D is a drug containing a carboxylic acid group, can be prepared in accordance with a process involving the reaction steps depicted in the following Scheme 3.

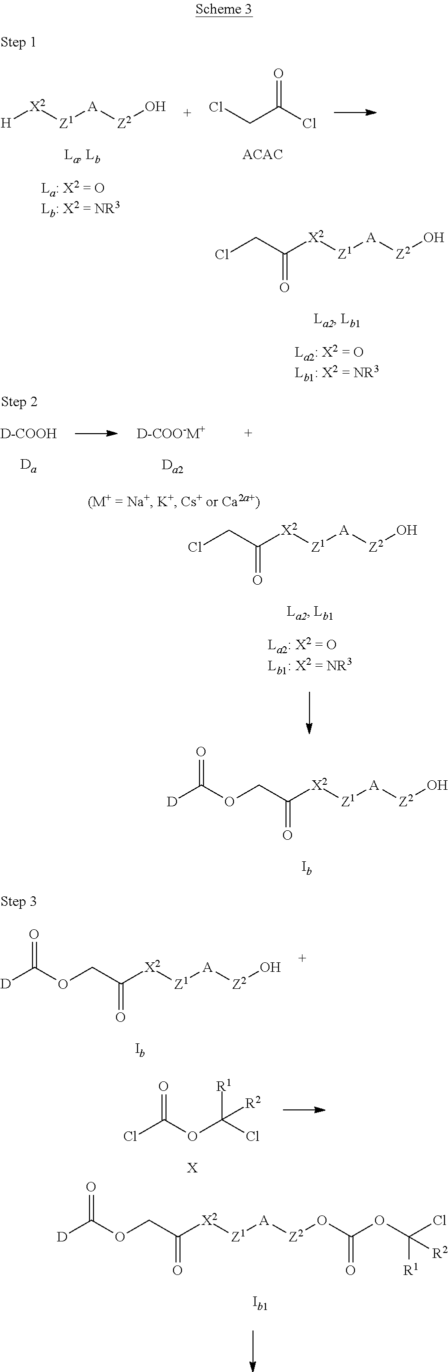

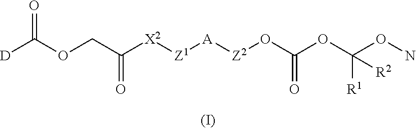

### Step 1

In this step, the linkers of formula Lₐ (X²═O) and L_{b} (X²═NR³, wherein R³ is as defined above) is reacted with α-chloro acetyl chloride (ACAC) in the presence of a base, for example, triethylamine and a solvent, for example, dichloromethane (DCM), to obtain the respective compounds of formula Lₐ₂ (X²═O) and L_{b1} (X²═NR³, wherein R³ is as defined above).

### Step 2

The drug Dₐ is treated with a metal carbonate, for example, cesium carbonate or calcium carbonate, in the presence of an organic solvent, for example, N,N-dimethylformamide (DMF), to form the corresponding cesium or calcium salt of the drug (designated as Dₐ₂). The resultant cesium or calcium salt of the drug (Dₐ₂) is directly coupled with the compounds of formula Lₐ₂ and L_{b1} as obtained in the above step 1, in the presence of an organic solvent, for example DMF, to obtain an intermediate compound (I_{b}) (wherein X²═O or NR³, wherein R³ is as defined above).

### Step 3

The compound of formula (I_{b}) as obtained in step 2 above is further reacted with the chloroformate (X) (as obtained in step 1 of Scheme 1) to obtain another intermediate compound (I_{b1}). The intermediate compound (I_{b1}) is further subjected to nitration in the presence of silver nitrate and acetonitrile to obtain the compound of formula (I).

In scheme 3, the variables D, R¹, R², Z¹, Z² and A are as defined in any of the embodiments of the present invention with reference to the compounds of formula (I) wherein D constitutes a drug containing carboxylic acid group.

Another process for the preparation of compound of formula (I), wherein D is a drug containing a carboxylic acid group, can be prepared in accordance with a process involving the reaction steps depicted in the following Scheme 4.

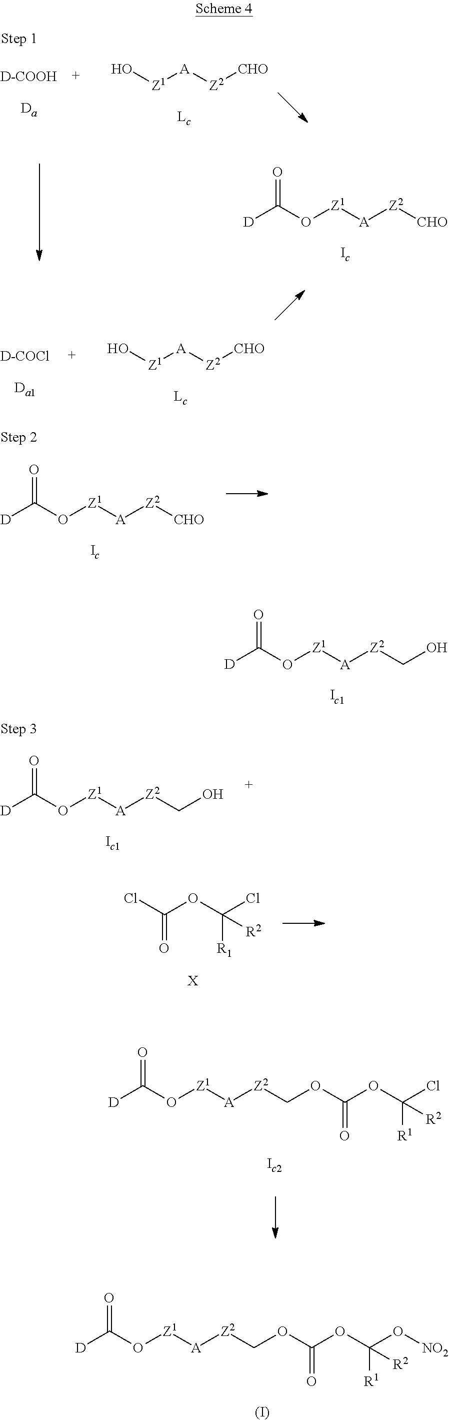

### Step 1

In this step, the drug Dₐ or its reactive carbonyl chloride derivative (Dₐ₁) (as obtained in step 2 of Scheme 1) is coupled with the compound of formula (L_{c}) in the presence of a coupling agent, for example, N,N-dicyclohexylcarbodiimide (DCC) or an organic base, for example, triethylamine to obtain an intermediate compound (I_{c}).

### Step 2

The intermediate compound (I_{c}) as obtained in step 1 above is subjected to reduction using sodium borohydride in the presence of a solvent, for example, methanol, to form intermediate compound (I_{c1}).

### Step 3

The compound intermediate (I_{c1}) is further reacted with the chloroformate (X) (as obtained in step 1 of Scheme 1) in the presence of an organic solvent, for example, dichloromethane (DCM), and an organic base, for example, pyridine, to obtain an intermediate compound (I_{c2}). The intermediate compound (I_{c2}) is subjected to nitration using silver nitrate and in the presence of an organic solvent, for example, acetonitrile to form a compound of formula (I).

In scheme 4, the variables D, R¹, R², Z¹, Z² and A are as defined in any of the embodiments of the present invention with reference to the compounds of formula (I) wherein D constitutes a drug containing carboxylic acid group.

An alternative process for the preparation of compound of formula (I), wherein D is a drug containing a carboxylic acid group and the variable A is D-isosorbide skeleton, can be prepared in accordance with a process involving the reaction steps depicted in the following Scheme 5.

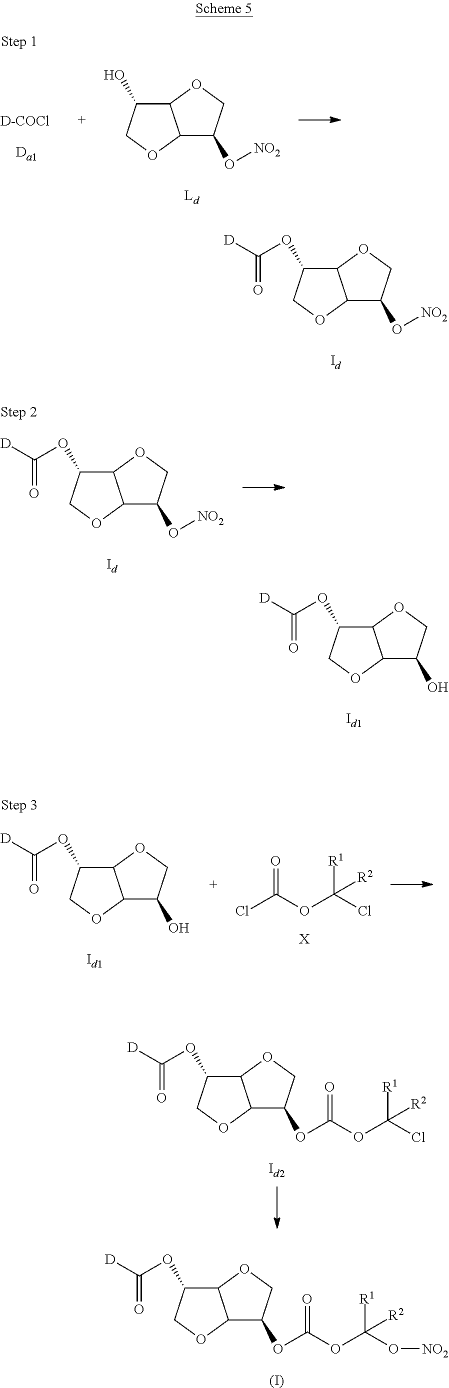

### Step 1

In this step, the reactive carbonyl derivative i.e. the acid chloride Dₐ₁ of the drug Dₐ (as obtained in step 2 of Scheme 1) is reacted with isosorbide-5-mononitrate (L_{d}) in the presence of an organic base, for example, triethylamine and an organic solvent, for example, toluene at a temperature of 0°-15° C. for a period of 24 hours according to the method described in the reference J. F. Gilmar et al., Eur J Pharm Sci 2001, 14, 221-227, to form the intermediate compound (I_{d}). The cited reference is incorporated herein by reference.

### Step 2

The intermediate compound (I_{d}) as obtained in step 1 above is further subjected to reduction using a hydrogenation catalyst, 10% palladium/carbon (10% Pd on C) in the presence of an organic solvent selected from methanol or ethyl acetate according to the procedure described in the reference L M Moriarty et al., J Med Chem 2008, 51, 7991-7999, to obtain another intermediate compound (I_{d1}). The cited reference is incorporated herein by reference.

### Step 3

The compound intermediate (I_{d1}), as obtained in step 2 above, is further reacted with α-chloroformate (X) (as obtained in step 1 of scheme 1) in the presence of an organic solvent, for example, dichloromethane (DCM) and an organic base, for example, pyridine to produce the intermediate compound (I_{d2}). The intermediate compound (I_{d2}) is subjected to nitration using silver nitrate and in the presence of an organic solvent, for example, acetonitrile to obtain the compound of formula (I).

In scheme 5, the variables D, R¹, R², Z¹ and Z² are as defined in any of the embodiments of the present invention with reference to the compounds of formula (I) wherein D constitutes a drug containing a carboxylic acid group.

Another process for the preparation of compound of formula (I), wherein D is a drug containing a carboxylic acid group and the variable A is S, SO or SO₂, can be carried out in accordance with the reaction steps depicted in the following Scheme 6.

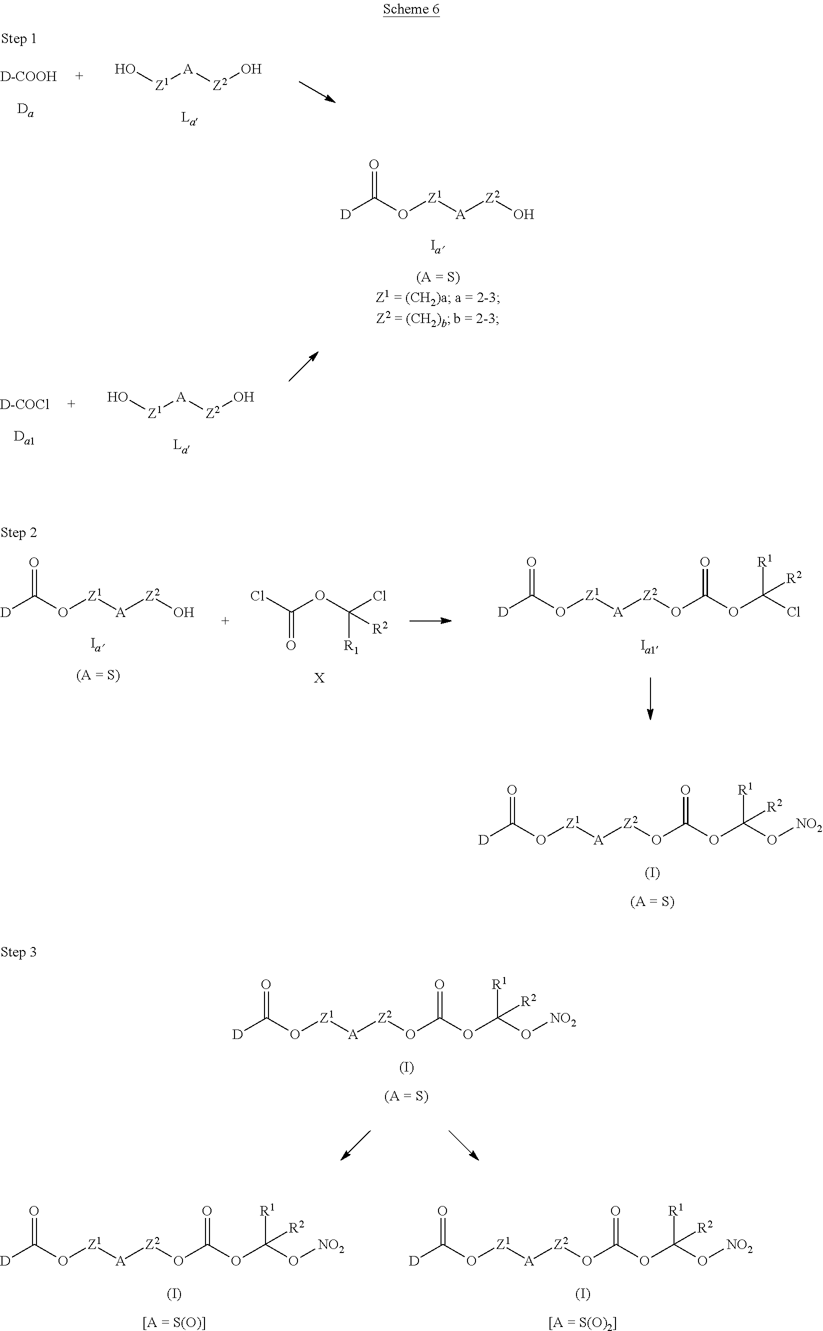

### Step 1

In this step, the drug Dₐ or its reactive carbonyl chloride derivative Dₐ₁ (as obtained in step 2 of Scheme 1) is coupled with the compound of formula (Lₐ) (wherein, A is S) in the presence of a coupling agent, for example, N,N-dicyclohexylcarbodiimide (DCC), an organic base, for example, dimethylaminopyridine (DMAP) and a solvent selected from dichloromethane (DCM) or tetrahydrofuran (THF) to obtain the intermediate compound (I_{a′}) (wherein, A is S).

### Step 2

The compound intermediate (I_{a′}) as obtained in step 1 above is reacted with the chloroformate (X) (as obtained in step 1 of Scheme 1) in the presence of a base, for example, pyridine and a solvent, for example, dichloromethane (DCM) to obtain an intermediate compound (I_{a′}). The intermediate compound (I_{a′}) is subjected to nitration using silver nitrate, in the presence of an organic solvent, for example, acetonitrile, to obtain the compound of formula (I) (wherein A=S).

### Step 3

The compound of formula (I) (wherein A=S) as obtained in step 2 above is subjected to oxidation in the presence of an oxidising agent, for example, sodium periodate in water in the presence of an organic solvent selected from methanol or acetone, to obtain the compound of formula (I) (wherein A=SO). Alternatively, the compound of formula (I) (wherein A=S) is treated with oxone in the presence of an organic solvent, for example, methanol, to obtain the compound of formula (I) (wherein A=SO₂).

In scheme 6, the variables D, R¹, R², Z¹ and Z² are as defined in any of the embodiments of the present invention with reference to the compounds of formula (I) wherein D constitutes a drug containing carboxylic acid group.

A process for the preparation of the compound of formula (I), wherein D is a drug containing one or more functional groups independently selected from an amino, a hydroxy or a sulfhydryl group, can be carried out in accordance with the reaction steps depicted in the following Scheme 7.

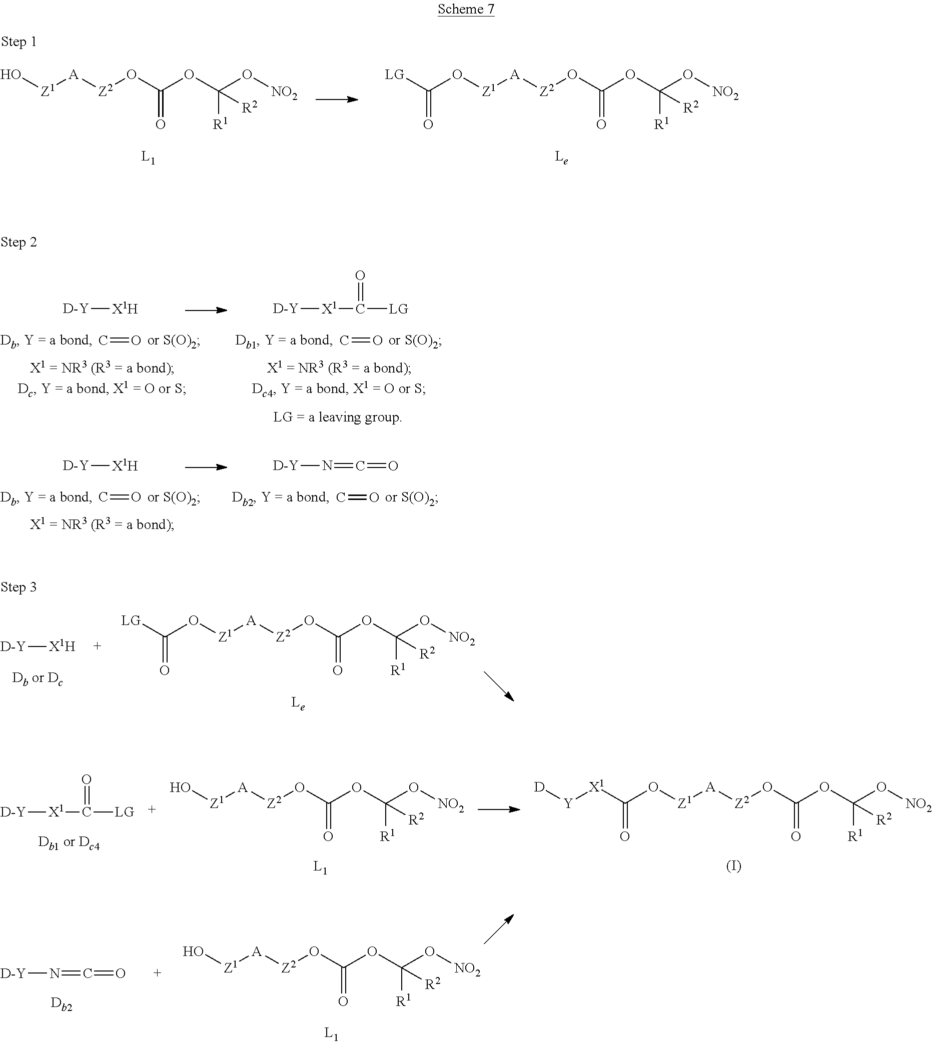

### Step 1

In this step, the linker (L₁) (as obtained in step 1 of Scheme 2) is reacted with phosgene or its equivalent selected from diphosgene, triphosgene, N,N′-carbonyldiimidazole (CDI), N,N′-disuccinimidyl carbonate (DSC) or 4-nitrophenyl chloroformate in the presence of a base, for example, pyridine or triethylamine and a solvent, for example, dichloromethane (DCM) to obtain the corresponding alkoxycarbonyl derivative of the linker L₁, designated herein as Lₑ, wherein LG is a suitable leaving group selected from halide, imidazole, N-hydroxysuccinimide or 4-nitrophenyl group.

### Step 2

The drug containing an amino group D_{b} (D-Y—X¹H, wherein Y=a bond, C═O or S(O)₂; X¹═NR³, wherein R³ is a bond) or the drug containing a hydroxyl or sulfhydryl group D_{c} (D-Y—X¹H, wherein Y=a bond; X¹═O or S) is reacted with phosgene or its equivalent selected from: diphosgene, triphosgene, N,N′-carbonyldiimidazole (CDI), N,N′-disuccinimidyl carbonate (DSC) or 4-nitrophenyl chloroformate in the presence of a base, for example, triethylamine and a solvent, for example, dichloromethane (DCM) to obtain the corresponding reactive carbonyl derivative of the drug D_{b} or D_{c} designated herein as D_{b1} and D_{c4} respectively wherein LG is a suitable leaving group selected from halide, imidazole, N-hydroxysuccinimide or 4-nitrophenyl group.

Similarly, the drug containing an amino group D_{b} (D-Y—X¹H, wherein Y=a bond, C═O or S(O)₂; X¹═NR³, wherein R³ is H) is converted to its reactive isocyanate derivative D_{b2} by methods known to those skilled in the art i.e., either by the reaction of corresponding primary amine-containing drug D_{b} (D-Y—X¹H, wherein Y=a bond; X¹═NR³, wherein R³ is H) with phosgene or its equivalent (Reference: Shriner, R. L. et al., Org. Synth. Coll. Vol. 2, (1943), 453) or by the reaction of corresponding amide/sulfonamide-containing drug D_{b} (D-Y—X¹H, wherein Y═C(═O) or S(O)₂; X¹═NR³, wherein R³ is H) with oxalyl chloride (Reference: Speziale, A. J. et al., J. Org. Chem. 1962, 27, 3742 and Speziale, A. J. et al., J. Org. Chem. 1963, 28, 1805-1811).

### Step 3

The drug containing an amino group D_{b} (D-Y—X¹H, wherein Y=a bond, C═O or S(O)₂; X¹═NR³, wherein R³ is a bond or H) or the drug containing a hydroxyl or sulfhydryl group D_{c} (D-Y—X¹H, wherein Y=a bond; X¹═O or S) is reacted with the compound (Lₑ) (as obtained in step 1 above) or the reactive carbonyl derivative D_{b1} or D_{c4} (as obtained in Step 2 above) of the drugs D_{b} and D_{c} respectively is reacted with the linker (L₁) in the presence of a base, for example, triethylamine and a solvent, for example, dichloromethane (DCM) to obtain the compound of formula (I).

Alternatively, the reactive isocyanate derivative D_{b2} (as obtained in Step 2 above) of the drug D_{b} is reacted with the linker L₁ in the presence of a base, for example, triethylamine and a solvent, for example, dichloromethane (DCM) to obtain the desired compound of formula (I).

In scheme 7, the variables D, A, R¹, R², Z¹ and Z² are as defined in any of the embodiments of the present invention with reference to the compounds of formula (I) wherein D constitutes a drug containing a hydroxyl, a sulfhydryl or an amino group.

A process for the preparation of the compound of formula (I), wherein D is a drug containing one or more functional groups independently selected from an amino, a hydroxyl or a sulfhydryl group, can be carried out in accordance with the reaction steps depicted in the following Scheme 8.

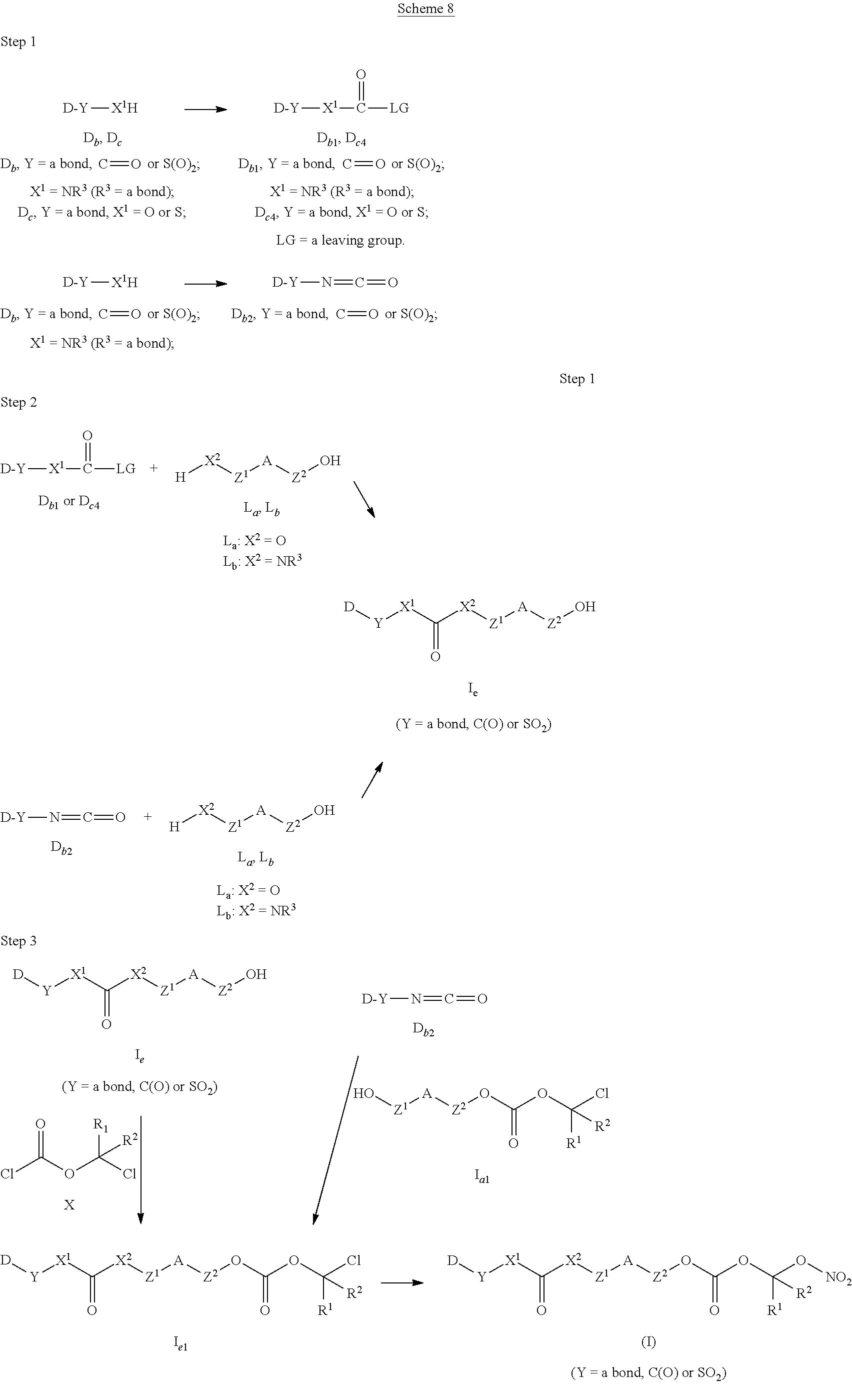

The drug containing an amino group D_{b} (D-Y—X¹H, wherein Y=a bond, C═O or S(O)₂; X¹═NR³, wherein R³ is a bond) or the drug containing a hydroxyl or sulfhydryl group D_{c} (D-Y—X¹H, wherein Y=a bond; X¹═O or S) is converted to its corresponding reactive carbonyl derivative designated herein as D_{b1} and D_{c4} respectively (as depicted in Step 2, Scheme 7). Similarly, the drug containing an amino group D_{b} (D-Y—X¹H, wherein Y=a bond, C═O or S(O)₂; X¹═NR³, wherein R³ is H) is converted to its reactive isocyanate derivative D_{b2} (as depicted in Step 2, Scheme 7).

### Step 2

In this step, the reactive carbonyl derivative D_{b1} [of the drug containing an amino group D_{b} (D-Y—X¹H, wherein Y=a bond, C═O or S(O)₂; X¹═NR³, wherein R³ is a bond)] or D_{c4} [of the drug containing a hydroxyl or a sulfhydryl group D_{c} (D-Y—X¹H, wherein Y=a bond; X¹═O or S)] as obtained in Step 1 above, is reacted with the compound of formula (Lₐ) (X²═O) or the compound of formula (L_{b}) (X²═NR³, wherein R³ is as defined above) to obtain the intermediate compound (Iₑ). Similarly, the reactive isocyanate derivative D_{b2} of the drug containing an amino group D_{b} (D-Y—X¹H, wherein Y=a bond, C═O or S(O)₂; X¹═NR³, wherein R³ is H) is reacted with the compound of formula Lₐ (X²═O) or the compound of formula L_{b} (X²═NR³, wherein R³ is as defined above) to obtain the intermediate compound (Iₑ).

### Step 3

The intermediate compound (Iₑ) as obtained in step 2 above is then reacted with the chloroformate (X) (as obtained in step 1 of Scheme 1) to obtain the intermediate compound (Iₑ₁), which is subjected to nitration using silver nitrate, in the presence of an organic solvent, for example, acetonitrile, to obtain the compound of formula (I). Alternatively, the reactive isocyanate derivative D_{b2} of the drug containing an amino group D_{b} (D-Y—X¹H, wherein Y=a bond, C═O or S(O)₂; X¹═NR³, wherein R³ is H) as obtained in Step 1 above, is reacted with the compound of formula Lₐ₁ in the absence or presence of a base, for example, triethylamine and a solvent, for example, dichloromethane (DCM) to obtain the compound of formula (Iₑ₁) which is finally nitrated using silver nitrate in the presence of an organic solvent, for example acetonitrile to form the compound of formula (I).

In scheme 8, the variables D, A, R¹, R², Z¹ and Z² are as defined in any of the embodiments of the present invention with reference to the compounds of formula (I) wherein D constitutes a drug containing hydroxyl, sulfhydryl or amino group.

A process for the preparation of the compound of formula (I), wherein D is a drug containing one or more functional groups independently selected from an amino, a hydroxyl or a sulfhydryl group, can be prepared in accordance with a process involving the reaction steps depicted in the following Scheme 9.

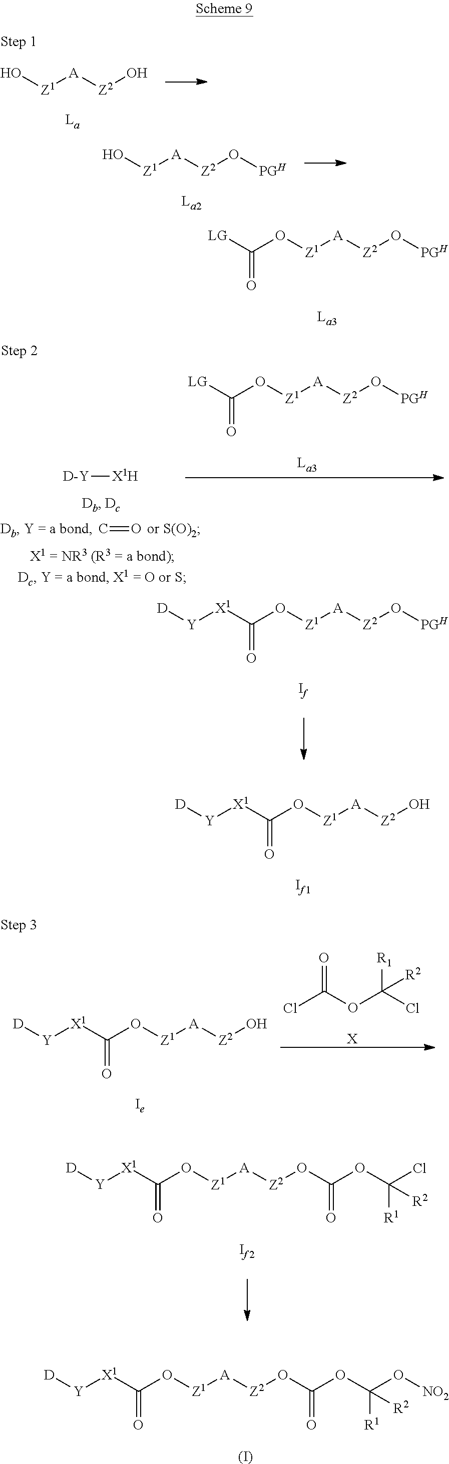

### Step 1

In this step, one of the hydroxyl groups of the linker diol (Lₐ) is selectively protected by a suitable hydroxyl protecting group by a standard method to obtain the corresponding monoprotected compound of formula (Lₐ₂). The resultant compound of formula (Lₐ₂) is further treated with phosgene or its equivalents: diphosgene, triphosgene, N,N′-carbonyldiimidazole (CDI), N,N′-disuccinimidyl carbonate (DSC) or 4-nitrophenyl chloroformate in the presence of a base, for example, pyridine or triethylamine and a solvent, for example, dichloromethane (DCM) to obtain the compound of formula (Lₐ₃).

### Step 2

In this step, the drug containing an amino group D_{b} (D-Y—X¹H, wherein Y=a bond, C═O or S(O)₂; X¹═NR³, wherein R³ is a bond or H) or the drug containing a hydroxyl or sulfhydryl group D_{c} (D-Y—X¹H, wherein Y=a bond; X¹═O or S) is reacted with the compound of formula (Lₐ₃) as obtained in step 1 above in the presence of a suitable base, for example, triethylamine and a solvent, for example, dichloromethane (DCM) to form the intermediate compound (I_{f}). Removal of hydroxyl protecting group from the intermediate compound (I_{f}) is carried out using a standard procedure in the art to obtain the intermediate compound (I_{f1}).

### Step 3

In this step, the intermediate compound (I_{f1}) is reacted with the chloroformate (X) (as obtained in step 1 of Scheme 1) to obtain the intermediate compound (I_{f2}). The intermediate compound (I_{f2}) is further subjected to nitration using silver nitrate in the presence of an organic solvent, for example, acetonitrile, to form the compound of formula (I).

In scheme 9, the variables D, A, R¹, R², Z¹ and Z² are as defined in any of the embodiments of the present invention with reference to the compounds of formula (I) wherein D constitutes a drug containing a hydroxyl, a sulfhydryl or an amino group.

An alternative process for the preparation of the compound of formula (I), wherein D is a drug containing one or more functional groups independently selected from a hydroxyl or a sulfhydryl group and the variable A is selected from the groups consisting of 1,2-, 1,3-, and 1,4-phenylene and both, Z¹ and Z² represent bond, can be prepared in accordance with the process involving the reaction steps depicted in the following Scheme 10.

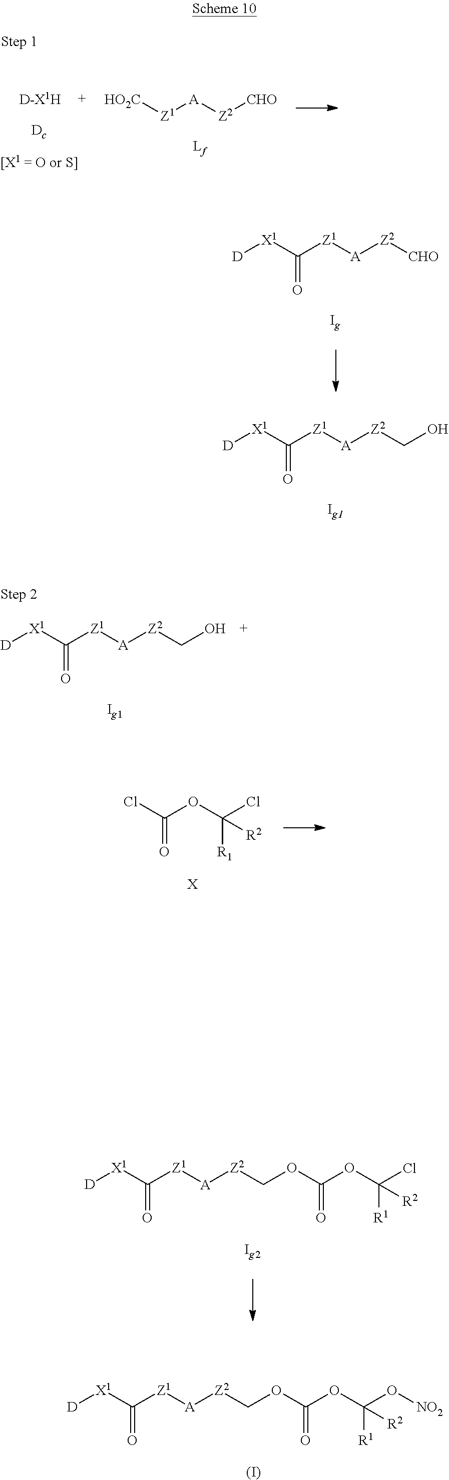

### Step 1

In this step, the drug containing hydroxyl or sulfhydryl functional group D_{c} (D-Y—X¹H, wherein Y=a bond; X¹═O or S) is directly coupled with the compound of formula (L_{f}) (wherein A=1,2-, 1,3-, or 1,4-phenylene and Z¹ and Z²=bond) in the presence of a coupling agent, for example, N,N-dicyclohexylcarbodiimide (DCC) and in the presence of an organic base, for example, dimethylaminopyridine (DMAP) and a solvent, for example, dichloromethane (DCM) to obtain an intermediate compound (I_{g}). The intermediate compound (I_{g}) is further subjected to reduction in the presence of a reducing agent, for example, sodium borohydride and in a solvent, for example, methanol to obtain another intermediate compound (I_{g1}).

### Step 2

The intermediate compound (I_{g1}) is further reacted with the chloroformate (X) (as obtained in step 1 of Scheme 1) to obtain another intermediate compound (I_{g2}). The intermediate compound (I_{g2}) is further subjected to nitration using silver nitrate in the presence of an organic solvent, for example, acetonitrile, to form the compound of formula (I).

In scheme 10, the variables D, R¹, R² are as defined in any of the embodiments of the present invention with reference to the compounds of formula (I) wherein D constitutes a drug containing a hydroxyl or a sulfhydryl group. It has already been indicated hereinabove that A=1,2-, 1,3-, and 1,4-phenylene and Z¹ and Z²=bond.

An alternative process for the preparation of the compound of formula (I), wherein D is a drug containing carboxylic acid group and the variable Y is a spacer group Y_{b}=

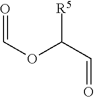

=(wherein R⁵ is as defined above), can be prepared in accordance with a process involving the reaction steps depicted in the following Scheme 11.

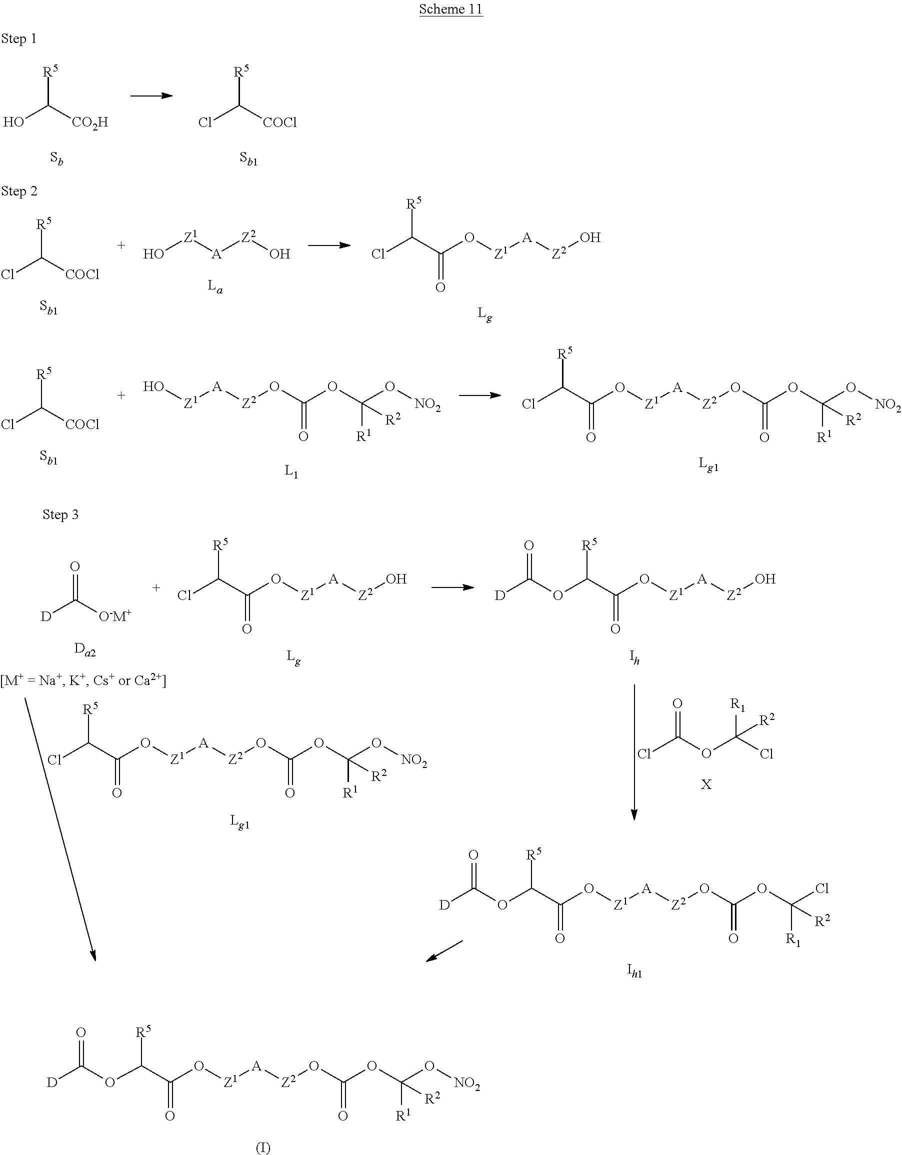

### Step 1

In this step, the compound of formula (S_{b}) is reacted with phosphorous pentachloride or sulphonyl chloride to obtain the compound of formula (S_{b1}).

### Step 2

The compound (S_{b1}) as obtained in step 1 above is further reacted with the compound of formula (Lₐ) or the linker (L₁) in the presence of a base, for example, triethylamine and a solvent, for example, dichloromethane (DCM) to obtain the respective compound of formula (L_{g}) or (L_{g1}).

### Step 3

In this step, the metallic salt Dₐ₂ (wherein M⁺═Na⁺, K⁺, Ca²⁺ or Cs⁺) of the drug containing carboxylic acid group Dₐ is directly coupled with the compound of formula (L_{g}) as obtained in step 2 above in the presence of an organic solvent, for example, N,N-dimethylformamide (DMF) to obtain an intermediate compound (Iₕ). The intermediate compound (Iₕ) is further reacted with the chloroformate (X) (as obtained in step 1 of Scheme 1) to obtain another intermediate compound (Iₕ₁). The intermediate compound (Iₕ₁) is then subjected to nitration using silver nitrate in the presence of an organic solvent, for example, acetonitrile, to form the compound of formula (I). Alternatively, the metallic salt Dₐ₂ (wherein M⁺═Na⁺, K⁺, Ca²⁺ or Cs⁺) of the drug containing carboxylic acid group Dₐ is reacted with the compound of formula (L_{g1}) in the presence of an organic solvent, for example, N,N-dimethylformamide (DMF) to obtain the compound of formula (I).

In scheme 11, the variables D, Z¹, Z², A, R¹ and R² are as defined in any of the embodiments of the present invention with reference to the compounds of formula (I) wherein D constitutes a drug containing carboxylic acid group or its metallic salt as specified above.

A process for the preparation of the compound of formula (I), wherein D is a drug containing one or more functional groups independently selected from hydroxyl or sulfhydryl group and Y is a spacer group, Y_{c}=

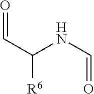

(wherein R⁶ is as defined above) can be prepared in accordance with a process involving the reaction steps depicted in the following Scheme 12.

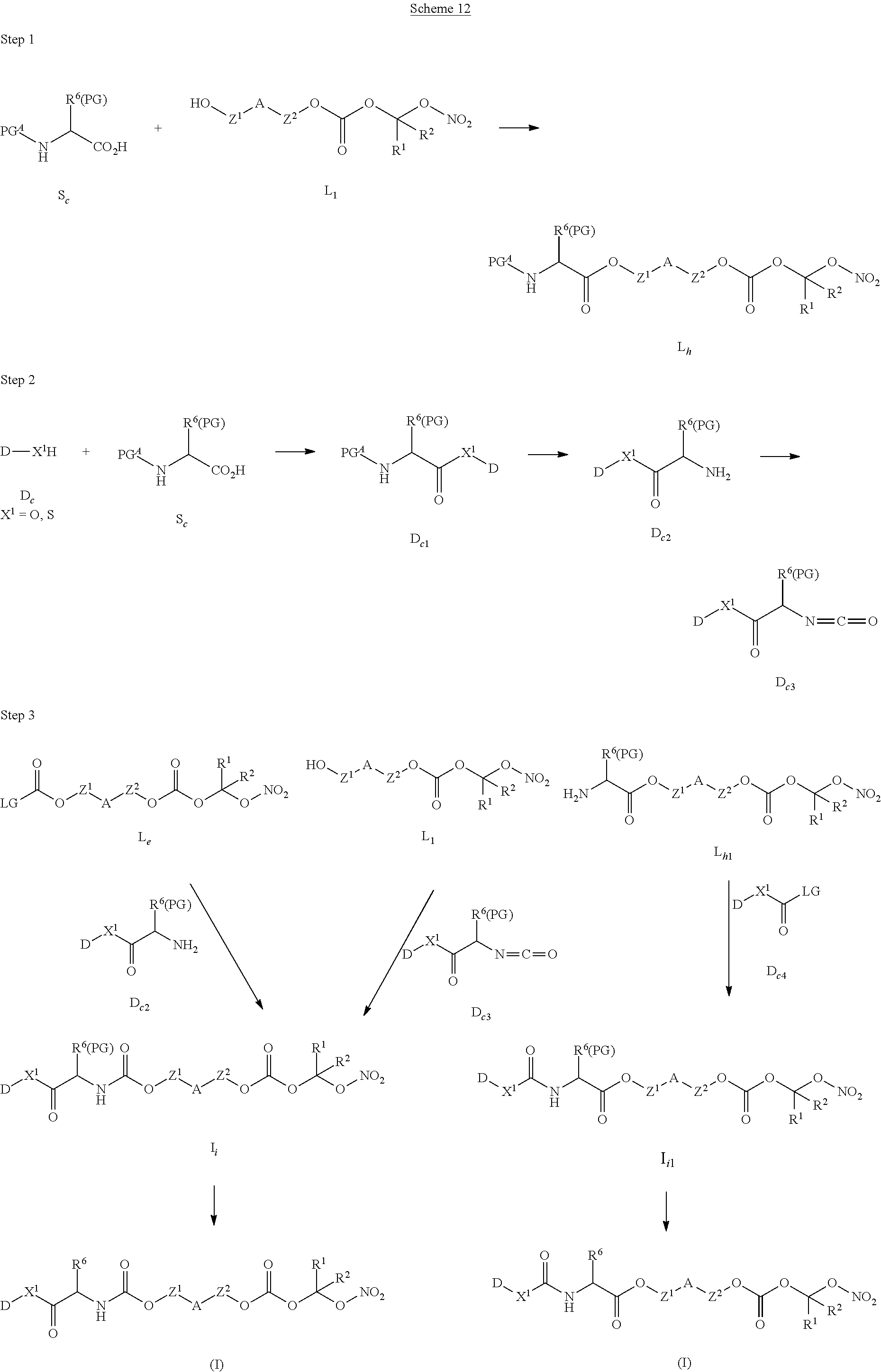

### Step 1

In this step, the compound of formula (S_{c}) (wherein PG^{A} is an amino protecting group as defined above and R⁶ is as defined above) is reacted with the linker (L₁) in the presence of a coupling agent, for example, N,N-dicyclohexylcarbodiimide (DCC) and in the presence of an organic base, for example, dimethylaminopyridine (DMAP), and an organic solvent, for example, dichloromethane (DCM) to obtain the compound of formula (Lₕ). The removal of the amino protecting group PG^{A} in the compound of formula (Lₕ) is carried out by a standard procedure known in the art to form compound of formula (Lₕ₁).

### Step 2

In this step, the drug containing a hydroxyl or sulfhydryl group D_{c} (D-Y—X¹H, wherein Y=a bond; X¹═O or S) is reacted with the compound of formula (S_{c}) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain a reactive drug derivative of formula (D_{c1}). Removal of the amino protecting group PG^{A} from the reactive drug derivative (D_{c1}) is carried out using a standard procedure known in the art to obtain another reactive compound intermediate (D_{c2}). The drug derivative (D_{c2}) is further treated with phosgene or its equivalent selected from diphosgene, triphosgene, N,N′-carbonyldiimidazole (CDI), N, N′-disuccinimidyl carbonate (DSC) or 4-nitrophenyl chloroformate in the presence of a base, for example, triethylamine and a solvent, for example, dichloromethane (DCM) to obtain another reactive isocyanate intermediate (D_{c3}).

### Step 3

In this step, the drug derivative (D_{c2}) as obtained in step 2 above is reacted with the compound (Lₑ) (as obtained in step 1 of Scheme 7) in the presence of a base, for example, triethylamine and a solvent, for example, dichloromethane (DCM) to obtain the intermediate compound (Iᵢ). Removal of the protecting group from the intermediate compound (Iᵢ) is carried out using a standard procedure known in the art to form the compound of formula (I). Alternatively, the drug derivative D_{c3} as obtained in Step 2 above is reacted with the linker (L₁) to form the compound of formula (I) after removal of the protecting group from the protected intermediate of the formula (Iᵢ) thus obtained. Alternatively, the drug derivative D_{c4}, as obtained in Step 2, Scheme 7 (wherein, Y=a bond; X¹═O or S) is reacted with the compound (Lₕ₁) produced in reaction step 1 above to form a compound intermediate (Iᵢ₁). The removal of protecting group PG in the compound intermediate (Iᵢ₁) is carried out using any standard procedure known in the art to form the compound of formula (I).

In scheme 12, the variables D, A, Z¹, Z², R¹ and R² are as defined in any of the embodiments of the present invention with reference to the compounds of formula (I) wherein D constitutes a drug containing hydroxyl or sulfhydryl group.

An alternative process for the preparation of the compound of formula (I), wherein D is a drug containing one or more functional groups independently selected from hydroxyl or sulfhydryl group and Y is a spacer group selected from Y_{f}=

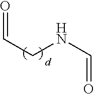

(wherein d is as defined above) can be carried out in accordance with the reaction steps as depicted in the following Scheme 13.

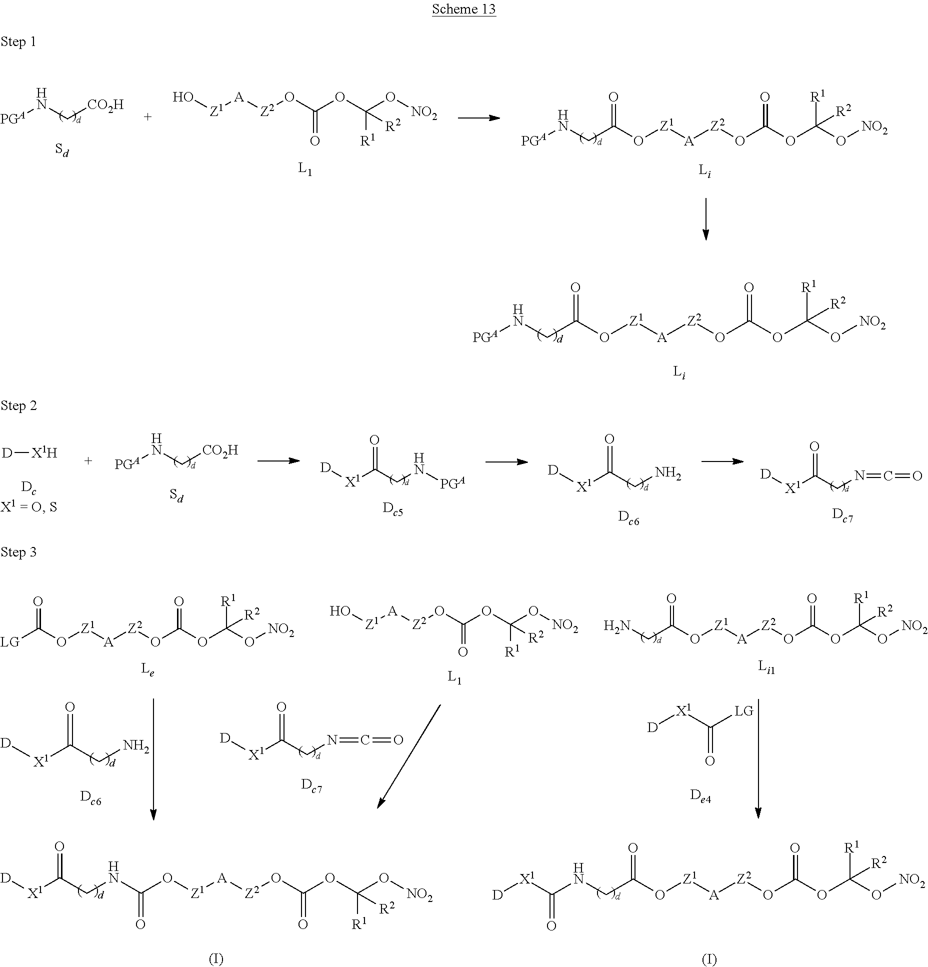

### Step 1

In this step, the compound of formula (S_{d}) (wherein PG^{A} is an amino protecting group as defined above) is reacted with the linker (L₁) in the presence of a coupling agent, for example, N,N-dicyclohexylcarbodiimide (DCC) and in the presence of an organic base, for example, dimethylaminopyridine (DMAP), and organic solvent, for example, dichloromethane (DCM) to obtain the compound of formula (Lᵢ). Removal of the amino protecting group PG^{A} in compound of formula (Lᵢ) is carried out by a standard procedure known in the art to form the compound of formula (Lᵢ₁).

### Step 2

The drug containing a hydroxyl or sulfhydryl group D_{c} (D-Y—X¹H, wherein Y=a bond; X¹═O or S) is reacted with the compound of formula (S_{d}) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain the corresponding derivative of the drug (D_{c5}). Removal of the amino protecting group PG^{A} from the drug derivative (D_{c5}) is carried out using a standard procedure known in the art to form another reactive free amine drug derivative (D_{c6}). The resulting free amine derivative (D_{c6}) is further treated with phosgene or its safe equivalent selected from diphosgene, triphosgene, N,N′-carbonyldiimidazole (CDI), N, N′-disuccinimidyl carbonate (DSC), 4-nitrophenyl chloroformate in the presence of a base, for example, triethylamine and a solvent, for example, dichloromethane to form another reactive compound i.e. the intermediate isocyanate compound (D_{c7}).

### Step 3

The resulting drug derivative (D_{c6}) is reacted with the compound of formula (Lₑ) (as obtained in step 1 of Scheme 7) in a solvent, for example, dichloromethane (DCM) to obtain the compound of formula (I). Alternatively, the drug isocyanate derivative (D_{c7}) as obtained in step 2 above is reacted with the linker (L₁) in a solvent, for example, dichloromethane (DCM) to form the nitrate ester prodrug of formula (I). In an alternative synthesis, the drug derivative (D_{c4}) (as obtained in Step 2, scheme 7, wherein Y=a bond) is reacted with the compound (Lᵢ₁) as obtained in step 1, to form the nitrate ester prodrug of formula (I).

In scheme 13, the variables D, Z¹, Z², A, R¹ and R² are as defined in any of the embodiments of the present invention with reference to the compounds of formula (I) wherein D constitutes a drug containing a hydroxyl or a sulfhydryl group.

Another process for the preparation of the compound of formula (I), wherein D is a drug containing one or more functional groups independently selected from a hydroxyl or a sulfhydryl group and Y is a spacer group selected from Yᵢ=

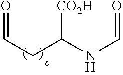

(wherein c is as defined above) can be carried out in accordance with the reaction steps as depicted in Scheme 14.

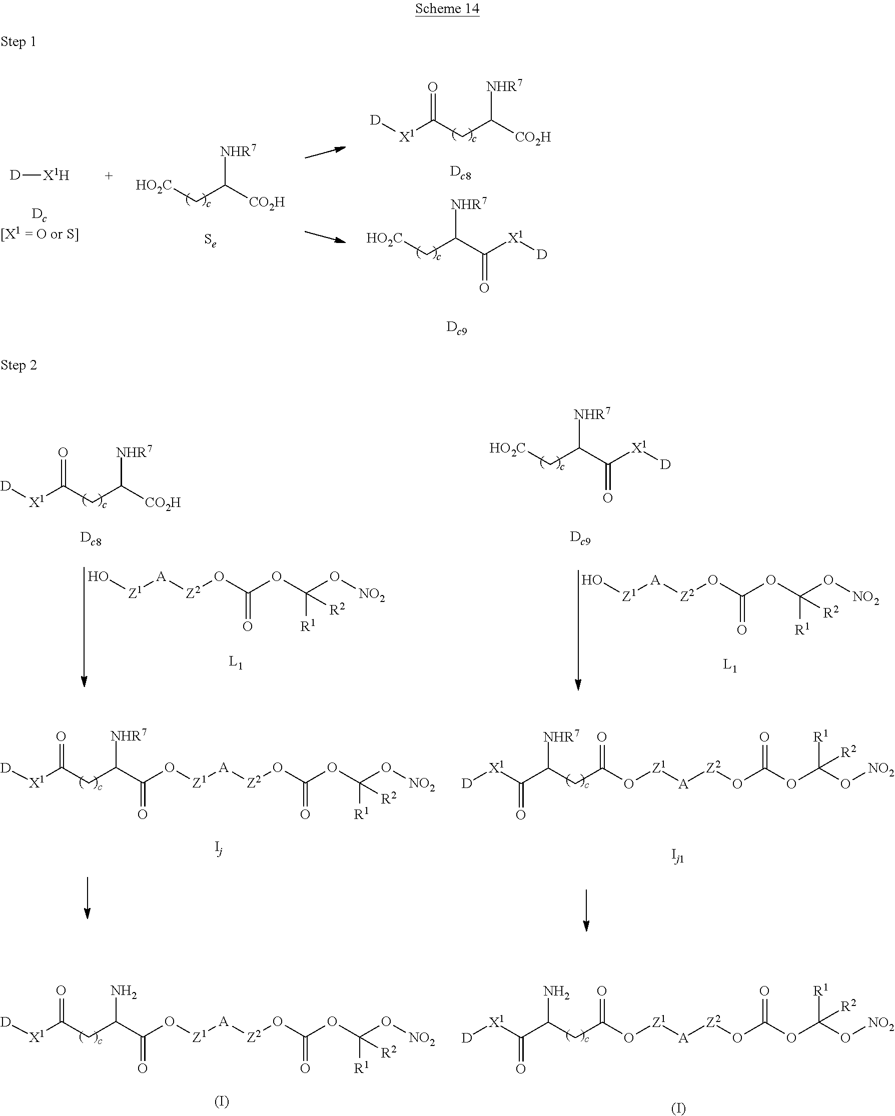

### Step 1

In this step, the drug containing a hydroxyl or sulfhydryl group D_{c} (D-Y—X¹H, wherein Y=a bond; X¹═O or S) is reacted with the compound of formula Sₑ (wherein R⁷ is an amino protecting group (PG^{A}) as defined above) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain a reactive drug derivative (D_{c8}) and/or a reactive drug derivative (D_{c9}).

### Step 2

The reactive drug derivative of formula D_{c8} or the reactive drug derivative of formula D_{c9} (as obtained in step 1 above) is directly coupled with the nitrate ester containing linker (L₁) (formed in reaction step 1 of Scheme 2) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain the intermediate compound (Iⱼ) and the intermediate compound (Iⱼ₁) respectively. Removal of the amino protecting group R⁷ from each of the intermediate compounds (Iⱼ) and (Iⱼ₁) is carried out by a standard procedure known in the art to obtain the respective compounds of formula (I).

In scheme 14, the variables D, Z¹, Z², A, R¹ and R² are as defined in any of the embodiments of the present invention with reference to the compounds of formula (I) wherein D constitutes a drug containing hydroxyl or sulfhydryl group.

Another process for the preparation of the compound of formula (I), wherein D is a drug containing one or more functional groups selected from a hydroxyl or a sulfhydryl group and Y is a spacer group selected from

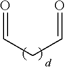

(wherein d is as defined above) can be carried out in accordance with the reaction steps as depicted in the following Scheme 15.

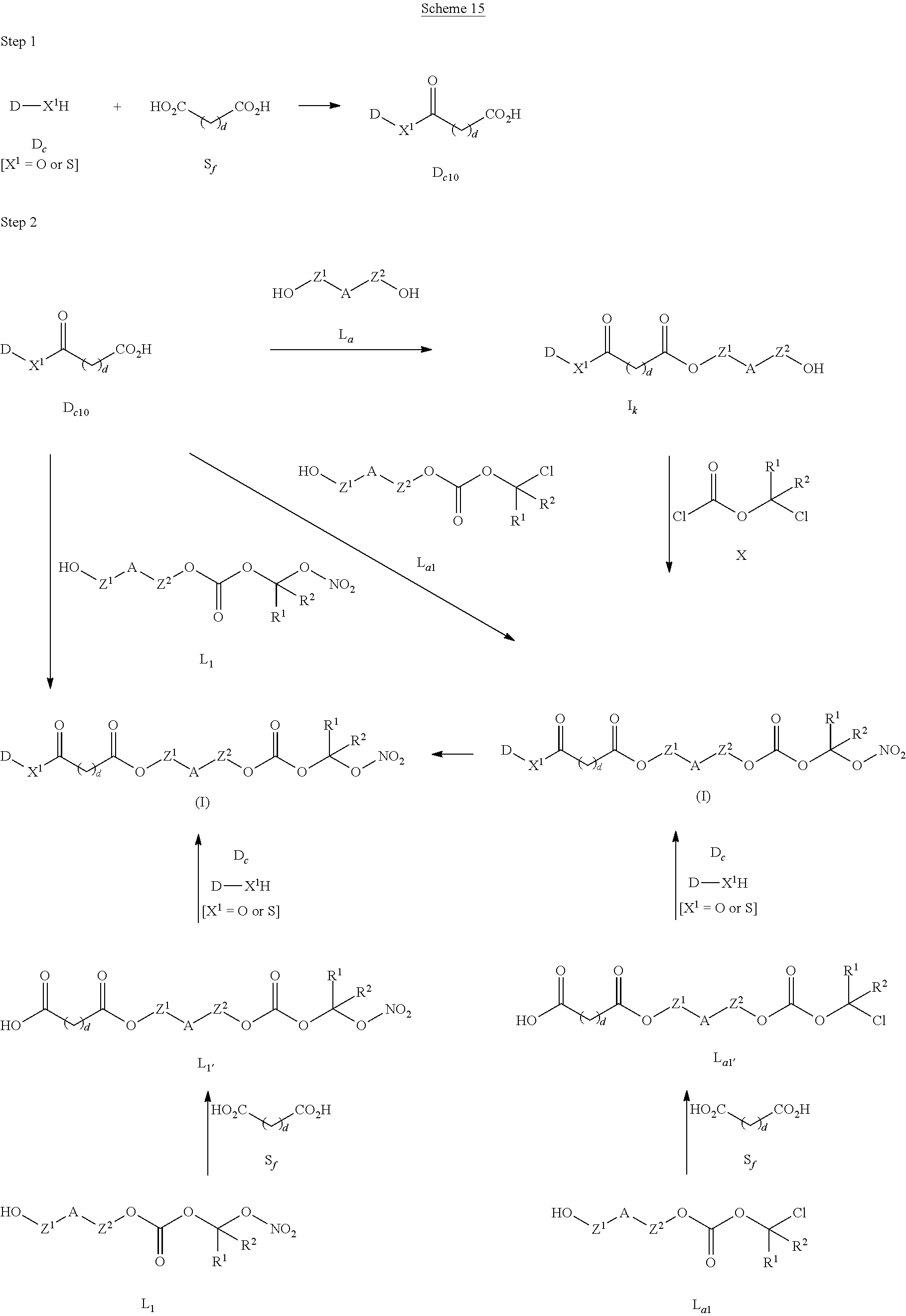

### Step 1

In step 1, the drug containing a hydroxyl or sulfhydryl group D_{c} (D-Y—X¹H, wherein Y=a bond; X¹═O or S) is reacted with a dicarboxylic acid compound of formula (S_{f}) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain the corresponding reactive drug derivative (D_{c10}).

### Step 2

The reactive drug derivative (D_{c10}) as obtained in step 1 above is further coupled with the linker compound of formula (Lₐ) in the presence of a coupling agent, for example, N,N-dicyclohexylcarbodiimide (DCC), a suitable base, for example, dimethylaminopyridine (DMAP) to obtain the intermediate compound (Iₖ). The resulting intermediate compound (Iₖ) is further reacted with the chloroformate (X) (as obtained in step 1 of Scheme 1) to obtain another intermediate compound of formula (Iₖ₁). The intermediate compound of formula (Iₖ₁) is then subjected to nitration using silver nitrate in the presence of an organic solvent, for example, acetonitrile, to obtain the compound of formula (I). Alternatively, the drug derivative (D_{c10}) is coupled directly with the linker (Lₐ₁) (as obtained in Step 1′, Scheme 1) to obtain the intermediate chloro compound of formula (Iₖ₁) which is converted to the nitrate compound of the formula (I) as described above. In a more direct approach, the drug derivative (D_{c10}) is coupled directly with the nitrate containing linker L₁ (as obtained in Step 1, Scheme 2) to obtain the final compound of formula (I). In another approach, the chloro compound of the formula (Lₐ₁) is coupled first with a dicarboxylic acid of the formula (S_{f}) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain the corresponding derivative (Lₐ₁'). This compound of formula (L_{a1′}) is further coupled with a drug containing a hydroxyl or sulfhydryl group D_{c} (D-Y—X¹H, wherein Y=a bond; X¹═O or S) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain the corresponding reactive drug derivative (Iₖ₁). The intermediate compound of the formula (Iₖ₁) is coverted to the final compound of formula (I) as described above. In yet another approach, the nitrate linker of formula (L₁) is coupled with dicarboxylic acid of the formula (S_{f}) followed by the drug containing a hydroxyl or sulfhydryl group D_{c} (D-Y—X¹H, wherein Y=a bond; X¹═O or S) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain the final nitrate compound of formula (I).

In scheme 15, the variables D, Z¹, Z², A, R¹ and R² are as defined in any of the embodiments of the present invention with reference to the compounds of formula (I) wherein D constitutes a drug containing a hydroxyl or a sulfhydryl group.

Alternative process for the preparation of the compound of formula (I), wherein D is a drug containing one or more functional groups independently selected from a hydroxyl or a sulfhydryl group and Y is a spacer group selected from Yₕ=

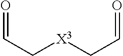

(wherein X³ is as defined above) can be carried out in accordance with the reaction steps as depicted in the following Scheme 16.

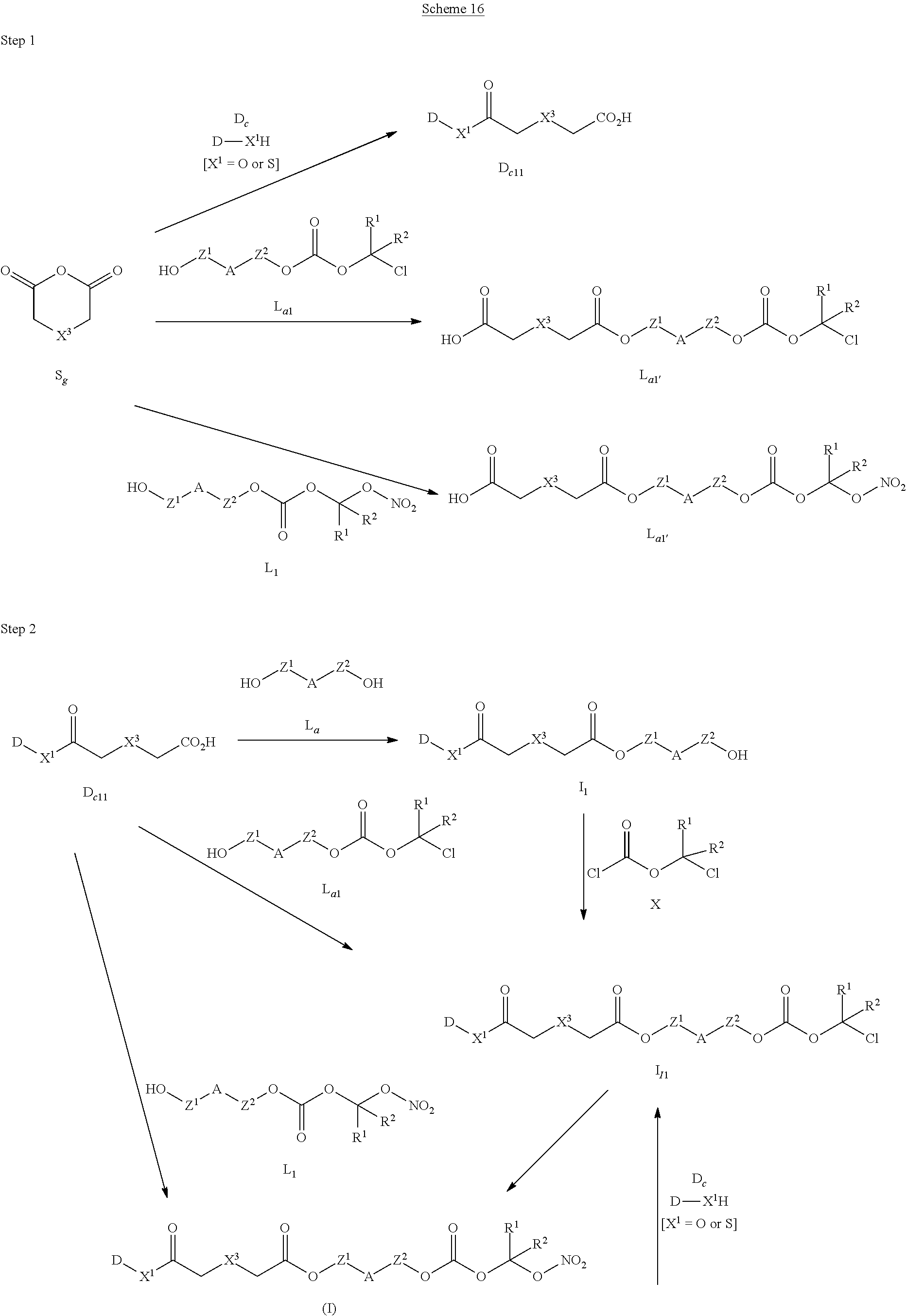

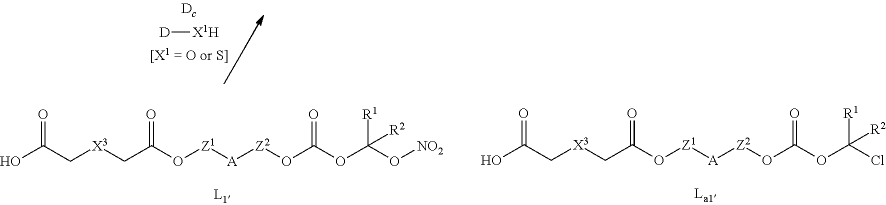

### Step 1

The drug containing a hydroxyl or sulfhydryl group D_{c} (D-Y—X¹H, wherein Y=a bond; X¹═O or S) is reacted with the compound of formula (S_{g}) in the presence of a suitable base, for example, dimethylaminopyridine (DMAP) and a solvent, for example, dichloromethane (DCM) to form the reactive drug derivative (D_{c11}). Similarly, reactions of the linkers of formula (Lₐ₁) or (L₁) with cyclic anhydride compound of formula (S_{g}) in the presence of a suitable base, for example, dimethylaminopyridine (DMAP) and an organic solvent, for example, dichloromethane (DCM) afforded the respective linker intermediates of formula (L_{a1′}) and (L_{1′}) respectively.

### Step 2

The reactive drug derivative (D_{c11}) as obtained in step 1 above is then coupled with the linker of formula (Lₐ) in the presence of a coupling agent, for example, N,N-dicyclohexylcarbodiimide (DCC), a suitable base, for example, dimethylaminopyridine (DMAP) and an organic solvent, for example, dichloromethane (DCM) to form the compound intermediate (Iₗ). The compound intermediate (Iₗ) is further reacted with the chloroformate (X) (as obtained in step 1 of Scheme 1) to obtain another intermediate compound of formula (Iₗ₁). The intermediate compound of formula (Iₗ₁) is then subjected to nitration using silver nitrate in the presence of an organic solvent, for example, acetonitrile, to obtain the compound of formula (I). In another approach, the compound of formula (D_{c11}) is reacted with the linker intermediate of formula (Lₐ₁) in the presence of a coupling agent, for example, N,N-dicyclohexylcarbodiimide (DCC), a suitable base, for example, dimethylaminopyridine (DMAP) and an organic solvent, for example, dichloromethane (DCM) to form the compound intermediate (Iₗ₁), which is converted to the final compound of formula (I) as described above. Alternatively, the compound of formula (D_{c11}) is reacted with the linker intermediate of formula (L₁) in the presence of a coupling agent, for example, N,N-dicyclohexylcarbodiimide (DCC), a suitable base, for example, dimethylaminopyridine (DMAP) and an organic solvent, for example, dichloromethane (DCM) to directly afford the compound of formula (I). In another approach, the drug containing a hydroxyl or sulfhydryl group D_{c} (D-Y—X¹H, wherein Y=a bond; X¹═O or S) is coupled with the linker compound of formula (L_{a1′}) in the presence of a coupling agent, for example, N,N-dicyclohexylcarbodiimide (DCC), a suitable base, for example, dimethylaminopyridine (DMAP) and an organic solvent, for example, dichloromethane (DCM) to form the compound intermediate (Iₗ₁), which is finally converted to the compound of formula (I) as described above. In yet another approach, reaction of the drug containing a hydroxyl or sulfhydryl group D_{c} (D-Y—X¹H, wherein Y=a bond; X¹═O or S) with the linker compound of formula (L_{1′}) in the presence of a coupling agent, for example, N,N-dicyclohexylcarbodiimide (DCC), a suitable base, for example, dimethylaminopyridine (DMAP) and an organic solvent, for example, dichloromethane (DCM) directly afforded the final compound formula (I).

In scheme 16, the variables D, Z¹, Z², A, R¹ and R² are as defined in any of the embodiments of the present invention with reference to the compounds of formula (I) wherein D constitutes a drug containing hydroxyl or sulfhydryl group.

An alternative process for the preparation of the compound of formula (I), wherein D is a drug containing one or more functional groups independently selected from a hydroxyl a sulfhydryl group and Y is a spacer group selected from Yₑ=

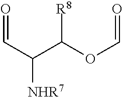

(wherein R⁷ and R⁸ is as defined above) can be carried out in accordance with the reaction steps as depicted in Scheme 17.

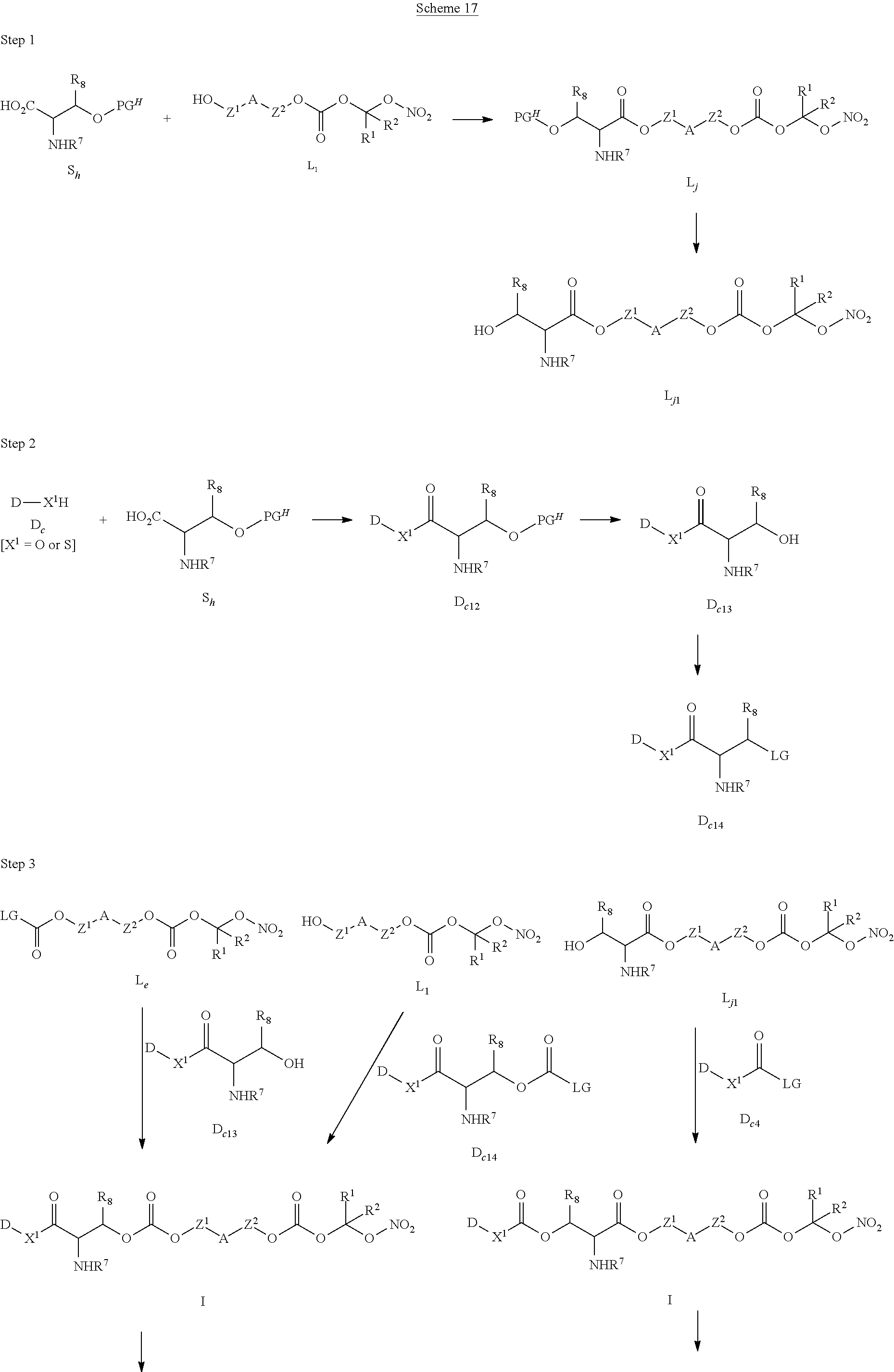

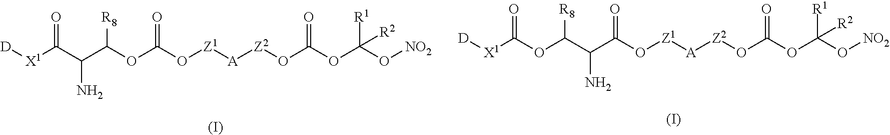

### Step 1

The compound of formula (Sₕ) (wherein PG^{H} is a hydroxyl protecting group defined above and R⁷ and R⁸ are as defined above) is reacted with the linker (L₁) in the presence of a coupling agent, for example, N,N-dicyclohexylcarbodiimide (DCC), a suitable base, for example, dimethylaminopyridine (DMAP), and organic solvent, for example, dichloromethane (DCM) to form the compound of formula (Lⱼ). Removal of the protecting group PG^{H} in the compound of formula (Lⱼ) is carried out by a standard procedure known in the art to afford compound of formula (Lⱼ₁).

### Step 2

The drug containing a hydroxyl or sulfhydryl group D_{c} (D-Y—X¹H, wherein Y=a bond; X¹═O or S) is reacted with the compound of formula (Sₕ) in the presence of a coupling agent, for example, N,N-dicyclohexylcarbodiimide (DCC), a suitable base, for example, dimethylaminopyridine (DMAP) and an organic solvent, for example, dichloromethane (DCM) to obtain a reactive drug derivative of formula D_{c12} (wherein R⁷ is hydrogen or an amino protecting group as defined in the first embodiment herein above). Removal of the protecting group PG^{H} from the drug derivative (D_{c12}) is carried out using a standard procedure known in the art to obtain another reactive drug derivative of formula (D_{c13}). The resulting drug derivative (D_{c13}) is further treated with phosgene or its equivalent selected from diphosgene, triphosgene, N,N′-carbonyldiimidazole (CDI), N,N′-disuccinimidyl carbonate (DSC) or 4-nitrophenyl chloroformate in the presence of a base, for example, triethylamine and a solvent, for example, dichloromethane (DCM) to afford another reactive drug derivative of formula (D_{c14}).

### Step 3

The drug derivative (D_{c13}) as obtained in step 2 above is reacted with the compound (Lₑ) (as obtained in reaction step 1 of Scheme 7) in the presence of a base, for example, triethylamine and a solvent, for example, dichloromethane (DCM) to obtain the compound of formula (I) (wherein Y is a spacer of formula Yₑ and R⁷ is an amino protecting group as defined above). Alternatively, the drug derivative (D_{c14}) is reacted with the linker (L₁) in the presence of a base, for example, triethylamine and a solvent, for example, dichloromethane (DCM) to obtain the compound (I) (wherein Y is a spacer group of formula Yₑ and R⁷ is an amino protecting group as defined above). The compound of formula (I), (wherein Y is a spacer group of formula Yₑ and R⁷ is an amino protecting group as defined above) can alternatively be obtained by reacting the drug derivative (D_{c4}) as obtained in Step 2, Scheme 7 (wherein Y=a bond; X¹ is O or S, LG=Leaving group) with the compound (Lⱼ₁) as obtained in reaction step 1 above, in the presence of a suitable base, for example, triethylamine and a solvent, for example, dichloromethane (DCM). Removal of the amino protecting group R⁷ in the compound of formula (I), (wherein Y is a spacer group of formula Yₑ and R⁷ is an amino protecting group as defined above) is carried out using any standard procedure known in the art to obtain the compound of formula (I) (wherein R₇=hydrogen).

An alternative process for the preparation of the compound of formula (I), wherein D is a drug containing one or more functional groups selected from a hydroxyl or a sulfhydryl group (D_{b}″) and Y is a spacer group selected from Yᵢ=

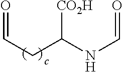

(wherein c is as defined above) can be carried out in accordance with the reaction steps as depicted in Scheme 18.

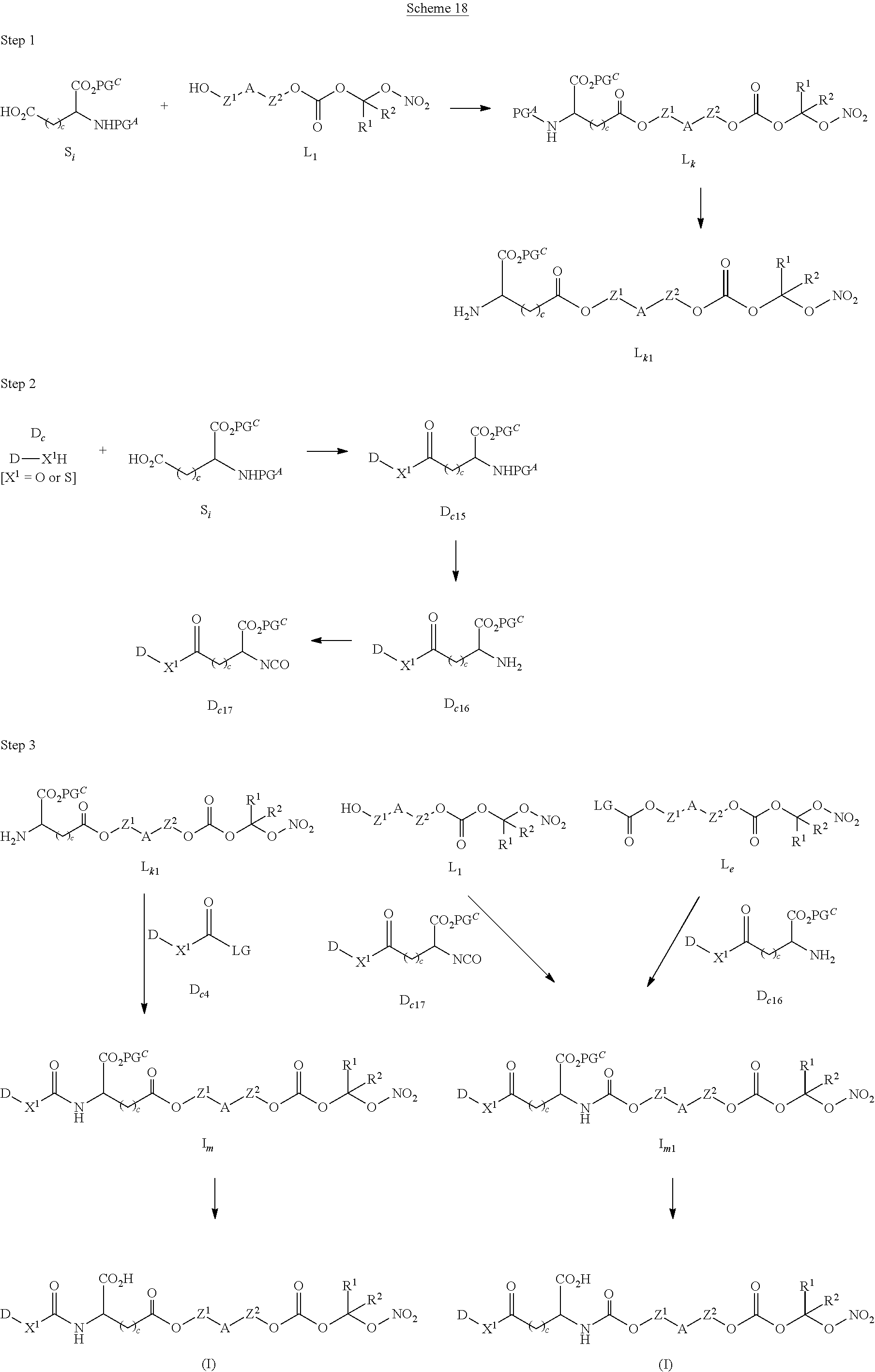

### Step 1

In this step, the compound of formula (Sᵢ) (wherein PG^{C} is a suitable carboxyl protecting group and PG^{A} is a suitable amino protecting group as defined above and c is as defined above) is reacted with the linker (L₁) in the presence of a coupling agent, for example, dicyclohexylcarbodiimide (DCC), a suitable base, for example, dimethylaminopyridine (DMAP) and an organic solvent, for example, dichloromethane (DCM) to obtain the compound of formula (Lₖ). Removal of the protecting group PG^{A} in the compound of formula (Lₖ) is carried out by a standard procedure known in the art to get the compound of formula (Lₖ₁).

### Step 2

The drug containing a hydroxyl or sulfhydryl group D_{c} (D-Y—X¹H, wherein Y=a bond; X¹═O or S) is reacted with the compound of formula (Sᵢ) in the presence of a coupling agent, for example, dicyclohexylcarbodiimide (DCC), a suitable base, for example, dimethylaminopyridine (DMAP) and an organic solvent, for example, dichloromethane (DCM) to get the corresponding reactive drug derivative of formula D_{c15}, wherein PG^{C} is a carboxylic acid protecting group as defined above). The removal of the protecting group PG^{C} from the drug derivative (D_{c15}) is carried out using a standard procedure known in the art to obtain another reactive drug derivative of formula (D_{c16}). The drug derivative (D_{c16}) is further treated with phosgene or its safe equivalent selected from diphosgene, triphosgene, N,N′-carbonyldiimidazole (CDI), N,N′-disuccinimidyl carbonate (DSC) or 4-nitrophenyl chloroformate in the presence of a base, for example, triethylamine and a solvent, for example, dichloromethane(DCM) to afford another reactive drug derivative of formula (D_{c17}).

### Step 3

The drug derivative (D_{c16}) is reacted with the compound of formula (Lₑ) (as obtained in step 1 of Scheme 7) in the presence of a base, for example, triethylamine and a solvent, for example, dichloromethane (DCM) or alternatively, the drug derivative (D_{c17}) is reacted with the linker (L₁) in the presence of a base, for example, triethylamine and a solvent, for example, dichloromethane (DCM) to get the compound intermediate (Iₘ₁). Alternatively, the drug derivative (D_{c4}) as obtained in Step 2, Scheme 7 (wherein Y=a bond; X¹ is O or S, LG=Leaving group) is coupled with the compound of formula Lₖ₁ (as obtained in Step 1 above) to obtain an intermediate compound (Iₘ). Removal of the carboxylic acid protecting group PG^{C} in the intermediate compound (Iₘ) or the compound intermediate (Iₘ₁) is carried out using a standard procedure known in the art to obtain the compound of formula (I).

An alternative method for obtaining the compound of formula (I), wherein D is a drug containing one or more functional groups selected from a hydroxyl or a sulfhydryl group and Y is a spacer group selected from Yⱼ=

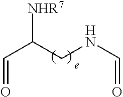

(wherein the group R⁷ is an amino protecting group as defined above and e is also defined above) involves the reaction steps depicted in the following Scheme 19.

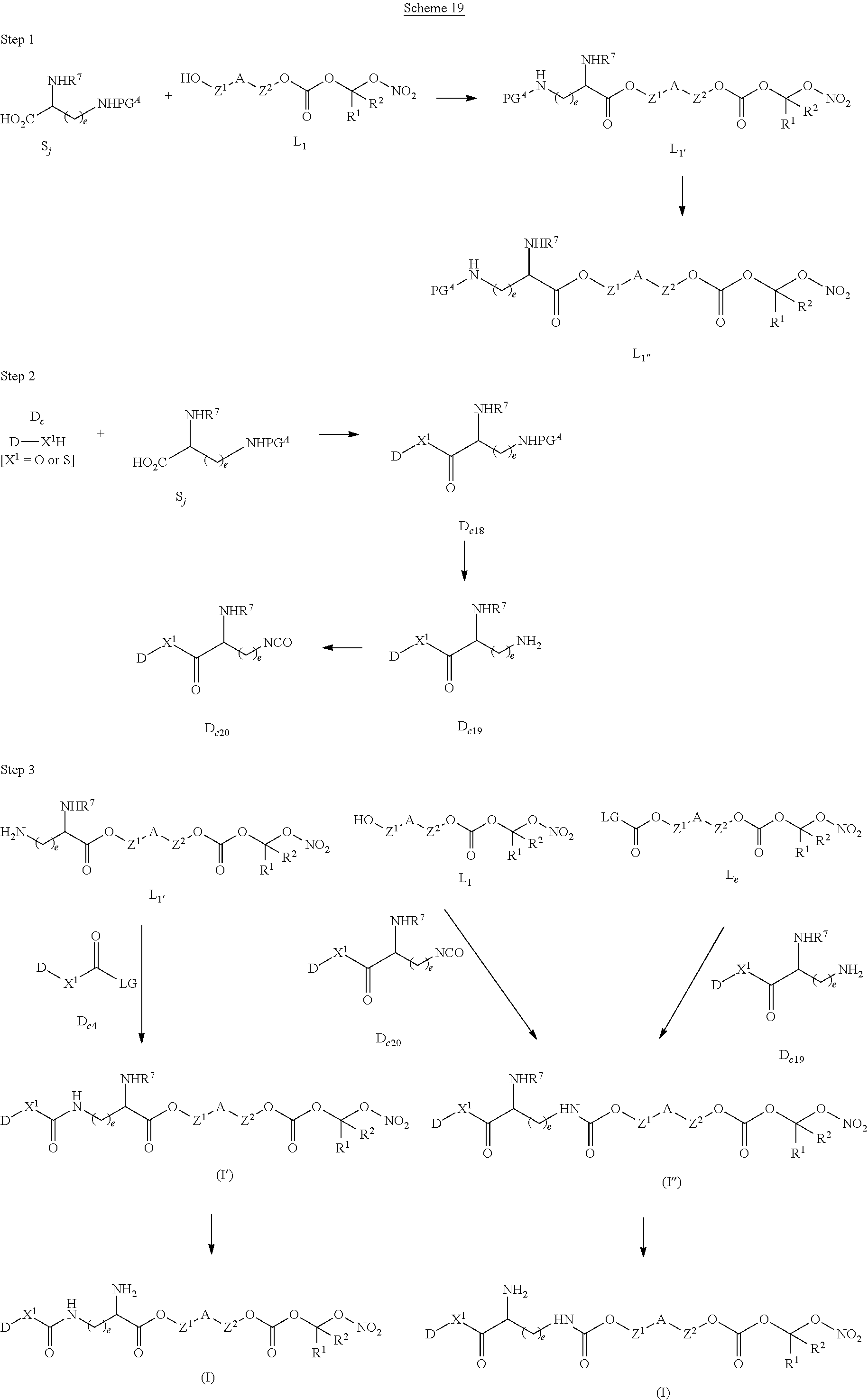

### Step 1

In this step, the compound of formula (Sⱼ) (wherein R⁷ and PG^{A} are suitable amino protecting groups) is reacted with the linker (L₁) in the presence of a coupling agent, for example, dicyclohexylcarbodiimide (DCC), a suitable base, for example, dimethylaminopyridine (DMAP) and an organic solvent, for example, dichloromethane (DCM) to yield the compound of formula (L_{1′}). Selective removal of the protecting group PG^{A} in compound of formula (L_{1′}) is carried out by a standard procedure known in the art to afford the compound of formula (L_{1′}).

### Step 2

The drug containing a hydroxyl or sulfhydryl group D_{c} (D-Y—X¹H, wherein Y=a bond; X¹═O or S) is reacted with the compound of formula (Sⱼ) in the presence of a coupling agent, for example, dicyclohexylcarbodiimide (DCC), a suitable base, for example, dimethylaminopyridine (DMAP) and an organic solvent, for example, dichloromethane (DCM) to form a reactive drug derivative of formula (D_{c18}). Selective removal of the protecting group PG^{A} from the drug derivative (D_{c18}) is carried out using a standard procedure known in the art to give another reactive drug derivative of formula (D_{c19}). The drug derivative (D_{c19}) is further treated with phosgene or its equivalent selected from diphosgene, triphosgene, N,N′-carbonyldiimidazole (CDI), N,N′-disuccinimidyl carbonate (DSC) or 4-nitrophenyl chloroformate in the presence of a base, for example, triethylamine and a solvent, for example, dichloromethane to afford another reactive drug isocyanate derivative of formula (D_{c20}. Step 3

The drug derivative (D_{c19}) as obtained in step 2 above is reacted with the compound of formula (Lₑ) (as obtained in step 1 of Scheme 7) in the presence of a base, for example, triethylamine and a solvent, for example, dichloromethane (DCM) or the drug derivative (D_{c20}) is reacted with the linker (L₁) in the presence of a base, triethylamine for example, and a solvent, for example, dichloromethane (DCM) to yield the compound of formula (I″) (wherein Y is a spacer group of formula Yⱼ, wherein R⁷ is an amino protecting group as defined above). Alternatively, the drug derivative (D_{c4}) as obtained in Step 2, Scheme 7 (wherein Y=a bond; X¹ is O or S; LG=Leaving group) is coupled with the compound of formula L_{1′}, (as obtained in Step 1 above) to obtain a compound of formula (I′) (wherein Y═Yⱼ, wherein R⁷ is an amino protecting group as defined above). Removal of the amino protecting group R⁷ in the compounds of formulae (I′) and (I″) (wherein Y is a spacer of formula Yⱼ, wherein R⁷ is an amino protecting group as defined above) is carried out using a standard procedure known in the art to obtain the respective compounds of formula (I) (wherein R⁷=hydrogen as defined above).

An alternative process for the preparation of the compound of formula (I), wherein D is a drug containing one or more functional groups independently selected from a hydroxyl or a sulfhydryl group and Y is a spacer group selected from Yₖ=

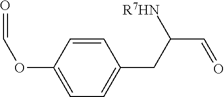

(wherein R⁷ is as defined above) can be carried out in accordance with the reaction steps as depicted in the following Scheme 20.

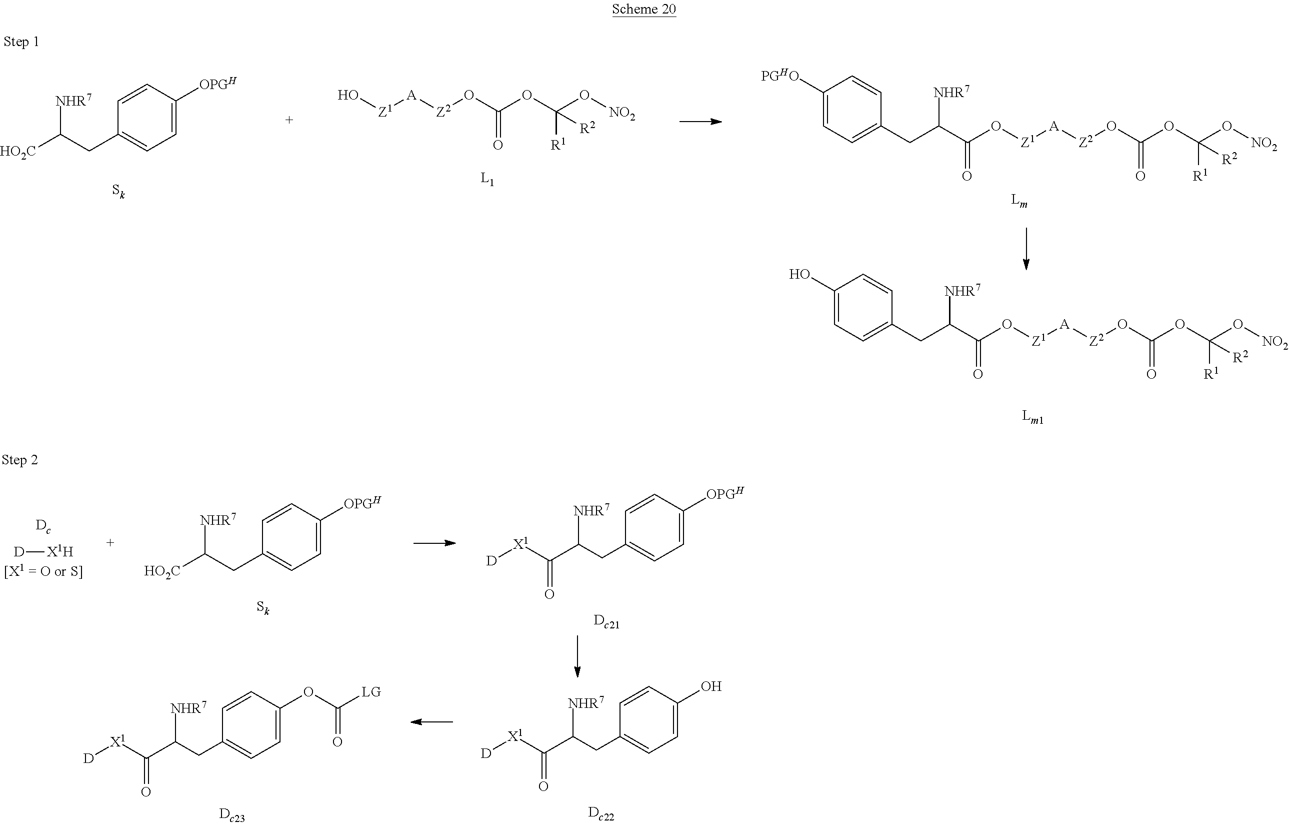

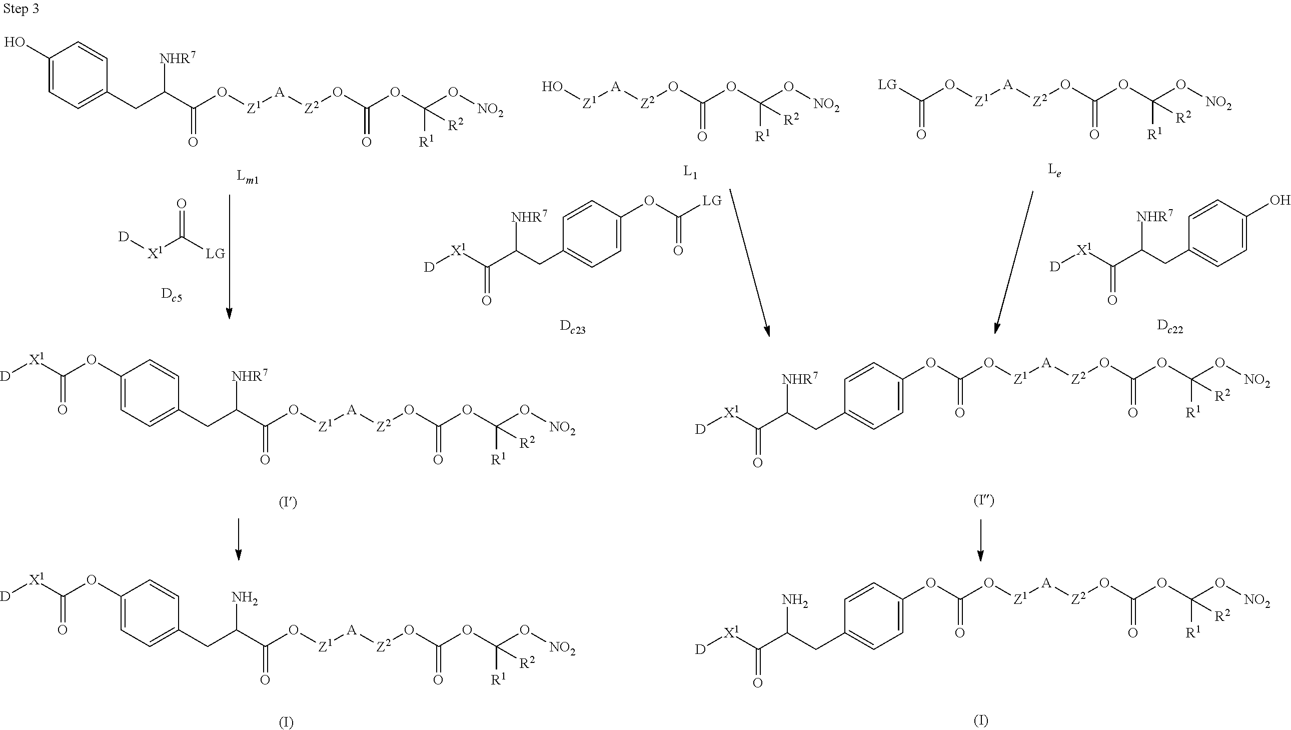

### Step 1

In this step, the compound of formula (Sₖ) (wherein PG^{H} is a suitable hydroxyl protecting group and R⁷ is a suitable amino protecting group) is reacted with the linker (L₁) in the presence of a coupling agent, for example, dicyclohexylcarbodiimide (DCC), a suitable base, for example, dimethylaminopyridine (DMAP) and an organic solvent, for example, dichloromethane (DCM) to obtain the compound of formula (Lₘ). Removal of the protecting group PG^{H} in compound of formula (Lₘ) is carried out by a standard procedure known in the art to obtain the compound of formula (Lₘ₁).

### Step 2

The drug containing a hydroxyl or sulfhydryl group D_{c} (D-Y—X¹H, wherein Y=a bond; X¹═O or S) is reacted with the compound of formula (Sₖ) in the presence of a coupling agent, for example, dicyclohexylcarbodiimide (DCC), a suitable base, for example, dimethylaminopyridine (DMAP) and an organic solvent, for example, dichloromethane (DCM) to obtain the corresponding reactive derivative of the drug represented by formula (D_{c21}). Removal of the protecting group PG^{H} from the drug derivative (D_{c21}) is carried out using a standard procedure known in the art to obtain another reactive drug derivative of formula (D_{c22}). The drug derivative (D_{c22}) is further treated with phosgene or its equivalent selected from diphosgene, triphosgene, N,N′-carbonyldiimidazole (CDI), N,N′-disuccinimidyl carbonate (DSC) or 4-nitrophenyl chloroformate in the presence of a base, for example, triethylamine and a solvent, for example, dichloromethane (DCM) to obtain another reactive drug derivative of formula (D_{c23}).

### Step 3

The drug derivative (D_{c22}) is reacted with the compound of formula (Lₑ) (as obtained in reaction step 1 of Scheme 7) in the presence of a base, for example, triethylamine and a solvent, for example, dichloromethane (DCM) or alternatively, the drug derivative (D_{c23}) is reacted with the linker (L₁) in the presence of a base, for example, triethylamine and a solvent, for example, dichloromethane (DCM) to obtain a compound of formula (I″) (wherein Y is a spacer group of formula Yₖ, wherein R⁷ is as defined above). Alternatively, the drug derivative (D_{c4}) as obtained in Step 2, Scheme 7 (wherein Y=a bond; X¹ is O or S; LG=Leaving group) is coupled with the compound of formula Lₘ₁ (as obtained in Step 1 above) to obtain the compound of formula (I′) (wherein Y is a spacer group of formula Yₖ, wherein R⁷ is an amino protecting group as defined above). Removal of the amino protecting group R⁷ in the compounds of formulae (I′) and (I″) (wherein Y is a spacer group of formula Yₖ) is carried out using a standard procedure known in the art to yield the nitrate ester prodrug of formula (I) (wherein R⁷=hydrogen as defined above).

An alternative method for the preparation of the compound of formula (I), wherein D is a drug containing one or more functional groups selected from a hydroxyl or a sulfhydryl group and Y is a spacer group selected from Yₗ

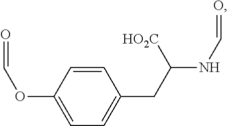

involves the reaction steps as depicted in the following Scheme 21.

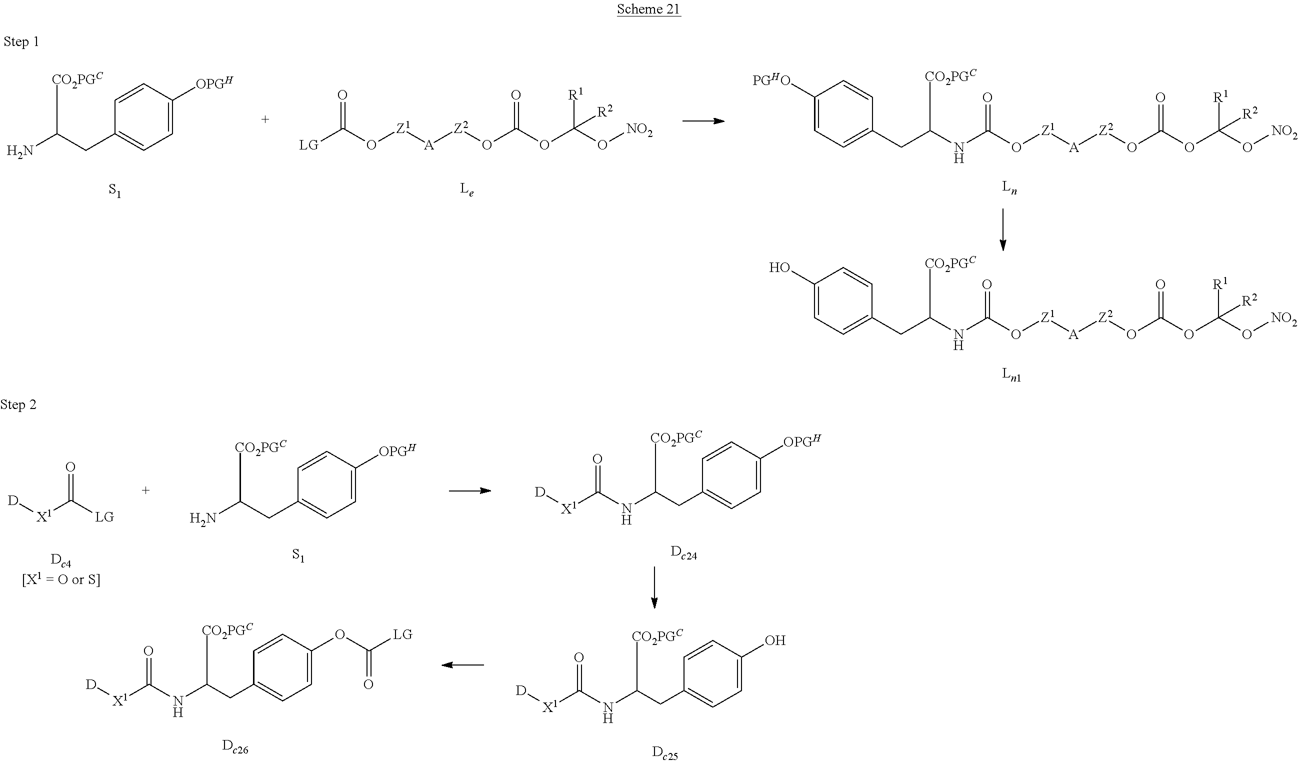

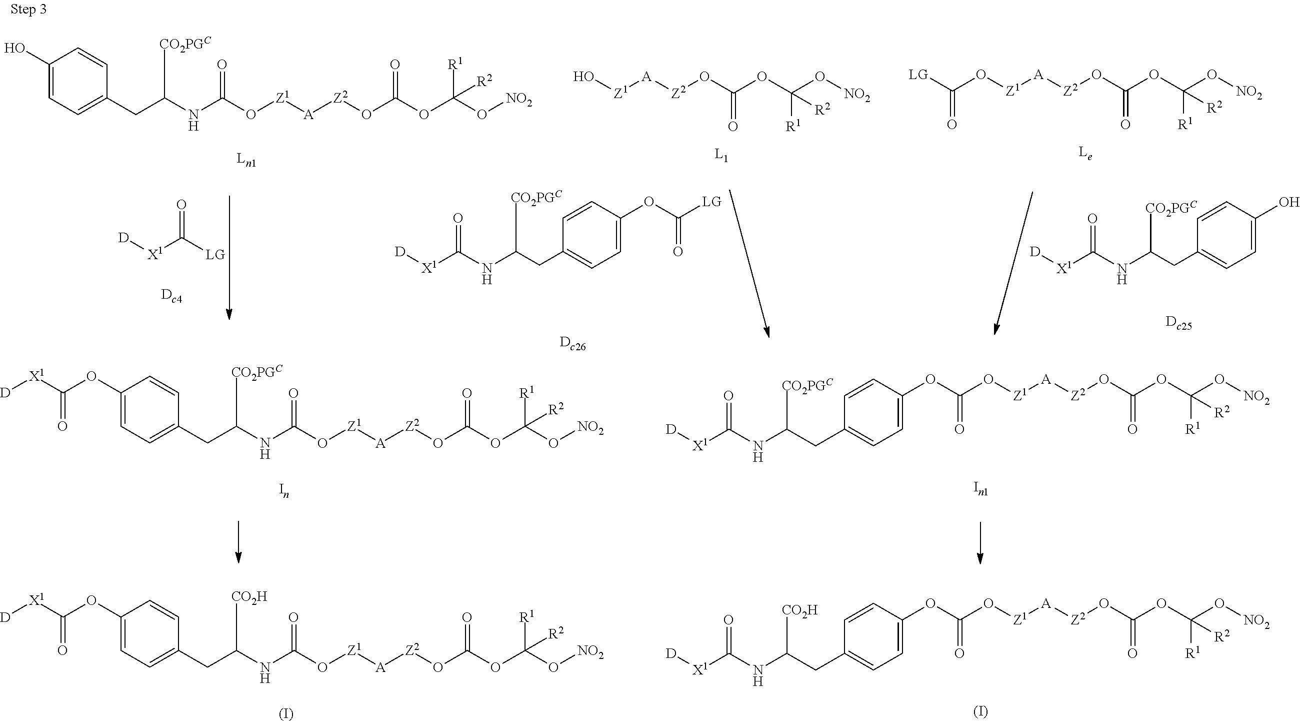

### Step 1

In this step, the compound of formula (Sₗ) (wherein PG^{H} is a suitable hydroxyl protecting group and PG^{C} is a suitable carboxylic acid protecting group) is reacted with the linker Lₑ (as obtained in Step 1, Scheme 7) in the presence of a coupling agent, for example, dicyclohexylcarbodiimide (DCC), a suitable base, for example, dimethylaminopyridine (DMAP) and an organic solvent, for example, dichloromethane (DCM) to obtain the compound of formula (Lₙ). Selective removal of the protecting group PG^{H} in the compound of formula (Lₙ) is carried out by a standard procedure known in the art to obtain the compound of formula (Lₙ₁).

### Step 2

The drug derivative D_{c4} as obtained in Step 2, Scheme 7 (wherein Y=a bond; X¹ is O or S; LG=Leaving group) is reacted with the compound of formula (Sₗ) in the presence of a suitable base, for example, triethylamine and an organic solvent, for example, dichloromethane (DCM) to obtain a reactive drug derivative of formula (D_{c24}). Removal of the protecting group PG^{H} from the drug derivative (D_{c24}) is carried out using a standard procedure known in the art to obtain another reactive derivative of the drug represented by formula (D_{c25}). The drug derivative (D_{c25}) is further treated with phosgene or its safe equivalent selected from diphosgene, triphosgene, N,N′-carbonyldiimidazole (CDI), N,N′-disuccinimidyl carbonate (DSC) or 4-nitrophenyl chloroformate in the presence of a base, for example, triethylamine and a solvent, for example, dichloromethane (DCM) to form another reactive drug derivative of formula (D_{c26}).

### Step 3

The drug derivative (D_{c25}) as obtained in step 2 above is reacted with the compound of formula (Lₑ) (as obtained in step 1 of Scheme 7) in the presence of a base, triethylamine and a solvent, for example, dichloromethane (DCM) or alternatively, the drug derivative (D_{c26}) is reacted with the linker (L₁) in the presence of a base, for example, triethylamine and a solvent, for example, dichloromethane (DCM) to obtain an intermediate compound (Iₙ₁). Similarly, the drug derivative D_{c4} as obtained in Step 2, Scheme 7 (wherein Y=a bond; X¹ is O or S; LG=Leaving group), is reacted with the compound of formula Lₙ₁ (as obtained in Step 1 above) to get another intermediate compound (Iₙ). Removal of the carboxylic acid protecting group PG^{C} in the intermediate compound (Iₙ) or in the intermediate compound (Iₙ₁) is carried out using a standard procedure known in the art to obtain the respective compounds of formula (I).

Although not specified in the above general synthetic schemes, it is understandable to any person skilled in the art that if the said drugs or therapeutic agents contain one or more additional derivatizable functional groups such as amino, carboxyl, hydroxyl (including phenolic), or sulfhydryl groups, those functional groups may need to be selectively protected, if it is necessary, by any widely used suitable protecting groups and subsequently deprotected, if it is necessary, at appropriate stages of the processes for the preparation of the compound of formula (I), which are the nitric oxide releasing prodrugs of known drugs or therapeutic agents, and such selective protection and deprotection reactions are carried out as described in Theodora W. Greene and Peter G. M. Wuts, “Protective Groups in Organic Synthesis”, 3^{rd} edition, John Wiley and Sons, Inc. New York (1999), the disclosure of the relevant portion is incorporated herein by reference. To illustrate this feature, conversion of a drug containing two or more types of functional groups, for example atorvastatin, to the corresponding nitric oxide-releasing prodrug of atorvastatin (NO-Atorvastatin) of formula (I) via selective protection and if necessary, deprotection of the hydroxyl groups of the drug at appropriate stages of their synthesis as shown in Scheme 22.

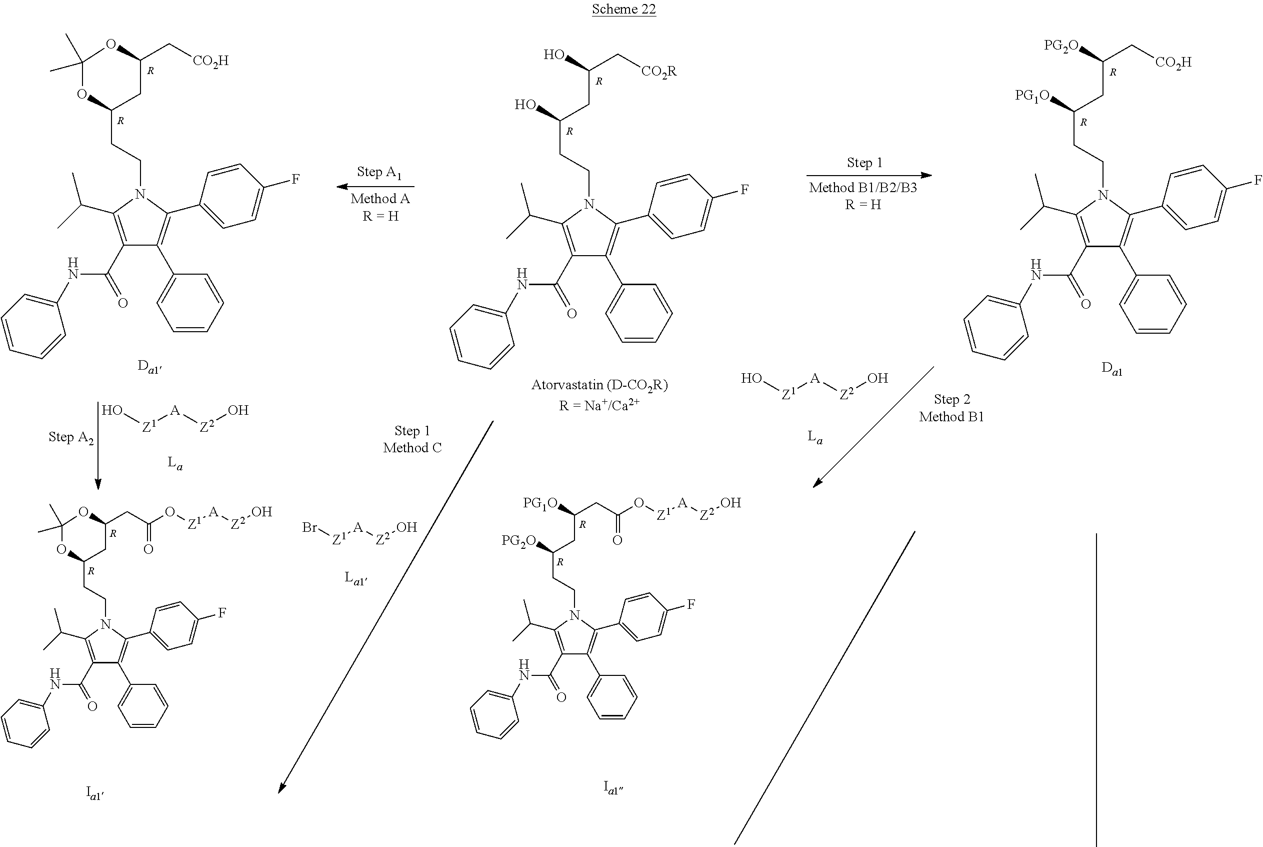

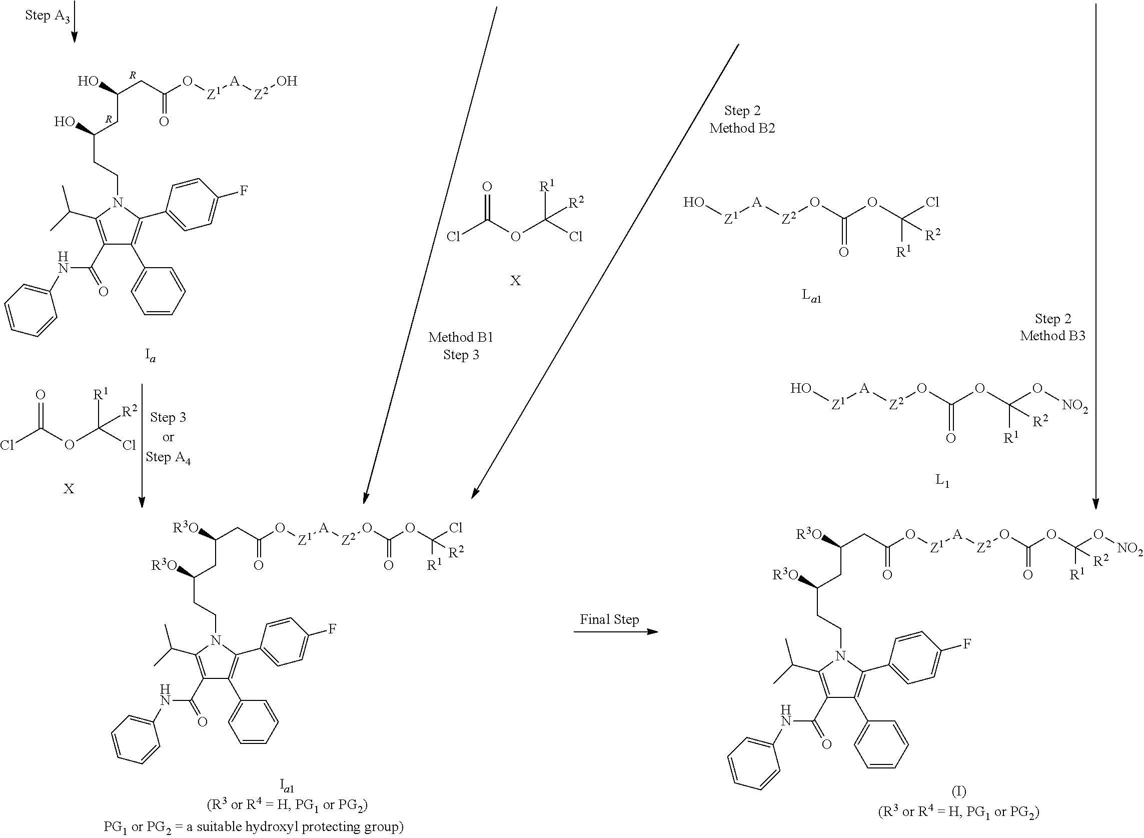

### Method A:

In Step A₁, two hydroxyl groups of the drug, for example Atorvastatin (D-CO₂H) are protected by a suitable protecting group, for example as an acetonide, by a generally known procedure, to obtain the partially protected drug (Dₐ₁'). In Step A₂, the partially protected drug (D_{a1′}) as obtained in step A₁ above is coupled with a linker diol (Lₐ) by a method described in Step 2 of Scheme 1, to afford the intermediate alcohol (I_{a1′}). In Step A₃, the acetonide protecting group in the intermediate (I_{a1′}) is removed by a method generally known to those skilled in the art to obtain the intermediate triol (Iₐ). In Step A₄, the intermediate alcohol (Iₐ) as obtained in step A₃ above is further reacted with α-chloroformate (X) in the presence of an organic base, for example, pyridine and an organic solvent, for example, dichloromethane (DCM) to obtain the selectively acylated intermediate compound (Iₐ₁). In the final Step, the intermediate chloride (Iₐ₁) is subjected to nitration using silver nitrate in the presence of an organic solvent, for example, acetonitrile to form the compound of formula (I), and if desired, the compound of formula (I) is converted to its pharmaceutically acceptable salt.

### Method B1/B2/B3:

In Step 1, the two hydroxyl groups of the drug, for example Atorvastatin (D-CO₂H) are protected by any suitable hydroxyl protecting groups that are likely to be cleaved in vivo (i.e., under biological conditions), by a method generally known to those skilled in the art, to obtain a partially protected drug (Dₐ₁), which can be converted to the compounds of formula (I) by any of the following methods:

Method B1: In Step 2, the partially protected drug (Dₐ₁), as obtained in step 1 above, is coupled with a linker diol (Lₐ) by a method described in Step 2 of Scheme 1, to afford the intermediate alcohol (I_{a1″}). In Step 3, the intermediate alcohol (I_{a1″}) as obtained in step 2 above is further reacted with α-chloroformate (X) in the presence of an organic base, for example, pyridine and an organic solvent, for example, dichloromethane (DCM) to obtain the intermediate compound (Iₐ₁). In the final Step, the intermediate (Iₐ₁) is also converted to the compound of formula (I) as described in the final step of Method A.

Method B2: In Step 2, the partially protected carboxyl-containing drug (Dₐ₁) as obtained in step 1 above is coupled with a linker intermediate (Lₐ₁) by a method described in Step 3 (i.e., Method A) of Scheme 2, to afford the intermediate chloride (Iₐ₁), which is finally converted to the compound of formula (I) as described in the Final step of Method A.

Method B3: In Step 2, the partially protected Atorvastatin (Dₐ₁) as obtained in step 1 above is directly coupled with the nitrate containing linker (L₁) by a method described in Step 3 (i.e., Method B) of Scheme 2, to afford the compound of formula (I), and if desired, the compound of formula (I) is converted to its pharmaceutically acceptable salt.

Method C: In Step 1, sodium or calcium salt of the atorvastain (D-CO₂R, wherein, R=Na⁺ or Ca²⁺) is reacted with linker bromide (L_{a1′}) in the presence of an organic solvent, for example DMF to afford the intermediate alcohol of the formula (Iₐ). The resulting intermediate alcohol (Iₐ) is converted to the compound of formula (I), as already described above in Method A. If desired, the compound of formula (I) thus obtained is converted to its pharmaceutically acceptable salt.

The organic base, used in any reaction steps of the processes for the preparation of the compound of formula (I) as depicted in the aforementioned schemes, may be selected from but not limited to triethylamine, diisopropylethylamine, 4-(dimethylamino) pyridine (DMAP), pyridine or mixtures thereof.

The organic solvent used in any reaction steps of the processes for the preparation of the compound of formula (I) may be selected from but not limited to dichloromethane (DCM), chloroform, dimethylformamide (DMF), tetrahydrofuran (THF), acetonitrile, ethyl acetate, diethyl ether or mixtures thereof.

The coupling agent used in a reaction step involving coupling for the preparation of the compound of formula (I) may be selected from but not limited to N,N′-dicyclohexylcarbodiimide (DCC), O-(Benzotriazol-1-yl)-N,N,N′,N′-tetramethyluronium hexafluorophosphate (HBTU), benzotriazol-1-yl-oxy-tris(dimethylamino)phosphonium hexafluorophosphate (BOP), O-(benzotriazol-1-yl)-N,N,N′,N′-tetramethyluronium tetrafluoroborate (TBTU), N,N′-dicyclohexylcarbodiimide/N-hydroxybenzotriazole (DCC/HOBT), 1-Ethyl-(3-dimethylaminopropyl)carbodiimide hydrochloride (EDAC. HCl) and benzotriazol-1-yl-oxy-tris(dimethylamino)phosphonium hexafluorophosphate (BOP) and EDAC. HCl/HOBT,

The present invention also relates to the process of resoluting the racemic mixture of the compound of formula (I) or a pharmaceutically acceptable salt thereof:

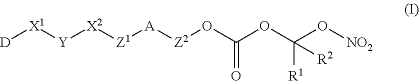

wherein D, X¹, Y, X², A, Z¹, Z², R¹ and R² are as defined above, the process of resoluting the racemic mixture comprises reacting the racemic compound of formula (I) with a chiral auxiliary in the presence of a solvent, crystallising out the required diastereoisomeric salt and subsequently treating it with a base to obtain the desired enantiomer of the compound of formula (I)

It has been indicated herein that the prodrugs [the compounds of formula (I)] of the present invention would undergo enzymatic cleavage in a manner such that the parent drugs and effective amounts of nitric oxide are released in vivo. On this basis, the inventor provides herein a plausible mechanism of cleavage of nitric oxide-releasing prodrugs (the compound of formula I). The plausible mechanism by which the parent drug(s) designated herein as D and nitric oxide (i.e., possibly in nitrate form) can be released in vivo from the NO-Prodrugs as shown in Scheme M1. In the scheme that depicts plausible mechanism of cleavage of nitric oxide-releasing prodrugs (the compound of formula I), disulfide linker (L1)-containing NO-Prodrug is used for illustrative purpose only.

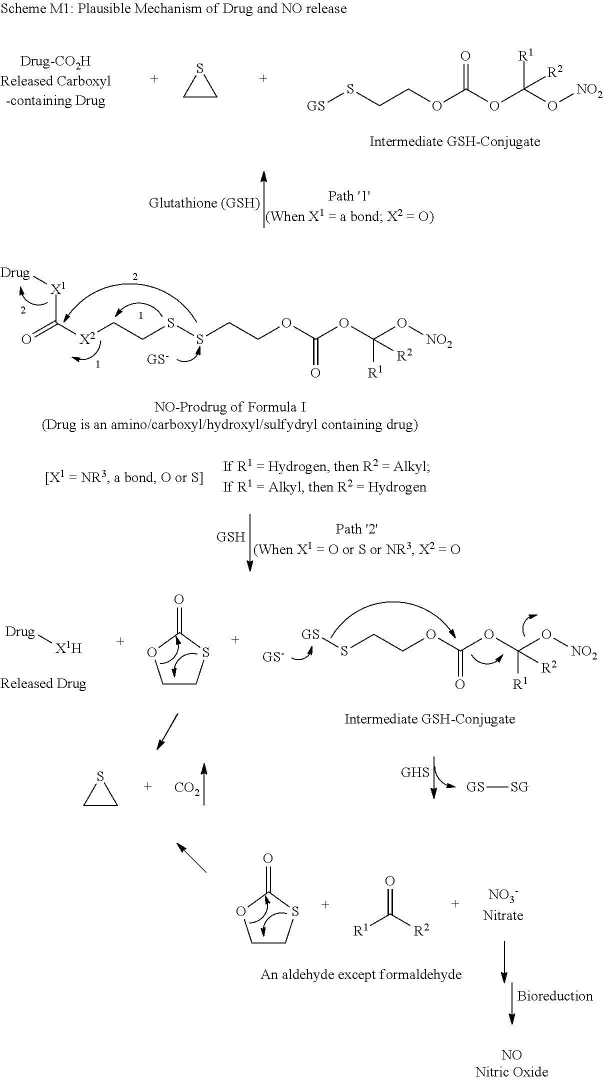

Understandably, the release of parent drug and nitric oxide (i.e., in the form of nitrate/nitrite) from NO-prodrugs containing non-disulfide linkers as found in many other examples of formula I is expected to occur via enzymatic cleavage of linkages between the drug and linker as shown in the following Scheme M2.

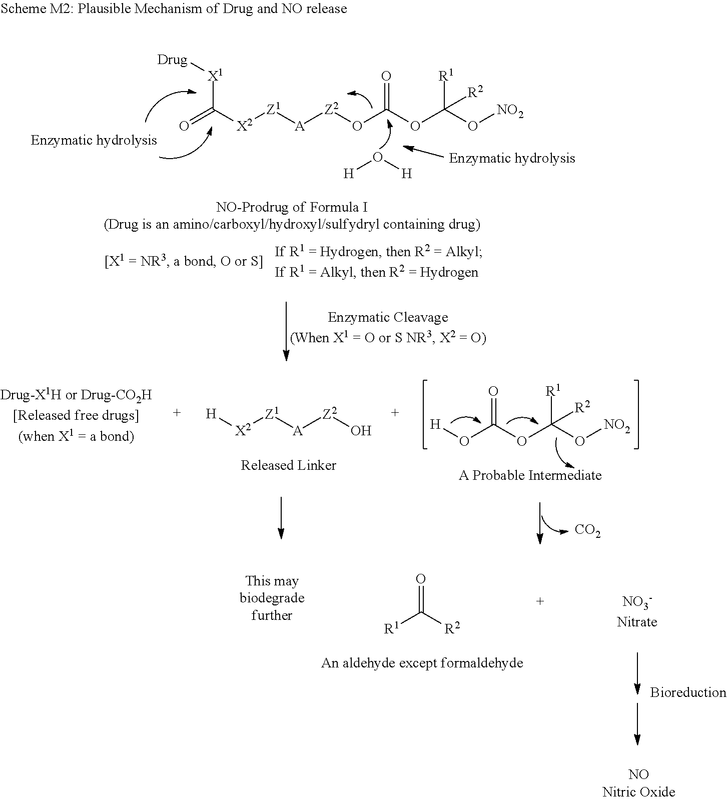

The nitrate ion (NO₃⁻) thus released from the NO-prodrug would get reduced to nitrite in vivo by the action of oral bacteria (i.e., by bacterial nitrate reductase) or Xanthine Oxidase in tissues as shown in the following equation:

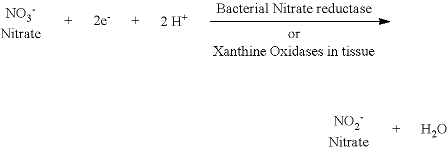

Further reduction of nitrite to nitric oxide (NO) would readily occur in many different ways. Under non-enzymatic acidic conditions in the human body (in stomach or tissue) or by Xanthine Oxidase in tissues or by Cytochromes in liver/tissues nitrite would get converted to nitrous acid which would further dissociateto water and dinitrogen trioxide which in turn would dissociate further to nitrogen dioxide and NO as shown in the following equations:

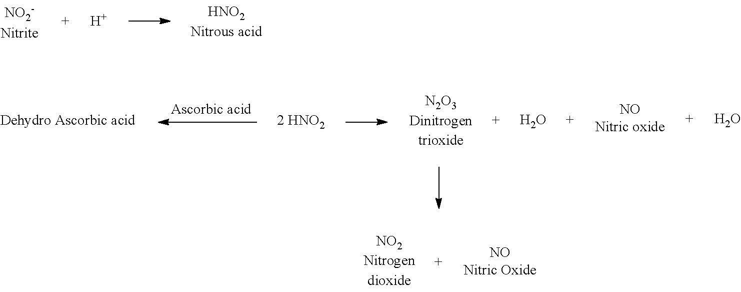

As shown in the above equation, in the presence of vitamin C (Ascorbic acid) and polyphenols, nitrous acid thus generated is directly reduced to NO without yielding nitrogen dioxide (Green L C, et al., Nitrate biosynthesis in man. Proc Natl Acad Sci USA 1981, 78, 7764-8).

The released NO has an extremely short half-life (less then 1 s) in circulating blood (Kelm M. Nitric oxide metabolism and breakdown, Biochim Biophys Acta 1999, 1411, 273-289). NO and nitrite react with oxyhemoglobin to yield nitrate and methemoglobin (Doyle M P, et al., Oxidation of nitrogen oxides by bound dooxygen in hemoproteins. J lnorg Biochem 1981, 14, 351-358; Dyle M P, et al., Kinetics and mechanism of the oxidation of human deoxyhemoglobin in nitrites. J Biol Chem 1981, 256, 12393-8). The half-life of nitrite in blood is 20-30 minutes (Dejam A. et al., Nitrite infusion in humans and nonhuman primates: endocrine effects, pharmacokinetics, and tolerance formation, Circulation 2007, 116, 1821-31) whereas nitrate has a circulating half-life of several hours (Tannenbaum S R. Nitrate and nitrite: origin in humans. Science 1979, 205, 1332, 1334-7; Green L C, et al., Nitrate biosynthesis in man. Proc Natl Acad Sci USA 1981, 78, 7764-8).

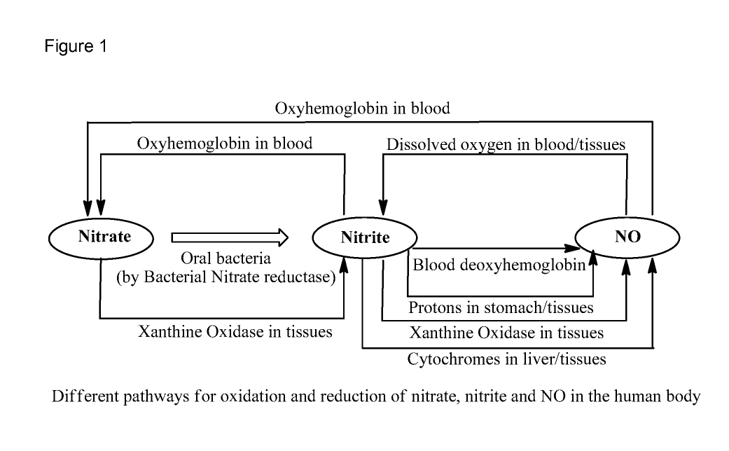

As mentioned in the above equations, there are several pathways for oxidation and reduction of nitrate, nitrite and NO in the body and some of them are summarized in the following diagram (Joel Petersson, 2008. Nitrate, Nitrite and Nitric oxide in Gastric Mucosal Defense, Doctoral Dissertation, 2008, pages 17-18 and the relevant references cited therein)—See FIG. 1.

It is reported that most of the circulating plasma nitrate is excreted through the kidneys (Green L C, et al., Nitrate biosynthesis in man. Proc Natl Acad Sci USA 1981, 78, 7764-8), but about 25% of the plasma nitrate is recycled in the human body to yield nitrite and NO (Tannenbaum S R, et al., The effect of nitrate intake on nitrate formation in human saliva. Food Cosmet Toxicol 1976, 14, 549-52) as shown in the above FIGURE.

The present invention furthermore relates to a pharmaceutical composition containing an effective amount of the compound of formula (I) which is a nitric oxide releasing prodrug of a known drug or a therapeutic agent or its physiologically tolerable salts, along with a pharmaceutically acceptable carrier, and to a process for the production of the pharmaceutical composition, which comprises converting the compound of formula (I) into a suitable administration form using an appropriate pharmaceutically acceptable and physiologically tolerable excipient, and if appropriate, using further suitable active compounds, additives or auxiliaries.

The compound of formula (I), which are the nitric oxide releasing prodrugs of known drugs or therapeutic agents, can be administered to a subject in need thereof in a variety of routes such as oral, for example in the form of pills, tablets, coated tablets, capsules, granules or elixirs. Administration, however, can also be carried out rectally, for example in the form of suppositories, or parentally, for example, intravenously, intramuscularly or subcutaneously, in the form of injectable sterile solutions or suspensions, or topically, for example in the form of solutions or transdermal patches, or in other ways, for example in the form of aerosols or nasal sprays.

The pharmaceutical composition according to the invention is prepared in a manner known per se, and by utilizing methods well-known to one skilled in the art. Pharmaceutically acceptable inert inorganic and/or organic carriers and/or additives can be used in addition to the prodrug compound of formula (I) and/or its pharmacologically acceptable salts. For the production of pills, tablets, coated tablets and hard gelatin capsules it is possible to use, for example, lactose, corn starch or derivatives thereof, gum arabic, magnesia or glucose, etc. Carriers for soft gelatin capsules and suppositories are, for example, fats, wax, natural or hardened oils, etc. Suitable carriers for the production of solutions, for example, injection solutions, or of emulsions or syrups are, for example, water, physiological sodium chloride solution or alcohols, for example, ethanol, propanol, or glycerol, sugar solutions, such as glucose solutions or mannitol solutions, or a mixture of the various solvents which have been mentioned.

The pharmaceutical composition of the invention also contains additives such as, for example, antioxidants, emulsifiers, preservatives, colouring agents and flavouring agents. The pharmaceutical composition may also contain two or more prodrug compounds of formula (I) and/or their physiologically tolerable salts. Furthermore, in addition to at least one prodrug compound of formula (I) or (II) and/or its physiologically tolerable salts, the pharmaceutical composition can also contain one or more other therapeutically or prophylactically active ingredients.

It would be understood by persons skilled in the art that the amount of the compound of formula I (prodrugs of known drugs or therapeutic agents) that is contained in the pharmaceutical composition will depend upon the amount of the parent drug molecule included therein. Generally, the amount of the prodrug used in the treatment methods is that amount which effectively achieves the desired therapeutic effect in subjects being treated for a particular disease. Naturally, the dosages of the various prodrugs encompassed in the compounds of formula (I) will vary somewhat depending upon the parent drug molecule, rate of in vivo drug hydrolysis etc.

The pharmaceutical composition contains about 1 to 99, preferably about 1 to 80% and most preferably from about 10 to 70% by weight of the prodrug compound of formula (I) and/or the physiologically tolerable salts of prodrug compound of formula (I). The effective amount of the active ingredient of prodrug compound of formula (I) and/or its physiologically tolerable salts in the pharmaceutical composition in order to obtain a desired therapeutic effect varies from 1 to 5000 mg. The desirable dosage of the pharmaceutical composition to be administered can vary over a wide range. The selected dosage level can be readily determined by a skilled medical practitioner in the light of the relevant circumstances, including the condition (diseases or disorder) to be treated, the chosen route of administration depending on a number of factors, such as age, weight and physical health and response of the individual patient, pharmacokinetics, severity of the disease and the like, factors known in the medical art. However, in order to obtain desirable effects, it would be recommended to administer the pharmaceutical composition in the form of oral tablets (tablets, capsules) for a day/week/month and in a dosage ranging from 1 mg to 5000 mg, preferably 1 mg to 2000 mg, in a single dosage form or a multi-dosage form.

The range set forth above is illustrative and those skilled in the art will be able to determine the optimal dosing of the prodrugs, the compounds of formula (I) of the present invention selected based on clinical experience and the medical indication or disease to be treated in a subject in need of the treatment.

Another aspect of the present invention is to provide methods for the treatment of various medical conditions or diseases or disorders in a subject comprising administering to a subject in need thereof a therapeutically effective amount of a compound of formula (I). It has already been indicated herein above that the compounds of formula (I) of the present invention are prodrugs of known drugs or therapeutic agents containing a functional group independently selected from a carboxylic acid, an amino, a hydroxyl or a sulfhydryl group. The specific class of therapeutic agents encompassed within the scope of the invention are described herein above. According to the present invention, the diseases or disorders or the medical conditions for the treatment of which the compounds of formula (I) of the present invention are used are those for which the parent drug molecule (represented by the variable D which encompasses specific therapeutic agents) is conventionally used by a medical practitioner. For instance, when the drug or the parent drug molecule contained in the compounds of formula (I) is an anti-inflmmatory and analgesic agent which are known for their use in the treatment of inflammatory disorders or inflammatory conditions, the compounds of formula (I) of the present invention can be used for the treatment of inflammatory conditions or disorders selected from: inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, gout, atherosclerosis, vasculitis, amyloidosis, chronic recurrent uveitis, ulcerative colitis, cachexia, psoriasis, plasmocytoma, endometriosis, Behcet's disease, Wegenrer's granulomatosis, autoimmune disease, immune deficiency, common variable immunodeficiency (CVID), chronic graft-versus-host disease, trauma and transplant rejection, adult respiratory distress syndrome, pulmonary fibrosis, ankylosing spondylitis, skin delayed type hypersensitivity disorders, Alzheimer's disease, systemic lupus erythematosus or allergic asthma. Further, for instance, when the drug or the parent drug molecule contained in the compounds of formula (I) is a cardiovascular agent which is known for its use in the treatment of cardiovascular diseases such as the coronary artery diseases, atheroscerosis, angina, rheumatic heart disease and other related disorders such as hypertension, the compounds of formula (I) of the present invention can also be used for the treatment of similar diseases or conditions.

Thus, the diseases or disorders that can be treated using the compounds of formula (I) of the present invention include but are not limited to inflammatory conditions or disorders, cardiovascular diseases, cancer, allergies, psychological disorders, neurological disorders, cerebrovascular disorders, convulsions, eye diseases, ear diseases, nose and oropharynx diseases, diseases of respiratory system, diseases of gastrointestinal tract system, diseases of genito-urinary system, skin diseases, musculo-skeletal diseases, endocrinal disorders, metabolism disorders such as diabetes, infectious diseases such as bacterial infections and fungal infections, viral infections etc.

Accordingly, in one aspect the present invention is related to a method of treating a disease or disorder where a chronic, sustained and selective release of the constituent drug or therapeutic agent D or nitric oxide contained in the compounds of formula (I) is beneficial; comprising administering to a mammal or a human in need of the treatment a therapeutically effective amount of the compound of formula (I).

In another aspect, the present invention also relates to a method of treating a disease in a human or mammal where a chronic, sustained and selective release of the constituent drug or therapeutic agent D or nitric oxide contained in the compounds of formula (I) is beneficial; comprising administering to said mammal a therapeutically effective amount of the pharmaceutical composition containing the compounds of formula (I).

Moreover, the compounds of formula (I), which are the prodrugs of known drugs or therapeutic agents, in all likelihood are advantageous over the parent drug molecules or prodrugs of the parent molecule known hitherto in the prior art in terms of increased bioavailability, reduced adverse effect, for instance, gastric irritability caused by NSAIDS etc. Moreover, representative compounds encompassed in the compounds of formula (I) have been found to be devoid of genotoxicity at a concentration at which the compounds are expected to be used for the treatment of the medical conditions or diseases for the treatment of which the parent drug molecule is used.

It is understood that modifications that do not substantially affect the activity of the various embodiments of this invention are included within scope of the invention disclosed herein. Accordingly, the following examples are intended to illustrate but not to limit scope of the present invention.

### EXPERIMENTAL

The abbreviations and terms that are used herein:

BOP: Benzotriazol-1-yl-oxy-tris(dimethylamino)phosphonium hexafluorophosphate

### DMF: N,N-Dimethylformamide

DSC: N,N′-Disuccinimidyl carbonate

### CDI: N,N′-Carbonyldiimidazole

### DCC: N,N′-Dicyclohexylcarbodiimide

### DMAP: 4-Dimethylaminopyridine

EDAC. HCl: 1-Ethyl-(3-dimethylaminopropyl)carbodiimide hydrochloride
HBTU: 0-(Benzotriazol-1-yl)-N,N,N′,N′-tetramethyluronium hexafluorophosphate
TBTU: 0-(Benzotriazol-1-yl)-N,N,N′,N′-tetramethyluronium tetrafluoroborate

### EtOH: Ethanol

### LAH: Lithium Aluminum Hydride

Et₂O: Diethyl ether

### THF: Tetrahydrofuran

DMSO: Dimethyl sulfoxide

### TEA: Triethylamine

### DIPEA: N,N-Diisopropylethylamine

### DCM: Dichloromethane

EtOAc or EA: Ethyl acetate

### DME: Dimethoxyethane

### MeOH: Methanol

PE: Petroleum ether
RT: Room temperature
TFA: Trifluoroacetic acid

### HOBT: N-Hydroxybenzotriazole

### HPLC: High Performance Liquid Chromatography

### TLC: Thin Layer Chromatography

### RT: Room Temperature

Examples of the compounds of formula I which are the prodrugs of the drugs containing an carboxylic acid group:

### Example 1

### (2S)-2-((2-((1-(nitrooxy)ethoxy)carbonyloxy)ethyl)disulfanyl)ethyl 2-(6-methoxynaphthalen-2-yl)propanoate (I-CD1-L1-R1)

This compound was synthesized in 3 steps as shown in Scheme 1 and the experimental procedure is described below:

### Step 1: Preparation of (S)-2-((2-hydroxyethyl)disulfanyl)ethyl 2-(6-methoxy-naphthalen-2-yl) propanoate [NO-Naproxen (CD1-L1-OH)]

A solution of DCC (13.0 g, 62.6 mmol) in DCM (25 mL) was added drop-wise over 5 minutes to a stirred solution of naproxen (CD1, 12.0 g, 52.2 mmol), bis(2-hydroxyethyl) disulfide (HO-L1-OH, 13.4 g, 104.3 mmol) and DMAP (1.3 g, 10.4 mmol) in 250 mL of DCM at 0° C. and the mixture was stirred for 3 h when TLC analysis of the mixture indicated completion of the reaction. The mixture was filtered and the filtrate was washed with water (2×100 mL) and brine (1×100 mL). The organic layer was separated, dried over Na₂SO₄ and concentrated in vacuo to give the crude product which was purified by column chromatography (600 g of silica gel, 150-300 mesh). The expected bis-naproxen derivative (i.e., CD1-L1-CD1), which was formed as a minor undesired product, was eluted with 10% EtOAc in petroleum ether. The desired mono-acylated title compound, which was eluted with 20% EtOAc in petroleum ether, was obtained as a white solid. Yield: 12.0 g (63.1%); ¹H NMR (CDCl₃, 300 MHz): δ 1.58 (d, J=7.2 Hz, 3H), 2.77 (t, J=5.7 Hz, 2H), 2.86 (t, J=6.9 Hz, 2H), 3.77 (t, J=5.7 Hz, 2H), 3.87 (q, J=7.2 Hz, 1H), 3.91 (s, 3H), 4.28-4.42 (m, 2H), 7.08-7.17 (m, 2H), 7.40 (dd, J=8.4, 1.5 Hz, 1H), 7.64-7.73 (m, 3H); MS m/z: 384.1 [M+NH₄]⁺.

### Step 2: Preparation of (2S)-2-((2-((1-chloroethoxy)carbonyloxy)ethyl) disulfanyl)ethyl 2-(6-methoxynaphthalen-2-yl)propanoate (CD1-L1-R1-CI)

α-chloroethyl chloroformate (CI-R1-CI, 1.1 mL, 11.5 mmol) was added drop-wise to a solution of (S)-2-((2-hydroxyethyl)disulfanyl)ethyl 2-(6-methoxynaphthalen-2-yl)-propanoate (CD1-L1-OH, 3.5 g, 9.6 mmol) in 30 mL of DCM at 0° C. under nitrogen atmosphere. To this stirred mixture was added a solution of pyridine (1.2 mL, 14.3 mmol) in 5 mL of DCM over 5 minutes. The mixture was stirred at 0° C. under nitrogen atmosphere for 30 minutes when TLC analysis of the mixture indicated completion of the reaction. The mixture was diluted with DCM (˜65 mL), washed with 1N HCl (3×100 mL), saturated sodium bicarbonate (1×100 mL) and brine (2×50 mL). The organic layer was dried over anhydrous Na₂SO₄ and concentrated in vacuo to afford a yellow oily residue which was purified by column chromatography (200 g silica gel, 200-400 mesh, eluted with 10% EtOAc in petroleum ether) to afford the title compound, CD1-L1-R1-CI as a slight greenish yellow colored oil. Yield: 3.2 g (70.8%); ¹H NMR (CDCl₃, 300 MHz): δ 1.58 (d, J=6.9 Hz, 3H), 1.82 (d, J=5.7 Hz, 3H), 2.83-2.93 (m, 4H), 3.87 (q, J=7.2 Hz, 1H), 3.91 (s, 3H), 4.27-4.43 (m, 4H), 6.41 (q, J=6.0 Hz, 1H), 7.10-7.18 (m, 2H), 7.40 (dd, J=8.4, 1.5 Hz, 1H), 7.67 (br s, 1H), 7.71 (d, J=8.4 Hz, 2H); MS m/z: 491.25 [M+NH₄]⁺.

### Step 3: Preparation of (2S)-2-((2-((1-(nitrooxy)ethoxy)carbonyloxy)ethyl)disulfanyl)-ethyl 2-(6-methoxynaphthalen-2-yl)propanoate (I-CD1-L1-R1)

Silver nitrate (1.4 g, 8.1 mmol) was added to a solution of (2S)-2-((2-((1-chloroethoxy)carbonyloxy)ethyl)disulfanyl)ethyl 2-(6-methoxynaphthalen-2-yl)propanoate (CD1-L1-R1-CI, 3.2 g, 6.8 mmol) in 35 mL of ACN and the mixture was refluxed in dark at 85-90° C. for 30 minutes when TLC analysis of the mixture indicated complete conversion. The mixture was cooled and filtered through celite. The filtrate was concentrated and the residue (˜3.5 g) was purified by column chromatography (150 g of silica gel, 200-400 mesh, eluted with 10% EtOAc in petroleum ether) to afford 2.3 g of slightly impure product which was purified again by column chromatography [100 g of silica gel, 200-400 mesh, eluted with petroleum ether/DCM (2:3)] to afford the pure title compound (I-CD1-L1-R1) as greenish oil. Yield: 1.8 g (84%); ¹H NMR (CDCl₃, 300 MHz): δ 1.56-1.61 (m, 6H), 2.81-2.90 (m, 4H), 3.87 (q, J=7.2 Hz, 1H), 3.91 (s, 3H), 4.30-4.38 (m, 4H), 6.91 (q, J=5.7 Hz, 1H), 7.10-7.17 (m, 2H), 7.40 (dd, J=8.4, 1.8 Hz, 1H), 7.65-7.73 (m or distorted t, 3H); ¹³C NMR (CDCl₃, 75.47 MHz): δ 17.5, 18.6, 30.5, 30.8, 45.5, 55.4, 64.1, 67.4, 96.4, 105.7, 119.2, 126.1, 126.3, 127.3, 129.0, 129.4, 133.8, 135.5, 152.6, 157.8, 174.5; MS m/z: 522.1 [M+Na]⁺; HRMS ESI (m/z): [M+Na]⁺ calculated for C₂₁H₂₅N₁Na₁O₉S₂: 522.0863; Found: 522.0869 (Mass Accuracy: 1.15 ppm).

### Example 2

### 2-((2-((1-(nitrooxy)butoxy)carbonyloxy)ethyl)disulfanyl)ethyl 2-acetoxybenzoate [NO-Aspirin/Salicylic acid (I-CD2-L1-R2)]

This compound was synthesized in 3 steps as shown in Scheme 1 and the experimental procedure is described below:

### Step 1: Synthesis of 2-((2-hydroxyethyl)disulfanyl)ethyl 2-acetoxybenzoate (CD2-L1-OH)

A solution of aspirin acid chloride (CD2-CI, 7.0 g, 35.3 mmol, freshly prepared from aspirin by using oxalyl chloride/DMF/DCM method) in 20 mL of DCM was added drop-wise to a stirred solution of 2-hydroxyethyl disulfide (HO-L1-OH, 10.9 g, 70.5 mmol) and Triethylamine (7.35 mL, 52.89 mmol) in 50 mL of DCM at 0° C. under nitrogen atmosphere and the mixture was stirred at RT for overnight, when TLC analysis of the mixture indicated completion of the reaction. The mixture was diluted with 25 mL of water and 100 mL of DCM. The organic layer was separated and washed with aqueous sodium bicarbonate (2×100 mL) and brine (1×100 mL), dried over Na₂SO₄ and concentrated in vacuo to give 10.0 g of crude oil which was purified by column chromatography (225.0 g of silica gel, 150-300 mesh, eluted with 5-30% ethyl acetate in petroleum ether) to afford the title compound (CD2-L1-OH) as yellow oil. Yield: 5.2 g (46.6%); ¹H NMR (CDCl₃, 300 MHz): δ 2.26 (bt, J=4.2 Hz, 1H), 2.38 (s, 3H), 2.90 (t, J=6.0 Hz, 2H), 3.05 (t, J=6.6 Hz, 2H), 3.69 (distorted q or m, 2H), 4.57 (t, J=6.6 Hz, 2H), 7.13 (dd, J=8.1, 0.9 Hz, 1H), 7.34 (dt, J=7.8 Hz, 1H), 7.60 (dt, J=7.8, 1.5 Hz, 1H), 8.06 (dd, J=7.8, 1.5 Hz); MS m/z: 339.0 [M+Na]⁺.

### Step 2: Synthesis of 2-((2-((1-chlorobutoxy)carbonyloxy)ethyl)disulfanyl)ethyl 2-acetoxybenzoate (CD2-L1-R2-CI)

Pyridine (73.0 μL, 0.9 mmol) followed by diphosgene (1.1 mL, 9.3 mmol) were added to a stirred solution of butyraldehyde (1.0 g, 13.9 mmol) in 3 mL of dry DCM at RT under a nitrogen atmosphere and the mixture was stirred at RT for 3 h. About 50% of the solvent was distilled off in vacuo and kept the mixture under nitrogen atmosphere. To this stirred mixture at 0° C. under nitrogen was added a solution of 2-((2-hydroxyethyl)disulfanyl)ethyl 2-acetoxybenzoate (CD2-L1-OH, 1.5 g, 4.6 mmol) in 4 mL of dry DCM followed by pyridine (1.1 mL, 13.9 mmol) and the mixture was stirred at 0° C. for 30 minutes when TLC analysis of the mixture indicated completion of the reaction. The mixture was diluted with 10 mL of DCM, washed with 1N HCl (3×15 mL), saturated sodium bicarbonate (3×15 mL) and brine (2×10 mL). The organic layer was dried over anhydrous Na₂SO₄ and concentrated in vacuo to give a oily residue (2.5 g) which was purified by column chromatography (40.0 g of silica gel, 200-400 mesh; eluted with 5-8% of EtOAc in petroleum ether) to afford the title compound as yellow oil. Yield: 1.6 g (80%); ¹H NMR (CDCl₃, 300 MHz): δ 0.98 (t, J=7.5 Hz, 3H), 1.47-1.62 (m, 2H), 1.96-2.17 (m, 2H), 2.38 (s, 3H), 3.00 (t, J=6.6 Hz, 2H), 3.06 (t, J=6.6 Hz, 2H), 4.48 (t, J=6.6 Hz, 2H), 4.56 (t, J=6.6 Hz, 2H), 6.32 (t, J=6.0 Hz, 1H), 7.13 (dd, J=7.8, 0.9 Hz, 1H), 7.34 (dt, J=7.8, 0.9 Hz, 1H), 7.59 (dt, J=7.8, 1.5 Hz, 1H), 8.06 (dd, J=7.8, 1.5 Hz, 1H); MS m/z: 474.0 [M+Na]⁺.

### Step 3: Synthesis of 2-((2-((1-(nitrooxy)butoxy)carbonyloxy)ethyl)disulfanyl)ethyl 2-acetoxybenzoate (I-CD2-L1-R2)

Silver nitrate (0.9 g, 5.0 mmol) was added to a solution of 2-((2-((1-chlorobutoxy)-carbonyloxy)ethyl) disulfanyl)ethyl 2-acetoxybenzoate (CD2-L1-R2-CI, 1.5 g, 3.3 mmol) in 15 mL of ACN at RT under a nitrogen atmosphere (covered the reaction flask with aluminum foil to minimize exposure of reaction mixture to light) and the mixture was stirred at RT for overnignt (˜16 h). HPLC analysis of the mixture indicated complete conversion. The mixture was diluted with 10 mL of DCM and filtered through a small pad of celite to remove the insoluble salts. The filtrate was concentrated to give 2.0 g of oily residue which was purified by column chromatography (50.0 g of silica gel, 200-400 mesh, eluted with 8% EtOAc in petroleum ether) to afford the title compound as yellow oil. Yield: 0.4 g (27%). (Note: additional ˜0.35 g (˜19%) of impure product (˜80% pure by HPLC) was also obtained); ¹H NMR (CDCl₃, 300 MHz): δ 1.00 (t, J=7.5 Hz, 3H), 1.43-1.58 (m, 2H), 1.83-1.92 (m, 2H), 2.38 (s, 3H), 2.99 (t, J=6.6 Hz, 2H), 3.05 (t, J=6.6 Hz, 2H), 4.47 (t, J=6.6 Hz, 2H), 4.55 (t, J=6.6 Hz, 2H), 6.85 (t, J=6.0 Hz, 1H), 7.13 (dd, J=7.8, 0.9 Hz, 1H), 7.34 (dt, J=7.8, 0.9 Hz, 1H), 7.59 (dt, J=7.8, 1.5 Hz, 1H), 8.05 (dd, J=7.8, 1.8 Hz); ¹³C NMR (CDCl₃, 75.47 MHz): δ 12.9, 16.2, 20.6, 32.7, 36.2, 36.6, 62.3, 65.8, 97.9, 122.4, 123.3, 125.6, 131.3, 133.6, 150.3, 152.3, 163.6, 169.1; MS m/z: 477.1 [M+H]⁺, 500.1 [M+Na]⁺.

### Example 3

### (2S)-((Z)-4-((1-(nitrooxy)ethoxy)carbonyloxy)but-2-enyl) 2-(6-methoxynaphthalen-2-yl)propanoate [NO-Naproxen (I-CD1-L2-R1)]

This compound was synthesized in 4 steps as shown in Scheme 1 and the experimental procedure is described below:

### Step 1: Preparation of (S)-2-6-methoxynaphthalen-2-yl)propanoyl chloride (CD1-CI)

DMF (˜3-4 drops) followed by oxalyl chloride (11.0 mL, 130.4 mmol) were added drop-wise to a stirred solution of naproxen (DC1, 25.0 g, 108.7 mmol) in 200 mL of DCM at RT under a nitrogen atmosphere over 10 minutes. The mixture was stirred at RT under nitrogen atmosphere for 3 h. The mixture was concentrated in vacuo to afford crude naproxen acid chloride as a yellow solid, which was used as such in the next step. Yield: 27.0 g (quantitative).

### Step 2: Preparation of (S,Z)-4-hydroxybut-2-enyl 2-(6-methoxynaphthalen-2-yl)-propanoate (CD1-L2-OH)

A solution of naproxen chloride (5.0 g, 20.0 mmol) in 10 mL of DCM was added to a stirred solution of cis-2-butene-1,4-diol (HO-L2-OH, 5.3 mL, 60.0 mmol) in 100 mL of DCM at 0° C. under a nitrogen atmosphere. To this stirred mixture was added triethylamine (4.2 mL, 30.0 mmol) drop-wise over 15 minutes and the resulting mixture was stirred at 0° C. for 30 minutes and at RT for overnight (˜12 h), when TLC analysis of the mixture indicated formation of two product spots (i.e., mono and bis-acylated products). The mixture was washed with saturated sodium bicarbonate (3×100 mL), brine (3×100 mL), dried over anhydrous Na₂SO₄ and concentrated to afford 7.0 g of crude oily residue which was purified by column chromatography (150.0 g of silica gel, 200-400 mesh, eluted with 10% EtOAc in petroleum ether to isolate the bis-acylated compound and with 20% EtOAc in petroleum ether to isolate the desired mono-acylated product). The title compound was obtained as a white solid. Mp: 69-71° C.; Yield: 4.5 g (75%); ¹H NMR (CDCl₃, 300 MHz): δ 1.57 (d, J=6.9 Hz, 3H), 1.99 (br s, 1H), 3.85 (q, J=6.9, 7.2 Hz, 1H), 3.91 (s, 3H), 4.18 (t, J=4.8 Hz, 2H), 4.60-4.73 (m, 2H), 5.50-5.62 (m, 1H), 5.75-5.85 (m, 1H), 7.09-7.17 (m, 2H), 7.38 (dd, J=8.4, 1.5 Hz, 1H), 7.65 (br s, 1H), 7.70 (d, J=8.7 Hz, 2H); MS m/z: 323.1 [M+Na]⁺.

### Step 3: Preparation of (2S)-((Z)-4-((1-chloroethoxy)carbonyloxy)but-2-enyl) 2-(6-methoxynaphthalen-2-yl)propanoate (CD1-L2-R1-CI)

α-chloroethyl chloroformate (CI-R1—C1, 1.6 mL, 16.4 mmol) was added drop-wise to a stirred solution of (S,Z)-4-hydroxybut-2-enyl 2-(6-methoxynaphthalen-2-yl)propanoate (CD1-L2-OH, 4.1 g, 13.7 mmol) in 50 mL of DCM at 0° C. under a nitrogen atmosphere. To this stirred mixture was added a solution of pyridine (1.7 mL, 20.4 mmol) in 5 mL of DCM over 5 minutes. The mixture was stirred at 0° C. under nitrogen for 30 minutes and at RT for 3 h when TLC analysis of the mixture indicated completion of the reaction. The mixture was diluted with DCM (˜75 mL), washed with 1N HCl (3×100 mL), saturated sodium bicarbonate (1×100 mL) and brine (2×100 mL). The organic layer was dried over anhydrous Na₂SO₄ and concentrated in vacuo to give a greenish oily residue which was used as such in the next step as its purity was >90% (by HPLC) and its proton NMR and mass spectral data was consistent with the expected structure. Yield: 5.0 g (89.9%); ¹H NMR (CDCl₃, 300 MHz): δ 1.57 (d, J=7.5 Hz, 3H), 1.81 (d, J=5.7 Hz, 3H), 3.86 (q, J=7.2 Hz, 1H), 3.91 (s, 3H), 4.61-4.82 (m, 4H), 5.66-5.79 (m, 2H), 6.39 (dq, J=1.2, 6.0 Hz, 1H), 7.10-7.17 (m, 2H), 7.38 (dd, J=8.4, 1.5 Hz, 1H), 7.65 (d, J=1.2 Hz, 1H), 7.70 (d, J=8.7 Hz, 2H); MS m/z: 429.1 [M+Na]⁺.

### Step 4: Preparation of (2S)-((Z)-4-((1-(nitrooxy)ethoxy)carbonyloxy)but-2-enyl 2-(6-methoxynaphthalen-2-yl)propanoate (I-CD1-L2-R1)

Silver nitrate (3.1 g, 18.8 mmol) was added to a solution of (2S)-((Z)-4-((1-chloroethoxy)carbonyloxy)but-2-enyl) 2-(6-methoxynaphthalen-2-yl)propanoate (CD1-L2-R1-Cl, 5.0 g, 12.3 mmol) in 50 mL of ACN and the mixture was refluxed in dark at 85-90° C. for ˜40 min when TLC analysis of the mixture indicated complete conversion. The mixture was cooled, diluted with DCM (˜70 mL) and filtered through celite. The filtrate was concentrated and the residue was re-dissolved in DCM (˜50 mL) and the separated silver salt was filtered again through celite. The filtrate was concentrated to give 7.0 g of residue which was purified by column chromatography (150.0 g of silica gel 200-400 mesh, eluted with 7-10% EtOAc in petroleum ether) to afford the title compound as a white solid. Mp: 76-78° C.; Yield: 5.0 g (93.7%); ¹H NMR (CDCl₃, 300 MHz): δ 1.55-1.62 (m, 6H), 3.86 (q, J=7.2 Hz, 1H), 3.91 (s, 3H), 4.65-4.72 (m, 4H), 5.65-5.79 (m, 2H), 6.89 (q, J=5.7 Hz, 1H), 7.10-7.18 (m, 2H), 7.40 (dd, J=8.4, 1.8 Hz, 1H), 7.64-7.74 (m or distorted t, 3H); ¹³C NMR (CDCl₃, 75.47 MHz): δ 17.5, 18.6, 45.5, 55.4, 60.3, 64.1, 96.3, 105.7, 119.2, 126.1, 126.3, 126.7, 127.4, 129.0, 129.3, 129.4, 133.9, 135.5, 152.6, 157.8, 174.4; MS m/z: 456.1 [M+Na]⁺; HRMS ESI (m/z): [M+Na]⁺ calculated for C₂₁H₂₃N₁Na₁O₉: 456.1265; Found: 456.1266 (Mass Accuracy: 0.88 or ˜0.22 ppm).

### Example 4

### (Z)-4-((1-(nitrooxy)ethoxy)carbonyloxy)but-2-enyl nicotinate [NO-Niacin (I-CD3-L2-R1)]

The title compound was synthesized in 3 steps as shown in Scheme 2 and the experimental procedure is described below:

### Step 1: Preparation of (Z)-1-chloroethyl 4-hydroxybut-2-enyl carbonate (HO-L2-R1-CI)

α-Chloroethyl chloroformate (CI-R1-CI, 20.0 mL, 187.0 mmol) was added drop-wise to a stirred solution of cis-2-butene-1,4-diol (HO-L2-OH, 15.0 g, 170.2 mmol) and pyridine (27.0 mL, 340.0 mmol) in 200 mL of DCM at 0° C. over a period of 10 minutes and the mixture was stirred at 0° C. for 1 h. TLC analysis of the mixture indicated completion of the reaction. The mixture was diluted with 100 mL of DCM and washed with 1N HCl (2×200 mL), water (2×150 mL), and brine (2×150 mL). The organic layer was dried over Na₂SO₄ and concentrated in vacuo to obtain an oil which was purified by column chromatography (silica gel 100-200 mesh, eluted with 5% EtOAc in petroleum ether) to afford the title compound as a colorless oil. Yield: 16.0 g (48.5%); ¹H NMR (CDCl₃, 300 MHz): δ 1.85 (d, J=5.7 Hz, 3H), 4.30 (d, J=6.3 Hz, 2H), 4.75-4.90 (m, 2H), 5.65-5.76 (m, 1H), 5.90-5.99 (m, 1H), 6.43 (q, J=5.7 Hz, 1H); ¹³C NMR (CDCl₃, 125.77 MHz): δ 25.2, 58.4, 64.1, 84.7, 124.1, 134.7, 152.9; MS m/z: 217.1 [M+Na]⁺.

### Step 2: Preparation of (Z)-4-hydroxybut-2-enyl 1-(nitrooxy)ethyl carbonate (HO-L2-R1)

Silver nitrate (15.7 g, 92.5 mmol) was added to a solution of (Z)-1-chloroethyl 4-hydroxybut-2-enyl carbonate (HO-L2-R1-CI, 12.0 g, 61.7 mmol) in acetonitrile (120 mL) and the mixture was stirred at 80° C. for 2 h. HPLC analysis of the mixture indicated completion of conversion. The mixture was cooled to RT and filtered through celite. The filtrate was concentrated to give the residue which was re-dissolved in 200 mL of DCM and filtered through celite to remove the separated silver chloride. The filtrate was washed with water (2×100 mL) and brine (2×100 mL), dried over Na₂SO₄ and concentrated in vacuo to give the crude product as yellow oil, which was used as such in the next step. Yield: 8.9 g (65.3%); ¹H NMR (CDCl₃, 300 MHz): δ 1.60 (d, J=6.0 Hz, 3H), 1.99 (s, 1H), 4.28 (t, J=6.3 Hz, 2H), 4.80 (d, J=7.2 Hz, 2H), 5.64-5.75 (m, 1H), 5.90-5.98 (m, 1H), 6.93 (q, J=5.7 Hz, 1H).

### Step 3: Preparation of (Z)-4-((1-(nitrooxy)ethoxy)carbonyloxy)but-2-enyl nicotinate (I-CD3-L2-R1)

A solution of (Z)-4-hydroxybut-2-enyl 1-(nitrooxy)ethyl carbonate (HO-L2-R1, 0.6 g, 2.8 mmol) and pyridine (0.5 mL, 5.6 mmol) in 4 mL of DCM was added drop-wise to a stirred suspension of nicotinoyl chloride hydrochloride (CD3-CI. HCl, 0.5 g, 2.8 mmol) in 6 mL of DCM at 0° C. under a nitrogen atmosphere over 10 minutes and the resulting mixture was stirred at RT for 2 h when TLC analysis of the mixture indicated completion of the reaction. The mixture was diluted with 20 mL of DCM, washed with water (2×20 mL), dried over Na₂SO₄ and concentrated to give the crude residue which was purified by column chromatography (silica gel, eluted with 30-50% EtOAc in petroleum ether) to afford the title compound as colorless oil. Yield: 0.6 g (60%); ¹H NMR (CDCl₃, 300 MHz): δ 1.62 (d, J=5.7 Hz, 3H), 4.69-4.80 (m, 1H), 4.87-4.93 (m, 2H), 4.99 (d, J=6.6 Hz, 1H), 5.83-6.13 (m, 2H), 6.95 (q, J=5.7 Hz, 1H), 7.42 (dd, J=8.1, 5.1 Hz, 1H), 8.30-8.37 (m, 1H), 8.81 (d, J=4.8 Hz, 1H), 9.23-9.28 (m, 1H); MS m/z: 327.1 [M+H]⁺.

### Example 5

### (Z)-4-(1-(nitrooxy)ethoxy)carbonyloxy)but-2-enyl2-(2-(4-((4-chlorophenyl) (phenyl)methyl)piperazin-1-yl)ethoxy)acetate [NO-Cetirizine (I-CD4-L2-R1)]

This compound was synthesized as shown in Scheme 2 and the experimental procedure is described below:

Triethylamine (TEA, 0.9 mL, 4.5 mmol) followed by a solution of (Z)-4-hydroxybut-2-enyl 1-(nitrooxy)ethyl carbonate (HO-L2-R1, 0.5 g, 2.3 mmol) in DCM (5 mL) was added to a stirred suspension of cetirizine dihydrochloride (CD4. 2HCl, 1.0 g, 2.3 mmol) in 15 mL of DCM. To this stirred mixture was added DCC (0.6 g, 2.7 mmol) followed by DMAP (50 mg, ˜0.4 mmol) and the resulting mixture was stirred for 2 h when TLC analysis of the mixture indicated formation of a new product. The mixture was diluted with 10 mL of DCM and filtered. The filtrate was washed with saturated sodium bicarbonate (10 mL) and brine (10 mL). The organic layer was dried over Na₂SO₄ and concentrated on rotavap to give a crude residue which was purified by column chromatography (silica gel, eluted with EtOAc/petroleum ether gradient) to afford the title compound as a yellow gum/highly viscous oil. Yield: 0.4 g (32.6%); ¹H NMR (CDCl₃, 300 MHz): δ 1.61 (d, J=5.7 Hz, 3H), 2.35-2.64 (m, 8H), 2.68 (t, J=5.7 Hz, 2H), 3.69 (t, J=5.7 Hz, 2H), 4.13 (s, 2H), 4.22 (s, 1H), 4.64-4.83 (m, 4H), 5.80-5.93 (m, 2H), 6.91-6.98 (m, 1H), 7.16-7.41 (m, 9H); MS m/z: 592 [M+H]⁺.

The compounds of the examples 6-11 were prepared by following the experimental procedure for the compound exemplified in example 5. The characterization data for the compounds of examples 6-11 is described below:

### Example 6

### (2R)-((Z)-4-((1-(nitrooxy)ethoxy)carbonyloxy)but-2-enyl) 2-((1S,4S)-4-isopropyl-cyclohexanecarboxamido)-3-phenylpropanoate [NO-Nateglinide (I-CD5-L2-R1)]

The title compound was obtained as a pale yellow gum. Yield (last step): 22.0%; ¹H NMR (CDCl₃, 300 MHz): δ 0.87 (d, J=6.9 Hz, 6H), 1.62 (d, J=5.7 Hz, 3H), 0.89-2.09 (m, 11H), 3.06-3.22 (m, 2H), 4.62-4.97 (m, 5H), 5.72-5.95 (m, 3H), 6.94 (q, J=5.7 Hz, 1H), 7.07-7.13 (m, 2H), 7.23-7.35 (m, 3H); MS m/z: 521.2 [M+H]⁺, 543.2 [M+Na]⁺.

### Example 7

### (Z)-4-((1-(nitrooxy)ethoxy)carbonyloxy)but-2-enyl 2-(2-(2,6-dichlorophenylamino) phenyl)acetate [NO-Diclofenac (I-CD6-L2-R1)]

The title compound was obtained as light red oil. Yield (last step): 89.9%; ¹H NMR (CDCl₃, 300 MHz): δ 1.61 (d, J=5.7 Hz, 3H), 3.85 (s, 2H), 4.79 (distorted t, J=6.0 Hz, 4H), 5.74-5.92 (m, 2H), 6.57 (d, J=7.8 Hz, 1H), 6.86 (br s, 1H), 6.94 (q, J=5.7 Hz, 1H), 6.99 (q, J=8.1 Hz, 2H), 7.16 (dt, J=7.8, 1.5 Hz, 1H), 7.25 (dd, J=7.5, 1.5 Hz, 1H), 7.37 (d, J=8.1 Hz, 2H).

### Example 8

### (Z)-4-((1-(nitrooxy)ethoxy)carbonyloxy)but-2-enyl 2-(1-(4-chlorobenzoyl)-5-methoxy-2-methyl-1H-indol-3-yl)acetate [NO-Indomethacin (I-CD7-L2-R1)]

The title compound was obtained as yellow viscous oil. Yield (last step): 92.7%; ¹H NMR (CDCl₃, 300 MHz): δ 1.60 (d, J=5.4 Hz, 3H), 2.41 (s, 3H), 3.70 (s, 2H), 3.85 (s, 3H), 4.75 (distorted dd, J=10.2, 5.4 Hz, 4H), 5.73-5.88 (m, 2H), 6.69 (dd, J=9.0, 2.4 Hz, 1H), 6.88 (d, J=9.0 Hz, 1H), 6.93 (q merged with adjacent doublets, J=5.7 Hz, 1H), 6.96 (d, J=2.4 Hz, 1H), 7.49 (distorted d, J=8.4 Hz, 2H), 7.69 (distorted d, J=8.7 Hz, 2H);

MS m/z: 561.1 [M+H]⁺, 583.1 [M+Na]⁺, 599 [M+K]⁺.

### Example 9

### 4-((1-(nitrooxy)ethoxy)carbonyloxy)butyl 2-(6-methoxynaphthalen-2-yl)propanoate [NO-Naproxen (I-CD1-L3-R1)]

The title compound was also obtained as oil. Yield (last step): 86.9%; ¹H NMR (CDCl₃, 300 MHz): δ 1.56-1.62 (m, 6H), 1.64-1.72 (m, 4H), 3.87 (q, J=7.2 Hz, 1H), 3.94 (s, 3H), 4.09-4.18 (m, 4H), 6.92 (q, J=5.7 Hz, 1H), 7.11-7.19 (m, 2H), 7.41 (dd, J=8.4, 1.5 Hz, 1H), 7.68 (br s, 1H), 7.72 (d, J=8.4 Hz, 2H); MS m/z: 458.1 [M+Na]⁺, 474.1 [M+K]⁺; HRMS ESI (m/z): [M+Na]⁺ calculated for C₂₁H₂₅N₁Na₁O₉: 458.1422; Found: 458.1431 (Mass Accuracy: −1.96 ppm).

### Example 10

### (2S)-3-((1-(nitrooxy)ethoxy)carbonyloxy)propyl 2-(6-methoxynaphthalen-2-yl) propanoate [NO-Naproxen (I-CD1-L4-R1)]

The title compound was also obtained as oil. Yield (last step): 68.0%; ¹H NMR (CDCl₃, 300 MHz): δ 1.53-1.64 (m, 6H), 1.91-2.02 (m, 2H), 3.86 (q, J=7.2 Hz, 1H), 3.92 (s, 3H), 4.13-4.21 (m, 4H), 6.85-6.93 (m, 1H), 7.10-7.18 (m, 2H), 7.41 (d, J=8.4 Hz, 1H), 7.66 (br s, 1H), 7.70 (d, J=8.4 Hz, 2H); MS m/z: 444.1 [M+Na]⁺.

### Example 11

### (2S)-2,2-dimethyl-3-((1-(nitrooxy)ethoxy)carbonyloxy)propyl 2-(6-methoxy-naphthalen-2-yl) propanoate [NO-Naproxen (I-CD1-L5-R1)]

The title compound was also obtained as yellow oil. Yield (last step): 96.0%; ¹H NMR (CDCl₃, 300 MHz): δ 0.86 (s, 3H), 0.90 (s, 3H), 1.53-1.64 (m, 6H), 3.83-3.97 (m buried under —OCH₃ singlet, 5H), 3.95 (s, 3H), 6.83-6.94 (m, 1H), 7.11-7.19 (m, 2H), 7.42 (bd, J=8.7 Hz, 1H), 7.68 (br s, 1H), 7.72 (d, J=8.7 Hz, 2H); MS m/z: 472.1 [M+Na]⁺.

### Example 12

### (3R,5R)-((Z)-4-((1-(nitrooxy)ethoxy)carbonyloxy)but-2-enyl) 7-(2-(4-fluorophenyl)-5-isopropyl-3-phenyl-4-(phenylcarbamoyl)-1H-pyrrol-1-yl)-3,5-dihydroxyheptanoate [NO-Atorvastatin (I-CD8-L2-R1)]

This compound was synthesized in 5 steps as shown in Scheme 22 (via method A) and the experimental procedure is described below:

### Step 1: 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetic acid [CD8(PG^{H})]

To a stirred suspension of atorvastatin calcium salt (10.0 g, 8.7 mmol) and 2,2-dimethoxypropane (5.3 mL, 43.3 mmol) in acetone (500 mL) at 0° C. was added concentrated sulfuric acid (˜0.5 mL added drop wise) and the mixture was stirred at 0° C. for 3 h and at RT for additional 2 h. TLC of the mixture indicated ˜90% conversion to the acetonide. The mixture was concentrated in vacuo and about half of the residue (˜7.0 g) was used as such in the next step. The remaining half of the crude product (˜8.0 g) was purified by column chromatography on silica gel (200-400 mesh) using 5% acetone in DCM to yield the pure title compound as white solid; Purity by HPLC: 99.29% at 210 nm. ¹H NMR (CDCl₃, 300 MHz): δ 1.34 (s, 3H), 1.39 (s, 3H), 1.25-1.42 (m, 2H), 1.54 (d, J=7.2 Hz, 6H), 1.63-1.73 (m, 2H), 2.47 (dq, J=15.9, 10.0, 6.6 Hz, 2H), 3.45-3.65 (m, 1H), 3.67-3.75 (m, 1H), 3.80-3.93 (m, 1H), 4.05-4.25 (m, 2H), 6.89 (br s, 1H), 6.98-7.21 (m, 14H); MS m/z: 599.3 [M+H]⁺, 621.3 [M+Na]⁺.

### Step 2: (Z)-4-hydroxybut-2-enyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate [CD8(PG^{H})-L2-OH]

1,1′-Carbonyldiimidazole (CDI, 3.4 g, 21.0 mmol) was added as solid (in one lot) to a solution of 7.0 g (11.7 mmol) of 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetic acid [CD8(PG^{H})] in DCM (100 mL) at RT and the mixture was stirred at RT for 1.5 h when TLC analysis indicated formation of the corresponding CDI intermediate. This mixture was added to a suspension of cis-2-butene-1,4-diol (HO-L2-OH, 4.2 g, 48.0 mmol) in DCM (150 mL) at 0° C. over a period of 20 minutes and the mixture was stirred at 0° C. for 4 h and at RT for 2 days. TLC analysis of the mixture indicated completion of the reaction. The mixture was washed with water (3×200 mL), brine (2×100 mL), dried over anhydrous sodium sulfate and concentrated in vacuo to afford 6.5 g of a crude semisolid which was purified by column chromatography on silica gel (150-300 mesh) using 2% acetone in DCM as eluent. The pure title compound was obtained as a white solid, Yield: 4.7 g (58.8%); ¹H NMR (CDCl₃, 300 MHz): δ 1.31 (s, 3H), 1.37 (s, 3H), 1.30-1.45 (m, 2H), 1.54 (d, J=6.9 Hz, 6H), 1.62-1.73 (m, 2H), 2.42 (dq, J=15.6, 8.7, 6.0 Hz, 2H), 3.50-3.65 (m, 1H), 3.66-3.77 (m, 1H), 3.78-3.92 (m, 1H), 4.05-4.23 (m, 3H), 4.26 (d, J=6.6 Hz, 2H), 4.70 (d, J=6.9 Hz, 2H), 5.57-5.68 (m, 1H), 5.83-5.93 (m, 1H), 6.88 (br s, 1H), 6.98-7.19 (m, 14H); MS m/z: 669.3 [M+H]⁺, 691.3 [M+Na]⁺.

### Step 3: (3R,5R)-((Z)-4-hydroxybut-2-enyl) 7-(2-(4-fluorophenyl)-5-isopropyl-3-phenyl-4-(phenylcarbamoyl)-1H-pyrrol-1-yl)-3,5-dihydroxyheptanoate (CD8-L2-OH)

Montmorillonite Clay K-10 powder (1.8 g) was added to a solution of (Z)-4-hydroxybut-2-enyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate [CD8(PG^{H})-L2-OH] (4.5 g, 6.7 mmol) in 150 mL of methanol and the mixture was stirred at RT for 10 days when TLC analysis of the mixture indicated ˜90% conversion. The mixture was filtered through celite and the filtrate was concentrated and the crude residue (˜4.0 g) thus obtained was used as such in the next step. For obtaining analytical sample, a small amount (˜100 mg) of this crude product was purified by column chromatography on silica gel (200-400 mesh) using 2% acetone in DCM as eluent. The pure title compound was obtained as a white solid. ¹H NMR (CDCl₃, 300 MHz): δ 1.22-1.32 (m, 2H), 1.55 (d, J=6.9 Hz, 6H), 1.65-1.72 (m, 2H), 2.20 (br s, 2H), 2.43 (d, J=6.0 Hz, 2H), 3.01 (br s, 1H), 3.54-3.65 (m, 1H), 3.70-3.80 (m, 1H), 3.90-4.03 (m, 1H), 4.06-4.22 (m, 2H), 4.27 (d, J=6.6 Hz, 2H), 4.73 (d, J=6.9 Hz, 2H), 5.60-5.70 (m, 1H), 5.82-5.95 (m, 1H), 6.87 (br s, 1H), 6.97-7.26 (m, 14H); MS m/z: 629.3 [M+H]⁺, 651.3 [M+Na]⁺.

### Step 4: (3R,5R)-((Z)-4-((1-chloroethoxy)carbonyloxy)but-2-enyl) 7-(2-(4-fluorophenyl)-5-isopropyl-3-phenyl-4-(phenylcarbamoyl)-1H-pyrrol-1-yl)-3,5-dihydroxyheptanoate (CD8-L2-R1-CI)

α-Chloroethyl chloroformate (CI-R1-CI, 0.8 mL, 7.2 mmol) was added drop-wise to a solution of (3R,5R)-((Z)-4-hydroxybut-2-enyl) 7-(2-(4-fluorophenyl)-5-isopropyl-3-phenyl-4-(phenylcarbamoyl)-1H-pyrrol-1-yl)-3,5-dihydroxyheptanoate (CD8-L2-OH, 3.8 g, 6.0 mmol) in 100 mL of DCM at 0° C. under a nitrogen atmosphere. To this stirred mixture was added pyridine (6.2 mL, 76.4 mmol) and the mixture was stirred at 0° C. under nitrogen for 1 h and at RT for overnight. TLC analysis of the mixture indicated ˜30% completion. Additional amounts of α-Chloroethyl chloroformate (0.8 mL) and pyridine (0.9 mL) were added to the mixture at RT and mixture was stirred at RT for 1 h when TLC analysis of the mixture indicated about 40% conversion. Another 1.6 mL of α-Chloroethyl chloroformate (total added: 3.2 mL) and 1.8 mL of pyridine (total amount of pyridine added: 3.6 mL) were added and the mixture was stirred for additional 3 h when TLC analysis of the mixture indicated ˜70% product formation. The mixture was washed with 1N HCl (3×100 mL), aqueous sodium bicarbonate (3×100 mL) and brine (2×100 mL). The organic layer was dried over Na₂SO₄ and concentrated in vacuo to afford the crude product as a sticky semisolid which was purified by column chromatography on silica gel (200-400 mesh) using 2% acetone in DCM as eluent. The pure title compound was obtained as a light blue colored semisolid. Yield: 2.0 g (45.4%); ¹H NMR (CDCl₃, 300 MHz): δ 1.25-1.60 (m, 2H), 1.56 (d, J=6.9 Hz, 6H), 1.60-1.80 (m, 2H), 1.88 (d, J=8.4 Hz, 3H), 2.20 (s, 1H), 2.44 (d, J=6.0 Hz, 2H), 3.50-3.67 (m, 1H), 3.72-3.82 (m, 1H), 3.90-4.05 (m, 1H), 4.10-4.30 (m, 2H), 4.73 (d, J=4.5 Hz, 2H), 4.82 (d, J=4.8 Hz, 2H), 5.82 (t, J=4.5 Hz, 2H), 6.43 (q, J=5.7 Hz, 1H), 6.88 (s, 1H), 6.97-7.27 (m, 15H); MS m/z: 735.3 [M+H]⁺.

### Step 5: (3R,5R)-((Z)-4-((1-(nitrooxy)ethoxy)carbonyloxy)but-2-enyl) 7-(2-(4-fluorophenyl)-5-isopropyl-3-phenyl-4-(phenylcarbamoyl)-1H-pyrrol-1-yl)-3,5-dihydroxyheptanoate [NO-Atorvastatin (I-CD8-L2-R1)]

Silver nitrate (0.7 g, 3.9 mmol) was added to a solution of (3R,5R)-((Z)-4-((1-chloroethoxy)carbonyloxy)but-2-enyl) 7-(2-(4-fluorophenyl)-5-isopropyl-3-phenyl-4-(phenylcarbamoyl)-1H-pyrrol-1-yl)-3,5-dihydroxyheptanoate (CD8-L2-R1-CI, 1.9 g, 2.6 mmol) in 50 mL of ACN and the mixture was refluxed in dark at 85-90° C. for 3 h when HPLC analysis of the mixture indicated complete conversion (Note: Retention time (T_{R}) of starting material and product were the same and there was no precipitation of silver chloride in the reaction mixture!. It was for that reason that the mixture was refluxed for 3 h long). The mixture was cooled and filtered over celite. The filtrate was concentrated and the residue thus obtained was purified by column chromatography on silica gel (200-400 mesh) by using 4% acetone in DCM to afford the title compound as light yellow semisolid which solidified on standing. Mp: 56-58° C.; Yield: 1.5 g (76.0%); ¹H NMR (CDCl₃, 300 MHz): δ 1.25-1.35 (m, 2H), 1.56 (d, J=7.2 Hz, 6H), 1.61 (d, J=5.4 Hz, 3H), 1.45-1.80 (m, 3H), 2.44 (d, J=6.0 Hz, 2H), 3.50-3.67 (m, 2H), 3.72-3.82 (m, 1H), 3.90-4.20 (m, 1H), 4.08-4.25 (m, 2H), 4.74 (d, J=5.1 Hz, 2H), 4.80 (d, J=5.1 Hz, 2H), 5.80-5.85 (m, 2H), 6.88 (br s, 1H), 6.93 (q, J=5.7 Hz, 1H), 6.98-7.22 (m, 14H); MS m/z: 762.3 [M+H]⁺, 784.3 [M+Na]⁺.

### Example 13

### (2S)-2-(3-((1-(nitrooxy)ethoxy)carbonyloxy)propoxy)-2-oxoethyl 2-(6-methoxynaphthalen-2-yl)propanoate [NO-Naproxen (I-CD1-L6-R1)]

This compound was prepared in four steps as shown in Scheme 3 and the experimental procedure is described below:

### Step 1: Preparation of linker 3-hydroxypropyl 2-chloroacetate (CI-L6-OH)

2-Chloroacetyl chloride (5.0 g, 44.2 mmol) followed by TEA (9.2 mL, 66.4 mmol) were added drop-wise to a stirred solution of propane-1,3-diol (10.0 g, 132.7 mmol) in 150 mL of DCM at 0° C. under nitrogen over 15 min and the mixture was stirred at RT for 4 h when TLC analysis (H₂SO₄ spray) of the mixture indicated formation of a new product CI-L6-OH as the major product. The mixture was concentrated and the crude product thus obtained was used as such in the next step.

### Step 2: Preparation of (S)-2-(3-hydroxypropoxy)-2-oxoethyl 2-(6-methoxynaphthalen-2-yl)propanoate (CD1-L6-OH)

A solution of 3-hydroxypropyl 2-chloroacetate (CI-L6-OH, crude product obtained from the first step, ˜44.0 mmol) in 75 mL of DMF was added to naproxen cesium (10.0 g, 65.7 mmol, freshly prepared by treating naproxen with equimolar amount of cesium carbonate) in 25 mL of DMF and the mixture was stirred at RT for overnight (˜16 h) when TLC analysis of the mixture indicated formation of a new product. The mixture was diluted with DCM (˜200 mL), washed with cold water (4×100 mL), 1N sodium bicarbonate (3×100 mL), and brine (2×100 mL). The organic layer was dried over Na₂SO₄ and concentrated to give the crude product as yellow oil which was purified by column chromatography (silica gel 200-400 mesh, eluted with 20% EtOAc in petroleum ether) to afford the title compound as yellow oil. Yield: 8.0 g (52.5%); ¹H NMR (CDCl₃, 300 MHz): δ1.62 (d, J=7.2 Hz, 3H), 1.73-1.83 (m, 2H), 3.56 (t, J=6.0 Hz, 2H), 3.91 (s, 3H), 3.97 (q, J=7.2 Hz, 1H), 4.25 (t, J=6.0 Hz, 2H), 4.59 (q, J=20.4, 15.9 Hz, 2H), 7.09-7.17 (m, 2H), 7.42 (dd, J=8.4, 1.8 Hz, 1H), 7.69 (s, 1H), 7.72 (s, 1H), 8.00 (s, 1H); MS m/z: 369.1 [M+Na]⁺.

### Step 3: Preparation of (2S)-2-(3-((1-chloroethoxy)carbonyloxy)propoxy)-2-oxoethyl 2-(6-methoxynaphthalen-2-yl)propanoate (CD1-L6-R1-CI)

α-Chloroethyl chloroformate (CI-R1-CI, 2.7 mL, 27.7 mmol) followed by pyridine (2.8 mL, 34.5 mmol) were added drop-wise to a stirred solution of 2-(3-hydroxypropoxy)-2-oxoethyl 2-(6-methoxynaphthalen-2-yl)propanoate (CD1-L6-OH, compound from step b above, 8.0 g, 23.0 mmol) in 50 mL of HPLC grade DCM at 0° C. under nitrogen over 10 minutes and the mixture was stirred for ˜40 minutes when TLC analysis of the mixture indicated completion of the reaction. After the usual aqueous work-up as described in analogues experimental step above, the crude product was used as such in the next reaction. Yield: 10.0 g (96.0%); ¹H NMR (CDCl₃, 300 MHz): δ 1.62 (d, J=6.9 Hz, 3H), 1.82 (d, J=5.7 Hz, 3H), 1.87-1.98 (m, 2H), 3.91 (s, 3H), 3.97 (q, J=7.2 Hz, 1H), 4.13-4.26 (m, 4H), 4.59 (q, J=21.3, 15.9 Hz, 2H), 6.40 (q, J=5.7 Hz, 1H), 7.09-7.19 (m, 2H), 7.42 (dd, J=8.4, 1.8 Hz, 1H), 7.70 (s, 1H), 7.71 (d mixed with singlet, J=8.1 Hz, 2H); MS m/z: 475.1 [M+Na]⁺.

### Step 4: Preparation of (2S)-2-(3-((1-(nitrooxy)ethoxy)carbonyloxy)propoxy)-2-oxoethyl 2-(6-methoxynaphthalen-2-yl)propanoate (I-CD1-L6-R1)

Silver nitrate (5.3 g, 31.5 mmol) was added to a stirred solution of 2-(3-((1-chloroethoxy)carbonyloxy)propoxy)-2-oxoethyl 2-(6-methoxynaphthalen-2-yl)propanoate (CD1-L6-R1-CI, compound from step 3 above, 9.5 g, 20.9 mmol) in 70 mL of ACN and the mixture was refluxed gently (at 85-90° C.) for 40 minutes when HPLC analysis of the mixture indicated completion of the reaction. The mixture was cooled and diluted with DCM (˜200 mL) and filtered over celite. The filtrate was concentrated and the residue was re-dissolved in DCM (˜100 mL) and filtered to remove the precipitated silver salt. This process was repeated twice to remove most of the silver salt from the crude product. The residue thus obtained was purified by column chromatography (300.0 g silica gel, 200-400 mesh, eluted with 15-20% EtOAc in petroleum ether) to afford the title compound as light yellow oil. Yield: 7.3 g (72.5%); ¹H NMR (CDCl₃, 300 MHz): δ 1.60 (d, J=5.4 Hz, 3H), 1.64 (d, J=7.2 Hz, 3H), 1.89-1.98 (m, 2H), 3.94 (s, 3H), 3.99 (q, J=7.2 Hz, 1H), 4.16 (t, J=6.3 Hz, 2H), 4.21 (t, J=6.3 Hz, 2H), 4.63 (dq, J=21.0, 15.9, 1.2 Hz, 2H), 6.93 (q, J=5.7 Hz, 1H), 7.12-7.19 (m, 2H), 7.45 (dd, J=8.4, 1.8 Hz, 1H), 7.72 (s, 1H), 7.73 (d mixed with singlet, J=8.4 Hz, 2H); MS m/z: 502.1 [M+Na]⁺, 518.1 [M+K]⁺; HRMS ESI (m/z): [M+Na]⁺ calculated for C₂₂H₂₅N₁Na₁O₁₁: 502.1320; Found: 502.1330 (Mass Accuracy: −1.99 ppm).

The compounds of examples 14 and 15 were prepared by following the experimental procedure described for preparing the compound of example 13. The characterization data for the compounds of examples 14 and 15 is described below:

### Example 14

### (2S)-2-(4-((1-(nitrooxy)ethoxy)carbonyloxy)butoxy)-2-oxoethyl 2-(6-methoxynaphthalen-2-yl)propanoate [NO-Naproxen (I-CD1-L7-R1)]

The title compound was obtained as colorless viscous oil. Yield (last step): 51.0%; ¹H NMR (CDCl₃, 300 MHz): δ 1.55-1.68 (m, 10H), 3.91 (s, 3H), 3.97 (q, J=7.2 Hz, 1H), 4.07-4.17 (m, 4H), 4.59 (dd, J=21.0, 15.9 Hz, 2H), 6.92 (q, J=5.7 Hz, 1H), 7.10-7.17 (m, 2H), 7.43 (dd, J=8.4, 1.5 Hz, 1H), 7.70 (s, 1H), 7.71 (d mixed with singlet, J=8.1 Hz, 2H); MS m/z: 493.1 [M+H]⁺, 516.1 [M+Na]⁺.

### Example 15

### (2S,3aS,6aS)-2-(nitrooxy)-4,13-dioxo-3,5,12-trioxa-8,9-dithiatetradecan-14-yl 1-((S)-2-((S)-1-ethoxy-1-oxo-4-phenylbutan-2-ylamino)propanoyl)octahydrocyclo-penta[b]pyrrole-2-carboxylate [NO-Ramipril (I-CD9-L8-R1)]

The title compound was obtained as colorless oil. Yield (last step): 36.0%; ¹H NMR (CDCl₃, 500 MHz): δ 1.20-1.32 (m, 6H), 1.60 (d, J=5.5 Hz, 3H), 1.50-2.17 (m, 10H), 2.40-2.52 (m, 1H). 2.60-2.76 (m, 2H), 2.78-2.88 (m, 1H), 2.91-2.98 (m, 4H), 3.18 (t, J=6.5 Hz, 1H), 3.65 (q, J=6.5 Hz, 1H), 4.18 (q, J=7 Hz, 2H), 4.31 (q, J=7.5 Hz, 1H), 4.39-4.47 (m, 4H), 4.56 (d, J=16.0 Hz, 1H), 4.65-4.71 (m, 1H), 4.80 (d, J=16.0 Hz, 1H), 6.93 (q, J=5.5 Hz, 1H), 7.14-7.29 (m, 5H); MS m/z: 744.1 [M+H]⁺.

### Example 16

### 3-(((1-(nitrooxy)ethoxy)carbonyloxy)methyl)phenyl 2-acetoxybenzoate [NO-Aspirin/Salicylic acid (I-CD2-L9-R1)]

The title compound was synthesized in four steps as shown in Scheme 4 and the experimental procedure is described below:

### Step 1: Synthesis of 3-formylphenyl 2-acetoxybenzoate (CD2-L9-CHO)

A solution of 3-hydroxybenzaldehyde (HO-L9-CHO, 5.0 g, 40.9 mmol) and triethylamine (12.4 g/14.4 mL, 122.8 mmol) in 50 mL of DCM was added drop-wise to a stirred solution of aspirin acid chloride (freshly prepared from 14.7 g (81.9 mmol) of aspirin by using oxalyl chloride/DMF method) in 100 mL of DCM at 0° C. and the mixture was stirred at RT for overnight when TLC analysis of the mixture indicated completion of the reaction. The mixture was diluted with 100 mL of DCM and washed with water (100 mL) and brine (100 mL), dried over anhydrous Na₂SO₄ and concentrated in vacuo to give a solid residue which was purified by column chromatography (silica gel 100-200 mesh, eluted with a gradient of EtOAc in petroleum ether and finally with DCM) to afford the title compound as a white solid. Yield: 7.0 g (60.1%); ¹H NMR (DMSO-d₆, 300 MHz): δ 2.26 (s, 3H), 7.35 (dd, J=8.1, 0.9 Hz, 1H), 7.52 (dt, J=7.8, 0.9 Hz, 1H), 7.58-7.63 (two m, 1H), 7.73 (t, J=8.1 Hz, 1H), 7.77-7.84 (m, 2H), 7.89 (distorted d, J=7.5 Hz, 1H), 8.21 (dd, J=7.8, 1.5 Hz, 1H), 10.05 (s, 1H).

### Step 2: Synthesis of 3-(hydroxymethyl)phenyl 2-acetoxybenzoate (CD2-L9-OH)

Sodium borohydride (79 mg, 2.1 mmol) was added to a solution of 3-formylphenyl 2-acetoxybenzoate (CD2-L9-CHO, 2.8 g, 9.9 mmol) in 30 mL of THF/MeOH (9:1) at 0° C., and the mixture was stirred at that temperature for 20 minutes when TLC analysis of the mixture indicated completion of the reaction. The mixture was slowly poured into 10 mL of ice cold 1N HCl and extracted with ethyl acetate (2×100 mL). The organic layer was washed with brine (1×100 mL), dried over anhydrous Na₂SO₄ and concentrated in vacuo to give a solid residue which was purified by column chromatography (silica gel 100-200 mesh, eluted with a gradient of EtOAc in petroleum ether and finally with DCM) to afford the title compound as a white solid. Yield: 2.2 g (78.0%); ¹H NMR (DMSO-d₆, 300 MHz): δ 2.25, (s, 3H), 4.54 (d, J=5.7 Hz, 2H), 5.34 (t, J=5.7 Hz, 1H Exchangeable with D₂O), 7.08 (d, J=7.8 Hz, 1H), 7.16 (s, 1H), 7.25 (d, J=7.5 Hz, 1H), 7.33 (d, J=8.1 Hz, 1H), 7.43 (t, J=7.8 Hz, 1H), 7.51 (t, J=7.8 Hz, 1H), 7.77 (t, J=7.8 Hz, 1H), 8.15 (distorted dd, J=7.2, 1.5 Hz, 1H).

### Step 3: Synthesis of 3-(((1-chloroethoxy)carbonyloxy)methyl)phenyl 2-acetoxybenzoate (CD2-L9-R1-CI)

A solution of α-chloroethyl chloroformate (C-R1-CI, 0.18 mL, 1.3 mmol) in 1 mL of DCM was added drop-wise to a stirred solution of 3-(hydroxymethyl)phenyl 2-acetoxybenzoate (CD2-L9-OH, 0.3 g, 1.1 mmol) and pyridine (0.1 mL, 1.3 mmol) in dichloromethane (3 mL) at 0° C. The mixture was stirred at 0° C. for 30 minutes when TLC analysis of the mixture indicated completion of the reaction. The mixture was diluted with DCM (˜10 mLl), washed with water (1×10 mL) and brine (1×10 mL). The organic layer was dried over Na₂SO₄ and concentrated in vacuo to give an oily crude product which was purified by column chromatography on silica gel by eluting with a gradient of EtOAc in petroleum ether to afford the title compound as colorless viscous oil. Yield: 0.4 g (93.7%); ¹H NMR (CDCl₃, 300 MHz): δ 1.85 (d, J=5.7 Hz, 3H), 2.33 (s, 3H), 5.26 (AB q, J=12.3 Hz, 2H), 6.45 (q, J=5.7 Hz, 1H), 7.16-7.27 (m, 3H), 7.38-7.51 (d, J=7.5 Hz, 1H), 7.38-7.50 (m, 2H), 7.67 (dt, J=7.8, 1.5 Hz, 1H), 8.23 (dd, J=7.8, 1.5 Hz, 1H). MS m/z: 410.1 [M+NH₄]⁺, 415.0 [M+Na]⁺.

### Step 4: Synthesis of 3-(((1-(nitrooxy)ethoxy)carbonyloxy)methyl)phenyl 2-acetoxybenzoate (I-CD2-L9-R1)

Silver nitrate (0.2 g, 0.9 mmol) was added in one lot to a stirred solution of 3-(((1-chloroethoxy)carbonyloxy)methyl)benzyl 2-acetoxybenzoate (CD2-L9-R1-Cl, 1.4 g, 3.5 mmol) in ACN (20 mL) at RT and the mixture was stirred at 60-70° C. for 2 h when TLC analysis of the mixture indicated completion of the reaction The mixture was cooled to RT, diluted with 10 mL of DCM and filtered over celite pad. The filtrate was concentrated and the residue thus obtained was purified by column chromatography on silica gel and eluted with a gradient of EtOAc in petroleum ether to afford the title compound as yellow viscous oil. Yield: 0.3 g (81.2%); ¹H NMR (CDCl₃, 300 MHz): δ 1.62 (d, J=5.7 Hz, 3H), 2.33 (s, 3H), 5.24 (AB q, J=12.3 Hz, 2H), 6.97 (q, J=5.7 Hz, 1H), 7.18-7.27 (m, 3H), 7.31 (d, J=7.8 Hz, 1H), 7.37-7.53 (m, 2H), 7.67 (t, J=7.8, 1 H), 8.24 (dd, J=7.8, 1.5 Hz, 1H); MS m/z: 437.1 [M+NH₄]⁺, 442.1 [M+Na]⁺.

### Example 17

### 3-(((1-(nitrooxy)ethoxy)carbonyloxy)methyl)benzyl 2-acetoxybenzoate [NO-Aspirin/Salicylic acid (I-CD2-L10-R1)]

The title compound was synthesized in three steps as shown in Scheme 1 and the experimental procedure is described below:

### Step 1: Synthesis of 3-(hydroxymethyl)benzyl 2-acetoxybenzoate (CD2-L10-OH)

A solution of aspirin acid chloride (3.0 g, 16.7 mmol, freshly prepared from aspirin using oxalyl chloride/DMF method) in dichloromethane (15 mL) was added to a stirred solution of 1,3-benzenedimethanol (HO-L10-OH, 2.3 g, 16.6 mmol) and triethylamine (6.96 mL, 49.9 mmol) in dichloromethane (12 mL) at 0° C. The mixture was stirred at RT for 8 h when TLC analysis of the mixture indicated completion of the reaction. The mixture was concentrated and the residue was partitioned between ethyl acetate (100 mL) and water (50 mL). The organic layer was separated, washed with brine (1×50 mL), dried over anhydrous Na₂SO₄ and concentrated in vacuo to give the crude oily residue which was purified by column chromatography (silica gel, 150.0 g, 200-400 mesh, 30% EtOAc in hexane) to afford the title compound as colorless oil. Yield: 1.9 g (38.2%); ¹H NMR (CDCl₃, 300 MHz): δ 1.99 (t, J=5.7 Hz, 1H), 2.14 (s, 3H), 4.73 (d, J=5.4 Hz, 2H), 5.33 (s, 2H), 7.10 (d, J=8.1 Hz, 1H), 7.30-7.43 (m, 5H), 7.58 (dt, J=7.8, 1.5 Hz, 1H), 8.08 (dd, J=7.8, 1.5 Hz, 1H); MS m/z: 301.1 [M+H]⁺, 323.1 [M+Na]⁺.

### Step 2: Synthesis of 3-(((1-chloroethoxy)carbonyloxy)methyl)benzyl 2-acetoxybenzoate (CD2-L10-R1-CI)

α-Chloroethyl chloroformate (CI-R1-CI, 0.5 mL, 4.6 mmol) was added drop-wise to a stirred solution of 3-(hydroxymethyl)benzyl 2-acetoxybenzoate (CD2-L10-OH, 1.1 g, 3.8 mmol) and pyridine (0.6 mL, 7.6 mmol) in dichloromethane (12 mL) at 0° C. The mixture was stirred at 0° C. for 30 minutes when TLC analysis of the mixture indicated completion of the reaction. The mixture was concentrated in vacuo and the residue was partitioned between EtOAc (50 mL) and water (50 mL). The organic layer was separated and washed with brine (1×50 mL), dried over anhydrous Na₂SO₄ and concentrated in vacuo to give an oily crude product which was purified by column chromatography (silica gel, 60.0 g, 200-400 mesh, 30% EtOAc in hexane) to afford the title compound as colorless oil. Yield: 1.4 g (91.0%); ¹H NMR (CDCl₃, 300 MHz): δ 1.84 (d, J=5.7 Hz, 3H), 2.18 (s, 3H), 5.26 (d, J=2.1 Hz, 2H), 5.33 (s, 2H), 6.45 (q, J=5.7 Hz, 1H), 7.11 (dd, J=8.1, 0.6 Hz, 1H), 7.34 (dt, J=7.8, 0.9 Hz, 1H), 7.37-7.48 (m, 4H), 7.58 (dt, J=7.8, 1.5 Hz, 1H), 8.09 (d, J=1.5 Hz, 1H); MS m/z: 407.1 [M+H]⁺, 429.1 [M+Na]⁺.

### Step 3: Synthesis of 3-(((1-(nitrooxy)ethoxy)carbonyloxy)methyl)benzyl 2-acetoxybenzoate (I-CD2-L10-R1)

Silver nitrate (0.9 g, 5.2 mmol) was added in one lot to a stirred solution of 3-(((1-chloroethoxy)carbonyloxy)methyl)benzyl 2-acetoxybenzoate (CD2-L10-R1-CI, 1.4 g, 3.5 mmol) in ACN (20 mL) at RT and the mixture was stirred at 80° C. for 1.5 h. The mixture was cooled to RT and filtered over celite pad. The filtrate was concentrated and the residue thus obtained was partitioned between EtOAc (75 mL) and water (75 mL). The organic layer was washed with brine (1×75 mL), dried over anhydrous Na₂SO₄ and concentrated to give an oily crude residue which was purified by column chromatography (silica gel, 40.0 g, 200-400 mesh 30% EtOAc in hexane) to afford the title compound as yellow oil. Yield: 1.1 g (74.0%); ¹H NMR (CDCl₃, 300 MHz): δ 1.63 (d, J=6.0 Hz, 3H), 2.17 (s, 3H), 5.24 (s, 2H), 5.32 (s, 2H), 6.96 (q, J=6.0 Hz, 1H), 7.11 (d, J=9.0 Hz, 1H), 7.31-7.44 (m, 5H), 7.58 (dt, J=6.0 Hz, 1H), 8.07 (dd, J=1.8 Hz, 1H); MS m/z: 434.2 [M+H]⁺, 456.1 [M+Na]⁺.

The compounds of examples 18-20 were prepared by following the experimental procedure described for preparing the compound of example 17. The characterization data for the compounds of examples 18-20 is described below:

### Example 18

### (6-(((1-(nitrooxy)ethoxy)carbonyloxy)methyl)pyridin-2-yl)methyl 2-acetoxybenzoate [NO-Aspirin/Salicylic acid (I-CD2-L11-R1)]

The title compound was also obtained as yellow oil. Yield (last step): 68.0%; ¹H NMR (CDCl₃, 300 MHz): δ 1.64 (d, J=5.7 Hz, 3H), 2.27 (s, 3H), 5.29-5.34 (distorted AB quartet or m, 2H), 5.44 (s, 2H), 6.98 (q, J=5.7 Hz, 1H), 7.14 (dd, J=8.1, 0.9 Hz, 1H), 7.31-7.42 (m, 3H), 7.61 (dt, J=7.8, 1.8 Hz, 1H), 7.78 (t, J=7.8 Hz, 1H), 8.12 (dd, J=7.8, 1.8 Hz, 1H); MS m/z: 435.1 [M+H]⁺, 457.1 [M+Na]⁺.

### Example 19

### (4-(((1-(nitrooxy)ethoxy)carbonyloxy)methyl)cyclohexyl)methyl 2-(2-(2,6-dichlorophenylamino)phenyl)acetate [NO-Diclofenac (I-CD6-L12-R1)]

The title compound was obtained as pale yellow gum. Yield (last step): 54.0%; ¹H NMR (CDCl₃, 300 MHz): δ 0.85-1.98 (m, 10H), 1.61 (d, J=5.7 Hz, 3H), 3.83 (s, 2H), 3.97-4.16 (m, 4H), 6.57 (d, J=7.8 Hz, 1H), 6.91-7.04 (m, 4H), 7.14 (dt, J=7.8, 1.2 Hz, 1H), 7.25 (dd, J=7.5, 1.2 Hz, 1H), 7.36 (d, J=7.8 Hz, 2H); MS m/z: 555.1 [M+H]⁺, 577.1 [M+Na]⁺.

### Example 20

### 4-((1-(nitrooxy)ethoxy)carbonyloxy)cyclohexyl 2-(2-(2,6-dichlorophenylamino)phenyl)acetate [NO-Diclofenac (I-CD6-L13-R1)]

The title compound was obtained as pale yellow gum. Yield (last step): 66.0%; ¹H NMR (CDCl₃, 300 MHz): δ 1.63 (d, J=5.7 Hz, 3H), 1.67-2.00 (m, 8H), 3.82 (s, 2H), 4.73-4.83 (m, 1H), 4.89-4.98 (m, 1H), 6.57 (d, J=7.8 Hz, 1H), 6.89-7.04 (m, 4H), 7.14 (dt, J=7.8, 1.5 Hz, 1H), 7.25 (dd, J=7.5, 1.2 Hz, 1H), 7.36 (d, J=8.1 Hz, 2H); MS m/z: 527.6 [M+H]⁺.

### Example 21

### (2S)-4-((1-(nitrooxy)ethoxy)carbonyloxy)tetrahydrofuran-3-yl 2-(6-methoxynaphthalen-2-yl)propanoate [NO-Naproxen (I-CD1-L14-R1-A & I-CD1-L14-R1-B)] (Mixture of diastereomers)

The title compound was synthesized in 3 steps as shown in Scheme 1 and the experimental procedure is described below:

### Step 1: Synthesis of (2S)-4-hydroxytetrahydrofuran-3-yl 2-(6-methoxynaphthalen-2-yl)propanoate (CD1-L14-OH)

A solution of naproxen acid chloride (CD1-1, freshly prepared from 10.0 g (43.4 mmol) of naproxen using oxalyl chloride/DMF method) in 20 mL of DCM was added to a stirred solution of 1,4-anhydroerythritol (HO-L14-OH, 9.1 g (˜7.1 mL), 86.9 mmol) and TEA (18.0 mL, 130.0 mmol) in 20 mL of DCM at 0° C. over a period of 30 minutes and the mixture was stirred at 0° C. for 1.5 h when TLC analysis of the mixture indicated formation of a major mono adduct along with the expected minor bis-adduct. The mixture was diluted with saturated sodium bicarbonate (˜100 mL) and the organic layer was separated. The aqueous layer was extracted with DCM (2×100 mL). The organic extracts were combined, dried over anhydrous Na₂SO₄, filtered and concentrated to give 12.0 g of crude residue which was purified by column chromatography (silica gel 150-300 mesh, the bis-adduct and other non-polar impurities were eluted with 5-10% EtOAc in petroleum ether and the desired mono-adduct was eluted with 13-15% EtOAc in petroleum ether) to afford the title compound as a white solid. Yield: 8.0 g (58.2%); ¹H NMR (CDCl₃, 300 MHz) (Mixture of diastereomers): δ 1.609, 1.614 (two overlapping doublets, J=6.9, 7.2 Hz, 3H), 3.52-3.72 (m, 2H), 3.77-4.10 (m, 5H), 3.91 (s, 3H), 4.30 (q, J=5.7, 5.4 Hz, 0.5H), 4.40 (q, J=5.7, 5.4 Hz, 0.5H), 5.07-5.19 (m, 1H), 7.09-7.20 (m, 2H), 7.37-7.44 (m or distorted doublet, 1H), 7.66-7.76 (m, 3H); MS m/z: 317.1 [M+H]⁺, 339.1 [M+Na]⁺, 355.1 [M+K]⁺.

### Step 2: Synthesis of (2S)-4-((1-chloroethoxy)carbonyloxy)tetrahydrofuran-3-yl 2-(6-methoxynaphthalen-2-yl)propanoate (CD1-L14-R1-CI-A & CD1-L14-R1-CI-B) (Mixture of diastereomers A & B)

α-Chloroethyl chloroformate (CI-R1-CI, 1.0 g (0.8 mL), 7.6 mmol, 1.2 eqs.) followed by pyridine (0.8 mL, 9.5 mmol, 1.5 eqs.) were added drop-wise to a stirred solution of (2S)-4-hydroxytetrahydrofuran-3-yl 2-(6-methoxynaphthalen-2-yl)propanoate (CD1-L14-OH, 2.0 g, 6.3 mmol, 1.0 eq.) in 20 mL of DCM at 0° C. under nitrogen and the mixture was stirred at 0° C. for 2 h and at RT for 1 h when TLC analysis of the mixture indicated completion of the reaction. The mixture was diluted with 20 mL of DCM and washed with 1N HCl (3×40 mL), aqueous sodium bicarbonate (3×40 mL), dried over anhydrous Na₂SO₄ and concentrated to give 2.0 g of crude product as slightly yellow colored oil. Although TLC analysis of the crude product indicated two major new spots or products (CD1-L14-R1-CI-A and CD1-L14-R1-CI-B), HPLC analysis of the same crude product revealed 4 peaks. The crude product was purified by column chromatography (80.0 g of silica gel, 200-400 mesh, eluted with 10% EtOAc in petroleum ether) and the following two products were separated:

Less polar CD1-L14-R1-CI-A: HPLC analysis of this isolated product (single spot on TLC) was shown to contain two diastereomers with retention times (T_{R}) of 9.414 & 9.508 min (peak ratio: 42:54); Obtained as an oil. Yield: 1.1 g (43.0%). ¹HNMR (CDCl₃, 300 MHz, (mixture of diastereomers)): δ 1.42 (d, J=6.0 Hz, 1.5H), 1.57 (t, J=7.2 Hz, 3H), 1.74 (d, J=6.0 Hz, 1.5 Hz), 3.71-3.87 (m, 2H), 3.87-3.94 (m or q buried under methoxy singlet, 1H), 3.91 (s, 3H), 4.01-4.15 (m, 2H), 5.15-5.40 (m, 2H), 5.83, 6.35 (two q in ˜1:1, J=5.7 Hz, 1H (i.e., 0.5H each)), 7.15-7.19 (m, 2H), 7.38 (d, J=8.7 Hz, 1H), 7.64 (d, J=6.9 Hz, 1H), 7.69 (d, J=8.7 Hz, 2H); MS m/z: 445.1 [M+Na]⁺.

More polar CD1-L14-R1-CI-B: HPLC analysis of this isolated product (single spot on TLC) was shown to contain two diastereomers with retention times (T_{R}) of 9.386 and 9.476 min (43:56); Obtained as an oil. Yield: 1.0 g (38.5%). ¹HNMR (CDCl₃, 300 MHz, (mixture of diastereomers)): δ 1.59 (d, overlapping with the doublet at 1.61 ppm, J=7.2 Hz, 1.5H) 1.61 (d, overlapping with the doublet at 1.59 ppm, J=7.2 Hz, 1.5H), 1.73 (d, J=5.7 Hz, 3H), 1.85 (d, J=6.0 Hz, 3H), 3.61-4.17 (m, 5H), 3.93 (s, 3H), 5.24-5.34 (m, 2H), 6.30-6.45 (m, 1H), 7.10-7.18 (m, 2H), 7.42 (dt, J=1.5, 8.4 Hz, 1H), 7.66-7.77 (m, 3H); MS m/z: 445.1 [M+Na]⁺.

### Step 3: Synthesis of (2S)-4-((1-(nitrooxy)ethoxy)carbonyloxy)tetrahydrofuran-3-yl 2-(6-methoxynaphthalen-2-yl)propanoate (I-CD1-L14-R1-A or B) (Mixture of diastereomers A & B)

Silver nitrate (0.5 g, 3.2 mmol, 1.2 eqs.) was added to a solution of (2S)-4-((1-chloroethoxy)carbonyloxy)tetrahydrofuran-3-yl 2-(6-methoxynaphthalen-2-yl)propanoate (CD1-L14-R1-CI-A, Less polar product A, 1.1 g, 2.6 mmol, 1.0 eq.) in 10 mL of ACN and the mixture was refluxed at 85-90° C. for 2 h when TLC analysis of the mixture indicated completion of the reaction with the formation of two product spots (i.e., less polar (spot) product I-CD1-L14-R1-Aa and more polar (spot) product I-CD1-L14-R1-Ab). The reaction mixture was diluted with 10 mL of DCM, filtered over celite and the filtrate was concentrated and the residue was dissolved again in 20 mL of DCM and washed with water (3×20 mL), brine (1×20 mL), dried over anhydrous Na₂SO₄ and concentrated to give an oily residue which was purified by column chromatography (40.0 g of silica gel, 200-400 mesh, eluted with 5-8% EtOAc in petroleum ether) to afford the title compound as the following diastereomeric mixtures:

Less polar (spot) product I-CD1-L14-R1-Aa: HPLC analysis of this product was shown to contain two diastereomers with retention times (T_{R}) of 9.44 & 9.53 min (peak ratio: 43:56); obtained as a viscous oil. Yield: 0.2 g (14.4%); ¹HNMR (CDCl₃, 300 MHz, (mixture of diastereomers in ˜43:56)): δ 1.21-1.28 (m, 3H), 1.51-1.62 (m, 3H), 3.91 (s, 3H), 3.83-3.89 (m, 3H), 4.00-4.13 (m, 2H), 5.18-5.34 (m, 2H), 6.65 (q, J=5.7 Hz, 0.5H), 6.86 (q, J=5.7 Hz, 0.5H), 7.11-7.16 (m, 2H), 7.38 (d, J=8.4 Hz, 1H), 7.65-7.71 (m, 3H); MS m/z: 472.1 [M+Na]⁺.

More polar (spot) product I-CD1-L14-R1-Ab: HPLC analysis of this product was shown to contain two diastereomers with retention times (T_{R}) of 9.39 & 9.48 min (peak ratio: 43:56); Obtained as a green viscous oil. Yield: 0.7 g (55.9%); ¹HNMR (CDCl₃, 300 MHz, (mixture of diastereomers in ˜45:55)): δ 1.47-1.62 (m, 6H), 3.61-3.82 (m, 3H), 3.91 (s, 3H), 3.85-3.91 (m, buried under OCH₃ signal, 1H), 4.15-3.95 (m, 2H), 5.19-5.33 (m, 2H), 6.80 (q, J=5.7 Hz, 0.5H), 6.95 (q, J=5.7 Hz, 0.5H), 7.12-7.16 (m, 2H), 7.31-7.42 (m, 1H), 7.67-7.72 (m, 3H); MS m/z: 472.1 [M+Na]⁺.

The following isomers were obtained by following the same procedure involving the treatment of (2S)-4-((1-chloroethoxy)carbonyloxy)tetrahydrofuran-3-yl 2-(6-methoxynaphthalen-2-yl)propanoate (CD1-L14-R1-CI-B, the more polar product B, 1.0 g, 2.4 mmol, 1.0 eq.) with 0.5 g (2.8 mmol, 1.2 eqs.) of silver nitrate:

Less polar (spot) product I-CD1-L14-R1-Ba: HPLC analysis of this product was shown to contain two diastereomers with retention times (T_{R}) of 9.44 & 9.53 min (peak ratio: 43:56); Obtained as a viscous oil. Yield: 0.6 g (64.0%); ¹HNMR (CDCl₃, 300 MHz, (mixture of two diastereomers in ˜47:53)): δ 1.21-1.28 (m, 3H), 1.51-1.62 (m, 3H), 3.76-3.89 (m, 3H), 3.91 (s, 3H), 4.00-4.13 (m, 2H), 5.18-5.36 (m, 2H), 6.56 (q, J=5.7 Hz, 0.5H), 6.86, (q, J=5.7 Hz, 0.5H), 7.11-7.16 (m, 2H), 7.38 (d, J=8.4 Hz, 1H), 7.65 (s, 1H), 7.70 (d, J=8.4 Hz, 2H); MS m/z: 472.1 [M+Na]⁺.

More polar (spot) product I-CD1-L14-R1-Bb: HPLC analysis of this product was shown to contain two diastereomers with retention times (T_{R}) of 9.39 & 9.48 min (peak ratio: 43:56); Obtained as a green viscous oil. Yield: 0.3 g (25.0%); ¹HNMR (CDCl₃, 300 MHz, (mixture of two diastereomers in ˜39:45)): δ 1.42-1.61 (m, 6H), 3.61-3.89 (m, 3H), 3.91 (s, 3H), 3.86-4.15 (m, 4H), 5.21-5.35 (m, 2H), 6.80 (q, J=5.7 Hz, 0.5H), 6.95 (q, J=5.7 Hz, 0.5H), 7.12-7.15 (m, 2H), 7.37-7.42 (m, 1H), 7.66-7.72 (m, 3H); MS m/z: 472.1 [M+Na]⁺.

The compound of example 22 was prepared by following the experimental procedure described for preparing the compound of example 21. The characterization data of the compound is described below:

### Example 22

### 4-((1-(nitrooxy)ethoxy)carbonyloxy)tetrahydrofuran-3-yl 2-acetoxybenzoate [NO-Aspirin/Salicylic acid (I-CD2-L14-R1-A & I-CD2-L14-R1-B)] (Mixture of diastereomers)

As expected, the title compound was obtained as mixture of diastereomers, I-CD2-L14-R1-A or I-CD2-L14-R1-B and they were isolated and characterized as described below:

Less polar diastereomer I-CD2-L14-R1-A: Obtained as oil. Yield: 0.3 g (24.4%), T_{R}=3.95 min (HPLC Method: Isocratic at 1:1 ACN/water); ¹HNMR (CDCl₃, 300 MHz): δ 1.43 (d, J=5.7 Hz, 3H), 2.35 (s, 3H), 3.91-4.02 (m, 2H), 4.07-4.23 (m, 2H), 5.38 (q, J=5.4 Hz, 1H), 5.56 (q, J=5.4 Hz, 1H), 6.84 (q, J=5.7 Hz, 1H), 7.12 (d, J=8.1 Hz, 1H), 7.33 (distorted dt, J=7.8, 0.9 Hz, 1H), 7.59 (dt, J=7.8, 1.5 Hz, 1H), 8.02 (dd, J=7.8, 1.5 Hz, 1H), MS m/z: 422.1 [M+Na]⁺.

More polar diastereomer I-C2-L14-R1-B: Obtained as oil. Yield: 0.2 g (20.3%); T_{R}=3.56 min (HPLC Method: Isocratic at 1:1 ACN/water); ¹HNMR (CDCl₃, 300 MHz): δ 1.57 (d, J=5.7 Hz, 3H), 2.35 (s, 3H), 3.88, 3.92 (two doublets, ˜4:5, J=5.7, 5.4 Hz, respectively, 1H), 3.97, 4.00 (two doublets, ˜2:3, J=3.9 Hz each, 1H), 4.12, 4.16 (two doublets, ˜3:2, J=5.4 Hz each, 1H), 4.18, 4.22 (two doublets, ˜5:4, J=6.3 Hz each, 1H), 5.38 (q, J=5.4 Hz, 1H), 5.53 (q, J=5.7 Hz, 1H), 6.87 (q, J=5.7 Hz, 1H), 7.11 (dd, J=8.1, 0.6 Hz, 1H), 7.33 (dt, J=7.8, 1.2 Hz, 1H), 7.59 (dt, J=7.8, 1.5 Hz, 1H), 8.02 (dd, J=7.8, 1.5 Hz, 1H), MS m/z: 422.1 [M+Na]⁺.

### Example 23

### (3S,6R)-6-((1-(nitrooxy)ethoxy)carbonyloxy)hexahydrofuro[3,2-b]furan-3-yl2-acetoxybenzoate [NO-Aspirin/Salicylic acid (I-CD2-L15-R1) (Mixture of diastereomers)

The title compound was synthesized in 4 steps as shown in Scheme 5 and the experimental procedure is described below:

Steps 1 and 2: Synthesis of (3S,6R)-6-hydroxyhexahydrofuro[3,2-b]furan-3-yl 2-acetoxybenzoate (CD2-L15-OH)

This known compound (CD2-L15-OH) was synthesized according to the method described by Moriarty et al., J. Med. Chem. 51, 7991-7999, 2008. Thus, 6.0 g of 10% Pd/C was added to a solution of (3S,6R)-6-(nitrooxy)hexahydrofuro[3,2-b]furan-3-yl 2-acetoxybenzoate (CD2-L15-ONO₂, 6.3 g, 17.8 mmol; This known compound was prepared according to the method described by Gilmer et al., Eur. J. Pharm. Sci. 14, 221-227, 2001) in 100 mL of 1:1 MeOH and EtOAc and the mixture was stirred under one atmosphere of hydrogen for 16 h when TLC analysis of the mixture indicated completion of the reaction. The mixture was passed through a small pad of celite and solids were washed with 100 mL of fresh 1:1 mixture of MeOH and EtOAc. The used catalyst was disposed off carefully. The filtrate was concentrated to give 6.0 g of oily residue which was purified by column chromatography (60.0 g of silica gel, 200-400 mesh, eluted with DCM followed by 5% MeOH in DCM). The title compound (CD2-L15-OH) was obtained as colorless viscous oil. Yield: 5.4 g (98.0%); ¹HNMR (CDCl₃, 300 MHz) (Mixture of diastereomers): δ 2.36 (s, 3H), 3.59, 3.62 (two doublets in ratio of ˜4:5, J=6.0, 5.7 Hz, respectively, 1H), 3.90, 3.93 (two doublets in ratio of ˜5:4, J=6.0, 5.7 Hz, respectively, 1H), 4.06, 4.09 (two doublets in ratio of ˜3:7, J=3.3 Hz, 3.6 Hz, respectively, 1H), 4.14, 4.18 (two singlets in ratio of ˜7:3, 1H), 4.33 (q, J=11.7, 5.7 Hz, 1H), 4.57 (unsymmetrical d, J=4.2 Hz, 1H), 4.68 (t, J=4.8 Hz, 1H), 5.44 (d, J=3.3 Hz, 1H), 7.11 (dd, J=8.1, 0.6 Hz, 1H), 7.32 (dt, J=7.8, 0.9 Hz, 1H), 7.55-7.63 (m, 1H), 7.99 (dd, J=7.8, 1.8 Hz, 1H); MS m/z: 331.1 [M+Na]⁺.

### Step 3: Synthesis of (3S,6R)-6-((1-chloroethoxy)carbonyloxy)hexahydrofuro[3,2-b]furan-3-yl 2-acetoxybenzoate (CD2-L15-R1-CI-A or CD2-L15-R1-CI-B) (Mixture of diastereomers)

α-Chloroethyl chloroformate (CI-R1-CI, 0.4 mL, 3.9 mmol, 1.2 eqs.) followed by pyridine (0.4 mL, 4.9 mmol, 1.5 eqs.) were added drop-wise to a stirred solution of (3S,6R)-6-hydroxyhexahydrofuro[3,2-b]furan-3-yl 2-acetoxybenzoate (CD2-L15-OH, 1.0 g, 3.2 mmol, 1.0 eq.) in 5 mL of DCM at 0° C. under nitrogen (over ˜10 minutes) and the mixture was stirred at 0° C. for 30 minutes and at RT for 1 h when TLC analysis of the mixture indicated completion of the reaction. The mixture was diluted with 20 mL of DCM and 30 mL of 1N HCl. The layers were separated. The organic layer was washed with 1N HCl (1×20 mL), aqueous sodium bicarbonate (3×25 mL), brine (2×20 mL), dried over anhydrous Na₂SO₄ and concentrated to give 1.2 g of crude product as a gum. TLC analysis of the crude product indicated two major new spots or products (CD2-L15-R1-CI-A and CD2-L15-R1-CI-B). The crude product was purified by column chromatography (30.0 g of silica gel, 200-400 mesh, eluted with 15-20% EtOAc in petroleum ether) and the following two products were separated:

Less polar CD2-L15-R1-CI-A: HPLC analysis of this isolated less polar product showed single peak with retention time (T_{R}) of 4.546 min (HPLC Method: isocratic at 1:1 ACN/water); Obtained as a sticky solid. Yield: 0.6 g (42.4%); ¹HNMR (CDCl₃, 300 MHz): δ 1.85 (d, J=2.4 Hz, 3H), 2.35 (s, 3H), 3.85-4.18 (m, 4H), 4.57 (d, J=4.8 Hz, 1H), 4.95 (t, J=5.1 Hz, 1H), 5.14 (q, J=9.0, 4.5 Hz, 1H), 5.42 (d, J=3.0 Hz, 1H), 6.45 (q, J=11.4, 5.7 Hz, 1H), 7.10 (dd, J=8.1, 0.9 Hz, 1H), 7.31 (dt, J=7.5, 0.9 Hz, 1H), 7.55 (dt, J=7.8, 1.5 Hz, 1H), 7.98 (dd, J=8.1, 1.8 Hz, 1H); MS m/z: 437.0 [M+Na]⁺.

More polar CD2-L15-R1-CI-B: HPLC analysis of this isolated more polar product showed single peak with retention time (T_{R}) of 4.317 min (HPLC Method: isocratic at 1:1 ACN/water); Obtained as a sticky solid. Yield: 0.4 g (32.7%); ¹HNMR (CDCl₃, 300 MHz): δ 1.84 (d, J=6.0 Hz, 3H), 2.35 (s, 3H), 3.88-4.16 (m, 4H), 4.56 (d, J=4.8 Hz, 1H), 4.95 (t, J=5.1 Hz, 1H), 5.14 (q, J=9.0, 4.5 Hz, 1H), 5.42 (d, J=3.0 Hz, 1H), 6.43 (q, J=12.0, 6.0 Hz, 1H), 7.10 (d, J=7.8 Hz, 1H), 7.31 (t, J=7.5 Hz, 1H), 7.58 (dt, J=7.8, 1.5 Hz, 1H), 7.99 (dd, J=7.8, 1.5 Hz, 1H); MS m/z: 437.0 [M+Na]⁺.

### Step 4: Synthesis of (3S,6R)-6-((1-(nitrooxy)ethoxy)carbonyloxy)hexahydrofuro[3,2-b]furan-3-yl 2-acetoxybenzoate (I-CD2-L15-R1-A or I-CD2-L15-R1-B) (Mixture of diastereomers)

Silver nitrate (0.3 g, 1.7 mmol, 1.2 eqs.) was added to a solution of (3S,6R)-6-((1-chloroethoxy)carbonyloxy)hexahydrofuro[3,2-b]furan-3-yl 2-acetoxybenzoate (CD2-L15-R1-CI-A, Less polar product A, 0.6 g, 1.4 mmol, 1.0 eq.) in 15 mL of ACN and the mixture was refluxed at 85-90° C. for 3 h when TLC analysis of the mixture indicated completion of the reaction with the formation of the desired compound I-CD2-L15-R1-A as the major product. The reaction mixture was filtered and the filtrate was concentrated. The residue was diluted with 40 mL of DCM and washed with water (3×40 mL), brine (2×40 mL), dried over anhydrous Na₂SO₄ and concentrated to give a sticky solid residue which was purified by column chromatography (25.0 g of silica gel, 200-400 mesh, eluted with 20-25% EtOAc in petroleum ether) to afford the title compound as a sticky solid. HPLC analysis of this product has shown single peak with retention time (T_{R}) of 4.538 min (HPLC method: isocratic at 1:1 ACN/water); Yield: 0.3 g (43.3%); ¹HNMR (CDCl₃, 300 MHz): δ 1.61 (d, J=5.7 Hz, 3H), 2.36 (s, 3H), 3.87, 3.91 (two doublets in ratio of ˜1:2, J=5.4, 5.1 Hz, respectively, 1H), 3.95-4.17 (m, 3H), 4.54 (d, J=4.8 Hz, 1H), 4.94 (t, J=5.1 Hz, 1H), 5.00 (q, J=9.3, 1.2 Hz, 1H), 5.42 (d, J=3.0 Hz, 1H), 6.94 (q, J=5.7 Hz, 1H), 7.10 (d, J=8.1 Hz, 1H), 7.31 (t, J=7.8 Hz, 1H), 7.57 (dt, J=7.8, 1.5 Hz, 1H), 8.00 (dd, J=7.8, 1.5 Hz, 1H); MS m/z: 464.0 [M+Na]⁺.

The other diastereomer isomer I-CD2-L15-R1-B was also obtained by following the same experimental procedure involving treatment of (3S,6R)-6-((1-chloroethoxy)carbonyloxy)hexahydrofuro[3,2-b]furan-3-yl 2-acetoxybenzoate (CD2-L15-R1-CI-B, the more polar product B, 0.4 g, 1.1 mmol, 1.0 eq.) with 0.2 g (1.2 mmol, 1.2 eqs.) of silver nitrate. HPLC analysis of this product has shown single peak with retention time (T_{R}) of 4.792 min (HPLC method: isocratic at 1:1 ACN/water); this product was obtained as a sticky solid. Yield: 0.3 g (59.8%); ¹HNMR (CDCl₃, 300 MHz): δ 1.61 (d, J=5.7 Hz, 3H), 2.35 (s, 3H), 3.89, 3.92 (two doublets in ratio of ˜1:3, J=5.4 Hz each, 1H), 3.96, 4.00 (two doublets in ratio of −3:1, J=3.9 Hz each, 1H), 4.03, 4.06 (two doublets in ratio of ˜1:3, J=3.3 Hz each, 1H), 4.10, 4.13 (two singlets in ratio of ˜3:1, 1 H), 4.55 (d, J=4.8 Hz, 1H), 4.94 (t, J=5.1 Hz, 1H), 5.11 (distorted q, J=9.3, 3.9 Hz, 1H), 5.42 (d, J=3.0 Hz, 1H), 6.94 (q, J=5.7 Hz, 1H), 7.10 (dd, J=8.1, 0.6 Hz, 1H), 7.31 (dt, J=7.8, 0.9 Hz, 1H), 7.58 (dt, J=7.8, 1.5 Hz, 1H), 7.98 (dd, J=7.8, 1.5 Hz, 1H); MS m/z: 464.1 [M+Na]⁺.

### Example 24

### (2S)-2-(2-((1-(nitrooxy)ethoxy)carbonyloxy)ethylsulfinyl)ethyl 2-(6-methoxy naphthalen-2-yl)propanoate [NO-Naproxen (I-CD1-L16-R1)]

The above compound was synthesized in 4 steps as shown in Scheme 6 and the experimental procedure is described below:

### Step 1: Preparation of (S)-2-(2-hydroxyethylthio)ethyl 2-(6-methoxynaphthalen-2-yl)propanoate (CD1-L16S-OH)

A solution of freshly prepared naproxen acid chloride (CD1-CI, 16.0 g, 64.0 mmol) in DCM (˜50 mL) was added to a stirred solution of 2,2′-thiodiethanol (HO-L16S-OH, 26.0 g, 256.0 mmol, 3.3 eqs.) in 100 mL of DCM at 0° C. under nitrogen. To this stirred mixture was added triethylamine (TEA, 13.0 mL, 92.9 mmol, 1.5 eqs.) drop-wise over 30 minutes and the mixture was stirred at RT under nitrogen for overnight. TLC analysis of the mixture indicated completion of the reaction. The mixture was washed with saturated sodium bicarbonate (3×100 mL) and brine (2×100 mL) to remove the remaining un-reacted water-soluble linker. The organic layer was dried over anhydrous Na₂SO₄ and concentrated in vacuo to give 22.0 g of crude product which was purified by column chromatography (300.0 g of silica gel, 200-400 mesh). The expected bis-derivative was eluted with 10% EtOAc in petroleum ether. The desired compound was eluted with 15-25% EtOAc in petroleum ether. The pure title compound (CD1-L16S-OH) was obtained as light yellow oil which solidified at low temperature (<0° C.). Yield: 17.4 g (81.3%); ¹H NMR (CDCl₃, 300 MHz): δ 1.60 (d, J=6.9 Hz, 3H), 2.63 (t, J=6.0 Hz, 2H), 2.70 (t, J=6.9 Hz, 2H), 3.62 (t, J=5.7 Hz, 2H), 3.88 (q, J=7.2 Hz, 1H), 3.93 (s, 3H), 4.26 (t, J=6.9 Hz, 2H), 7.10-7.20 (m, 2H), 7.41 (dd, J=8.4, 1.5 Hz, 1H), 7.67-7.77 (m, 3H); MS m/z: 357.1 [M+Na]⁺. This intermediate was also synthesized in good yields by the reaction of naproxen with the corresponding diol in the presence of coupling agents such as DCC, DMAP in a suitable solvent such as DCM or DMF.

### Step 2: Preparation of (2S)-2-(2-((1-chloroethoxy)carbonyloxy)ethylthio)ethyl 2-(6-methoxynaphthalen-2-yl)propanoate (CD1-L16S-R1-CI)

α-Chloroethyl chloroformate (CI-R1-CI, 6.0 mL, 61.0 mmol) was added drop-wise to a solution of 2-(2-hydroxyethylthio)ethyl 2-(6-methoxynaphthalen-2-yl)propanoate (CD1-L16S-OH, 17.0 g, 50.9 mmol) in 100 mL of DCM at 0° C. under nitrogen. To this stirred mixture was added a solution of pyridine (6.2 mL, 76.4 mmol) in 50 mL of DCM over 5 minutes. The mixture was stirred at 0° C. under nitrogen for 1 h. TLC analysis of the mixture indicated completion of the reaction. The mixture was washed with 1N HCl (3×100 mL) and brine (2×100 mL). The organic layer was dried over Na₂SO₄ and concentrated in vacuo to afford the title compound (CD1-L16S-R1-CI) as yellow oil of sufficient purity to be used as such in the next step. Yield: 21.0 g (93.6%); ¹H NMR (CDCl₃, 300 MHz): δ 1.58 (d, J=6.6 Hz, 3H), 1.82 (d, J=6.0 Hz, 3H), 2.64-2.77 (m, 4H), 3.86 (q, J=7.2 Hz, 1H), 3.91 (s, 3H), 4.20 (t, J=6.9 Hz, 2H), 4.24 (t, J=6.9 Hz, 2H), 6.40 (q, J=5.7 Hz, 1H), 7.10-7.18 (m, 2H), 7.39 (dd, J=8.4, 1.5 Hz, 1H), 7.65-7.74 (m, 3H); MS m/z: 463.1 [M+Na].

### Step 3: Preparation of (2S)-2-(2-((1-(nitrooxy)ethoxy)carbonyloxy)ethylthio)ethyl 2-(6-methoxynaphthalen-2-yl)propanoate (CD1-L16S-R1)

Silver nitrate (12.1 g, 71.3 mmol) was added to a solution of 2-(2-((1-chloroethoxy)carbonyloxy)ethylthio)ethyl 2-(6-methoxynaphthalen-2-yl)-propanoate (CD1-L16S-R1-CI, 21.0 g, 47.6 mmol) in 175 mL of ACN and the mixture was refluxed in dark at 85-90° C. for ˜45 minutes when HPLC analysis of the mixture indicated complete conversion. The mixture was cooled and filtered through celite. The filtrate was concentrated and the residue was re-dissolved in DCM (˜100 mL) and filtered through celite to remove the precipitated silver chloride. The filtrate was concentrated in vacuo and the residue thus obtained was purified by column chromatography (400.0 g of silica gel, 200-400 mesh, eluted with 13% EtOAc in petroleum ether) to afford the title compound as yellow oil. Yield: 20.0 g (89.8%); ¹H NMR (CDCl₃, 300 MHz): δ 1.55-1.63 (m, 6H), 2.64-2.77 (m, 4H), 3.86 (q, J=7.2 Hz, 1H), 3.91 (s, 3H), 4.19 (t, J=6.9 Hz, 2H), 4.24 (t, J=6.6 Hz, 2H), 6.90 (q, J=5.7 Hz, 1H), 7.10-7.18 (m, 2H), 7.39 (dd, J=8.4, 1.5 Hz, 1H), 7.65-7.74 (m or distorted t, 3H); ¹³C NMR (CDCl₃, 75.47 MHz): δ 17.5, 18.6, 30.5, 30.8, 45.5, 55.4, 64.1, 67.4, 96.4, 105.7, 119.2, 126.1, 126.3, 127.3, 129.0, 129.4, 133.8, 135.5, 152.6, 157.8, 174.5; MS m/z: 490.1 [M+Na]⁺; HRMS ESI (m/z): [M+Na]⁺ calculated for C₂₁H₂₅N₁Na₁O₉S₁: 490.1142; Found: 490.1147 (Mass Accuracy: −1.02 ppm).

### Step 4: Preparation of (2S)-2-(2-((1-(nitrooxy)ethoxy)carbonyloxy)ethylsulfinyl)ethyl 2-(6-methoxynaphthalen-2-yl)propanoate (I-CD1-L16-R1)

A solution of sodium periodate (NaIO₄, 5.5 g, 25.6 mmol) in 25 mL of water was added drop-wise to a stirred solution of CD1-L16S-R1 (8.0 g, 17.0 mmol) in 100 mL of 3:1 methanol/acetone over 15 minutes and the resulting turbid mixture was stirred at RT for ˜4 h when TLC analysis of the mixture indicated >90% conversion. The mixture was concentrated and the residue thus obtained was diluted with 100 mL of DCM and washed with water (3×100 mL) and brine (1×100 mL). The organic layer was dried over anhydrous Na₂SO₄ and concentrated to give a crude product (˜9.0 g) which was triturated and sonicated with 40% EtOAc in petroleum ether to afford the title compound (I-CD1-L16-R1) as a white solid. Mp: 112-115° C.; Yield: 1.6 g (19.0%); ¹H NMR (CDCl₃, 300 MHz): δ 1.53-1.63 (m, 6H), 2.52-3.10 (m, 4H), 3.87 (q, J=6.9 Hz, 1H), 3.91 (s, 3H), 4.09-4.67 (m, 4H), 6.86-6.94 (m, 1H), 7.12 (s, 1H), 7.15 (d, J=9.0 Hz, 1H), 7.37 (d, J=8.4 Hz, 1H), 7.65 (s, 1H), 7.71 (d, J=8.1 Hz, 2H); ¹³C NMR (CDCl₃, 75.47 MHz): δ 16.9, 17.7, 44.9, 50.2, 50.3, 50.6, 50.8, 51.1, 54.9, 56.3, 56.4, 56.5, 60.5, 95.9, 96.0, 105.2, 118.8, 118.9, 125.7, 126.9, 128.4, 128.8, 133.3, 134.62, 151.7, 157.4, 157.4, 173.6; MS m/z: 484.0 [M+H]⁺, 506.0 [M+Na]⁺; HRMS ESI (m/z): [M+Na]⁺ calculated for C₂₁H₂₅N₁Na₁O₁₀S₁: 506.1091; Found: 506.1109 (Mass Accuracy: −3.56 ppm).

### Example 25

### (2S)-2-(2-((1-(nitrooxy)ethoxy)carbonyloxy)ethylsulfonyl)ethyl 2-(6-methoxy naphthalen-2-yl)propanoate [NO-Naproxen (I-CD1-L17-R1)]

The title compound was synthesized as shown in Scheme 6 and the experimental procedure is described below:

A solution of oxone (4.7 g, 7.7 mmol) in ˜20 mL of water was added to a stirred solution of CD1-L16S-R1 (7.5 g, 16.0 mmol) in 75 mL of 2:1 methanol/acetone at 0° C. over 10 minutes and the resulting turbid solution was stirred for overnight when TLC analysis of the mixture indicated formation of the intermediate sulfoxide. Additional 8.0 g (˜13.0 mmol) of oxone as solution in water (˜35 mL) was added to the mixture and the resulting turbid mixture was diluted with ˜80 mL of methanol and stirring was continued at RT for 1 h when TLC analysis of the mixture indicated formation of the sulfone product. The mixture was concentrated on rotavap and the residue thus obtained was dissolved in ˜300 mL of DCM and washed with water (3×100 mL) and brine (2×100 mL). The organic layer was dried over anhydrous Na₂SO₄ and concentrated on rotavap to give ˜10.0 g of yellow oil which was purified by column chromatography (300.0 g of silica gel, 200-400 mesh). The residual sulfide intermediate was eluted with 10-15% EtOAc in petroleum ether. Elution with 1:1 MeOH/DCM afforded the title compound as a slightly yellow colored solid. Mp: 98-100° C.; Yield: 5.0 g (62.5%); ¹H NMR (CDCl₃, 300 MHz): δ 1.55-1.63 (m, 6H), 2.43-2.74 (m, 2H), 3.10-3.33 (m, 2H), 3.82-3.95 (m, 2H), 3.92 (s, 3H), 4.05-4.16 (m, 1H), 4.38-4.48 (m, 1H), 4.51-4.62 (m, 1H), 6.83-6.94 (m, 1H), 7.10 (d, J=2.1 Hz, 1H), 7.16 (dd, J=9.0, 2.4 Hz, 1H), 7.33 (dd, J=8.4, 1.2 Hz, 1H), 7.63 (s, 1H), 7.65-7.75 (m, 2H); ¹³C NMR (CDCl₃, 75.47 MHz): δ 16.9, 17.7, 44.9, 52.0, 53.4, 54.9, 57.9, 58.0, 60.7, 96.0, 105.1, 119.2, 125.5, 125.8, 127.1, 128.3, 128.7, 133.3, 134.5, 134.6, 151.4, 157.6, 173.2; MS m/z: 498.8 [M−H]; HRMS ESI (m/z): [M+Na]⁺ calculated for C₂₁H₂₅N₁Na₁O₁₁S₁: 522.1041; Found: 522.1063 (Mass Accuracy: −4.21 ppm).

The compounds of the examples 26 and 27 were prepared by following the experimental procedure described for preparing the compound of example 25 except that 3,3′-thiodipropanol [CAS #: 10595-09-2] was used as the starting diol linker. The characterization data of the compounds of examples 26 and 27 is provided below.

### Example 26

### (2S)-3-(3-((1-(nitrooxy)ethoxy)carbonyloxy)propylthio)propyl 2-(6-methoxy naphthalen-2-yl)propanoate [NO-Naproxen (I-CD1-L18-R1)]

The title compound (I-CD1-L18-R1) was obtained as yellow oil. Yield (last step): 96.0%; ¹H NMR (CDCl₃, 300 MHz): δ 1.60 (d, J=7.2 Hz, 3H), 1.61 (d, J=5.7 Hz, 3H), 1.75-1.91 (m, 4H), 2.40 (t, J=7.2 Hz, 2H), 2.43 (t, J=7.2 Hz, 2H), 3.87 (q, J=7.2 Hz, 1H), 3.94 (s, 3H), 4.10-4.26 (m, 4H), 6.94 (q, J=5.7 Hz, 1H), 7.12-7.19 (m, 2H), 7.41 (dd, J=8.4, 1.5 Hz, 1H), 7.68 (d, J=1.2 Hz, 1H), 7.72 (unsymmetrical d, J=8.7 Hz, 2H); ¹³C NMR (CDCl₃, 75.47 MHz): δ 16.9, 17.9, 27.6, 27.7, 27.8, 28.1, 45.0, 54.8, 62.6, 66.7, 95.7, 105.1, 118.5, 125.4, 125.7, 126.6, 128.4, 128.7, 133.2, 135.2, 152.1, 157.2, 174.0; MS m/z: 495.1.1 [M+H]⁺, 518.1 [M+Na]⁺; HRMS ESI (m/z): [M+Na]⁺ calculated for C₂₃H₂₉N₁Na₁O₉S₁: 518.1455; Found: 518.1465 (Mass Accuracy: −1.93 ppm).

### Example 27

### (2S)-3-(3-((1-(nitrooxy)ethoxy)carbonyloxy)propylsulfinyl)propyl 2-(6-methoxynaphthalen-2-yl)propanoate [NO-Naproxen (I-CD1-L19-R1)]

The title compound (I-CD1-L19-R1) was obtained as yellow oil. Yield (last step): 1.4 g (70.0%). ¹H NMR (CDCl₃, 300 MHz) (Mixture of diastereomers): δ 1.60 (dd, J=5.7, 1.5 Hz, 6H), 1.92-2.11 (m, 4H), 2.23-2.55 (m, 4H), 3.87 (q, J=7.2 Hz, 1H), 3.93 (s, 3H), 4.09-4.37 (m, 4H), 6.94 (q, J=5.7 Hz, 1H), 7.13 (distorted d, J=2.4 Hz, 1H), 7.18 (dd, J=8.7, 2.4 Hz, 1H), 7.40 (dd, J=7.2, 1.2 Hz, 1H), 7.67 (br s, 1H), 7.71 (d, J=8.4 Hz, 2H); ¹³C NMR (CDCl₃, 75.47 MHz) (Mixture of diastereomers): δ 16.9, 17.6, 21.5, 21.6, 21.8, 44.9, 47.8, 48.3, 54.8, 62.2, 62.3, 66.4, 66.5, 95.8, 105.1, 118.7, 125.4, 125.7, 126.7, 128.3, 128.7, 133.2, 135.1, 135.2, 152.0, 157.3, 173.9; MS m/z 512.2 [M+H]⁺, 534.1 [M+Na]⁺. HRMS ESI (m/z): [M+H]⁺ calculated for C₂₃H₃₀N₁O₁₀S₁: 512.1585; Found: 512.1598 (Mass Accuracy: −2.17 ppm). Purity by HPLC @210 nm: 96.39%.

Examples of the compounds of formula I which are the prodrugs of the drugs containing an amino group:

### Example 28

### (Z)-3-ethyl 5-methyl 4-(2-chlorophenyl)-6-methyl-2-(15-(nitrooxy)-6,13-dioxo-2,7,12,14-tetraoxa-5-azahexadec-9-enyl)-1,4-dihydropyridine-3,5-dicarboxylate [NO-Amlodipine (I-AD1-L2-R1)]

This compound was synthesized in 2 steps as shown in Scheme 8 and the experimental procedure is described below:

### Step 1: Preparation of (Z)-3-ethyl 5-methyl 2-(15-chloro-6,13-dioxo-2,7,12,14-tetraoxa-5-azahexadec-9-enyl)-4-(2-chlorophenyl)-6-methyl-1,4-dihydropyridine-3,5-dicarboxylate (AD1-L2-R1-CI)

A solution of triphosgene (0.5 g, 1.7 mmol) in 4 mL of DCM was added to a stirred solution of amlodipine besylate (2.9 g, 5.1 mmol) and triethylamine (1.5 mL, 10.1 mmol) in 26 mL of DCM at RT and the mixture was stirred for 1.5 h to get the crude isocyanate intermediate AD1-IM1. To this stirred mixture was added a solution of (Z)-1-chloroethyl 4-hydroxybut-2-enyl carbonate (HO-L2-R1-CI, 1.0 g, 5.1 mmol, freshly prepared as described in Example 4) in 4 mL of DCM and the mixture was stirred at RT for 12 h when TLC analysis of the mixture indicated formation of a new product. The mixture was diluted with DCM (40 mL), washed with 0.5 N HCl (1×40 mL) & brine (1×50 mL). The organic layer was dried over MgSO₄ and concentrated on rotavap to give a residue which was purified by column chromatography on silica gel by eluting with 30% EtOAc in hexane to afford the title compound AD1-L2-R1-CI as yellow oil. Yield: 1.5 g (47.0%); ¹H NMR (CDCl₃, 300 MHz): δ 1.18 (t, J=4.2 Hz, 3H), 1.82 (d, J=3.6 Hz. 3H), 2.36 (s, 3H), 3.42-3.51 (m, 2H), 3.59-3.68 (m, 5H), 4.01-4.08 (m, 2H), 4.64-4.78 (m, 4H), 4.82 (d, J=3.6 Hz, 2H), 5.05 (br s, 1H), 5.40 (s, 1H), 5.74-5.87 (m, 2H), 6.42 (q, J=3.6 Hz, 1H), 7.04 (t, J=4.5 Hz, 1H), 7.14 (t, J=6.0 Hz, 1H), 7.20-7.28 (m, 2H), 7.37 (d, J=4.5 Hz, 1H); MS m/z: 628.2 [M+H]⁺, 651.2 [M+Na]⁺.

### Step 2: Preparation of the title compound NO-Amlodipine (I-AD1-L2-R1)

Silver nitrate (0.6 g, 3.3 mmol) was added to a stirred solution of the intermediate AD1-L2-R1-CI (1.4 g, 2.2 mmol) in 25 mL of ACN at RT and the mixture was stirred at ˜90° C. for 1.5 h when HPLC analysis of the mixture indicated completion of the reaction. The mixture was cooled and filtered through celite pad. The filtrate was concentrated and the residue obtained was partitioned between EtOAc (75 mL) and water (75 mL). The EtOAc layer was separated, washed with brine (1×75 mL), dried over anhydrous Na₂SO₄ and concentrated in vacuo to give the crude product which was purified by column chromatography on silica gel by eluting with 20% EtOAc in hexane to afford the title compound as yellow oil. Yield: 1.2 g (81.0%); ¹H NMR (CDCl₃, 300 MHz): δ 1.20 (t, J=7.2 Hz, 3H), 1.60 (d, J=5.4 Hz, 2H), 2.38 (s, 2H), 2.67 (s, 1H), 3.33-3.70 (m, 7H), 3.98-4.16 (m, 2H), 4.64-4.87 (m, 6H), 5.05 (br s, 0.7H), 5.32 (s, 0.37H), 5.42 (s, 0.7H), 5.55-5.63 (m, 0.25H), 5.70-5.90 (m, 2H), 6.94 (q, J=5.7 Hz, 1H), 7.02-7.47 (m, 5H); MS m/z: 654.2 [M−H]⁻.

### Example 29

### Ethyl 2-(1-(14-(nitrooxy)-3,12-dioxo-4,11,13-trioxa-7,8-dithia-2-azapentadecyl)cyclohexyl)acetate [NO-Gabapentin ethyl ester (I-AD2-L1-R1)]

This compound was synthesized in 4 steps as shown in Scheme 9 and the experimental procedure is described below:

### Step 1: Preparation of ethyl 2-(1-(3,12-dioxo-4,11-dioxa-7,8-dithia-2-azamidecyl)cyclohexyl)acetate (AD2-L1-OAc)

To a stirred solution of diphosgene (1.4 mL, 12.0 mmol) in 4 mL of dry DCM at 0° C. under nitrogen was added a solution of 2-((2-hydroxyethyl)disulfanyl)ethyl acetate (HO-L1-OAc, 0.8 g, 4.0 mmol, freshly prepared by mono-acetylation of 2-hydroxyethyl disulfide (HO-L1-OH)) and diisopropylethylamine (DIPEA, 3.5 mL, 19.9 mmol) in 4 mL of DCM over 20 minutes and the mixture was stirred at the same temperature for 40 minutes. The mixture was concentrated at RT to give the crude formyl chloride CI-L1-OAc. A mixture of gabapentin ethyl ester hydrochloride (0.9 g, 4.0 mmol, freshly prepared from gabapentin using thionyl chloride/ethanol method) and DIPEA (1.4 mL, 8.0 mmol) in 4 mL of DCM was added to the intermediate formyl chloride CI-L1-OAc at 0° C. under nitrogen and the mixture was stirred at RT for overnight (˜12 h). The mixture was concentrated and the residue was re-dissolved in 25 mL of ethyl acetate and washed with water (1×10 mL) and brine (1×10 mL). The organic layer was dried over Na₂SO₄ and concentrated in vacuo to get 2.9 g of crude product as yellow oil which was purified by column chromatography (silica gel, 90.0 g, 200-400 mesh, eluted with 30% EtOAc in hexane) to afford the title compound as colorless oil. Yield: 1.2 g (73.0%); ¹H NMR (CDCl₃, 300 MHz): δ 1.22 (t, J=7.3 Hz, 3H), 1.27-1.68 (m, 10H), 2.06 (s, 3H), 2.27 (s, 2H), 2.91 (t, J=6.6 Hz, 4H), 3.19 (d, J=6.7 Hz, 2H), 4.12 (q, J=7.2 Hz, 2H), 4.31 (q, J=6.4 Hz, 4H), 5.40 (br s, 1H); MS m/z: 422 [M+H]⁺, 444 [M+Na]⁺.

### Step 2: Preparation of ethyl 2-(1-(((2-((2-hydroxyethyl)disulfanyl)ethoxy)-carbonylamino)methyl)cyclohexyl)acetate (AD2-L1-OH)

To a stirred solution of AD2-L1-OAc (1.2 g, 2.8 mmol) in 10 mL of methanol at 0° C. was added an ice-cold solution of K₂CO₃ (0.6 g, 4.3 mmol) in 2 mL of water over a period of 30 minutes when TLC analysis of the mixture indicated consumption of all the starting material. The mixture was filtered and the solid residue was washed with methanol (10 mL). The filtrate was concentrated and the residue was re-dissolved in 30 mL of ethyl acetate and washed with water (1×10 mL) and brine (1×10 mL). The organic layer was dried over Na₂SO₄ and concentrated to give 0.9 g of crude product which was purified by column chromatography (silica gel, 30.0 g, 200-400 mesh, eluted with DCM) to afford the title compound (AD2-L1-OH) as yellow oil. Yield: 0.4 g (32.0%); ¹H NMR (CDCl₃, 300 MHz): δ 1.25 (t, J=7.2 Hz, 3H), 1.30-1.71 (m, 10H), 2.87-2.94 (m, 4H), 2.27 (s, 2H), 3.18 (d, J=6.6 Hz, 2H), 3.87 (t, J=5.7 Hz, 2H), 4.09-4.16 (q, J=7.1 Hz, 2H), 4.31 (t, J=6.6 Hz, 2H), 5.44 (br s, 1H); MS m/z: 380 [M+H]⁺, 402 [M+Na]⁺.

### Step 3: Preparation of ethyl 2-(1-(14-chloro-3,12-dioxo-4,11,13-trioxa-7,8-dithia-2-azapentadecyl)cyclohexyl)acetate (AD2-L1-R1-CI)

α-Chloroethyl chloroformate (CI-R1-CI, 0.2 mL, 2.1 mmol) was added drop-wise to a stirred solution of AD2-L1-OH (0.4 g, 1.1 mmol) and pyridine (0.2 mL, 2.1 mmol) in 10 mL of DCM at 0° C. under nitrogen and the mixture was stirred at RT for 45 minutes when TLC analysis of the mixture indicated formation of the desired product. The mixture was washed with 0.5 N HCl (1×10 mL) and brine (1×10 mL), dried over MgSO₄ and concentrated in vacuo to give a residue which was purified by column chromatography (silica gel, 15.0 g, 200-400 mesh eluted with 20% EtOAc in hexane) to afford the title compound (AD2-L1-R1-CI) as yellow oil. Yield: 0.4 g (83.0%); ¹H NMR (CDCl₃, 300 MHz): δ 1.28 (t, J=7.2 Hz, 3H), 1.34-1.60 (m, 10H), 1.85 (d, J=6.0 Hz, 3H), 2.30 (s, 2H), 2.92-3.03 (m, 4H), 3.22 (d, J=6.9 Hz, 2H), 4.15 (q, J=7.2 Hz, 2H), 4.30-4.38 (m, 2H), 4.48 (t, J=6.6 Hz, 2H), 5.42 (t, J=7.5 Hz, 1H), 6.44 (q, J=6.0 Hz, 1H); MS m/z: 508.1 [M+Na]⁺.

### Step 4: Preparation of NO-Gabapentin ethyl ester/Ethyl 2-(1-(14-(nitrooxy)-3,12-dioxo-4,11,13-trioxa-7,8-dithia-2-azapentadecyl)cyclohexyl)acetate (I-AD2-L1-R1)

Silver nitrate (0.2 g, 1.2 mmol) was added as a solid to a stirred solution of AD2-L1-R1-CI (0.4 g, 0.8 mmol) in 10 mL of ACN at RT and the mixture was stirred at 85-90° C. for 1.5 h. The mixture was allowed to attain RT, filtered through celite, the celite bed was washed with fresh ACN (15 mL). The filtrate and washings were combined and concentrated in vacuo to get a residue which was purified by column chromatography (silica gel, 15.0 g, 200-400 mesh, 20% EtOAc in hexane) to afford the title compound as yellow oil. Yield: 0.2 g (48.0%); ¹H NMR (CDCl₃, 300 MHz): δ 1.26 (t, J=4.2 Hz, 3H), 1.34-1.58 (m, 10H), 1.60 (d, J=3.3 Hz, 3H), 2.29 (s, 2H), 2.90-2.99 (m, 4H), 3.20 (d, J=3.9 Hz, 2H), 4.13 (q, J=4.2 Hz, 2H), 4.31 (t, J=3.9 Hz, 2H), 4.45 (t, J=3.9 Hz, 2H), 5.37-5.48 (m, 1H), 6.93 (q, J=3.3 Hz, 1H); MS (ESI⁻) m/z: 534.8 [M+Na]⁺.

### Example 30

(Z)-4-((1-(nitrooxy)ethoxy)carbonyloxy)but-2-enyl (S)-2-(2-oxopyrrolidin-1-yl)butanoylcarbamate [NO-Levetiracetam (I-AD3-L2-R1)]

This compound was synthesized as shown in Scheme 8 and the experimental procedure is described below:

Oxalyl chloride (1.2 mL, 14.0 mmol) was added to a solution of (S)-2-(2-oxopyrrolidin-1-yl)butanamide (AD3, levetiracetam, 2.0 g, 11.7 mmol) in 10 mL of 3:1 mixture of DCE/DCM and the mixture was refluxed for 8 h to yield the corresponding isocyanate AD3-IM1. To this cooled and stirred mixture was added drop-wise a solution of (Z)-4-hydroxybut-2-enyl 1-(nitrooxy)ethyl carbonate (HO-L2-R1, 2.5 g, 11.7 mmol, freshly prepared as described in Example 4) in 10 mL of DCM over 5 minutes and the mixture was stirred at RT for 12 h when TLC analysis of the mixture showed completion of the reaction. The mixture was concentrated to give a residue which was purified by column chromatography (silica gel 150-300 mesh, eluted with 40% EtOAc in petroleum ether) to afford the title compound (I-AD3-L2-R1) as yellow oil. Yield: 1.5 g (30.6%); ¹H NMR (CDCl₃, 300 MHz): δ 0.90 (t, J=7.2 Hz, 2.25H), 0.94 (t, J=7.2 Hz, 0.75H), 1.61 (d, J=5.4 Hz, 3H), 1.80-2.15 (m, 4H), 2.38-2.50 (m, 2H), 3.03-3.15 (m, 0.75H), 3.31-3.41 (m, 0.25H), 3.48-3.58 (m, 0.25H), 3.64-3.77 (m, 0.75H), 4.09 (m, 1H), 4.68-4.76 (m, 2H), 4.78-4.86 (m, 2H), 5.73-5.92 (m, 2H), 6.94 (q, J=5.4 Hz, 5.7 Hz, 1H), 8.04 (br s, 1H); MS m/z: 440.1 [M+Na]⁺.

The compounds of examples 31-33 were prepared by following the procedure as indicated in example 30. The characterization data for the compounds of examples 31-33 is provided below:

### Example 31

### (Z)-4-((1-(nitrooxy)ethoxy)carbonyloxy)but-2-enyl (Z)-5H-dibenzo[b,f]azepine-5-carbonylcarbamate [NO-Carbamazepine (I-AD4-L2-R1)]

The title compound (I-AD4-L2-R1) was obtained as an off-white gum. Yield: 0.6 g (55.4%); ¹H NMR (CDCl₃, 300 MHz): δ 1.59 (d, J=5.4 Hz, 3H), 4.72 (d, J=5.4 Hz, 2H), 4.77 (mixed d, J=5.1 Hz, 2H), 5.70-5.85 (m, 2H), 6.68 (br s, 1H), 6.93 (q, J=5.4 Hz, 1H), 6.98 (s, 2H), 7.37-7.45 (m, 4H), 7.47-7.53 (m, 4H); MS (EI⁺) m/z: 484.1 [M+H]⁺, 506.1 [M+Na]⁺.

### Example 32

### (Z)-4-((1-(nitrooxy)ethoxy)carbonyloxy)but-2-enyl 10-oxo-10,11-dihydro-5H-dibenzo[b,f]azepine-5-carbonylcarbamate [NO-Oxcarbazepine (I-AD5-L2-R1)]

The title compound (I-AD5-L2-R1) was obtained as an off-white gum. Yield: 30.6%; ¹H NMR (CDCl₃, 300 MHz): δ 1.60 (d, J=5.7 Hz, 3H), 3.89 (d, J=14.7 Hz, 1H), 4.45 (d, J=14.4 Hz, 1H), 4.75-4.79 (m, 4H), 5.76-5.83 (m, 2H), 6.92 (q, J=5.7 Hz, 1H), 7.07 (br s, 1H), 7.37-7.52 (m, 5H), 7.57-7.68 (m, 2H), 8.13 (d, J=7.5 Hz, 1H); MS (EI⁺) m/z: 500.1 [M+H]⁺, 522.1 [M+Na]⁺.

### Example 33

### (Z)-5-((4-((1-(nitrooxy)ethoxy)carbonyloxy)but-2-enyloxy)-carbonylcarbamoyl)-10,11-dihydro-5H-dibenzo[b,f]azepin-10-yl acetate [NO—O-Acetyl-licarbazepine (1-AD6-L2-R1)]

The title compound (I-AD6-L2-R1) was obtained as an off-white gum. Yield: 48.8%; ¹H NMR (CDCl₃, 300 MHz): δ 1.59, 1.60 (mixed doublets, J=5.4 Hz, 5.7 Hz, 3H), 2.09 (d, J=12.6 Hz, 3H), 3.05-3.26 (m, 1H), 3.58-3.68 (m, 1H), 4.65-4.87 (m, 4H), 5.72-6.07 (m, 3H), 6.38-6.45 (m, 0.5H), 6.92 (q, J=5.7 Hz, 1H), 7.00 (d, J=8.7 Hz, 1H), 7.22-7.54 (m, 8.5H); MS (ES⁺) m/z: 544.2 [M+H]⁺, 566.2 [M+Na]⁺.

### Example 34

### (Z)-4-((1-(nitrooxy)ethoxy)carbonyloxy)but-2-enyl 6-methoxy-2-((4-methoxy-3,5-dimethylpyridin-2-yl)methylsulfinyl)-1H-benzo[d]imidazole-1-carboxyla [NO-Omeprazole (I-AD7-L2-R1)]

This compound was synthesized as shown in Scheme 7 and the experimental procedure is described below:

Diphosgene (0.2 g, 1.3 mmol) was added drop-wise to a stirred solution of (Z)-4-hydroxybut-2-enyl 1-(nitrooxy)ethyl carbonate (HO-L2-R1, 0.5 g, 2.3 mmol, freshly prepared as described in Example 4) and triethylamine (0.1 mL, 1.4 mmol) in 5 mL of dry DCM at 0° C. under nitrogen and the mixture was stirred for 30 minutes. The reaction mixture was concentrated to get the corresponding formyl chloride, CI-L2-R1, as yellow residue. This residue was re-dissolved in DCM (5 mL) and the resulting solution was added to a stirred mixture of omeprazole (AD7, 0.4 g, 1.1 mmol)) and DMAP (0.3 g, 2.3 mmol) in DCM (5 mL) at 0° C. and the mixture was stirred for 1 h when TLC analysis of the mixture indicated formation of a major new product. The reaction mixture was diluted with DCM (15 mL), washed with water, dried over anhydrous Na₂SO₄, concentrated and purified by column chromatography on silica gel by eluting with methanol/dichloromethane gradient to afford the title compound I-AD7-L2-R1 as a brown gum. Yield: 0.3 g (45.0%); ¹H NMR (CDCl₃, 300 MHz, mixture of diastereomers, ˜0.55:0.45): δ 1.61 (d, J=5.7, 3H), 2.21 (s, 3H), 2.37 (s, 3H), 3.76 (unsymmetrical d, J=1.2 Hz, 3H), 3.88, 3.92 (two singlets, 3H), 4.65-4.94 (m, 4H), 5.02-5.21 (m, 2H), 5.90-6.10 (m, 2H), 6.93 (q, J=5.7 Hz, 1H), 7.03 (dd, J=2.4, 9.0 Hz, 0.5H), 7.09 (dd, J=2.4, 9.0 Hz, 0.5H), 7.33 (d, J=2.4 Hz, 0.45H), 7.49 (d, J=1.8 Hz, 0.55H), 7.75 (d, J=9.0 Hz, 0.55H), 7.83 (dd, J=9.0, 1.8 Hz, 0.45H), 8.06 (br s, 1H); MS (ES⁺) m/z: 593.2 [M+H]⁺, 615.1 [M+Na]⁺.

Examples of the compounds of formula I which are the prodruqs of the drugs containing hydroxyl group:

### Example 35

### NO-Paclitaxel Prodrug (I-HD1-L2-R1)

This compound was synthesized as shown in Scheme 7 and the experimental procedure is described below:

A solution of (Z)-4-hydroxybut-2-enyl 1-(nitrooxy)ethyl carbonate (HO-L2-R1, 0.1 g, 0.5 mmol, freshly prepared as described in Example 4) and DIPEA (0.3 mL, 1.8 mmol) in 3 mL of DCM was added drop-wise to a stirred solution of diphosgene (0.1 mL, 0.9 mmol) in 1 mL of DCM at 0° C. under nitrogen over 10 minutes and the resulting mixture was stirred for 45 minutes. The mixture was concentrated in vacuo and the corresponding dry formyl chloride, CI-L2-R1, thus obtained was re-dissolved in 3 mL of DCM and cooled to 0° C. under nitrogen. To this stirred solution was added drop-wise a solution of paclitaxel (0.08 g, 0.1 mmol) and diisopropylethylamine (0.03 mL, 0.2 mmol) in 2 mL of DCM and the mixture was stirred for 2 h when TLC analysis of the mixture indicated completion of the reaction. The mixture was diluted with 10 mL of DCM and washed with water (1×10 mL) and brine (1×10 mL). The organic layer was dried over MgSO₄ and concentrated in vacuo to give a residue which was purified by column chromatography on silica gel by eluting with 10% ACN in DCM to afford the title compound I-HD1-L2-R1 as a white solid. Mp: 141-143° C.; Yield: 0.07 g (75.0%); ¹H NMR (CDCl₃, 300 MHz): δ 1.16 (s, 3H), 1.31 (s, 3H), 1.41-1.43 (m, 1H), 1.58-1.64 (m, 2H), 1.71 (s, 3H), 1.81-1.94 (m, 2H), 1.95 (s, 3H), 2.02-2.08 (m, 2H), 2.25 (s, 3H), 2.37-2.65 (m, 3H), 2.49 (s, 3H), 2.58-2.61 (m, 1H), 3.83 (d, J=7.2 Hz, 1H), 4.04-4.19 (m, 1H), 4.22 (d, J=8.4 Hz, 1H), 4.34 (d, J=8.4 Hz, 1H), 4.42-4.52 (m, 1H), 4.62-4.84 (m, 4H), 4.92-5.05 (m, 2H), 5.44 (s, 1H), 5.71 (d, J=7.2 Hz, 1H), 5.76-5.86 (m, 1H), 5.91 (s, 1H), 6.01 (d, J=8.7 Hz, 1H), 6.26-6.36 (distorted t or m, 2H), 6.50-6.68 (m, 2H), 7.35-7.68 (m, 11H), 7.76 (d, J=7.5 Hz, 2H), 8.17 (d, J=7.2 Hz, 2H); MS m/z: 1123.4 [M+Na]⁺.

### Example 36

### NO-Metronidazole Prodrug (I-HD2-L2-R1)

The title compound was synthesized in 3 steps as shown in Scheme 8 and the experimental procedure is described below:

### Step 1: Synthesis of (Z)-4-hydroxybut-2-enyl 2-(2-methyl-5-nitro-1H-imidazol-1-yl)ethyl carbonate (HD2-L2-OH)

CDI (3.1 g, 19.3 mmol) was added to a stirred suspension of metronidazole (3.0 g, 17.5 mmol) in 50 mL of DCM at RT under nitrogen and the mixture (after the addition of CDI, the suspension slowly dissolved to form a clear solution in about 30 minutes) was stirred at RT for 2.5 h when TLC of the mixture indicated formation of a new product. The mixture was cooled to 0° C. To this stirred mixture was added a solution of 2-butene-1,4-diol (HO-L2-OH, 4.3 mL, 52.6 mmol) in DCM (25 mL) and the mixture was stirred at RT for overnight and at 70° C. for 3 h when TLC analysis of the mixture indicated formation of a new product. The mixture was diluted with 50 mL of DCM, washed with water (2×30 mL), dried over anhydrous Na₂SO₄ and concentrated on rotavap to give 5.0 g of crude product which was purified by column chromatography (50.0 g silica gel, 150-300 mesh, eluted with 2-5% MeOH in DCM to afford the title intermediate HD2-L2-OH as greenish oil. Yield: 4.2 g (84.3%); ¹H NMR (CDCl₃, 300 MHz): δ 2.36 (t, J=3.6 Hz, 1H), 2.50 (s, 3H), 4.23 (t, J=3.6 Hz, 2H), 4.50 (t, J=3.0 Hz, 2H), 4.60 (t, J=3.0 Hz, 2H), 4.68 (d, J=4.2 Hz, 2H), 5.57-5.66 (m, 1H), 5.87-5.94 (m, 1H), 7.96 (s, 1H); MS m/z: 286.1 [M+H]⁺, 308.1 [M+Na]⁺.

### Step 2: Synthesis of intermediate HD2-L2-R1-CI

α-Chloroethyl chloroformate (CI-R1—C1, 0.8 mL, 7.7 mmol) was added drop-wise to a solution of the intermediate HD2-L2-OH (2.0 g, 7.0 mmol) in 20 mL of DCM at 0° C. under nitrogen. To this stirred mixture was added pyridine (0.8 mL, 9.6 mmol) over 5 minutes. The mixture was stirred under nitrogen for 1 h while allowing it to attain RT. TLC analysis of the mixture indicated completion of the reaction. The mixture was washed with water (1×20 mL) and dried over Na₂SO₄ and concentrated on rotavap to afford 2.9 g of the crude product as red oil which was purified by column chromatography (34.0 g of silica gel, 150-300 mesh, eluted with DCM) to afford the title intermediate HD2-L2-R1-CI as red oil. Yield: 2.3 g (83.2%); ¹H NMR (CDCl₃, 300 MHz): δ 1.85 (d, J=5.7 Hz, 3H), 2.53 (s, 3H), 4.51 (t, J=4.8 Hz, 2H), 4.62 (t, J=4.8 Hz, 2H), 4.74 (d, J=5.7 Hz, 2H), 4.80 (s, 1H), 4.82 (d, J=2.1 Hz, 1H), 5.75-5.93 (m, 2H), 6.43 (q, J=6.0 Hz, 1H), 7.99 (s, 1H); MS (EI⁺) m/z: 392.1 [M+H]⁺, 414.1 [M+Na]⁺.

### Step 3: Synthesis of NO-Metronidazole (I-HD2-L2-R1)

Silver nitrate (12.1 g, 71.3 mmol) was added to a solution of the intermediate HD2-L2-R1-CI (1.5 g, 3.8 mmol) in 30 mL of ACN and the mixture was refluxed in dark at ˜90° C. for 2 h and at RT for overnight. HPLC analysis of the mixture indicated complete conversion. The mixture was cooled and filtered through celite. The filtrate was concentrated and the residue was re-dissolved in DCM (˜100 mL) and filtered through celite to remove the precipitated silver chloride. The filtrate was concentrated in vacuo and the residue thus obtained was purified by column chromatography (50.0 g of silica gel, 150-300 mesh, eluted with 20-80% EtOAc in petroleum ether) to afford the title compound (I-HD2-L2-R1) as red oil. Yield: 1.0 g (63.1%); ¹H NMR (CDCl₃, 300 MHz): δ 1.62 (d, J=5.7 Hz, 3H), 2.53 (s, 3H), 4.51 (t, J=4.8 Hz, 2H), 4.63 (t, J=4.8 Hz, 2H), 4.73 (d, J=5.4 Hz, 2H), 4.79 (d, J=5.4 Hz, 2H), 5.75-5.95 (m, 2H), 6.94 (q, J=5.7 Hz, 1H), 7.99 (s, 1H); MS (EI⁺) m/z: 441.1 [M+Na]⁺.

### Example 37

### NO-Zidovudine (I-HD3-L2-R1)

The above compound was synthesized as shown in Scheme 7 and the experimental procedure is described below:

Diphosgene (0.1 g, 0.5 mmol) was added drop-wise to a stirred solution of (Z)-4-hydroxybut-2-enyl 1-(nitrooxy)ethyl carbonate (HO-L2-R1, 0.2 g, 0.9 mmol, freshly prepared as described in Example 4) and triethylamine (0.3 mL, 1.8 mmol) in 5 mL of DCM at 0° C. under nitrogen and the mixture was stirred for 30 minutes. The mixture was concentrated in vacuo and the crude and dry CI-L2-R1 thus obtained was re-dissolved in 5 mL of DCM and cooled to 0° C. under nitrogen. This cold solution was added to a stirred solution of zidovudine (0.2 g, 0.9 mmol) and triethylamine (0.3 mL, 1.8 mmol) in 5 mL of DCM at 0° C. and the mixture was stirred for 3 h when TLC analysis of the mixture indicated formation of the product. The mixture was diluted with 10 mL of DCM and washed with water (1×10 mL) and brine (1×10 mL). The organic layer was dried over Na₂SO₄ and concentrated in vacuo to give a residue which was purified by column chromatography on silica gel by eluting with MeOH/DCM gradient to afford the title compound I-HD3-L2-R1 as yellow oil. Yield: 0.1 g (42.0%); ¹H NMR (CDCl₃, 300 MHz): δ 1.63 (d mixed with water peak, J=5.1 Hz, 3H), 1.93 (s, 3H), 2.35-2.57 (m, 2H), 4.05-4.12 (m, 1H), 4.29 (q, J=5.7, 5.4 Hz, 1H), 4.44 (dq, J=12.0, 2.7 Hz, 2H), 4.65-4.88 (mixed m, 4H), 5.83-6.05 (mixed m, 2H), 6.23 (t, J=6.0 Hz, 1H), 6.95 (q, J=5.7 Hz, 1H), 7.36 (s, 1H), 8.40 (br s, 1H); MS (EI⁻) m/z: 513.1 [M−H]⁻.

The compound of example 38 was prepared by following the experimental procedure described for preparing the compound of example 37. The characterization data for the compound of example 38 is described below:

### Example 38

### NO-Budesonide prodrug (I-HD4-L2-R1)

The title compound (I-HD4-L2-R1) was obtained as yellow semisolid. Yield: 8.2%; ¹H NMR (CDCl₃, 300 MHz): δ 0.83-1.08 (mixed m, 7H), 1.09-1.21 (m, 2H), 1.23-1.33 (m, 2H), 1.36-1.50 (m, 5H), 1.62 (d, 3H), 1.74-1.86 (m, 2H), 2.04-2.26 (mixed m, 4H), 2.32-2.38 (m, 1H), 2.53-2.64 (m, 1H), 4.48-4.66 (mixed m, 2H), 4.71 (s, 3H), 4.76-5.05 (mixed m, 3H), 5.13-5.20 (m, 1H), 5.80-5.99 (mixed m, 2H), 6.04 (s, 1H), 6.29 (d, J=10.2 Hz, 1H), 6.95 (q, J=5.7 Hz each, 1H), 7.24 (d, J=3.6 Hz, 1H); MS m/z: 678.3 [M+H]⁺, 700.3 [M+Na]⁺

### Example 39

### NO-Budesonide Prodrug (I-HD4-L20-R1)

The above compound was synthesized in 4 steps as shown in Scheme 10 and the experimental procedure is described below:

### Step 1: Synthesis of Intermediate HD4-L20-CHO

4-Formylbenzoic acid (HO₂C-L20-CHO, 0.2 g, 1.4 mmol) followed by DCC (0.3 g, 1.4 mmol) and DMAP (0.056 g, 0.5 mmol) were added to a stirred solution of budesonide (HD4, 0.5 g, 1.2 mmol) in dichloromethane (30 mL) and the mixture was stirred at RT for overnight. The mixture was filtered and the filtrate was washed with water (1×2 mL), 1N HCl solution (1×2 mL), brine (1×2 mL), dried over anhydrous Na₂SO₄ and concentrated in vacuo to give 0.8 g of crude product as a semisolid which was purified by column chromatography (40.0 g of silica gel, 200-400 mesh, eluted with 10-50% of ethyl acetate in petroleum ether) to afford the title Intermediate HD4-L20-CHO as a white gum. Yield: 0.6 g (93.0%); ¹H NMR (CDCl₃, 300 MHz): δ 0.97 (t, J=7.5 Hz, 3H), 1.05 (d, J=12.0 Hz, 3H), 1.11-1.33 (m, 4H), 1.38-2.32 (m, 13H), 2.35 (d, J=2.7 Hz, ˜0.4H), 2.40 (d, J=2.7 Hz, ˜0.6H), 2.60 (dt, J=13.5, 12.6, 5.1, 4.2 Hz, 1H), 4.56 (br s, 1H), 4.72 (t, J=4.5 Hz, 0.5H), 4.89 (d, J=4.5 Hz, 0.5H), 5.05 (d, J=13.2 Hz, ˜0.2H), 5.11 (d, J=12.9 Hz, ˜0.8H), 5.15-5.25 (m, 2H), 6.06 (br s, 1H), 6.30 (t, J=1.8 Hz, ˜0.5H), 6.33 (t, J=2.1 Hz, ˜0.5H), 7.29 (d, J=9.9 Hz, 1H), 8.00 (d, J=8.1 Hz, 2H), 8.26 (d, J=8.4 Hz, 2H), 10.14 (s, 1H).

### Step 2: Synthesis of intermediate HD4-L20-OH

Sodium borohydride (0.008 g, 0.2 mmol) was added to a stirred solution of aldehyde intermediate HD4-L20-CHO (0.3 g, 0.5 mmol) in 3 mL of THF at 0° C. and the mixture was stirred at 0° C. for 30 minutes. The reaction mixture was poured into 5 mL of ice cold 1N HCl solution (5 mL) and extracted with ethyl acetate (2×5 mL). The organic layer was washed with brine (1×2 mL), dried over anhydrous Na₂SO₄ and concentrated in vacuo to give 0.45 g of crude product as yellow oil which was purified by column chromatography (25.0 g of silica gel, 150-300 mesh, eluted with 10-60% ethyl acetate in petroleum ether) to afford the title HD4-L20-OH as white gum. Yield: 0.3 g (94.0%); ¹H NMR (CDCl₃, 300 MHz): δ 0.97 (t, J=7.2 Hz, 3H), 1.05 (d, J=12.0 Hz, 3H), 1.11-1.35 (m, 3H), 1.27 (s, 3H), 1.38-1.53 (m, 2H), 1.48 (s, 3H), 1.55-2.42 (m, 8H), 2.60 (dt, J=13.8, 5.1 Hz, 1H), 4.55 (br s, 1H), 4.71 (t, J=4.8 Hz, 0.5H), 4.81 (unsymmetrical d, J=5.1 Hz, 2H), 4.90 (unsymmetrical d, J=4.2 Hz, 0.5H), 4.98 (d, J=13.2 Hz, ˜0.3H), 5.04 (d, J=12.9 Hz, ˜0.7H), 5.12-5.25 (m, 2H), 6.05 (s, 1H), 6.29 (t, J=1.8 Hz, ˜0.5H), 6.32 (t, J=1.8 Hz, ˜0.5H), 7.29 (d buried under chloroform singlet, J=8.4 Hz, 1H), 7.48 (d, J=7.8 Hz, 2H), 8.08 (d, J=8.1 Hz, 2H); MS m/z: 565.3 [M+H]⁺.

### Step 3: Synthesis of intermediate HD4-L20-R1-CI

α-Chloroethyl chloroformate (CI-R1-CI, 0.063 g, 0.44 mmol) was added drop-wise to a stirred solution of the alcohol intermediate HD4-L20-OH (0.250 g, 0.44 mmol) and pyridine (0.035 g, 0.44 mmol) in 2 mL of DCM at 0° C. under nitrogen. The mixture was stirred under nitrogen for 30 minutes while allowing it to attain RT. TLC analysis of the mixture indicated completion of the reaction. The mixture was diluted with 5 mL of DCM and washed with water (1×2 mL) and brine (1×2 mL) and dried over Na₂SO₄ and concentrated in vacuo to afford 0.370 g of the crude product which was purified by column chromatography (20.0 g of silica gel, 150-300 mesh, eluted with 5-30% of EtOAc in petroleum ether) to afford the title intermediate HD4-L20-R1-CI as colorless oil. Yield: 0.246 g (82.0%); ¹H NMR (CDCl₃, 300 MHz): δ 0.97 (t, J=7.2 Hz, 3H), 1.05 (d, J=12.0 Hz, 3H), 1.11-1.33 (m, 4H), 1.38-1.83 (m, 9H), 1.86 (d, J=5.7 Hz, 3H), 1.89-2.43 (m, 5H), 2.60 (dt, J=13.8, 5.1 Hz, 1H), 4.56 (br s, 1H), 4.70 (t, J=4.5 Hz, 0.5H), 4.89 (unsymmetrical d, J=4.5 Hz, 0.5H), 5.00 (d, J=12.6 Hz, ˜0.3H), 5.06 (d, J=12.3 Hz, ˜0.7H), 5.12-5.24 (m, 2H), 5.31 (d, J=5.4 Hz, 2H), 6.05 (s, 1H), 6.29 (t, J=1.8 Hz, ˜0.5H), 6.33 (t, J=1.8 Hz, ˜0.5H), 6.46 (q, J=5.7 Hz, 1H), 7.29 (d buried under chloroform singlet, J=7.2 Hz, 1H), 7.50 (d, J=8.4 Hz, 2H), 8.11 (d, J=8.1 Hz, 2H); MS m/z: 671.3 [M+H]⁺.

### Step 4: Synthesis of NO-Budesonide (I-HD4-L20-R1)

Silver nitrate (0.8 g, 0.4 mmol) was added to a stirred solution of the chloro intermediate HD4-L20-R1-CI (0.2 g, 0.3 mmol) in 2 mL of ACN and the mixture was refluxed in dark at −70-75° C. for 2 h. HPLC analysis of the mixture indicated complete conversion. The mixture was cooled, diluted with 5 mL of DCM and filtered through celite. The filtrate was concentrated and the residue thus obtained (˜0.3 g) was purified by column chromatography (20.0 g of silica gel, 150-300 mesh, eluted with 5-30% EtOAc in petroleum ether) to afford the title compound (I-HD4-L20-R1) as white gum. Yield: 0.2 g (88.0%); ¹H NMR (CDCl₃, 300 MHz): δ 0.97 (t, J=7.5 Hz, 3H), 1.05 (d, J=12.0 Hz, 3H), 1.11-1.33 (m, 3H), 1.38-1.54 (m, 3H), 1.48 (s, 3H), 1.55-2.27 (m, 11H), 2.35 (d, J=3.3 Hz, ˜0.4H), 2.39 (d, J=2.7 Hz, ˜0.6H), 2.60 (dt, J=13.5, 12.6, 5.1, 4.2 Hz, 1H), 4.56 (br s, 1H), 4.71 (t, J=4.8 Hz, 0.5H), 4.89 (d, J=4.5 Hz, 0.5H), 5.00 (d, J=12.6 Hz, ˜0.2H), 5.06 (d, J=12.6 Hz, ˜0.8H), 5.12-5.24 (m, 2H), 5.28 (s, 2H), 6.05 (br s, 1H), 6.29 (t, J=1.8 Hz, ˜0.45H), 6.33 (t, J=1.8 Hz, ˜0.55H), 6.96 (q, J=5.7 Hz each, 1H), 7.30 (d, overlapped with chloroform singlet, 1H), 7.48 (d, J=8.4 Hz, 2H), 8.11 (d, J=8.1 Hz, 2H); MS m/z: 698.3 [M+H]⁺.

The compounds of examples 40-42 were prepared by following the experimental procedure described for example 39. The characterization data for the compounds of examples 40-42 is described below:

### Example 40

### NO-Paclitaxel prodrug (I-HD1-L20-R1)

The title compound (I-HD1-L20-R1) was obtained as a yellow solid. Mp: 117-119° C.; Yield (last step): 63.0%; ¹H NMR (300 MHz, CDCl₃): δ 1.16 (s, 3H), 1.20 (s, 3H), 1.27 (s, 1H), 1.62 (d, overlapped with water signal, 3H), 1.68 (s, 3H), 1.94 (s, 3H), 2.02 (s, 1H), 2.11-2.23 (m, 1H), 2.20 (s, 3H), 2.29-2.43 (m, 3H), 2.54 (s, 3H), 3.72 (dd, J=11.4, 3.3 Hz, 1H), 3.94 (d, J=7.5 Hz, 1H), 4.37-4.40 (m, 2H), 4.72 (d, J=11.4 Hz, 1H), 4.95 (dd, J=9.0, 3.6 Hz, 1H), 5.28 (s, 2H), 5.72-5.80 (m, 2H), 6.08 (dd, J=9.0, 3.9 Hz, 1H), 6.25 (t, J=7.5 Hz, 1H), 6.84 (s, 1H), 6.96 (q, J=5.7 Hz, 1H), 7.07 (d, J=9.3 Hz, 1H), 7.32-7.58 (m, 12H), 7.61-7.69 (m, 1H), 7.77 (d, J=7.2 Hz, 2H), 8.00 (d, J=8.1 Hz, 2H), 8.16 (d, J=7.2 Hz, 2H); MS m/z: 1121.4 [M+H]⁺, 1138.4 [M+NH₄]⁺, 1143.4 [M+Na]⁺

### Example 41

### 4-Acetamidophenyl 4-(((1-(nitrooxy)ethoxy)carbonyloxy)methyl)benzoate [NO-Paracetamol (HD5-L20-R1)]

The title compound (HD5-L20-R1) was obtained as a white solid. Mp: 149-152° C.; Yield (last step): 65.0%; ¹H NMR (300 MHz, CDCl₃): δ 1.63 (d, J=5.4 Hz, doublet partially overlapped with water signal, 3H), 2.20 (s, 3H), 5.31 (s, 2H), 6.97 (q, J=5.7 Hz, 1H), 7.18 (d, J=8.7 Hz, 2H), 7.37 (br s, 1H), 7.53 (d, J=8.1 Hz, 2H), 7.57 (d, J=8.7 Hz, 2H), 8.22 (d, J=8.4 Hz, 2H); MS m/z: 417.1 [M−H]⁻, 419.1 [M+H]⁺.

### Example 42

### (1-((2′-(1H-Tetrazol-5-yl)biphenyl-4-yl)methyl)-2-butyl-4-chloro-1H-imidazol-5-yl)methyl 4-(((1-(nitrooxy)ethoxy)carbonyloxy)methyl)benzoate [NO-Losartan (HD6-L20-R1)]

The title compound (HD6-L20-R1) was obtained as a pale yellow solid. Mp: 107-109° C.; Yield (last step): 64.0%; ¹H NMR (300 MHz, CDCl₃): δ 0.88 (t, J=7.5 Hz, 3H), 1.15-1.35 (m, 4H), 1.57 (d, J=5.7 Hz, 3H), 2.47 (t, J=7.8 Hz, 2H), 4.96 (s, 4H), 5.10 (s, 2H), 6.56 (d, J=7.8 Hz, 2H), 6.74 (d, J=7.8 Hz, 2H), 6.91 (q, J=5.7 Hz, 1H), 7.12 (d, J=7.8 Hz, 1H), 7.20 (d, J=7.8 Hz, 2H), 7.30-7.38 (m, 2H), 7.39-7.60 (m, 2H), 7.17 (d, J=8.7 Hz, 2H); MS m/z: 688.1 [M−H]⁻, 690.2 [M+H]⁺.

### Example 43

### Pharmacokinetic Data for the Compounds of the Invention

Representative compounds of formula (I) of the present invention that are the nitric oxide releasing prodrugs of known drugs or therapeutic agents, were subjected to pharmacokinetic study and the method and results of the study are presented herein below:

### General Procedures:

The oral pharmacokinetic profile of the compounds of the invention was studied in male Sprague-Dawley rats. For the purpose of the study, the nitric oxide releasing prodrugs of a drug containing a carboxylic acid functional group, e.g. naproxen that is encompassed in the compounds of formula (I), was selected. The release profile of naproxen from said nitric oxide releasing prodrugs was analysed using a HPLC system.

### Animals:

Male Sprague-Dawley rats weighing 150-220 g were used in the study. The rats were fed normal standard laboratory chow and maintained under standard environmental conditions (room temperature of 22±2° C.; 50±10% relative humidity; 12 hrs light-dark cycle.). All experimental procedures mentioned below were approved by the institutional animal ethics committee and were performed in accordance with standard guidelines of Committee for the purpose of control and supervision of experiments on animals (CPCSEA), Govt. of India for the experiment on animals.

HPLC Sample preparation and standard curve:
HPLC: Waters Alliance analytical HPLC equipped with 2996 PDA detector and Empower software were used to analyze the samples.
HPLC Column: Waters X-Terra RP-18 reversed phase column, 150×3.9 mm, 5 μm

### HPLC Method:

Flow: 1 ml/min,
detector set at 210 nm and at Maxplot (210-400 nm range).

### Solvent A: Acetonitrile;

Solvent B: 0.1% TFA in water.
Injection volume: 20 μl
Elution method: A linear gradient as specified below:

| Time in min | 0-2 | 2-10 | 10-13 | 13-14 | 14-18 |
|---|---|---|---|---|---|
| % A | 20 | 20-100 | 100 | 100-20 | 20 |

Blood samples were collected from the rats and the plasma was separated by centrifugation at 1000×g for 5 min at 4° C. A stock solution of naproxen was prepared by dissolving it in acetonitrile and working solutions of various concentrations (0.625, 1.25, 2.5, 5, 10, 20 μg/ml) were prepared by spiking the blood plasma with the naproxen stock solution. Each plasma sample (50 μl) was then transferred to a microcentrifuge tube containing acetonitrile (200 μl), mixed by vortex and centrifuged for 5 min (1000×g) at 4° C. The supernatant layer (150 μl) obtained after centrifugation was then transferred to HPLC vials. The sample solution (25 μl) was then injected in to HPLC for analysis. A linear calibration curve between the naproxen concentration in plasma (0.625, 1.25, 2.5, 5, 10, 20 μg/ml) and the peak area ratio was obtained. The rats were divided in to six groups of three each. Naproxen (10 mg/kg) was administered orally to one group of rats and the representative compounds of formula (I) i.e. the nitric oxide releasing prodrugs of naproxen (I-CD1-L1-R1, I-CD1-L2-R1, I-CD1-L3-R1, I-CD1-L4-R1, I-CD1-L16-R1, I-CD1-L17-R1 and I-CD1-L18-R1) (at a dose containing 10 mg/kg of naproxen) were administered orally to the remaining groups. Blood was collected from orbital plexus of the rats according to a specific schedule (0.25, 0.5, 1, 2, 4, 6 and 8 h after dosing) and the plasma was separated from each sample by centrifugation for 5 min (1000×g) at 4° C. Each collected plasma sample (50 μl) corresponding to naproxen and the aforementioned nitric oxide releasing prodrugs of naproxen was then transferred to a microcentrifuge tube containing acetonitrile (200 μl), mixed by vortex and centrifuged for 5 min (1000×g) at 4° C. The supernatant layer (150 μl) obtained after centrifugation was then transferred to HPLC vials. A (25 μl) volume of each sample solution was injected in to HPLC for analysis. The peak area values obtained for each of the plasma samples was compared with the naproxen standard curve to determine the plasma concentration of naproxen in rats after oral administration of naproxen and each of the nitric oxide releasing prodrugs of naproxen. The plasma concentration of naproxen in rats after oral administration of naproxen and each of the nitric oxide releasing prodrugs of naproxen versus time intervals was plotted and the area under the curve was determined by trapezoidal rule (Gibaldi, M. and Perrier, D., Pharmacokinetics, Second edition, 15:445-447) for each of the samples corresponding to naproxen and nitric oxide releasing prodrugs of naproxen. The AUC values for the nitric oxide releasing prodrugs of naproxen presented in Table 1 indicate that said prodrugs release a substantial amount of naproxen parent drug in the rat plasma.

TABLE 1
Pharmacokinetic study data

| | Plasma Naproxen |
|---|---|
| Compound¹ | AUC (μg*hr/ml) ± S.E.M |
| Naproxen | 202.77 ± 13.95 |
| (I-CD1-L1-R1) | 133.02 ± 31.75 |
| (I-CD1-L2-R1) | 187.73 ± 18.79 |
| (I-CD1-L3-R1) | 65.13 ± 11.36 |
| (I-CD1-L4-R1) | 122.95 ± 14.35 |
| (I-CD1-L6-R1) | 12.88 ± 3.85 |
| (I-CD1-L7-R1) | 9.85 ± 0.77 |
| (I-CD1-L16-R1) | 118.47 ± 8.01 |
| (I-CD1-L17-R1) | 93.85 ± 5.58 |
| (I-CD1-L18-R1) | 143.57 ± 2.60 |
| (I-CD1-L19-R1) | 153.37 ± 7.16 |

¹All the compounds were administered per oral at 10 mg/kg equivalent dose of naproxen.

### Example 44

### Estimation of Nitrate/Nitrite Release from the Compounds of the Invention in Plasma

Male Sprague-Dawley rats (180-220 g) were acclimatized for a week and fasted 12-14 hours prior to the commencement of the experiment. The representative compounds of formula (I) i.e. the nitric oxide releasing prodrugs of naproxen (I-CD1-L1-R1, I-CD1-L2-R1, I-CD1-L4-R1, and I-CD1-L18-R1) (at a dose of 10 mg/kg of naproxen) were administered orally to the rats. The blood sample was collected from the rats administered with each of the aforementioned nitric oxide releasing prodrugs of naproxen according to a specific schedule (0.5, 1, 2, 4, 6 and 8 hours) and the plasma was separated by centrifugation (1000×g) for 5 min at 4° C. The release profile of the nitrate/nitrite in the blood plasma which is an indirect measure of the nitric oxide released in the blood plasma was measured using Griess method by employing colorimetric nitrate/nitrite assay kit from Fluka.

The blood plasma samples were filtered using Millipore ultra-filtration 96-well plate to remove the plasma proteins having particle size of >10 kDa. The assay was performed in a 96-well plate according to standard procedure described in the kit. The method comprised adding to the well, standard (sodium nitrate) (80 μl) of various concentrations (0, 20, 40, 60, 80 and 100 μM) followed by the reagents, nitrate reductase (10 μl) and enzyme co-factor (10 μl). The plasma sample (80 μl) obtained from the blood sample collected at various time intervals from the rats (0.5, 1, 2, 4, 6 and 8 hours) were added to separate wells, followed by the reagents, nitrate reductase (10 μl) and enzyme co-factor (10 μl). The plate was incubated for 2 h at room temperature on orbital shaker (350-400 rpm). Griess reagent A (50 μl) was added to each well followed by incubation for 5 min and subsequently, Griess reagent B (50 μl) was added to each well followed by incubation for 10 min. The absorbance of assay plate was measured by using a 96-well plate reader (Bio-Tek instruments) at 540 nm. This procedure was carried out for each of the aforementioned nitric oxide releasing prodrugs of naproxen separately. A standard curve between the sodium nitrate concentration (μM) (0, 20, 40, 60, 80 and 100 μM) on X-axis versus absorbance values on Y-axis was plotted. The absorbance values of each of the plasma samples collected at different time intervals corresponding to the aforementioned nitric oxide releasing prodrugs of naproxen from the rats was compared with the standard curve to determine the plasma nitrate concentration in mice after oral administration of the aforementioned nitric oxide releasing prodrugs of naproxen. The plasma nitrate concentration in rats after oral administration of the aforementioned nitric oxide releasing prodrugs of naproxen versus time intervals was plotted and the area under the curve was determined for each of the samples corresponding to the aforementioned nitric oxide releasing prodrugs of naproxen as presented in the following Table 2. The results indicate that significant amounts of nitric oxide is released in the blood plasma by administering the aforementioned nitric oxide releasing prodrugs of naproxen.

TABLE 2
Estimation of nitrate/nitrite release from
the compounds of the invention in plasma

| | Plasma Nitrate/Nitrite |
|---|---|
| Compound¹ | AUC (μM*h) |
| Naproxen | 0 |
| I-CD1-L1-R1 | 709 |
| I-CD1-L2-R1 | 605 |
| I-CD1-L4-R1 | 811.6 |
| I-CD1-L18-R1 | 581.2 |
| I-CD1-L19-R1 | 961.4 |

¹All the compounds were administered per oral at 10 mg/kg equivalent dose of naproxen.

### Example 45

### Determination of the Anti-Inflammatory Activity of the Compounds of the Invention

The anti-inflammatory activity of naproxen and the nitric oxide releasing prodrug of naproxen, I-CD1-L2-R1 was assessed in carrageenan-induced rat paw edema model according to the procedure described in Takeuchi et al., J. Pharmacol. Exp. Ther. 1998, 286 (1), 115-121). Male Sprague-Dawley rats were divided into three groups of ten each. Naproxen (5 mg/kg) and the nitric oxide releasing prodrug of naproxen, I-CD1-L2-R1 (at a dose containing 5 mg/kg of naproxen), were dissolved in PEG 400 and administered orally to overnight fasted rats of different groups. One hour later, carrageenan (100 μl, 1% w/v) was injected in to their paws. The control group received PEG 400 (1 ml/kg). The paw volume of the group of rats administered with naproxen and those administered with naproxen prodrug were measured before carrageenan injection and also at a time period of 3 and 5 hours after the carrageenan was injected. The (%) inhibition of paw edema in rats administered with naproxen and the nitric oxide releasing prodrug of naproxen, I-CD1-L2-R1 after 3 and 5 hours respectively were calculated as compared to the control group and presented in Table 3. The results indicate that the nitric oxide releasing prodrug of naproxen, I-CD1-L2-R1 exhibited anti-inflammatory activity comparable to that of naproxen in the carrageenan-induced rat paw edema model.

### Ulcerogenic Activity

The ulcerogenic potential of the nitric oxide releasing prodrug of naproxen, I-CD1-L2-R1 in rats was assessed. Naproxen (100 mg/kg) and the nitric oxide releasing prodrug of naproxen, I-CD1-L2-R1 (at a dose containing 100 mg/kg of naproxen) was administered to overnight fasted rats of different groups. The animals were sacrificed after 5 h of drug administration. The stomachs of the animals treated were separated, perfused with 2% formalin (10 ml), and then a large curvature was excised. The severity of the mucosal damage was assessed on the basis of the size of the observed ulcer lesions in the images captured using a stereomicroscope attached to a digital camera (Stemi 2000, Zeiss, Germany). The Image Pro Plus software (version 5.1) was used to quantify the hemorrhagic/ulcer lesions in pixels and converted into mm². The total area of lesions were calculated for each treatment group and the measure of gastric ulcers (Mean±SEM) (mm²) presented in Table 3. The results indicate that none of the animals treated with the nitric oxide releasing prodrug of naproxen, I-CD1-L2-R1 showed any signs of development of ulcers. However, severe haemorrhagic lesions were found in rats administered with naproxen.

TABLE 3
Data: Anti-inflammatory and ulcerogenic
activity of naproxen and I-CD1-L2-R1

| | | | Gastric ulcers (Mean ± |
|---|---|---|---|
| | Anti-inflammatory activity¹ | | SEM) (mm²)¹ |
| | (% Inhibition) | | @ 100 mg/kg eq. |
| Compound | @ 3 hours | @ 5 hours | @ 5 hours |
| Naproxen | 65.45 ± 5.84 | 49.09 ± 10.35 | 75 ± 13 |
| I-CD1-L2-R1 | 54.27 ± 5.31 | 36.61 ± 8.92 | 0 ± 0 |

¹Mean ± SEM, n = 8

### Example 46

### AMES Genotoxicity Assay

AMES test or bacterial reverse mutation test uses five mutant strains (i.e., TA98, TA100, TA1535, TA1537, TA102) of Salmonella typhimurium to test the mutagenicity of chemical substances (Kristien Mortelmans and Errol Zeiger, The Ames Salmonella/microsome mutagenicity assay, Mutation Research 2000, 455, 29-60 and the relevant reference cited therein). These mutants are called his mutants because of their dependence on an external source of histidine to grow. The test also uses one trp mutant strain WP2 uvrA (which needs external supply of tryptophan for its growth) of Escherichia coli (Kristein Mortelmans and Edward S. Riccio, The bacterial tryptophan reverse mutation assay with Escherichia coli WP2, Mutation Research 2000, 455, 61-69 and the relevant references cited therein). If the bacteria are incubated in the presence of a mutagen, a reverse mutation is induced, and the bacteria will grow. However, if the chemical substance is not mutagenic, there will be no reversion and thus no growth. The result is thus obtained in the absence of metabolic activation. Because many chemicals that are poor mutagens become potent mutagens after they have passed through the liver, homogenate of rat liver, called the S9 extract, are added to the bacteria before incubation. The bacteria/S9 mixture is then plated on a medium containing no histidine (use of tryptophan-deficient medium in case of E. coli strain), and the test chemical is placed in the center of the plate. The result is thus obtained in the presence of metabolic activation. A second plate that contains a non-mutagenic solvent as a negative control and a third plate that contains a known mutagen as a positive control are also run simultaneously. All the 3 types of plates (each type of plate, in fact, run simultaneously in triplicate for obtaining statistically significant data) are incubated for 48 hours at 37° C. The above procedure is called the plate incorporation method. The presence of numerous colonies of revertants in the test disk indicates a positive result: that is, the chemical substance is a mutagen (i.e., mutagenic if the increase in revertants is >2 fold for TA98, TA100, TA102 and WP2/uvrA or >3 fold for TA1535 and TA1537). Also, a positive result would be considered reliable when there is a dose-dependent increase in revertants at any two consecutive non-toxic concentrations which can be in the range of 10-5000 μg/plate. The presence of only a few spontaneous revertant colonies indicates a negative result. If a negative or equivocal result is obtained, the pre-incubation method is performed in which the cells are exposed to the test compounds for 30 min before plating. Also, before Ames test is initiated, a toxicity test on the chemical substance is performed using TA100 strain in the concentration range of 10-5000 μg/plate. The above Ames mutagenicity test is initiated only when the test substance is non-toxic to TA100 in the concentration range of 10-5000 μg/plate. When the test substance is found to be toxic at higher concentration range then the genotoxicity of that material is tested only in the non-toxic lower concentration range.

When the nitric oxide releasing prodrugs of naproxen, (I-CD1-L1-R1, I-CD1-L2-R1, I-CD1-L4-R1, I-CD1-L16-R1 and I-CD1-L18-R1) were subjected to AMES test in AMES mutagenicity assay, said prodrugs were found to be non-toxic to TA100 and non-mutagenic in all the aforementioned six bacterial strains in the concentration range of 10-5000 μg/plate. The corresponding data is presented in the following Table 4.

TABLE 4
Genotoxicity data

Ames Test Results² (up to 5000 μg/plate)

| | Toxicity | Genotoxicity |
|---|---|---|
| Compound¹ | to TA100 | to 6 strains¹ |
| Naproxen | ND | ND |
| I-CD1-L1-R1 | Non-toxic | Non-genotoxic |
| I-CD1-L2-R1 | Non-toxic | Non-genotoxic |
| I-CD1-L4-R1 | Non-toxic | Non-genotoxic |
| I-CD1-L16-R1 | Non-toxic | Non-genotoxic |
| I-CD1-L17-R1 | Non-toxic | Non-mutagenic with TA 1535 |
| I-CD1-L18-R1 | Non-toxic | Non-genotoxic |
| I-HD4-L20-R1 | Non-toxic | Non-mutagenic with TA 1535 |

¹All the compounds were administered per oral at 10 mg/kg equivalent dose of naproxen.
²Salmonella strains TA100, TA98, TA1535, TA1537 and TA102 and Escherichia coli strain WP2 uvrA were used;
ND = Not Determined.

### Example 47

### In-Vitro Aspirin Release Study of No-Aspirin Prodrugs

The test compounds were dissolved in acetonitrile to get a concentration of 200 mM which was used as stock solution. Blood samples were obtained from rats or humans in heparinized centrifuge tubes. Plasma was separated by centrifugation of blood samples at 8000 rpm at 4° C. The plasma samples collected were stored at −20° C. till use. Plasma samples were incubated at 37° C. in an incubator-shaker. The reaction mixture (2000 μl) consisted of the compound stock solution (10 μl) spiked into the plasma sample (1990 μL) to obtain a final compound concentration of 2 mM. Aspirin at concentration of 2 mM was used as standard control. At different time points after addition of compound viz., 2, 5, 10, 20, 40 and 60 min, 60 μl of sample was removed from the reaction mixture. The samples were added to 200 μl of acetonitrile and vortexed for 1 min followed by centrifuging at 12,000 rpm for 15 min. The supernatant obtained was subjected to HPLC analysis to determine the amount of aspirin and salicylate in the samples. The HPLC analysis gave the amount (μM) of aspirin and salicylate present in the samples at their respective time-points. The percent release of aspirin was calculated based on the initial concentration (2 mM) of the compound in the reaction mixture versus the amount of aspirin released at different time-points.

TABLE 5
In-vitro aspirin release data of NO-aspirin prodrugs

| Compound | Plasma sample | % Release (max) | Time |
|---|---|---|---|
| I-CD2-L15-R1-A | Rat | 16.24 | 10 min |
| I-CD2-L15-R1-A | Human | 20.84 | 10 min |
| I-CD2-L15-R1-B | Rat | 10.28 | 10 min |
| I-CD2-L15-R1-B | Human | 12.53 | 20 min |

Having thus described in detail various embodiments of the present invention, it is to be understood that the invention defined by the above paragraphs is not to be limited to particular details set forth in the above description as many apparent variations thereof are possible without departing from the spirit or scope of the present invention.

1. A compound of formula (I), all its stereoisomeric forms or a pharmaceutically acceptable salt thereof; 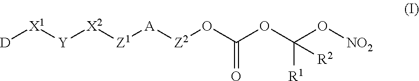
wherein,
D independently represents a drug comprising of one or more of the functional groups selected from a carboxylic acid, an amino, a hydroxyl or a sulfhydryl group that are capable of forming a covalent bio-cleavable linkage with a bio-cleavable linker represented by the formula (IA): 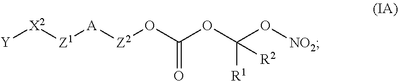
wherein,
X¹ is a bond, O, S, or NR³;
X² is a bond, O or NR³;
R³ is a bond or H;
Y is C═O or a spacer group selected from: 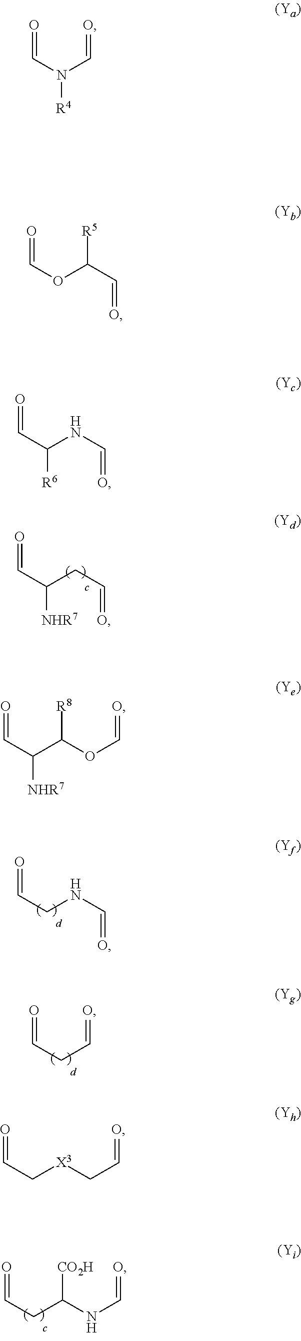 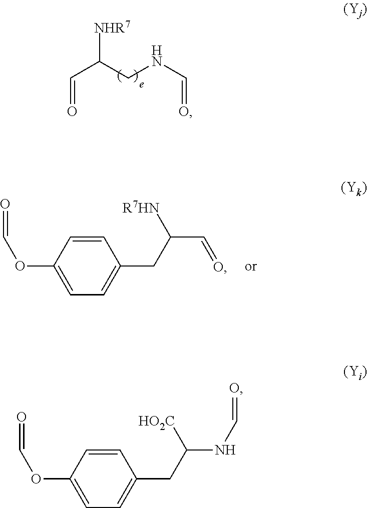
wherein:
R⁴ is a bond, H, alkyl or a metal ion;
R⁵ is H, C₁₋₆ alkyl or phenyl;
R⁶ is H or a group selected from:
—CH₃, —CH(CH₃)₂, —CH₂CH(CH₃)₂, —CH(CH₃)CH₂CH₃, —CH₂CO₂H, —CH₂CH₂CO₂H, —CH₂OH, —CH(CH₃)OH, —CH₂SH, —CH₂CH₂SCH3, —CH₂CH₂CH₂CH₂NH₂, —C₆H₅, —CH₂C₆H₅, —CH₂C₆H₄-p-OH, —CH₂CH₂CH₂NHC(═NH)NH₂, —CH₂C(═O)NH₂, —CH₂CH₂C(═O)NH₂, —CH₂-indol-3-yl or —CH₂-imidazole;
X³ is O, S, SO, SO₂ or NR³;
R⁷ is H or a group selected from: acetyl, benzoyl, alkyloxycarbonyl, benzyloxycarbonyl, 9-fluorenylmethyloxy carbonyl or its pharmaceutically acceptable ammonium salts;
R⁸ is H or C₁₋₆ alkyl;
c is an integer from 0 to 2;
d is an integer from 1 to 5;
e is an integer from 1 to 4;
Z¹ represents (CH₂)ₐ; where a is an integer from 0 to 3;
Z² represents (CH₂)_{b}; where b is an integer from 0 to 3;
A is selected from a bond, S, SO, SO₂, S—S, CH═CH, 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton, cycloalkylene, CR⁹R¹⁰, C₆-C₁₀-arylene, a 5- or 6-membered heteroarylene or a 5- or 6-membered heterocyclylene wherein, said arylene, heteroarylene and heterocyclylene may be unsubstituted or substituted by one or more substituent(s) independently selected from the group consisting of C₁₋₆ alkyl, C₁₋₆ alkoxy, hydroxyl, trifluoromethyl, cyano, amino and halogen;
R⁹ and R¹⁰ are independently selected from: H or C₁₋₆ alkyl; or R⁹ and R¹⁰ taken together with the carbon atom to which they are attached form a cycloalkyl or a heterocyclic ring;
R¹ is H; and R² is alkyl, cycloalkyl, aryl or aralkyl; or
R² is H; and R¹ independently is alkyl, cycloalkyl, aryl or aralkyl;
with the provisos that:
c) when A represents S, then a and b independently represent 3; or
d) when A represents D-isosorbide skeleton or 1,4-anhydroerythritol skeleton, then a and b independently represent 0.

2. The compound according to claim 1, wherein,
D is a drug containing a carboxylic acid group that is capable of forming a bio-cleavable covalent linkage with the linker of formula (IA);
X² is O;
R¹ is H and R² is C₁₋₆ alkyl; or
R² is H and R¹ is C₁₋₆ alkyl;
X¹ is a bond;
Y is C═O or a spacer group selected from: 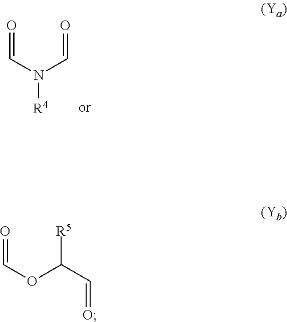
where R⁴ is a bond, H, alkyl or a metal ion; R⁵ is H, C₁₋₆ alkyl or phenyl;
A is selected from a bond, S, SO, SO₂, S—S, CH═CH, 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton, cycloalkylene and CR⁹R¹⁰, where R⁹ and R¹⁰ independently represent H or C₁₋₆ alkyl; with the provisos that:
e) when A is S, then a and b is 3; or
f) when A is D-isosorbide skeleton or 1,4-anhydroerythritol skeleton, then a and b is 0.

3. The compound according to claim 2, wherein D, the drug containing a carboxylic acid group, is selected from anti-inflammatory and analgesic agents, cardiovascular agents, anti-allergic agents, anti-cancer agents, anti-depressants, anti-convulsant agents, anti-bacterial agents, anti-fungal agents, anti-viral agents, anti-malarial agents, anti-diabetic agents, anti-ulcer agents, anti-oxidants or vitamins.

4. The compound according to claim 3, wherein the anti-inflammatory and analgesic agent is selected from opioids, steroids (glucocorticoids) or non-steroidal anti-inflammatory drugs (NSAIDs).

5. The compound according to claim 4, wherein the anti-inflammatory and analgesic drug is selected from aceclofenac, acemetacin, acetamidocaproic acid, acetylsalicylsalicylic acid, actarit, alclofenac, 3-alminoprofen, amfenac, 3-amino-4-hydroxybutyric acid, aspirin (acetylsalycilic acid), balsalazide, bendazac, benoxaprofen, bromprofen, bromfenac, 5-bromosalicylic acid acetate, bucloxic acid, bumadizone, butibufen, carprofen, cinchophen, cinmetacin, clidanac, clometacin, clonixin, clopirac, diacerein, diclofenac, diflunisal, dipyrocetyl, enfenamic acid, enoxolone, etodolac, felbinac, fenbufen, fenclozic acid, fendosal, fenoprofen, fentiazac, flufenamic acid, flunoxaprofen, fluocortolone-21-acid, flurbiprofen, fosfosal, gentisic acid, ibufenac, ibuprofen, indomethacin, indoprofen, isofezolac, isoxepac, ketoprofen, ketorolac, lonazolac, loxoprofen, meclofenamic acid, mefenamic acid, mesalamine, metiazinic acid, mofezolac, naproxen, niflumic acid, olsalazine, oxaceprol, oxaprozin, pirazolac, pirprofen, pranoprofen, protizinic acid, salicysulfuric acid, salicylamide o-acetic acid, salsalate, sulfasalazine, sulindac, suprofen, suxibuzone, tiaprofenic acid, tolfenamic acid, tolmetin, tropesin, ximoprofen, zaltoprofen or zomepirac.

6. The compound according to claim 3, wherein the cardiovascular agent is an anti-hypertensive agent selected from: angiotensin converting enzyme (ACE) inhibitors, beta-blockers, sartans (angiotensin II blockers), anti-thrombotic and vasoactive agents, anti-hyperlipidemic drugs (including HMG-CoA-reductase inhibitors i.e., statins), fibrates, anti-anginal agents, anti-arrhythmic agents, anti-hypotensive agents, calcium channel blockers, cardiotonic agents, cardioprotective agents, diuretics or vasodilators.

7. The compound according to claim 6, wherein the cardiovascular agent is selected from acifran, acipimox, acetylsalicylic acid, alacepril, gama-aminobutyric acid, angiotensin, argatroban, atorvastatin, benazepril, benfurodil hemisuccinate, beraprost, bezafibrate, bumetanide, candesartan, capobenic acid, captopril, carmoxirole, ceronapril, cerivastatin, chromocarb, cilazapril, ciprofibrate, clinofibrate, clofibric acid, dalteparin, daltroban, delapril, dextrothyroxine, eicosapentaenoic acid, eledoisin, enalapril, enalaprilat, enoxaparin, eprosartan, ethacrynic acid, fluvastatin, fosinopril, furosemide, gemfibrozil, iloprost, imidapril, indobufen, isbogrel, heparin, lamifiban, limaprost, lisinopril, lotrafiban, meglutol, melagatran, mercamphamide, mercaptomerin sodium, mercumallylic acid, mersalyl, methyldopa, moexipril, moveltipril, nadroparin, omapatrilat, ozagrel, oxiniacic acid, perindopril, piretanide, pitavastatin, pravastatin sodium, prostaglandin E₁, quinapril, ramipril, ramiprilate, reviparin sodium salt, ridogrel, sampatrilat, saralasin, satigrel, spirapril, taprostene, telmisartan, temocapril, thyropropic acid, ticrynafen, tinzaparin, tirofiban, trandolapril, triflusal, valsartan, xanthinol niacinate or xenbucin.

8. The compound according to claim 3, wherein the anti-allergic agent is selected from steroidal bronchodilators, mast cell stabilizers or anti-histamines.

9. The compound according to claim 8, wherein the anti-allergic agent is selected from acrivastine, amlexanox, bepotastine, cetirizine, fexofenadine, levocetirizine, lodoxamide, montelukast sodium, nedocromil, olopatadine, pentigetide or tranilast.

10. The compound according to claim 3, wherein the anti-cancer agent is selected from: acitretin (etretin), aminolevulinic acid, amsilarotene, butyric acid, eflornithine hydrochloride, melphalan, methotrexate, minodronate (minodronic acid), retinoic acids (including 13-cis retinoic and all trans-retinoic acids), sulindac, tamibarotene or valproic acid.

11. The compound according to claim 3, wherein the antidepressant is selected from anti-maniacs and anti-psychotics.

12. The compound according to claim 11, wherein the antidepressant is selected from amineptine, gabapentin, 5-hydroxytryptophan (oxitriptan), pregabalin, tianeptine, valproic acid or vigabatrin.

13. The compound according to claim 3, wherein the anticonvulsant is selected from carbamazepine, felbamate, gabapentin, lamotrigine, levetiracetam, licarbazepine, oxcarbazepine, pregabalin, topiramate, valpromide, vigabatrin, or zonisamide.

14. The compound according to claim 3, wherein the anti-bacterial is selected from: acediasulfone, amdinocillin, p-aminosalicylic acid, amoxicillin, amphomycin, ampicillin, apalcillin, apicycline, aspoxicillin, azidocillin, azlocillin, aztreonam, bacitracin, balofloxacin, benzoylpas, benzylpenicillin, betamipron, biapenem, carbenicillin, carindacillin, carumonam, cefaclor, cefadroxil, cefalexin, cefamandole, cefatiam, cefatrizine, cefazedone, cefazolin, cefbuperazone, cefclidin, cefdinir, cefditoren, cefepime, cefetamet, cefixime, cefmenoxime, cefmetazole, cefminox, cefodizime, cefonicid, cefoperazone, ceforanide, cefoselis, cefotaxime, cefotetan, cefotiam, cefoxitin, cefozopran, cefpimizole, cefpiramide, cefpirome, cefroxadine, cefsulodin, ceftazidime, cefteram, ceftezole, ceftibuten, ceftizoxime, ceftriaxone, cefprozil, cefuroxime, cefuzonam, cephacetrile sodium, cephalexin, cephaloglycin, cephaloridine, cephalosporin C, cephalothin, cephapirin sodium, cephradine, cilastatin, cinoxacin, ciproflaxacin, clavulinic acid, clavulanate, clinafloxacin, clometocillin, cyclacillin, dicloxacillin, difloxacin, enoxacin, epicillin, ertapenem, fenbenicillin, fleroxacin, flomoxef, floxacillin, flumequine, fosfomycin, fropenem, fusidic acid, garenoxacin, gatifloxacin, gemifloxacin, grepafloxacin, hetacillin, hydnocarpic acid, imipenem, lomefloxacin, loracarbef, lymecycline, merbromin, meropenem, metampicillin, methicillin, mezlocillin, miloxacin, moxalactam, moxifloxacin, nadifloxacin, nafcillin, nalidixic acid, negamycin, noprysulfamide, norfloxacin, ofloxacin, opiniazide, oxacillin, oxolinic acid, panipenem, pazufloxacin, pefloxacin, penicillin(s), penimepicycline, phenethicillin, phthalylsulfacetamide, phthalylsulfathiazole, pipemidic acid, piperacillin, piromidic acid, propicillin, prulifloxacin, quinacillin, ritipenem, rosoxacin, rufloxacin, salazosulfadimidine, salbactam, sitafloxacin, sparfloxacin, succinylsulfathiazole, succisulfone, sulbenicillin, sulfachrysoidine, sulfaloxic acid, 4-sulfanilamidosalicylic acid, sulfanilic acid, tazobactam, teicoplanin, temocillin, ticarcillin, tigemonam, tosufloxacin, trovafloxacin, tyrocidine or vancomycin.

15. The compound according to claim 3, wherein the antifungal agent is selected from: amphotericin B, azaserine, benzoic acid, candicidin, lucensomycin, natamycin, nystatin, propionic acid, salicylic acid or undecylenic acid (10-undecenoic acid).

16. The compound according to claim 3, wherein the antiviral agent is selected from foscarnet sodium or zanamivir.

17. The compound according to claim 3, wherein the anti-malarial agent is artesumate.

18. The compound according to claim 3, wherein the antidiabetic agent is selected from mitiglinide, nateglinide or repaglinide.

19. The compound according to claim 3, wherein, the anti-ulcer agent is selected from: acetoxolone, arbaprostil, carbenoxolone, cetraxate, ecabet, 5-methylmethionine, proglumide, rebamipide, rosaprostol, rotraxate, sofalcone or trimoprostil.

20. The compound according to claim 3, wherein the anti-oxidant is selected from: α-lipoic acid, L-Carnitine, N-acetyl L-cysteine, N-acetyl carnosine, raxofelast, tetomilast or SCMC-Lys (S-carboxymethyl-L-cysteine Lysine salt. H₂O).

21. The compound according to claim 3, wherein the vitamin is selected from: biotin (vitamin H or coenzyme R), folic acid (vitamin M), menadoxime, nicotinic acid (niacin), pantothenic acid or vitamin B₅ (a member of the B complex vitamins).

22. The compound according to claim 1, wherein,
D is a drug containing an amino group that is capable of forming a bio-cleavable covalent linkage with the linker of formula (IA);
X² is O;
R¹ is H and R² is C₁₋₆ alkyl; or
R² is H and R¹ is C₁₋₆ alkyl;
X¹ is NR³, where R³ is H or a bond;
Y is C═O or a spacer group: 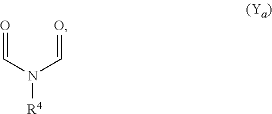
wherein, R⁴ represents a bond, H or a metal ion;
A is selected from a bond, S, SO, SO₂, S—S, CH═CH, 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton, cycloalkylene or CR⁹R¹⁰, where R⁹ and R¹⁰ independently represent H or C₁₋₆ alkyl with the provisos that:
g) when A is S, then a and b is 3; or
h) when A is D-isosorbide skeleton or 1,4-anhydroerythritol skeleton, then a and b is 0.

23. The compound according to claim 22 wherein D, the drug containing an amino group is selected from: anti-inflammatory and analgesic agents, cardiovascular agents, anti-allergic agents, anti-cancer agents, anti-depressants, anti-convulsant agents, anti-bacterial agents, anti-fungal agents, anti-viral agents, anti-malarial agents, anti-diabetic agents, anti-ulcer agents, anti-oxidants or vitamins.

24. The compound according to claim 23, wherein, the anti-inflammatory and analgesic drug is selected from: opioids, steroids (glucocorticoids) or non-steroidal anti-inflammatory drugs (NSAIDs).

25. The compound according to claim 24, wherein the anti-inflammatory and analgesic drug is selected from: aceclofenac, acetaminophen, acetaminosalol, actarit, alminoprofen, amfenac, aminochlorthenoxazin, 3-amino-4-hydroxybutyric acid, ampiroxicam, aminopropylon, anileridine, antrafenine, benorylate, benzpiperylon, p-bromoacetanilide, bromfenac, bucetin, bucolome, bufexamac, bumadizone, butacetin, capsaicine, carprofen, carsalam, celecoxib, clonixin, dezocine, diclofenac, difenamizole, difenpiramide, enfenamic acid, etersalate, ethenzamide, ethoxazene, etodolac, etofenamate, fepradinol, flipirtine, floctafenine, flufenamic acid, glafenine, ibuproxam, isoladol, isonixin, isoxicam, p-lactophenetide, lornoxicam, meclofenamic acid, mefenamic acid, meloxicam, mesalamine, mofebutazone, nifenazone, niflumic acid, nimesulide, norlevorphanol, normorphine, oxametacine, paranyline, parecoxib, parsalmide, phenacetin, phenazopyridine, phenocoll, phenopyrazone, phenylramidol, piketoprofen, piminodine, piperylone, piroxicam, piritramide, propacetamol, ramifenazone, salverine, salacetamide, salicylamide, salicylamide o-acetic acid, sulfasalazine, talniflumate, tenidap, terofenamate, tinoridine, tenoxicam, tolfenamic acid and valdecoxib.

26. The compound according to claim 23, wherein the cardiovascular agent is an anti-hypertensive agent selected from: angiotensin converting enzyme (ACE) inhibitors, beta-blockers, sartans (angiotensin II blockers), anti-thrombotic and vasoactive agents, anti-hyperlipidemic drugs (including HMG-CoA-reductase inhibitors i.e., statins), fibrates, anti-anginal agents, anti-arrhythmic agents, anti-hypotensive agents, calcium channel blockers, cardiotonic agents, cardioprotective agents, diuretics or vasodilators.

27. The compound according to claim 26, wherein the cardiovascular agent is selected from: acadesine, acebutolol, acecamide, adenosine, alacepril, alfuzosin, alprenolol, althiazide, amanozine, ambuside, amezinium methyl sulfate, amiloride, gama-aminobutyric acid, aminometradine, 2-amino-4-picoline, amisometradine, amlodipine, amosulalol, aminone, angiotensin, aranidipine, argatroban, arotinolol, atenolol, azosemide, bamethan, barnidipine, benazepril, bendazol, bendroflumethiazide, benfluorex, benidipine, benzalbutyramide, benzylhydrochlorothiazide, benzthiazide, betahistine, bethanidine, betaxolol, bevantolol, bidisomide, bisoprolol, bopindolol, bosentan, bradykinin, bucindolol, bucladesine, bucumolol, budralazine, bufeniode, bufetolol, bufuralol, bumetanide, bunazosin, bunitrolol, bupranolol, butalamine, butazolamide, buthiazide, butidrine, butofilolol, cadralazine, candesartan, capobenic acid, carazolol, cariporide, carmoxirole, caronapril, carteolol, carvedilol, celiprolol, cetamolol, chloraminophenamide, chlorazanil, chlormerodrin, chlorothiazide, chlorthalidone, ciclosidomine, cifenline, cilazapril, cilnidipine, cilostazol, clofenamide, clonidine, clopamide, cloranolol, clorexolone, cyclopenthiazide, cyclothiazide, debrisoquin, delapril, denopamine, diazoxide, dihydralazine, dilevalol, dimetofrine, disopyramide, disulfamide, dobutamine, docarpamine, dofetilide, dopamine, dopexamine, doxazosin, droprenilamine, edeserpidine, efonidipine, eledoisin, elgodipine, enalapril, enalaprilat, encamide, endralazine, enoxaparin, enoximone, epanolol, erythrophleine, esmolol, ethiazide, ethoxzolamide, etifelmin, etilefrin, etiroxate, fasudil, felodipine, fendiline, fenoldopam, fenquizone, flecamide, furosemide, gepefrine, guanabenz, guanacline, guanazodine, guanethidine, guanochlor, guanadrel, guanfacine, guanoxabenz, guanoxan, heptaminol, hydracarbazine, hydralazine, hydrochlorothiazide, hydroflumethiazide, ibopamine, imidapril, imolamine, indapamide, indecamide, indenolol, indoramin, irbesartan, isoxsuprine, isradipine, itramin tosylate, kallidin, ketanserin, labetalol, lacidipine, lamifiban, landiolol, lercanidipine, levosimendan, lidoflazine, lisinopril, lofexidine, loprinone, losartan, lotrafiban, manidipine, mebutamate, mecamylamine, mefruside, melagatran, meobentine, mephentermine, mepindolol, metaraminol, methazolamide, methoxamine, methyclothiazide, methyldopa, methyl 4-pridyl ketone thiosemicarbazone, meticrane, metipranolol, metolazone, metoprolol, mexiletine, mibefradil, midodrine, milrinone, minoxidil, moexipril, molsidomine, monatepil, moprolol, moricizine, moveltipril, moxonidine, muzolimine, nadolol, nadoxolol, nebivolol, nicardipine, nicorandil, nifedipine, nifenalol, nilvadipine, nimodipine, nipradilol, nisoldipine, nitrendipine, norepinephrine, nylidrin, olmesartan, oxprenolol, oxyfedrine, pamabrom, paraflutizide, penbutolol, pentisomide, perhexyline, perindopril, pheniprazine, phentolamine, pholedrine, picotamide, pildralazine, pilsicamide, pimethylline, pimobendan, pinacidil, pindolol, piretanide, plafibride, polythiazide, practolol, prazosin, prenalterol, prenylamine, procainamide, pronethalol, propafenone, propranolol, quinapril, quinethazone, ramipril, ranolazine, raubasine, rescimetol, rescinnamine, reserpiline, reserpine, rilmenidine, roxifiban, sampatrilat, saralasin, sematilide, sotalol, spirapril, sulfinalol, sulmazole, suloctidil, synephrine, syrosingopine, talinolol, tasosartan, teclothiazide, temocapril, terazosin, terodiline, tertatolol, theobromine, tiamenidine, tilisolol, timolol, tinofedrine, tirofiban, tocamide, todralazine, tolazoline, toliprolol, tolonidine, torsemide, trandolapril, triamterene, trichlormethiazide, trimazosin, trimetazidine, tripamide, urapidil, valsartan, vesnarinone, viquidil, xamoterol, xemilofiban, xibenolol, ximelagatran or xipamide.

28. The compound according to claim 23, wherein the anti-allergic agent is selected from steroidal bronchodilators, mast cell stabilizers or anti-histamines.

29. The compound according to claim 28, wherein the anti-allergic agent is selected from: amlexanox, antazoline, astemizole, bambuterol, cetoxime, clobenzepam, desloratadine, epinastine, mizolastine, oxatomide, pemirolast, pentigetide, pifatidine (roxatidine acetate hydrochloride), repirinast, salbutamol, salmeterol, suplatast, tazanolast, tranilast, tritoqualine or traxanox.

30. The compound according to claim 23, wherein, the anti-cancer agent is selected from: 9-aminocamptothecin, aminolevulinic acid, 3-aminopyridine-2-carboxaldehyde thiosemicarbazone (3-ap),3-aminopyridine-4-methyl-2-carboxaldehyde thiosemi-carbazone (3-amp/triapine/ocx-191/ocx-0191), amsacrine, ancitabine, anthramycin, azacitidine, bicalutamide, bisantrene, bleomycins, bropirimine, buserelin, carboplatin, carboquone, carmofur, carmustine, carubicin, chlorozotocin, cisplatin, cladribine, cyclophosphamide, cytarabine, dacarbazine, dactinomycin, daunorubicin, decitabine, defosfamide, demecolcine, diaziquone, 6-diazo-5-oxo-1-norleucine (don), docetaxel, doxorubicin, ecteinascidins, edatrexate, efaproxiral, eflornithine, eniluracil, epirubicin, erlotinib, fluorouracil, gefitinib, gemcitabine, goserelin, histamine, hydroxyurea, idarubicin, ifosfamide, imatinib, improsulfan, lanreotide, leuprolide, liarozole, lobaplatin, lomustine, lonafarnib, mannomustine, marimastat, melphalan, 6-mercaptopurine, methotrexate, methyl aminolevulinate, miboplatin, mitoguazone, mitoxantrone, nilutamide, nimustine, nolatrexed, oxaliplatin, pemetrexed, pentostatin, peplomycin, perfosfamide, phenamet, pirarubicin, piritrexim, prinomastat, procarbazine, puromycin, raltitrexed, tariquidar, temozolomide, thiamiprine, thioguanine, tiazofurin, tipifarnib, tirapazamine, troxacitabine, trimetrexate, uracil mustard (uramustine), vindesine or zorubicin.

31. The compound according to claim 23, wherein, the antidepressant is selected from an anti-maniac or anti-psychotic agent.

32. The compound according to claim 31, wherein, the antidepressant is selected from: S-adenosylmethionine, amineptine, amisulpride, amoxapine, aripiprazole, benperidol, caroxazone, carpipramine, clocapramine, clomacran, clospirazine, clozapine, demexiptiline, desipramine, droperidol, duloxetine, fencamine, fluoxetine, fluspirilene, fluvoxamine, 5-hydroxytryptophan (oxitriptan), indalpine, indeloxazine hydrochloride, iproclozide, iproniazid, isocarboxazid, levophacetoperane, maprotiline, metapramine, milnacipran, minaprine, moclobemide, molindone, mosapramine, nemonapride, nialamide, nomifensine, nortriptyline, octamoxin, olanzapine, oxypertine, paroxetine, pimozide, pipamperone, protriptyline, reboxetine, remoxipride, rolipram, roxindole, sertindole, sertraline, spiperone, sulpiride, sultopride, tianeptine, timiperone, tofenacin, tranylcypromine, viloxazine, benmoxine, rolicyprine or ziprasidone.

33. The compound according to claim 23, wherein the anticonvulsant is selected from: acetylpheneturide, albutoin, 4-amino-3-hydroxybutyric acid, atrolactamide, n-benzyl-3-chloropropionamide, buramate, carbamazepine, cinromide, clonazepam, decimemide, dimethadione, doxenitoin, ethosuximide, ethotoin, felbamate, fosphenyloin, gabapentin, lamotrigine, levetiracetam, licarbazepine, mephenyloin, mephobarbital, metharbital, methetoin, 5-methyl-5-(3-phenanthryl)hydantoin, 3-methyl-5-phenylhydantoin, nitrazepam, oxcarbazepine, oxicarbamazepine, phenacemide, phenetharbital, pheneturide, phenobarbital, phenylmethylbarbituric acid, phenyloin, phethenylate sodium, pregabalin, primidone, progabide, remacemide, rufinamide, suclofenide, sulthiame, talampanel, tetrantoin, topiramate, valpromide, vigabatrin or zonisamide.

34. The compound according to claim 23, wherein the anti-bacterial is selected from: acedapsone, acediasulfone, acetosulfone sodium, ambazone, amikacin, p-aminosalicylic acid, p-aminosalicylic acid hydrazide, amoxicillin, amphomycin, ampicillin, apalcillin, apicycline, arbekacin, aspoxicillin, azidamfenicol, azidocillin, azlocillin, aztreonam, bacampicillin, bacitracin, balofloxacin, bambermycins, benzoylpas, benzylsulfamide, betamipron, brodimoprim, 5-bromosalicylhydroxamic acid, butirosin, capreomycin, carbenicillin, carindacillin, carumonam, cefaclor, cefadroxil, cefamandole, cefatiam, cefatrizine, cefazedone, cefazolin, cefbuperazone, cefdinir, cefcapene pivoxil, cefclidin, cefditoren, cefepime, cefetamet, cefixime, cefmenoxime, cefmetazole, cefminox, cefodizime, cefonicid, cefoperazone, ceforanide, cefoselis, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin c, cephalothin, cephapirin sodium, cephradine, chloramine-B, chloramine-T, chloramphenicol, chlortetracycline, cilastatin, ciproflaxacin, clinafloxacin, clindamycin, clometocillin, clomocycline, cloxacillin, colistin, cyacetacide, cyclacillin, cycloserine, dalfopristin, dapsone, demeclocycline, deoxydihydrostreptomycin, dibekacin, dicloxacillin, dihydrostreptomycin, dirithromycin, doxycycline, enoxacin, enviomycin, epicillin, ertapenem, ethambutol, ethionamide, fenbenicillin, flomoxef, floxacillin, N2-forimicins, formylsulfisomidine, furazolium chloride, furonazide, garenoxacin, gatifloxacin, gemifloxacin, gentamycin, glyconiazide, n4-beta-d-glucosylsulfanilamide, gramicidin(s), grepafloxacin, guamecycline, hetacillin, imipenem, isepamicin, isoniazid, kanamycin(s), lenampicillin, lincomycin, linezolide, lomefloxacin, loracarbef, lymecycline, mafenide, meclocycline, meropenem, metampicillin, methacycline, methicillin, 4′-(methylsulfamoyl)sulfanilanilide, mezlocillin, micronomicin, mikamycin, minocycline, morphazinamide, moxalactam, moxifloxacin, nafcillin, negamycin, neomycin, netilmicin, nifuradene, nitrofurantoin, noprysulfamide, norfloxacin, novobiocin, opiniazide, oxacillin, oxytetracycline, panipenem, paromomycin, pazufloxacin, penamecillin, penethamate hydriodide, penicillin(s), penimepicycline, pexiganan, phenethicillin, phenyl aminosalicylate, phthalylsulfacetamide, phthalylsulfathiazole, picloxydine, pipacycline, pipemidic acid, piperacillin, pivampicillin, pivcefalexin, polymyxin, porfiromycin, primycin, pristinamycin, protionamide, pyrazinamide, quinacillin, quinupristin, ramoplanin, ribostamycin, rifabutin, rifalazil, rifamide, rifamycin sv, rifampin, rifapentine, rifaximin, ristocetin, ritipenem, rolitetracycline, salazosulfadimidine, salinazid, sancycline, sisomicin, sitafloxacin, solasulfone, sparfloxacin, spectinomycin, streptolydigin, streptomycin, streptonicozid, subathizone, 4,4′-succinylsulfathiazole, succisulfone, sulbenicillin, sulfachrysoidine, sulfanilic acid, 2-p-sulfanilylanilinoethanol, sulfinyldianiline, sulfoxone sodium, 4′-sulfanilylsulfanilamide, sulfoniazide, sulfabenzamide, sulfacetamide, sulfachlorpyridazine, sulfacytine, sulfadiazine, sulfadicramide, sulfadimethoxine, sulfadoxine, sulfaethidole, sulfaguanidine, sulfaguanole, sulfalene, sulfaloxic acid, sulfamerazine, sulfameter, sulfamethazine, sulfamethizole, sulfamethomidine, sulfamethoxazole, sulfamethoxypyridazine, sulfamethylthiazole, sulfametrole, sulfamidochrysoidine, sulfamoxole, sulfanilamide, 4-sulfanilamidosalicylic acid, p-sulfanilylbenzylamine, sulfanilylurea, n-sulfanilyl-3,4-xylamide, sulfaperine, sulfaphenazole, sulfaproxyline, sulfapyrazine, sulfasomizole, sulfasymazine, sulfathiazole, sulfathiourea, sulfisomidine, sulfisoxazole, sultamicillin, sulfatolamide, talampicillin, taurolidine, teicoplanin, temocillin, tetroxoprim, thiamphenicol, thiazosulfone, thiacetazone, thiostrepton, ticarcillin, tigemonam, tiocarlide, tobramycin, tosufloxacin, trimethoprim, trospectomycin, trovafloxacin, tuberactinomycin, tyrocidine, vancomycin, viomycin or virginiamycin.

35. The compound according to claim 23, wherein the antifungal agent is selected from: acrisorcin (9-aminoacrindine compound with 4-hexylresorcinol (1:1)), amphotericin B, anidulafungin, azaserine, bromosalicylchloranilide, buclosamide, candicidin, caspofungin, chlordantoin, exalamide, flucytosine, loflucarban, lucensomycin, magenta I, mepartricin, micafungin, natamycin, nystatin, perimycin, pyrroInitrin, salicylanilide or tubercidin.

36. The compound according to claim 23, wherein, the antiviral agent is selected from abacavir, acyclovir, adefovir, amantadine, amidinomycin, amprenavir, atazanavir, atevirdine, capravirine, cidofovir, delavirdine, didanosine, dideoxyadenosine, efavirenz, emtricitabine, entecavir, famciclovir, ganciclovir, imiquimod, indinavir, lamivudine, lopinavir, mantadine, methisazone, 5-(methylamino)-2-deoxyuridine (madu), moroxydine, nelfinavir, nevirapine, oseltamivir, penciclovir, resiquimod, ribavirin, rimantadine, ritonavir, saquinavir, stallimycin, tenofovir, tipranavir, trimetazidine, tromantadine, valacyclovir, valganciclovir, vidarabine, zalcitabine or zanamivir.

37. The compound according to claim 23, wherein, the antimalarial agent is selected from amodiaquine, chlorguanide, chloroquine, chlorproguanil, cycloguanil, hydroxychloroquine, mefloquine, 3-methylarsacetin, pamaquine, plasmocid, primaquine, pyronaridine, quinocide or tafenoquine.

38. The compound according to claim 23, wherein, the antidiabetic agent is selected from acetohexamide, buformin, carbutamide, chlorpropamide, fidarestat, glibornuride, gliclazide, glimepiride, glipizide, gliquidone, glisoxepid, glyburide, glybuthiazol(e), glybuzole, glyhexamide, glymidine, glypinamide, metformin, phenformin, pioglitazone, repaglinide, rosiglitazone, tolazamide, tolbutamide, tolcyclamide, troglitazone or voglibose.

39. The compound according to claim 23, wherein, the anti-ulcer agent is selected from: aldioxa, benexate HCl, carbenoxolone, cetraxate, cimetidine, ebrotidine, ecabapide, esaprazole, esomeprazole, famotidine, irsogladine, lafutidine, lansoprazole, leminoprazole, 5-methylmethionine, nizatidine, omeprazole, pantoprazole, pirenzepine, polaprezinc, rabeprazole, ranitidine, rebamipide, rotraxate, roxatidine, telenzepine or troxipide.

40. The compound according to claim 23, wherein the anti-oxidant is selected from: BTX-51072 (4,4-dimethyl-3,4-dihydro-2H-1,2-benzoselenazine), carnosine, melatonin, (+)-R-pramipexole, SCMC-Lys (S-carboxymethyl-L-cysteine Lysine salt H₂O), stobadine or zeatin.

41. The compound according to claim 23, wherein the vitamin is selected from: acetiamine (diacethiamine or D.A.T.), benfotiamine (s-benzoylthiamine monophosphate or BTMP), biotin (vitamin H or coenzyme R), bisbentiamine (O-benzoylthiamine disulfide), cetotiamine (O,S-dicarbethoxythiamine or DCET), cobamamide (vitamin B₂ coenzyme), cyanocobalamin (vitamin B₁₂), folic acid (vitamin M), fursultiamine (thiamine tetrahydrofurfuryl disulfide), hydroxocobalamin (vitamin B₁₂ₐ), nicotinamide, octotiamine, prosultiamine, thiamine (vitamin B₁) or vitamin K₅.

42. The compound according to claim 1, wherein,
D is a drug containing hydroxyl group that is capable of forming a bio-cleavable covalent linkage with the linker of formula (IA);
X² is O or bond;
R¹ is H and R² is C₁₋₆ alkyl; or R² is H and R¹ represents C₁₋₆ alkyl;
X¹ is O;
Y is C═O;
A is selected from: a bond, S, SO, SO₂, S—S, CH═CH, 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton, cycloalkylene and CR⁹R¹⁰;
R⁹ and R¹⁰ independently represent H or C₁₋₆ alkyl;
with the provisos that:
i) when A is S, then a and b is 3; or
j) when A is D-isosorbide skeleton or 1,4-anhydroerythritol skeleton, then a and b is 0.

43. The compound according to claim 42, wherein D the drug containing a hydroxyl group is selected from: anti-inflammatory and analgesic agents, cardiovascular agents, anti-allergic agents, anti-cancer agents, anti-depressants, anti-convulsant agents, anti-bacterial agents, anti-fungal agents, anti-viral agents, anti-malarial agents, anti-diabetic agents, anti-ulcer agents, anti-oxidants or vitamins.

44. The compound according to claim 43, wherein the anti-inflammatory and analgesic drug is selected from: opioids, steroids (glucocorticoids) or non-steroidal anti-inflammatory drugs (NSAIDs).

45. The compound according to claim 44, wherein the anti-inflammatory and analgesic drug is selected from: acetaminophen, acetaminosalol, 21-acetoxypregnenolone, alclometasone, alfa-aluminum bis(acetylsalicylate), algestone, amcinonide, 3-amino-4-hydroxybutyric acid, balsalazide, beclomethasone, benzylmorphine, betamethasone, bisabolol, bucetin, budesonide, bufexamac, buprenorphine, butorphanol, capsaicine, chlorobutanol, chloroprednisone, ciclesonide, ciramadol, clobetasol, clobetasone, clocortolone, cloprednol, corticosterone, cortisone, codeine, deflazacort, diflorasone, desomorphine, desonide, desoximetasone, dexamethasone, dezocine, diflorasone, diflucortolone, diflunisal, difluprednate, dihydrocodeine, dihydromorphine, dihydroxyaluminum acetylsalicylate, dimepheptanol, ditazol, enoxolone, eptazocine, ethylmorphine, etofenamate, eugenol, fendosal, fepradinol, floctafenine, fluazacort, fluocinolone acetonide, fluocinonide, fluocortin butyl, fluocortolone, fludrocortisone, flumethasone, fluperolone acetate, fluprednidene acetate, fluprednisolone, fluorometholone, flurandrenolide, fluticasone, formocortal, gentisic acid, glafenine, glucametacin, halcinonide, halobetasol propionate, halometasone, halopredone acetate, hydrocortamate, hydrocortisone, hydromorphone, hydroxypethidine, ibuproxam, isoladol, isoxicam, ketobemidone, p-lactophenetide, levorphanol, lornoxicam, loteprednol etabonate, mazipredone, medrysone, meloxicam, meprednisone, meptazinol, mesalamine, metazocine, methylprednisolone, metopon, mometasone furoate, morphine, nalbuphine, norlevorphanol, normorphine, olsalazine, oxaceprol, oxametacine, oxycodone, oxymorphone, oxyphenbutazone, paramethasone, pentazocine, perisoxal, piroxicam, phenazocine, phenoperidine, phenylramidol, phenylsalicylate, prednicarbate, prednisolone, prednisolone 21-diethylaminoacetate, prednisone, prednival, prednylidene, rimexolone, salacetamide, salicin, salicylamide, salsalate, sulfasalazine, tenoxicam, tixocortol, tramadol, triamcinolone acetonide, viminol or ximoprofen,

46. The compound according to claim 43, wherein the cardiovascular agent is an anti-hypertensive agent selected from: angiotensin converting enzyme (ACE) inhibitors, beta-blockers, sartans (angiotensin II blockers), anti-thrombotic and vasoactive agents, anti-hyperlipidemic drugs (including HMG-CoA-reductase inhibitors i.e., statins), fibrates, anti-anginal agents, anti-arrhythmic agents, anti-hypotensive agents, calcium channel blockers, cardiotonic agents, cardioprotective agents, diuretics orvasodilators.

47. The compound according to claim 46, wherein the cardiovascular agent is selected from: acadesine, acebutolol, ajmaline, alprenolol, ambuside, amosulalol, angiotensin, arotinolol, atenolol, atorvastatin, bamethan, benzarone, benziodarone, beraprost, betaxolol, bevantolol, bisoprolol, bosentan, bradykinin, brovincamine, bucindolol, bucumolol, bufeniode, buflomedil, bufuralol, bunitrolol, bupranolol, butofilolol, cadralazine, calcifediol, calcitriol, canrenone (hydroxyl of its ketoxime), carazolol, l-carnitine (levocarnitine), carteolol, carvedilol, celiprolol, cerivastatin, cetamolol, chlorthalidone, chromocarb, cicletanine, clobenfurol, clobenoside, convallatoxin, cyclandelate, denopamine, deslanoside, digitalin, dihydrotachysterol, dilevalol, dimetofrine, diosmin, dobesilate calcium, dobutamine, dopamine, dopexamine, efloxate, eledoisin, enoximone, epanolol, erythrophleine, escin, etafenone, ethacrynic acid, etilefrin, ezetimibe, fenofibrate, fenoldopam, fluvastatin, furazabol, gepefrine, gitoxin, guanoxabenz, heptaminol, ibudilast, ifenprodil, iloprost, indenolol, ipriflavone, isosorbide, isoxsuprine, kallidin, khellin, labetalol, lanatosides, leucocyanidin, levcromakalim, limaprost, losartan, lovastatin, meglutol, mannitol, mepindolol, metaraminol, methoxamine, methyldopa, metipranolol, metoprolol, mevastatin, midodrine, moprolol, nadolol, naftopidil, nebivolol, neriifolin, nicomol, nicotinyl alcohol, nifenalol, nipradilol, norepinephrine, nylidrin, oleandrin, olmesartan, oxprenolol, oxyfedrine, penbutolol, pentrinitrol, perhexyline, phenactropinium chloride, phentolamine, pholedrine, pildralazine, pindolol, pirifibrate, pitavastatin, pravastatin sodium, prenalterol, probucol, pronethalol, propranolol, proscillaridin, prostaglandin e¹, protheobromine, protoveratrines, ouabain, quercetin, ranolazine, rescimetol, resibufogenin, rutin sampatrilat, scillaren, scillarenin, simvastatin, sotalol, spironolactone, sulfinalol, suloctidil, synephrine, talinolol, tertatolol, thyropropic acid, ticrynafen, timolol, tinofedrine, toliprolol, tricromyl, trimazosin, troxerutin, ubiquinones, vincamine, viquidil, xamoterol, xanthinol niacinate or xipamide.

48. The compound according to claim 43, wherein the anti-allergic agent is selected from steroidal bronchodilators, mast cell stabilizers or anti-histamines.

49. The compound according to claim 48, wherein the anti-allergic agent is selected from: amlexanox, bambuterol, beclomethasone, cetoxime, ciclesonide, ebastine, fexofenadine, flunisolide, fluticasone and its approved esters, n-hydroxyethylpromethazine chloride, hydroxyzine, ibudilast, methyl prednisolone, montelukast sodium, pentigetide, repirinast, roxatidine, salbutamol, salmeterol, suplatast, terfenadine or tranilast.

50. The compound according to claim 43, wherein the anti-cancer agent is selected from: aclacinomycins, ancitabine, anthramycin, arzoxifene, azacitidine, bicalutamide, bleomycins, bropirimine, broxuridine, buserelin, calusterone, capecitabine, carubicin, CC-1065 (NSC 298223), chlorozotocin, chromomycins, cladribine, cytarabine, daunorubicin, decitabine, defosfamide, diethylstilbestrol, docetaxel, doxifluridine, doxorubicin, droloxifene, dromostanolone, ecteinascidins, enocitabine, epirubicin, epitiostanol, estramustine, etanidazole, etoposide, fenretinide, flavopiridol, formestane, fosfestrol, fulvestrant, gemcitabine, hydroxyurea, idarubicin, irinotecan, leuprolide, marimastat, melengestrol, menogaril, 6-mercaptopurine, miltefosine, minodronate (minodronic acid), mitobronitol, mitolactol, mopidamol, nitracrine, nogalamycin, nordihydroguaiaretic acid (masoprocol), olivomycins, paclitaxel and other known paclitaxel analogs, pentostatin, peplomycin, perfosfamide, pirarubicin, podophyllotoxin, prinomastat, puromycin, ranimustine, resveratrol, roquinimex, rubitecan, seocalcitol, streptonigrin, streptozocin, temoporfin, teniposide, tenuazonic acid, tiazofurin, topotecan, troxacitabine, valrubicin, vinblastine, vincristine, vindesine, vinorelbine, zorubicin or zosuquidar.

51. The compound according to claim 43, wherein the antidepressant is selected from anti-maniacs or anti-psychotics.

52. The compound according to claim 51, wherein, the antidepressant is selected from: acetophenazine, S-adenosylmethionine, befloxatone, bromperidol, bupropion, butaperazine, carphenazine, clopenthixol (cis-isomer), clospirazine, dixyrazine, fenpentadiol, fluanisone, flupentixol (cis-form), fluphenazine, fluspirilene, haloperidol, 5-hydroxytryptophan (oxitriptan), hypericin, melperone, moperone, mosapramine, opipramol, penfluridol, pericyazine, perimethazine, perphenazine, pipamperone, piperacetazine, pipotiazine, pyrisuccideanol, quetiapine, roxindole, spiperone, sultopride, timiperone, toloxatone, tramadol, trifluperidol or venlafaxine.

53. The compound according to claim 43, wherein the anticonvulsant is selected from 4-amino-3-hydroxybutyric acid, atrolactamide, buramate or ganaxolone.

54. The compound according to claim 43, wherein the anti-bacterial is selected from: amikacin, p-aminosalicylic acid, p-aminosalicylic acid hydrazide, amoxicillin, apalcillin, apicycline, arbekacin, aspoxicillin, azidamfenicol, azithromycin, bambermycins, benzoylpas, biapenem, 5-bromosalicylhydroxamic acid, butirosin, cefadroxil, cefamandole, cefatrizine, cefbuperazone, cefdinir, cefminox, cefonicid, cefoperazone, cefoselis, cefpiramide, cefprozil, chloramphenicol, chloroxylenol, chlorquinadol, chlortetracycline, clofoctol, clomocycline, cloxacillin, cloxyquin, clarithromycin, clindamycin, colistin, dalfopristin, demeclocycline, deoxydihydrostreptomycin, diathymosulfone, dibekacin, dihydrostreptomycin, dirithromycin, doxycycline, enviomycin, ertapenem, erythromycin and its ester derivatives, ethambutol, flomoxef, forimicins, fropenem, fusidic acid, gentamycin, glyconiazide, glucosulfone sodium, n4-beta-d-glucosylsulfanilamide, gramicidin(s), guamecycline, imipenem, isepamicin, josamycin, kanamycin(s), leucomycins, lincomycin, lymecycline, meclocycline, merbromin, meropenem, methacycline, micronomicin, midecamycins, mikamycin, minocycline, miokamycin, moxalactam, nadifloxacin, neomycin, netilmicin, nifurpirinol, nifurtoinol, nitroxoline, novobiocin, oleandomycin, oxytetracycline, panipenem, paromomycin, phenyl aminosalicylate, pipacycline, polymyxin, primycin, pristinamycin, quinupristin, ramoplanin, ribostamycin, rifabutin, rifalazil, rifamide, refampicin, rifamycin sv, rifampin, rifapentine, rifaximin, ristocetin, ritipenem, rokitamycin, rolitetracycline, rosaramicin, roxarsone, roxithromycin, salazosulfadimidine, salinazid, sancycline, sisomicin, spectinomycin, spiramycin, streptolydigin, streptomycin, streptonicozid, sulfaloxic acid, 4-sulfanilamidosalicylic acid, 2-p-sulfanilylanilinoethanol, teicoplanin, telithromycin, thiamphenicol, thiostrepton, tobramycin, trospectomycin, tuberactinomycin, tyrocidine, vancomycin, viomycin, virginiamycin, xanthocillin or xibornol.

55. The compound according to claim 43, wherein the antifungal agent is selected from: acrisorcin (9-aminoacrindine compound with 4-hexylresorcinol (1:1)), amphotericin B, anidulafungin, bromosalicylchloranilide, buclosamide, candicidin, caspofungin, chlorphenesin, ciclopirox, dermostatin, griseofulvin, filipin, fluconazole, fungichromin, mepartricin, micafungin, natamycin, nystatin, lucensomycin, pecilocin, perimycin, posaconazole, ravuconazole, rubijervine, salicylanilide, siccanin, 2,4,6-tribromo-m-cresol, tubercidin, viridian or voriconazole.

56. The compound according to claim 43, wherein the anti-viral agent is selected from abacavir, acyclovir, adefovir, amprenavir, atazanavir, cidofovir, didanosine, dideoxyadenosine, edoxudine, emtricitabine, entecavir, floxuridine, ganciclovir, idoxuridine, indinavir, kethoxal, lamivudine, lopinavir, 5-(methylamino)-2-deoxyuridine (madu), nelfinavir, nevirapine, penciclovir, podophyllotoxin, resiquimod, ribavirin, ritonavir, saquinavir, sorivudine, stavudine, tenofovir, tipranavir, trifluridine, tromantadine, valganciclovir, vidarabine, zalcitabine, zanamivir or zidovudine.

57. The compound according to claim 43, wherein the anti-malarial agent is selected from: amodiaquine, arteflene, artemisinin alcohol, bebeerines, cinchonidine, cinchonine, dihydroartemisinin, fosmidomycin, gentiopicrin, halofantrine, hydroxychloroquine, lumefantrine, mefloquine, pyronaridine, quinine or yingzhaosu A.

58. The compound according to claim 43, wherein the antidiabetic agent is selected from acarbose, acetohexamide, miglitol, troglitazone and voglibose.

59. The compound according to claim 43, wherein the anti-ulcer agent is selected from arbaprostil, enprostil, misoprostol, ornoprostil, gama-oryzanol A, plaunotol, rebamipide, rioprostil, rosaprostol, spizofurone (i.e., hydroxyl of its oxime derivative), telenzepine, teprenone (i.e., hydroxyl of its oxime derivative) or trimoprostil.

60. The compound according to claim 43, wherein the anti-oxidant is selected from: N-acetyl carnosine, ascorbic acid, BN-82451, L-carnitine (levocarnitine), curcumin, dexanabinol, edaravone, (−) epigallocatechin gallate, emoxipin, hydroxytyrosol, idebenone, luteolin, nicanartine, NZ-419, oxyresveratrol, probucol (including probucol prodrugs such as AGI-1067 and AGI-1096), quercetin, reductic acid, silybin, SCMC-Lys, tempol (4-hydroxy-tempo), alfa-tocopherol (vitamin E) or zeatin.

61. The compound according to claim 43, wherein, the vitamin is selected from: ascorbic acid, cobamamide (vitamin B₂ coenzyme), cyanocobalamin (vitamin B₁₂), ergosterol (provitamine D), fursultiamine (thiamine tetrahydrofurfuryl disulfide), hydroxocobalamin (vitamin B₁₂ₐ), 1-hydroxycholecalciferol, (1-hydroxyvitamin D₃), inositol (vitamin B complex), menadiol (dihydrovitamin K₃), menaquinones or vitamin K₂ (hydroxyl of its ketoxime), methylcobalamin, octotiamine, pantothenic acid (vitamin B₅), phylloquinone (hydroxyl of its ketoxime), prosultiamine (dithiopropylthiamine or DTPT or TPD), pyridoxine hydrochloride (vitamine B₆ hydrochloride), pyridoxal 5-phosphate, riboflavin (vitamin B₂ or vitamin G or lactoflavin), riboflavin monophosphate (vitamin B₂ phosphate), vitamin A, vitamin D₂, vitamin D₃, vitamin K₅, thiamine (vitamin B₁), thiamine disulfide (vitamin B₁ disulfide) or α-tocopherol (vitamin E supplement).

62. A compound according to claim 1, wherein:
D is a drug containing sulfhydryl group that is capable of forming a bio-cleavable covalent linkage with the linker of formula (IA);
X² is O;
R¹ is H and R² is C₁₋₆ alkyl or R² is H and R¹ is C₁₋₆ alkyl;
X¹ is S;
Y is C═O;
A is selected from a bond S, SO, SO₂, S—S, CH═CH, 1,2-phenylene, 1,3-phenylene, 1,4-phenylene, 2,3-pyridine, 3,4-pyridine, 2,4-pyridine, 2,5-pyridine, 2,6-pyridine, D-isosorbide skeleton, 1,4-anhydroerythritol skeleton, cycloalkylene or CR⁹R¹⁰;
R⁹ and R¹⁰ independently represent H or C₁₋₆ alkyl;
with the provisos that:
k) when A represents S, then a and b independently represent 3; or
l) when A represents D-isosorbide skeleton or 1,4-anhydroerythritol skeleton, then a and b independently represent 0.

63. The compound according to claim 62, wherein D, the drug containing sulfhydryl group is selected from cardiovascular agents or glucocorticoids.

64. The compound according to claim 63, wherein, the cardiovascular agent is selected from captopril or omapatrilat.

65. The compound according to claim 63, wherein, the glucocorticoid is tixocortol.

66. A compound according to claim 1, wherein the biocleavable linker of formula (IA) is selected from: 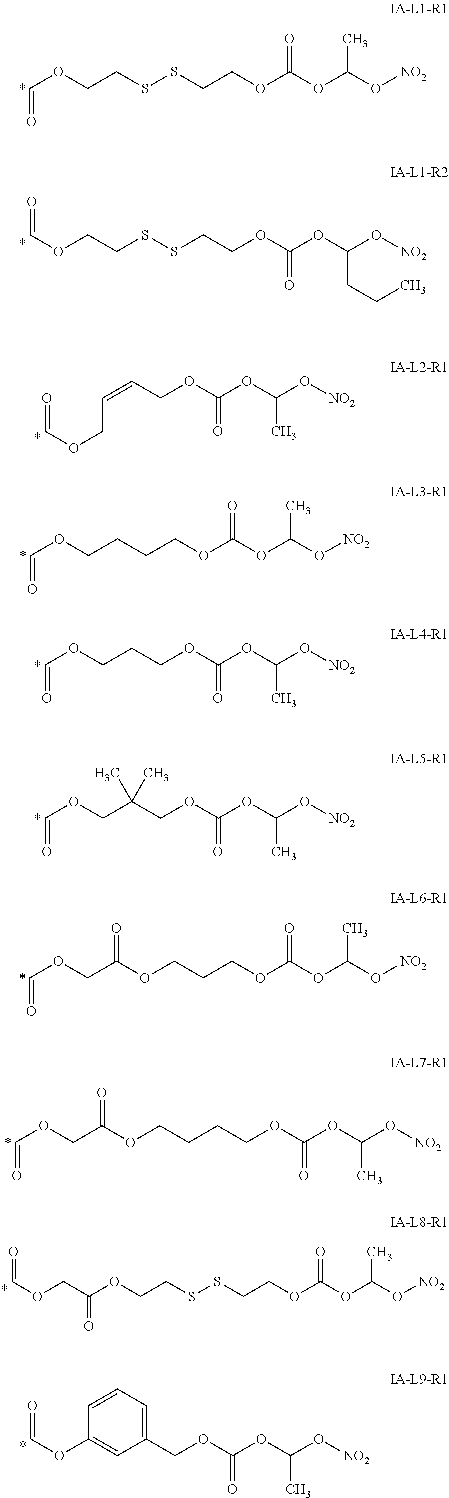 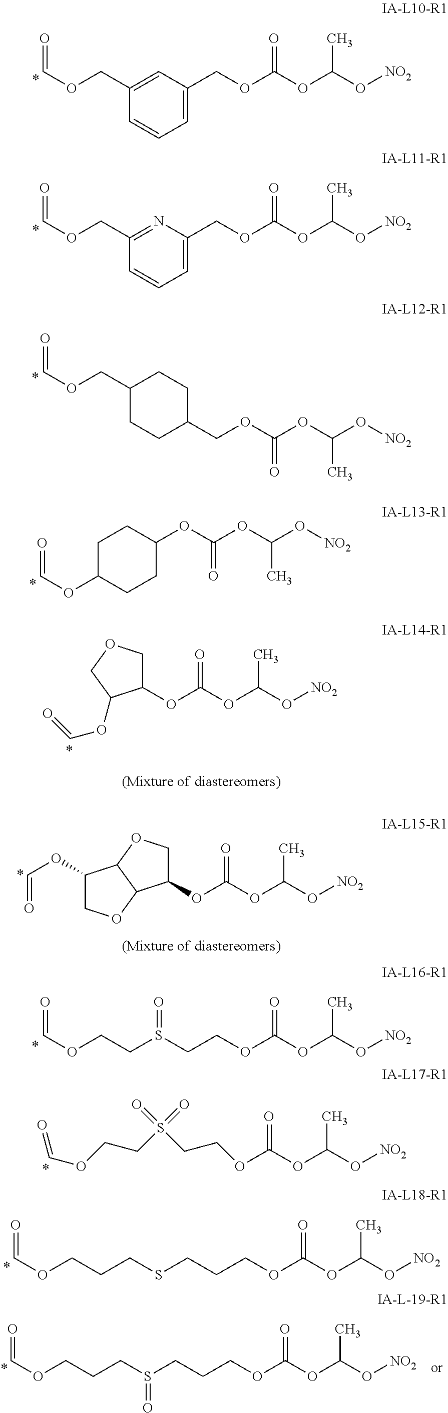 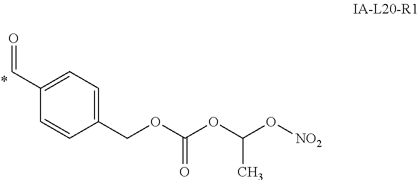
* Point of attachment to a suitable drug residue.

67. The compound according to claim 1, wherein the compound of formula (I) is selected from:

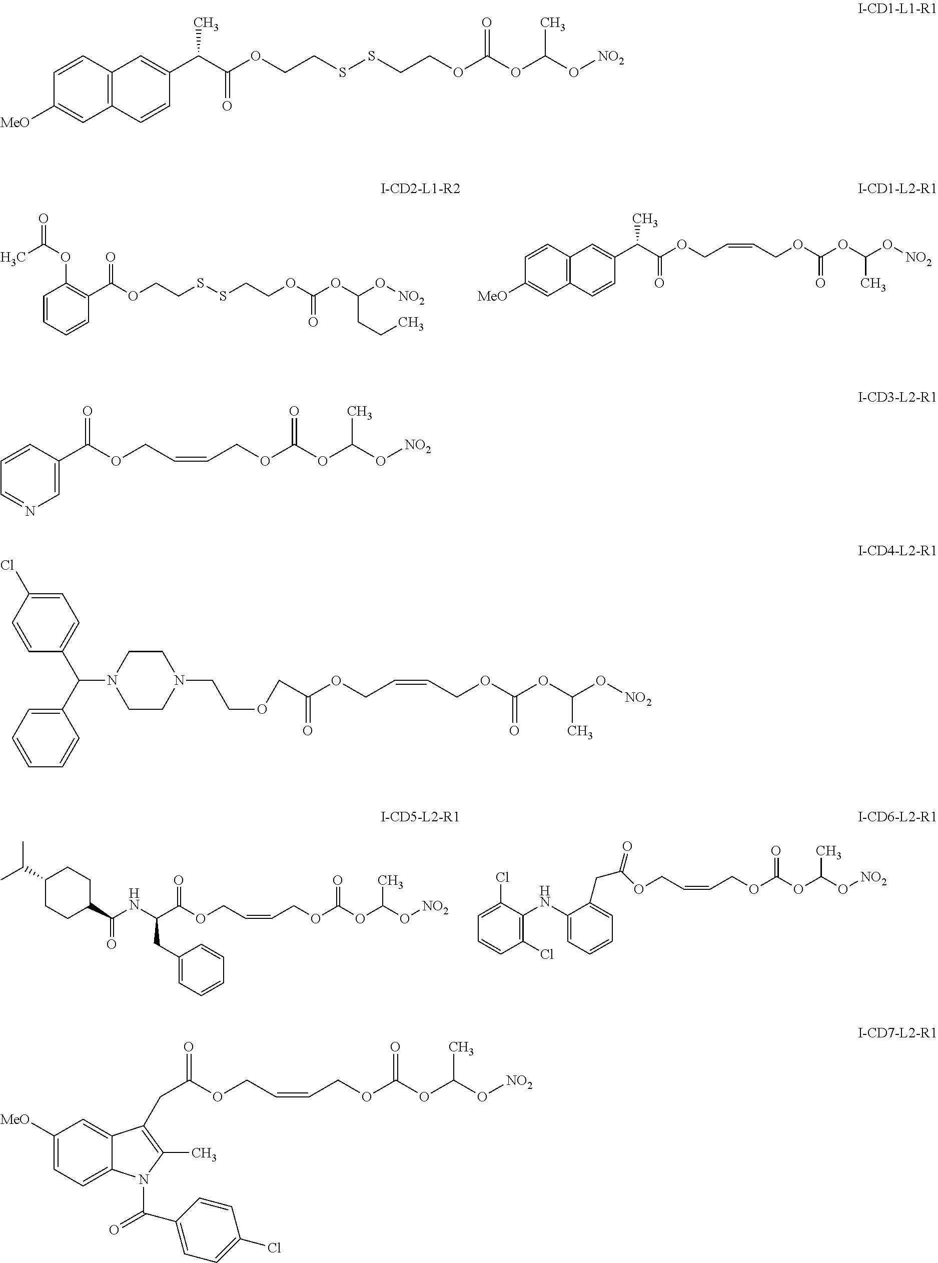

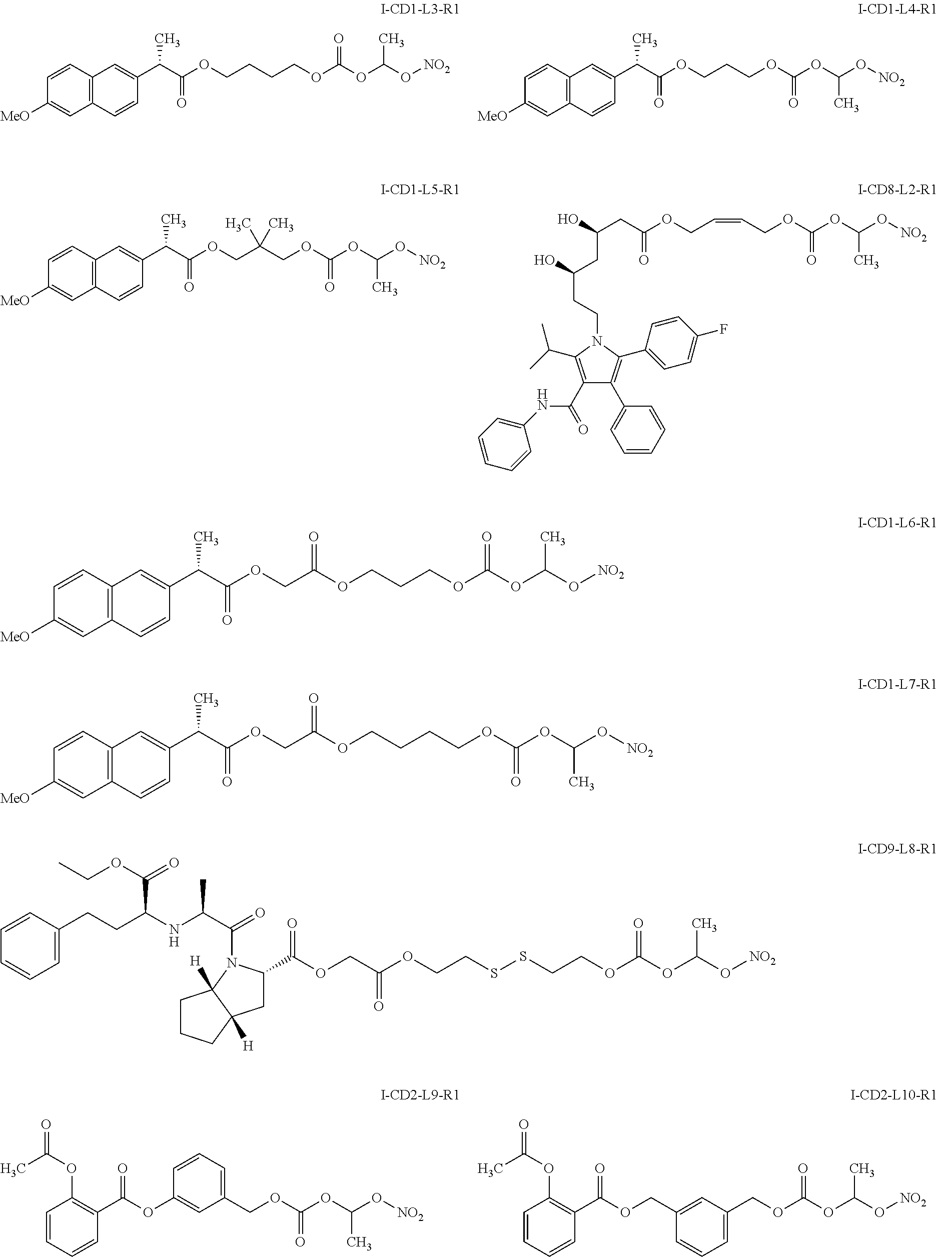

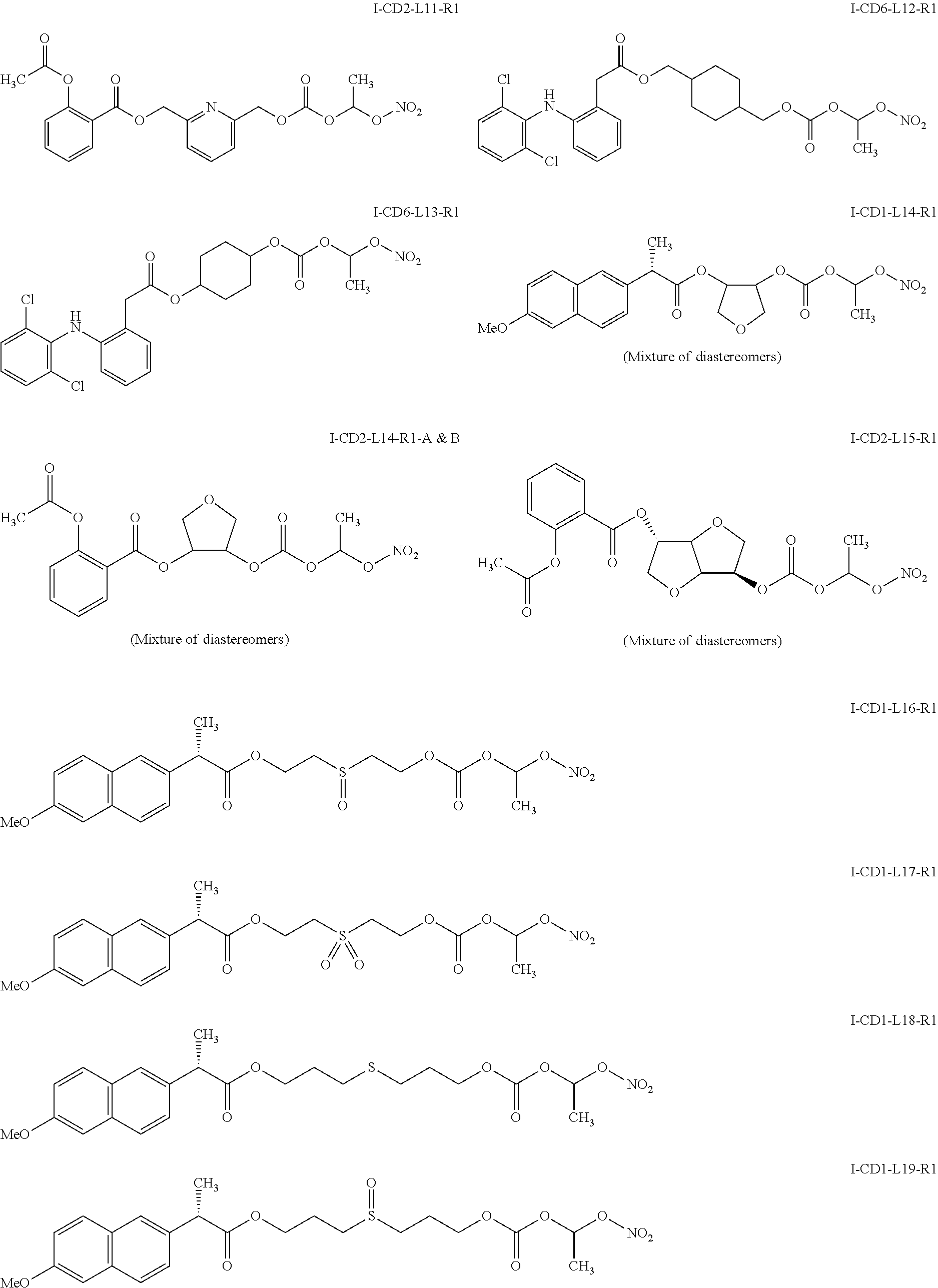

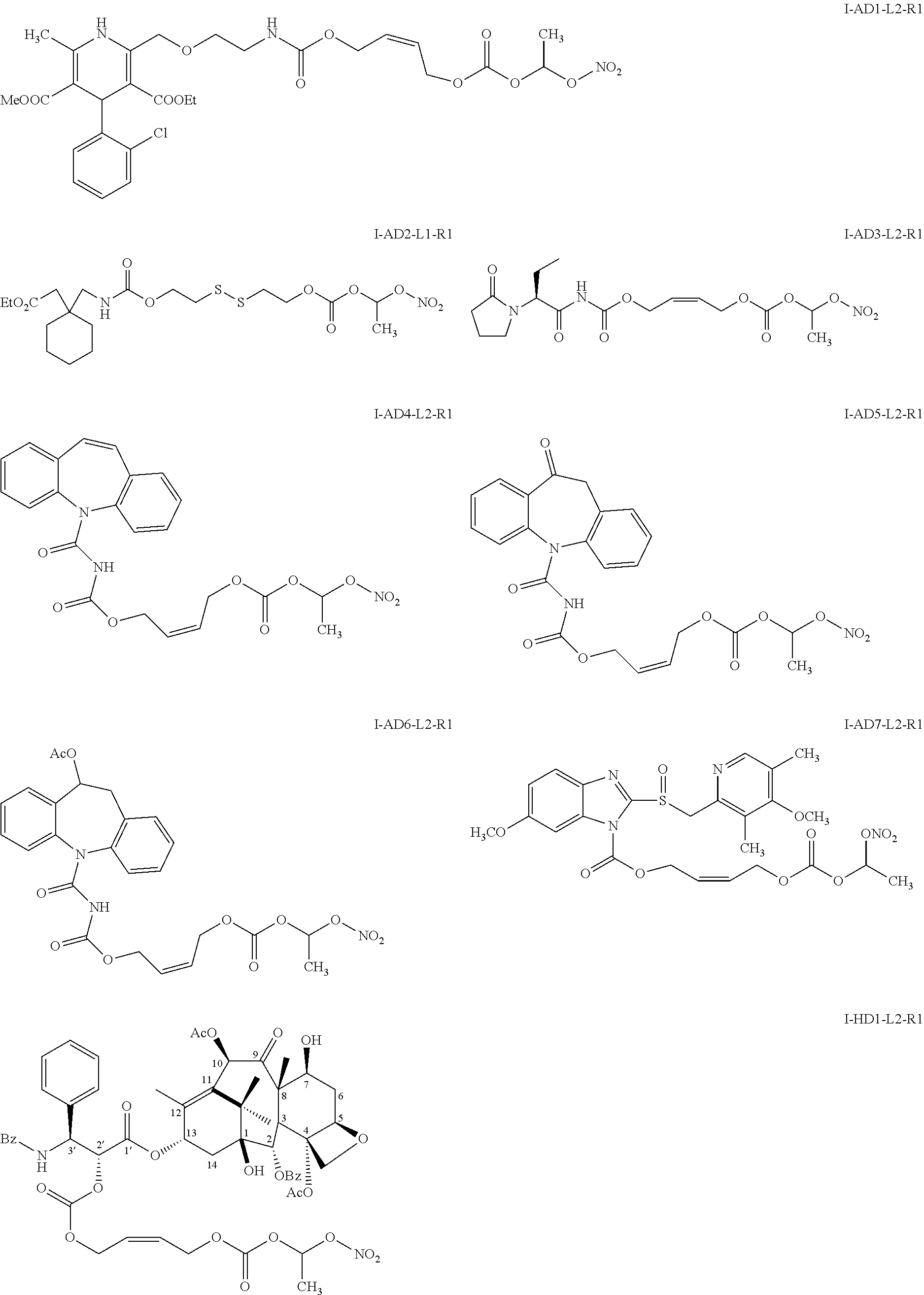

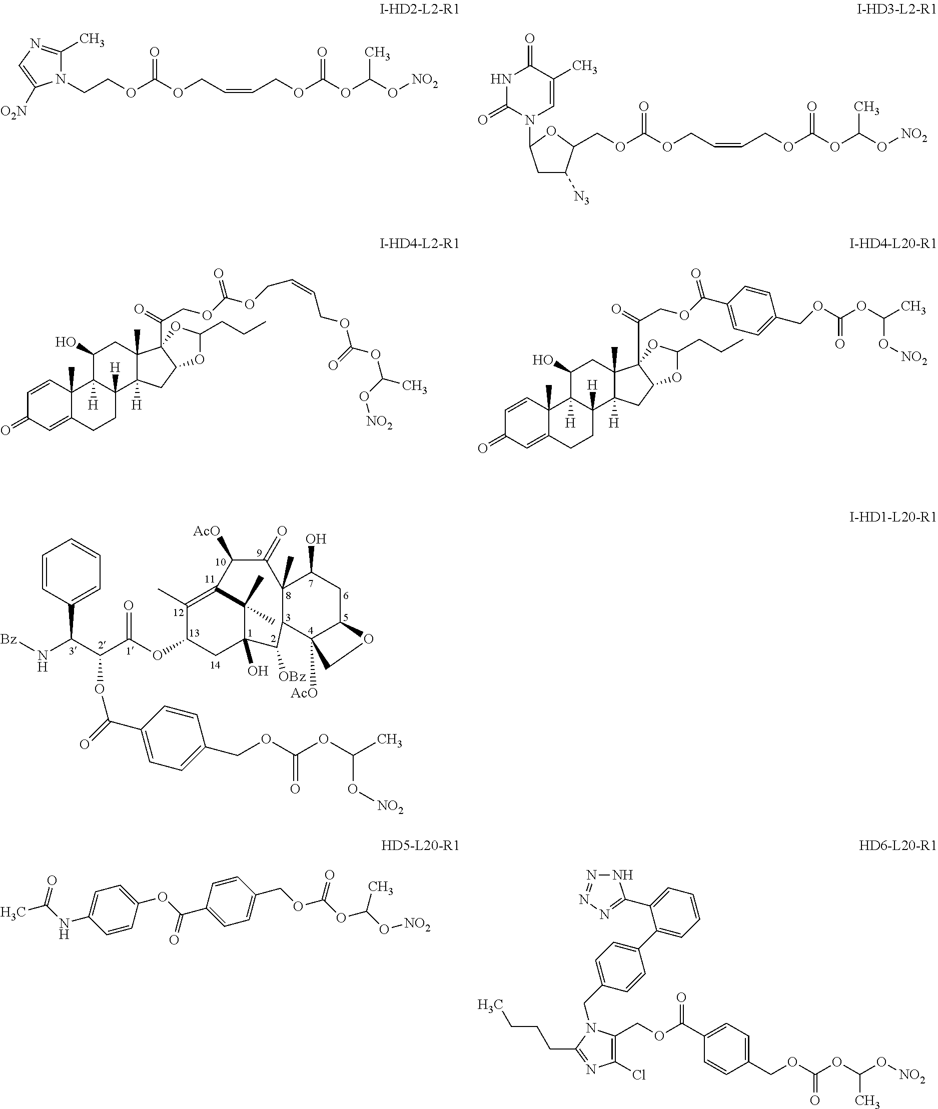

68. A pharmaceutical composition comprising a therapeutically effective amount of the compound of claim 1, or a pharmaceutically acceptable salt thereof and one or more of pharmaceutically acceptable carriers, vehicles or diluents.

69. A pharmaceutical composition comprising a therapeutically effective amount of the compound of claim 67, or a pharmaceutically acceptable salt thereof and one or more of pharmaceutically acceptable carriers, vehicles or diluents.

70. A method of treating a disease or disorder in a human or mammal where a chronic, sustained and selective release of the constituent drug or therapeutic agent D or nitric oxide is beneficial; comprising administering to a mammal or a human in need of the treatment a therapeutically effective amount of the compound of formula (I) as claimed in claim 1.

71. A method of treating a disease or disorder in a human or mammal where a chronic, sustained and selective release of the constituent drug or therapeutic agent D or nitric oxide is beneficial; comprising administering to said mammal a therapeutically effective amount of the pharmaceutical composition as claimed in claim 68.

72. A process for the preparation of a compound of formula (I), or a pharmaceutically acceptable salt thereof, 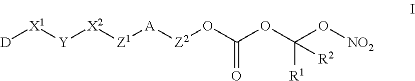
wherein D is a drug containing a carboxylic acid group; X¹, Y, X², Z¹, A, Z², R¹ and R² are as defined in claim 1;
wherein the process is selected from:
Process 1-1: A) reacting an aldehyde Sₐ (R¹—C(O)—R²) with phosgene or its equivalents in the presence of a base and a solvent to yield chloroformate of formula X (wherein, R¹ and R² are as defined in claim 1); 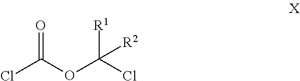
B) reacting a carboxyl-containing drug Dₐ (D-COON, appropriately protected if the drug has any additional reactive functional groups) with a linker Lₐ (wherein, Z¹, A and Z² are as defined in claim 1) in the presence of a coupling agent and a base in a solvent to yield the intermediate alcohol of formula Iₐ (wherein, Z¹, A and Z² are as defined in claim 1); or 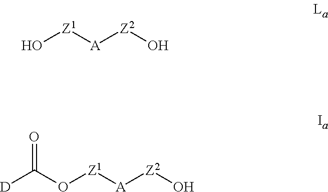
converting the carboxyl-containing drug Dₐ (appropriately protected if the drug has any additional reactive functional groups) into its carboxyl halide, Dₐ₁ (D-COCL) and reacting the resulting compound Dₐ₁ with the linker Lₐ (wherein, Z¹, A and Z² are as defined in claim 1) in the presence of a base in a solvent to yield the intermediate alcohol of formula Iₐ; or
reacting the carboxyl-containing drug Dₐ (appropriately protected if the drug has any additional reactive functional groups) with a base in a solvent to yield the corresponding carboxylate salt of the drug, Dₐ₂ (D-COO⁻M⁺) and reacting the resulting Dₐ₂ with the linker of formula Lₐ₁; 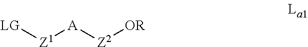
wherein LG is a leaving group (LG) and R is as defined) in the presence of a base in a solvent to yield the intermediate alcohol of formula Iₐ;
C) reacting the intermediate alcohol of formula Iₐ (as obtained in Step B above) with the chloroformate X (obtained in step A above) in the presence of a base and a solvent to obtain the intermediate of formula Iₐ₁; 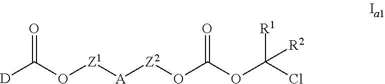
wherein, Z¹, A, Z², R¹ and R² are as defined in claim 1;
D) optionally subjecting the intermediate of formula Iₐ₁ (as obtained in Step C above) to nitration using silver nitrate (AgNO₃) in the presence of a solvent to yield the compound of formula (I), and optionally, converting the compound of formula (I) to its pharmaceutically acceptable salt;
Process 1-2: subjecting the compound of formula (I) (wherein A=S) (as obtained in Process 1-1 above) to oxidation with an oxidizing agent in the presence of a solvent to obtain the corresponding compound of formula (I) (wherein A=SO or SO₂), and optionally, converting the compound of formula (I) to its pharmaceutically acceptable salt;
Process 1-3: A) reacting the chloroformate of formula X (as obtained in Step A of Process 1-1 above) with the linker of formula Lₐ (as defined in Step B of Process 1-1 above) in the presence of a base and a solvent to yield the linker intermediate of formula Lₐ₁ (wherein, Z¹, A, Z², R¹ and R² are as defined above). 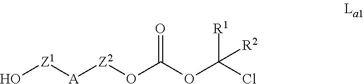
B) subjecting the intermediate of formula Lₐ₁ (as obtained in Step A above) to nitration using silver nitrate in the presence of a solvent to yield the linker intermediate of formula L₁ (wherein, Z¹, A, Z², R¹ and R² are as defined above). 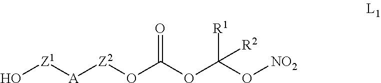
C) the carboxyl-containing drug Dₐ is directly coupled with the linker intermediate of formula Lₐ₁ (as obtained in Step A above) in the presence of a coupling agent; or the reactive drug acid halide Dₐ₁ (as obtained in Step B of Process 1-1) is coupled with the linker intermediate Lₐ₁ (as obtained in Step A above) in the presence of a base and in a solvent to yield the compound of formula (Iₐ₁), which is subjected to nitration using silver nitrate in the presence of a solvent to yield the compound of formula (I), and optionally converting the compound of formula (I) to its pharmaceutically acceptable salt; or
the carboxyl-containing drug Dₐ is directly coupled with the linker intermediate of formula L₁ (as obtained in step B above) in the presence of a coupling agent or the reactive drug acid halide Dₐ₁ (as obtained in Step B of Process 1-1) is coupled with the linker intermediate L₁ (as obtained in step B above) in the presence of a base and in a solvent to yield the compound of formula (I), and optionally converting the compound of formula (I) to its pharmaceutically acceptable salt;
Process 1-4: A) reacting the linker of formula Lₐ (as defined in Step B of Process 1-1 above) or the linker of formula L_{b} (wherein, X²═NH; Z¹, A and Z² are as defined above) with α-chloroacetyl chloride (ACAC) in the presence of a base and in a solvent to obtain a chloroacetate of formula Lₐ₂ or a chloroacetamide of formula L_{b1} (wherein, X², Z¹, A and Z² are as defined above). 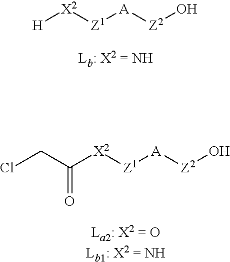
B) coupling the drug carboxylate salt Dₐ₂ (as obtained in Step B of Process 1-1) with the chloroacetate of formula Lₐ₂ or the chloroacetamide of formula L_{b1} (as obtained in Step A above) in the presence of a base and in a solvent to obtain an intermediate compound of formula I_{b} (wherein, X², Z¹, A and Z² are as defined above). 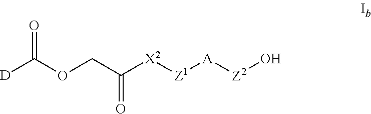
C) reacting the intermediate I_{b} (as obtained in Step B above) with the chloroformate X (as obtained in Step A of Process 1-1) in the presence of a base and in a solvent to obtain the intermediate compound of formula I_{b1} (wherein, X², Z¹, A, Z², R¹ and R² are as defined above); 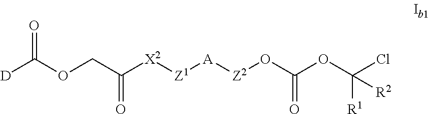
D) subjecting the intermediate compound of formula I_{b1} (as obtained in Step C above) to nitration using silver nitrate in a solvent to obtain the compound of formula (I), and optionally converting the compound of formula (I) to its pharmaceutically acceptable salt; or
Process 1-5: A) reacting a carboxyl-containing drug Dₐ (appropriately protected if the drug has any additional reactive functional groups) with a linker of formula L_{c} (wherein, Z¹, A and Z² are as defined above) in the presence of a coupling agent and in a solvent to obtain the intermediate of formula I_{c} (wherein, Z¹, A and Z² are as defined above); 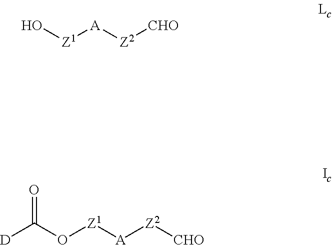
or the drug acid halide Dₐ₁ (as obtained in Step B of Process 1-1) is reacted with the intermediate of formula L_{c} (wherein, Z¹, A and Z² are as defined above) in the presence of a base and in a solvent to obtain the intermediate compound of formula I_{C};
B) reducing the intermediate of formula I_{c} (as obtained in Step A above) using a reducing agent in the presence of a solvent to yield the intermediate compound I_{c1} (wherein, Z¹, A and Z² are as defined above); 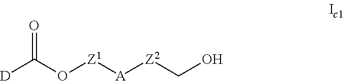
C) reacting the intermediate of formula I_{c1} with the chloroformate X (as obtained in Step A of Process 1-1 above) in the presence of a base and in a solvent to obtain the intermediate compound of formula I_{ce} (wherein, Z¹, A, Z², R¹ and R₂ are as defined above); 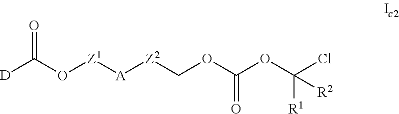
D) subjecting the intermediate compound of formula I_{c2} (as obtained in Step C above) to nitration using silver nitrate in the presence of a solvent to yield the compound of formula (I), and optionally converting the compound of formula (I) to its pharmaceutically acceptable salt.

73. A process for the preparation of a compound of formula (I), or a pharmaceutically acceptable salt thereof, 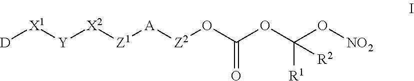
wherein D is a drug containing an amino, a hydroxyl or a sulfhydryl group; X¹, Y, X², Z¹, A, Z², R¹ and R² are as defined in claim 1;
wherein the process is selected from:
Process 2-1: A) reacting the linker of formula L₁ 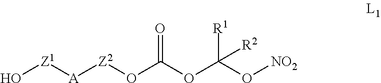
with phosgene or its equivalent in the presence of a base and in a solvent to obtain the corresponding formyl halide of formula Lₑ (wherein, Z¹, A, Z², R¹ and R² are as defined in claim 1; LG is a leaving group); 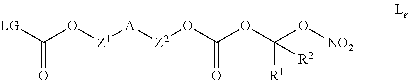
B) reacting a drug containing an amino group D_{b} (D-Y—X¹H, wherein Y=a bond, C═O or S(O)₂; X¹═NR³, wherein R³ is a bond) or a drug containing a hydroxyl or sulfhydryl group D_{c} (D-Y—X¹H, wherein Y=a bond; X¹═O or S) with phosgene or its equivalent in the presence of a base and a solvent to obtain the corresponding reactive formyl halide of the drug of formula D_{b1} and D_{c4} respectively wherein LG is a leaving group; or reacting an amino-containing drug D_{b} (D-Y—X¹H, wherein Y=a bond, C═O or S(O)₂; X¹═NR³, wherein R³ is H) with phosgene or its equivalents in the presence of a base and in a solvent to yield the corresponding isocyanate of formula D_{b2} [wherein, Y=bond, C(═O) or S(O)₂, X¹═N]; 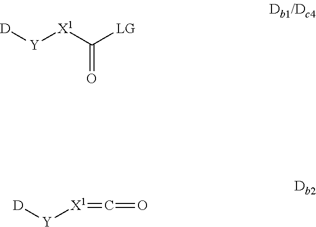
C) reacting the drug containing an amino group D_{b} (D-Y—X¹H, wherein Y=a bond, C═O or S(O)₂; X¹═NR³, wherein R³ is a bond or H) or the drug containing a hydroxyl or sulfhydryl group D_{c} (D-Y—X¹H, wherein Y=a bond; X¹═O or S) with the compound of formula Lₑ) (as obtained in step A above) to obtain the compound of formula (I), and optionally converting the compound of formula (I) to its pharmaceutically acceptable salt; or
reacting the carbonyl derivative of formula D_{b1} or D_{c4} (as obtained in Step B above) of the drugs D_{b} and D_{c} respectively with the linker of formula L₁ in the presence of a base and a solvent to obtain the compound of formula (I), and optionally converting the compound of formula (I) to its pharmaceutically acceptable salt; or reacting the reactive isocyanate derivative D_{b2} (as obtained in Step B above) of the drug D_{b} with the linker of formula L₁ in the presence of a base and a solvent to obtain the compound of formula (I), and optionally converting the compound of formula (I) to its pharmaceutically acceptable salt;
Process 2-2: A) selectively protecting one hydroxyl group of the linker Lₐ (as defined in Step B of Process 1-1 above) with a suitable protecting group (PG^{H}) to yield the mono-protected linker of formula Lₐ₂ (wherein, Z¹, A and Z² are as defined above). 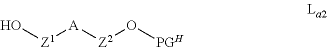
B) reacting the mono-protected linker of formula Lₐ₂ (as obtained in step A above) with phosgene or its equivalents in the presence of a base and in a solvent to obtain the intermediate of formula Lₐ₃ (wherein, Z¹, A and Z² are as defined above; LG is a suitable leaving group, PG^{H} is a suitable protecting group). 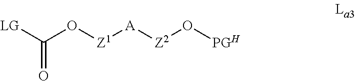
C) reacting the drug containing an amino group D_{b} (D-Y—X¹H, wherein Y=a bond, C═O or S(O)₂; X¹═NR³, wherein R³ is a bond or H) or the drug containing a hydroxyl or sulfhydryl group D_{c} (D-Y—X¹H, wherein Y=a bond; X¹═O or S) with the linker intermediate of formula Lₐ₃ (as obtained in Step B above) in the presence of a base and in a solvent to yield an intermediate of formula I_{f} (wherein, X¹, Z¹, A and Z² are as defined above, PG^{H} is a suitable protecting group). 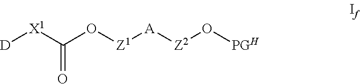
D) removing the hydroxyl protecting group (PG^{H}) from the intermediate of formula I_{f} (as obtained in step C above) to yield an intermediate of formula I_{f1} (wherein, X¹, Z¹, A and Z² are as defined above). 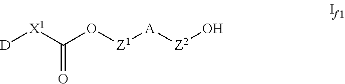
E) reacting the intermediate of formula I_{f1} (as obtained in step D above) with the chloroformate of formula X 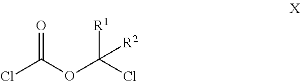
in the presence of a base and in a solvent to obtain the intermediate of formula I_{f2} (wherein, X¹, Z¹, A, Z², R¹ and R² are as defined above). 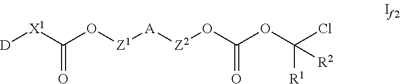
F) subjecting the intermediate I_{f2} (as obtained in Step E above) to nitration using silver nitrate in the presence of a solvent to yield the compound of formula (I), and optionally converting the compound of formula (I) to its pharmaceutically acceptable salt;
Process 2-3: A) reacting the formyl halide D_{b1} or D_{c4} (as obtained in Step B of Process 2-1 above) with the compound of formula Lₐ; 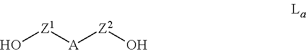
wherein Z¹, A and Z² are as defined above, or with the compound of formula L_{b}; 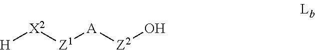
wherein Z¹, A and Z² are as defined above
in the presence of a base in a solvent to obtain the intermediate of formula Iₑ 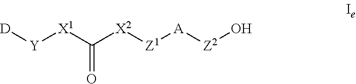
wherein, Y, X¹, X², Z¹, A and Z² are as defined above; or
reacting the isocyanate D_{b2} (as obtained in Step B of Process 2-1 above) with the linker Lₐ or with linker L_{b} in the presence of a base in a solvent to obtain the intermediate of formula Iₑ;
B) reacting the intermediate Iₑ (as obtained in step A above) with the chloroformate X in the presence of a base and in a solvent to yield the intermediate compound of formula Iₑ₁, 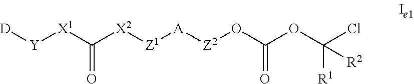
wherein, Y, X¹, X², Z¹, A and Z² are as defined above,
D) subjecting the intermediate Iₑ₁ (as obtained in Step C above) to nitration using silver nitrate in the presence of a solvent to obtain the compound of formula (I), and optionally, converting the compound of formula (I) to its pharmaceutically acceptable salt;
Process 2-4: A) reacting the formyl halide of formula D_{b1} (as obtained in Step B of Process 2-1) with the linker intermediate of formula Lₐ₁ 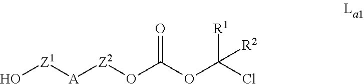
wherein, Z¹, A, Z², R¹ and R² are as defined in claim 1;
in the presence of a base and in a solvent to yield the intermediate of formula Iₑ₁;
B) subjecting the intermediate of formula Iₑ₁ (as obtained in Step A above) to nitration using silver nitrate in the presence of a solvent to obtain the compound of formula (I), and optionally converting the compound of formula (I) to its pharmaceutically acceptable salt; or
Process 2-5: A) reacting the drug isocyanate D_{b2} (as obtained in Step B of Process 2-1) with the linker intermediate of formula Lₐ₁ in the presence of a base and in a solvent to yield the intermediate of formula Iₑ₁;
B) subjecting the intermediate Iₑ₁ (as obtained in Step A above) to nitration using silver nitrate in the presence of a solvent to obtain the compound of formula (I), and optionally converting the compound of formula (I) to its pharmaceutically acceptable salt.

74. A process for the preparation of a compound of formula (I), or a pharmaceutically acceptable salt thereof, 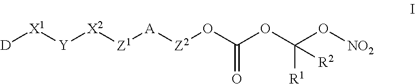
wherein D is a drug containing a hydroxyl or a sulfhydryl group; X¹, Y, X², Z¹, A, Z², R¹ and R² are as defined in claim 1;
wherein said process comprises the steps of:
A) coupling of a drug containing a hydroxyl or sulfhydryl group D_{c} (D-Y—X¹H, wherein Y=a bond; X¹═O or S) with the compound of formula L_{f}, 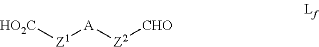
wherein A=1,2-, 1,3-, or 1,4-phenylene and Z¹ and Z²=bond in the presence of a coupling agent, a base and in a solvent to obtain an intermediate I_{g}; 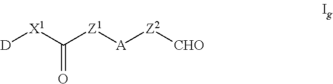
wherein, X¹, Z¹, A and Z² are as defined above;
B) subjecting the intermediate of formula I_{g} in the presence of a reducing agent in a solvent to obtain the intermediate of formula I_{g1}, 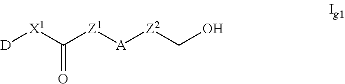
wherein, X¹, Z¹, A and Z² are as defined above;
C) reacting the intermediate I_{g1} with the chloroformate of formula X, 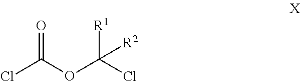
in the presence of a base and in a solvent to obtain further intermediate of formula I_{g2}; 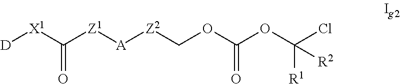
wherein, X¹, Z¹, A, Z², R¹ and R² are as defined above,
D) subjecting the intermediate I_{g2} (as obtained in Step C above) to nitration using silver nitrate in the presence of a solvent to yield the compound of formula (I), and optionally converting the compound of formula (I) to its pharmaceutically acceptable salt.
